(19) United States
(12) Patent Application Publication
(10) Pub. No.: US 2026/0207755 A1
(43) Pub. Date: Jul. 23, 2026

(54) TARGETED PROTEIN DEGRADERS OF INDOLEAMINE 2,3-DIOXYGENASE 1 (IDO1)

(71) Applicant: Northwestern University, Evanston, IL (US)

(72) Inventors: Gary E. Schiltz, Evanston, IL (US); Paige Justine Monsen, Evanston, IL (US); Derek Alan Wainwright, Evanston, IL (US)

(21) Appl. No.: 19/144,050

(22) PCT Filed: Dec. 27, 2023
(86) PCT No.: PCT/US2023/085964

§ 371 Date: Jun. 27, 2025

Related U.S. Application Data
(60) Provisional application No. 63/477,316, filed on Dec. 27, 2022.

(51) Int. Cl. A61K 47/55; A61K 45/06

(52) U.S. Cl. CPC A61K 47/55; A61K 45/06

(57) ABSTRACT

Disclosed herein is a molecule, or a pharmaceutically acceptable salt thereof, that has a formula M1DO1-L-ME3, M1DO1 is a moiety that binds to IDO1, L is a linker covalently attaching M1DO1 and ME3, and ME3 is a moiety that binds to an E3 ubiquitin ligase. Disclosed herein are also the uses of the molecule, or a pharmaceutically acceptable salt thereof, and a pharmaceutical composition comprising the same, in a method of treating cancer in a subject in need thereof.

### CROSS-REFERENCE TO RELATED APPLICATIONS

This application claims benefit of priority to U.S. Application Ser. No. 63/477,316, filed Dec. 27, 2022, the content of which is incorporated by reference in its entirety.

### STATEMENT REGARDING FEDERALLY FUNDED RESEARCH

This invention was made with government support under grant numbers NS097851 awarded by the National Institutes of Health. The government has certain rights in the invention.

### BACKGROUND

The indoleamine 2,3-dioxygenase (IDO) protein plays a crucial role in the progression of cancers. For example, the median survival of primary glioblastoma multiforme (GBM) patients following aggressive surgical intervention, radiotherapy, chemotherapy, and tumor treating fields (TTF) is only ~14-16 months; IDO protein plays a critical immunosuppressive role in the progression of GBM which is independent of IDO's enzyme activity. Enzymatic inhibition of IDO failed in a phase 3 ECHO-301/KEYNOTE-252 clinical trial suggesting a futility of IDO enzyme inhibitor treatment in patients with cancer. Although the mechanism for IDO's contribution to progression of GBM is not understood and remains as an active area of investigation, a more effective IDO neutralizing pharmacologic is required for abolishing IDO's immunosuppressive effect(s).

Proteolysis-targeting chimeric molecules (PROTACs) are an emerging technology that may be utilized to target previously “undruggable” targets, transcription factors and non-enzymatic proteins. (See, e.g., An et al., “Small-molecule PROTACs: An emerging and promising approach for the development of targeted therapy drugs,” EBioMedicine. 2018 October; 36: 553-562; and Gu et al., “PROTACs: An Emerging Targeting Technique for Protein Degradation in Drug Discovery,” Bioessays. 2018 April; 40(4):el 700247, the contents of which are incorporated herein by reference in their entireties). PROTACs are chimeric molecules that may be characterized as “hetero-bifunctional” in that PROTACs include a ligand for recruiting an E3 ubiquitin ligase, a linker, and another ligand to bind with the protein targeted for degradation. Designed as such, PROTACs “hijack” the E3 ubiquitin ligase to the protein which is targeted for protein degradation via ubiquitination, even if the targeted protein is not a physiological substrate for degradation via the ubiquitin-proteasome system.

As such, novel PROTACs that induce degradation of IDO protein, as well as their use for treating cancers (e.g., glioblastoma) need to be developed.

### SUMMARY OF THE INVENTION

Disclosed herein is a molecule having a formula M_{1DO1}-L-M_{E3} and methods of using the same.

One aspect of the invention provides for a molecule that has a formula M_{1DO1}-L-M_{E3}, or a pharmaceutically acceptable salt thereof. M_{1DO1} is a moiety that binds to indoleamine 2,3-dioxygenase 1 (IDO1), L is a linker covalently attaching M_{1DO1} and M_{E3}, and M_{E3} is a moiety that binds to an E3 ubiquitin ligase. L comprises a piperidinylene moiety, with the proviso that (1) M_{E3} does not comprise

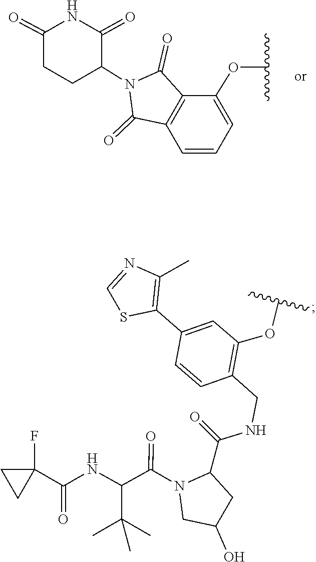 [SMILES: *Oc1cccc2c1C(=O)N(C1CCC(=O)NC1=O)C2=O ; *Oc1cc(-c2scnc2C)ccc1CNC(=O)C1CC(O)CN1C(=O)C(NC(=O)C1(F)CC1)C(C)(C)C]

and (2) the molecule is not (2R)-N-(4-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide or (2R)-N-(4-((1-(3-(2-((2-(2,6-dioxopiperidin-3-yl)-1,3-dioxoisoindolin-4-yl)amino)ethoxy)propanoyl)piperidin-4-yl)oxy)phenyl)-2-((1s,4S)-4-(6-fluoroquinolin-4-yl)cyclohexyl)propanamide.

Another aspect of the invention provides for a pharmaceutical composition comprising a therapeutically effective amount of the molecule as described herein and a pharmaceutically acceptable carrier, excipient, or diluent.

Another aspect of the invention provides for a method of treating cancer in a subject in need thereof. The method comprises administering to the subject the molecule, or a pharmaceutically acceptable salt thereof, or the pharmaceutical composition as described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 demonstrates representative Western blots for identifying a lead IDO1-PROTAC in U87 cells. IDO1 protein was induced in U87 cells with 50 ng/mL IFNγ for 24 hours followed by the treatment with IDO1-PROTAC at 1 and 10 μM for 24 hours before protein samples were prepared for Western blotting analysis of IDO1 and GAPDH.

FIG. 2 demonstrates representative Western blots for identifying a lead IDO1-PROTAC in U87 cells. IDO1 protein was induced in U87 cells with 50 ng/mL IFNγ for 24 hours followed by the treatment with IDO1-PROTAC at 0.01, 0.1, 1, and 10 μM for 24 hours before protein samples were prepared for Western blotting analysis of IDO1 and GAPDH.

FIG. 3 demonstrates representative Western blots for identifying a lead IDO1-PROTAC in U87 cells. IDO1 protein was induced in U87 cells with 50 ng/mL IFNγ for 24 hours followed by the treatment with IDO1-PROTAC at 0.1, 0.5, 1, and 10 μM for 24 hours before protein samples were prepared for Western blotting analysis of IDO1 and GAPDH.

FIG. 4 shows representative Western blots for identifying a lead IDO1-PROTAC in U87 cells. IDO1 protein was induced in U87 cells with 50 ng/mL IFNγ for 24 hours followed by the treatment with IDO1-PROTAC at 0.1, 0.5, 1, and 10 μM for 24 hours before protein samples were prepared for Western blotting analysis of IDO1 and GAPDH.

FIG. 5 shows representative Western blots for identifying a lead IDO1-PROTAC in U87 cells. IDO1 protein was induced in U87 cells with 50 ng/mL IFNγ for 24 hours followed by the treatment with IDO1-PROTAC at 0.1, 0.5, 1, and 10 μM for 24 hours before protein samples were prepared for Western blotting analysis of IDO1 and GAPDH.

FIG. 6 shows that cells were treated with representative concentrations of IDO1-PROTACs in the presence of 50 ng/mL IFNγ for 24 hours and cell culture supernatants were collected to measure IFNγ-induced kynurenine levels using Ehrlich's reagent. Cells cultured in the absence of IFNγ served as the control group.

FIG. 7 illustrates that U87 cells were treated with an extended dose range of NU227047 for 24 hours and protein samples were analyzed by Western blotting. Cell culture supernatants were collected to measure IFNγ-induced kynurenine levels using Ehrlich's reagent. Cells cultured in the absence of IFNγ served as the control group.

FIG. 8 shows that U87 cells were treated with an extended dose range of NU227164 for 24 hours and protein samples were analyzed by Western blotting. Cell culture supernatants were collected to measure IFNγ-induced kynurenine levels using Ehrlich's reagent. Cells cultured in the absence of IFNγ served as the control group.

FIG. 9 shows that U87 cells were treated with an extended dose range of NU227168 for 24 hours and protein samples were analyzed by Western blotting.

FIG. 10 shows that U87 cells were treated with an extended dose range of NU227192 for 24 hours and protein samples were analyzed by Western blotting.

FIG. 11 shows that U87 cells were treated with an extended dose range of NU227194 for 24 hours and protein samples were analyzed by Western blotting.

FIG. 12 shows that U87 cells were treated with an extended dose range of NU227199 for 24 hours and protein samples were analyzed by Western blotting.

FIGS. 13A-13C show mechanistic understanding of IDO1-PROTAC-induced IDO1 degradation. FIG. 13A is BLI sensorgrams showing association and dissociation of NU227164 to IDO1 protein; IDO1 was pre-immobilized on NI NTA sensors, the interaction of the protein with compound at the compound concentrations showed in the legend was monitored in real time by BLI. Data sets fitted globally with a 1:1 kinetic model. FIG. 13B is BLI sensorgrams showing association and dissociation of compound NU227164 to CRBN protein pre-immobilized on AR2G sensors. Binding experiments and data fitting were performed as described above. FIG. 13C is BLI sensorgrams for the IDO1-NU227164-CRBN ternary complex. IDO1 protein was loaded onto NiNTA biosensors. IDO1 was pre-immobilized on NI NTA sensors, and binding of compound NU227164 preincubated with CRBN was monitored in the association step; next, the sensors were moved into reaction buffer in order monitor dissociation. As indicated in the legend, each trace corresponds to a different concentration of NU227164. Data sets fitted globally with a 1:1 kinetic model. No binding was detected between CRBN and IDO1 in the absence of NU227164 (trace “CRBN only”).

FIGS. 14A-14C illustrate mechanistic understanding of DO1-PROTAC-induced IDO1 degradation. FIG. 14A is BLI sensorgrams showing association and dissociation of NU227168 to IDO1 protein; IDO1 was pre-immobilized on NI NTA sensors, the interaction of the protein with compound at the compound concentrations showed in the legend was monitored in real time by BLI. The equilibrium dissociation constant (K_{d}) was obtained by fitting the steady state data (Req as a function of compound concentration) with a 1:1 binding model. FIG. 14B is BLI sensorgrams showing association and dissociation of compound NU227168 to CRBN protein pre-immobilized on AR2G sensors. Binding experiments and data fitting were performed as described above. FIG. 14C is BLI sensorgrams for the IDO1-NU227168-CRBN ternary complex. IDO1 protein was loaded onto NiNTA biosensors. IDO1 was pre-immobilized on NI NTA sensors, and binding of compound NU227168 preincubated with CRBN was monitored in the association step; next, the sensors were moved into reaction buffer in order monitor dissociation. As indicated in the legend, each trace corresponds to a different concentration of NU227168. Data sets fitted globally with a 1:1 kinetic model. No binding was detected between CRBN and IDO1 in the absence of NU227168 (trace “CRBN only”).

FIGS. 15A-15C show mechanistic understanding of IDO1-PROTAC-induced IDO1 degradation. FIG. 15A is BLI sensorgrams showing association and dissociation of NU227191 to IDO1 protein; IDO1 was pre-immobilized on NI NTA sensors, the interaction of the protein with compound at the compound concentrations showed in the legend was monitored in real time by BLI. The equilibrium dissociation constant (K_{d}) was obtained by fitting the steady state data (Req as a function of compound concentration) with a 1:1 binding model. FIG. 15B is BLI sensorgrams showing association and dissociation of compound NU227191 to CRBN protein pre-immobilized on AR2G sensors. Binding experiments and data fitting were performed as described above. FIG. 15C is BLI sensorgrams for the IDO1-NU227191-CRBN ternary complex. IDO1 protein was loaded onto NiNTA biosensors. IDO1 was pre-immobilized on NI NTA sensors, and binding of compound NU227191 preincubated with CRBN was monitored in the association step; next, the sensors were moved into reaction buffer in order monitor dissociation. As indicated in the legend, each trace corresponds to a different concentration of NU227191. Data sets fitted globally with a 1:1 kinetic model. No binding was detected between CRBN and IDO1 in the absence of NU227191 (trace “CRBN only”).

FIGS. 16A-16C show mechanistic understanding of IDO1-PROTAC-induced IDO1 degradation. FIG. 16A is BLI sensorgrams showing association and dissociation of NU227199 to IDO1 protein; IDO1 was pre-immobilized on NI NTA sensors, the interaction of the protein with compound at the compound concentrations showed in the legend was monitored in real time by BLI. The equilibrium dissociation constant (K_{d}) was obtained by fitting the steady state data (Req as a function of compound concentration) with a 1:1 binding model. FIG. 16B is BLI sensorgrams showing association and dissociation of compound NU227199 to CRBN protein pre-immobilized on AR2G sensors. Binding experiments and data fitting were performed as described above. FIG. 16C is BLI sensorgrams for the IDO1-NU227199-CRBN ternary complex. IDO1 protein was loaded onto NiNTA biosensors. IDO1 was pre-immobilized on NI NTA sensors, and binding of compound NU227199 preincubated with CRBN was monitored in the association step; next, the sensors were moved into reaction buffer in order monitor dissociation. As indicated in the legend, each trace corresponds to a different concentration of NU227199. Data sets fitted globally with a 1:1 kinetic model. No binding was detected between CRBN and IDO1 in the absence of NU227199 (trace “CRBN only”).

FIG. 17 shows representative Western blots for NU227426 and NU227427 to measure IDO1 in U87 cells. IDO1 protein was induced in U87 cells with 50 ng/mL IFNγ for 24 hours followed by the treatment with IDO1-PROTAC at the indicated concentrations for 24 hours before protein samples were prepared for Western blotting analysis of IDO1 and GAPDH.

FIG. 18 demonstrates representative Western blots for NU227326, NU227428, and NU223618 to measure IDO1 in U87 cells. IDO1 protein was induced in U87 cells with 50 ng/mL IFNγ for 24 hours followed by the treatment with IDO1-PROTAC at the indicated concentrations for 24 hours before protein samples were prepared for Western blotting analysis of IDO1 and GAPDH.

FIG. 19 shows representative Western blots for NU227424 and NU227425 to measure IDO1 in U87 cells. IDO1 protein was induced in U87 cells with 50 ng/mL IFNγ for 24 hours followed by the treatment with IDO1-PROTAC at the indicated concentrations for 24 hours before protein samples were prepared for Western blotting analysis of IDO1 and GAPDH.

FIGS. 20A-20E show biological activity of compounds NU227326 and NU227327. FIG. 20A demonstrates Western blotting of indicated compounds and concentrations in U87 cells showing a potent and dose-dependent degradation of IDO1. FIG. 20B shows concentration of kynurenine in U87 cells after treatment with either NU227326 or NU227327 at the indicated doses of compound which shows a strong reduction of cellular kynurenine. FIG. 20C shows Western blotting of NU227326 at indicated concentrations in GBM43 cells showing a potent and dose-dependent degradation of IDO1. FIG. 20D demonstrates levels of IDO1 from U87 cells quantified and plotted to show a DC50=10.4 nM. FIG. 20E shows levels of IDO1 from GBM43 cells quantified and plotted to show a DC50=20.3 nM.

FIG. 21 shows BLI sensograms of compound NU227326 and NU227327. Binding is shown as a binary complex with IDO1 or CRBN and kinetic parameters are given. Bottom row shows the binding sensograms and parameters for the ternary complex between the indicated compounds and CRBN and IDO1.

FIGS. 22A-22D show determination of IDO1 degrading potency of NU227326. FIG. 22A shows dose-dependent IDO1 degradation by compound NU227326 in U87 cells for 24 h and analyzed by western blotting. FIG. 22B shows dose-dependent IDO1 degradation by compound NU227326 in GBM43 cells for 24 h and analyzed by western blotting. FIG. 22C demonstrates analysis of the data from FIG. 22A to determine the DC₅₀. FIG. 22D demonstrates analysis of the data from FIG. 22B to determine the DC₅₀.

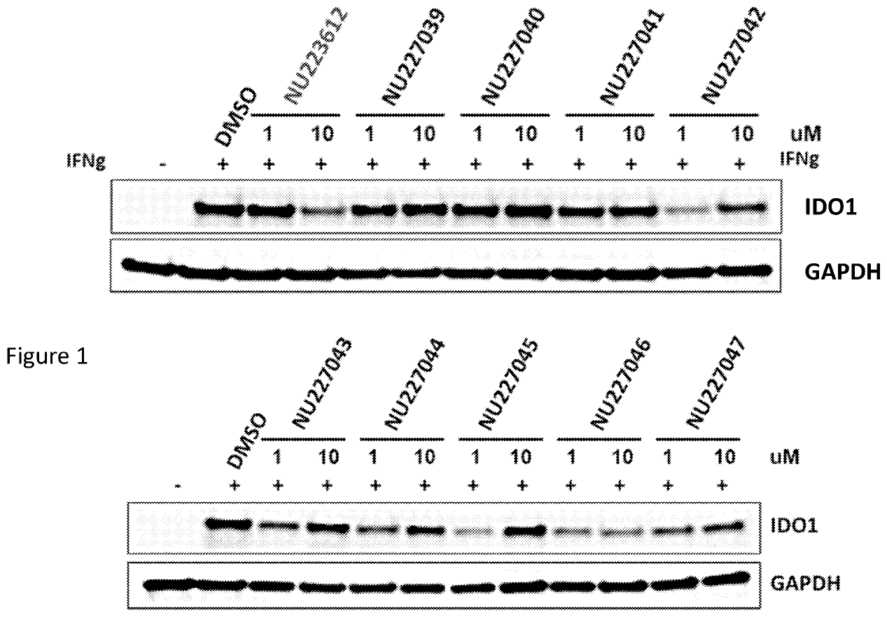

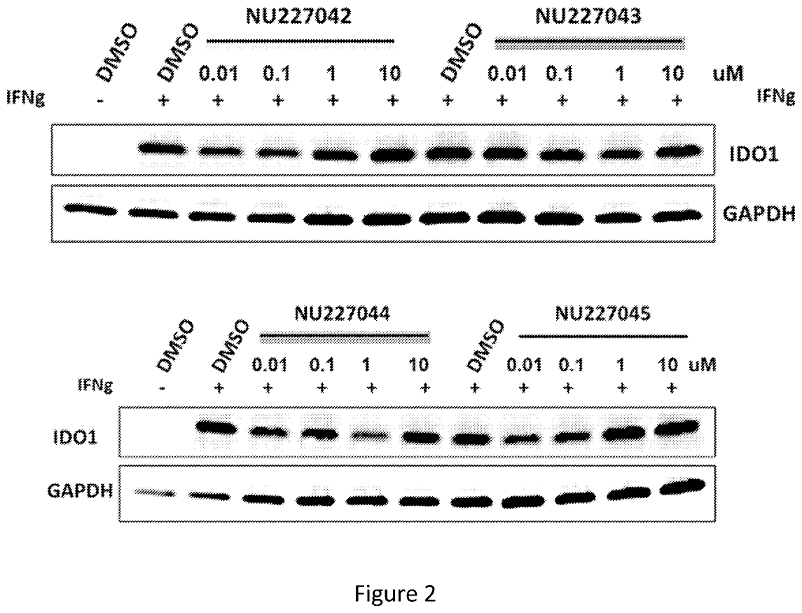

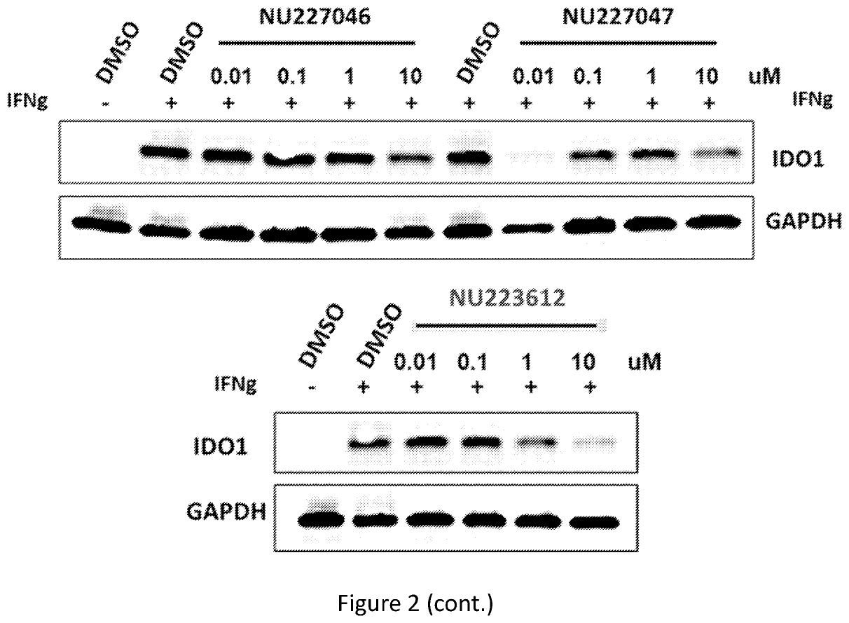

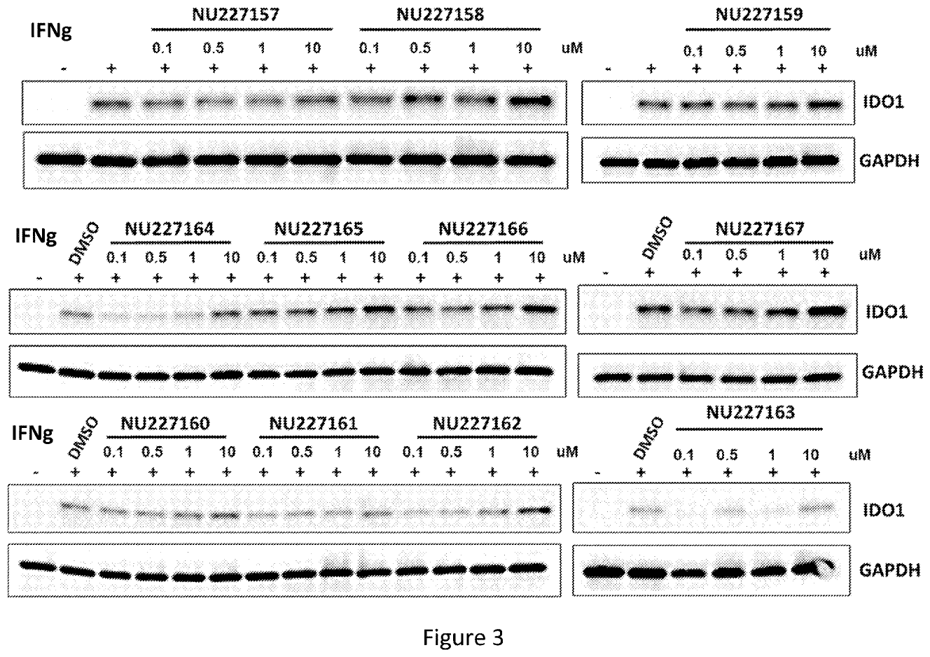

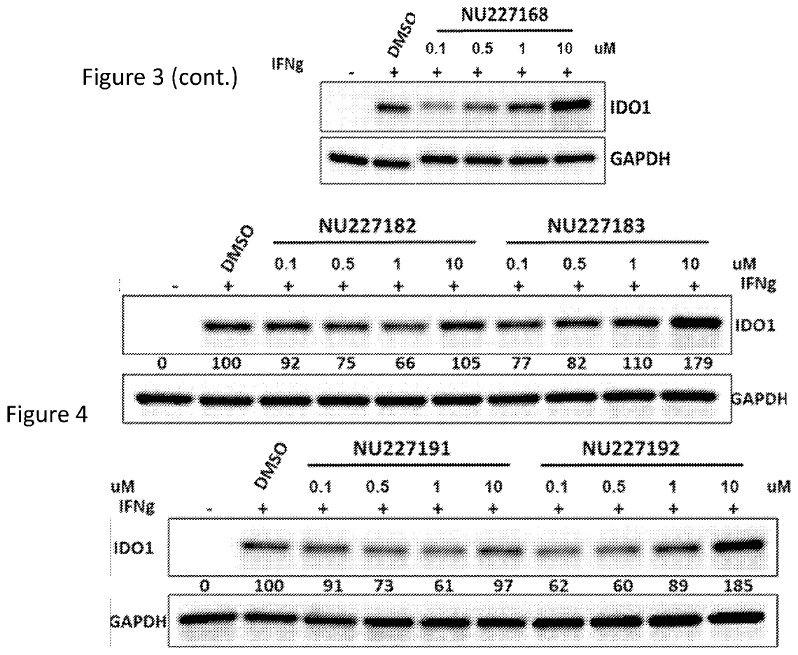

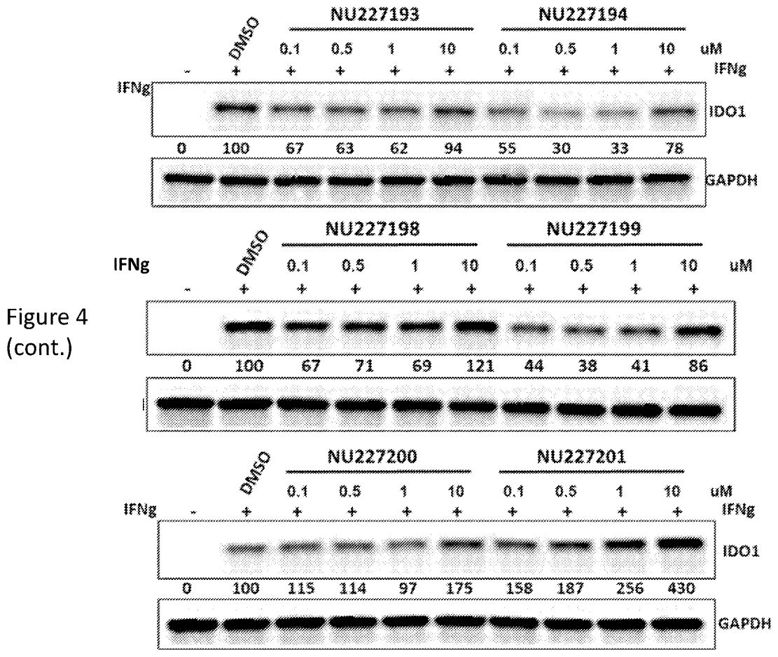

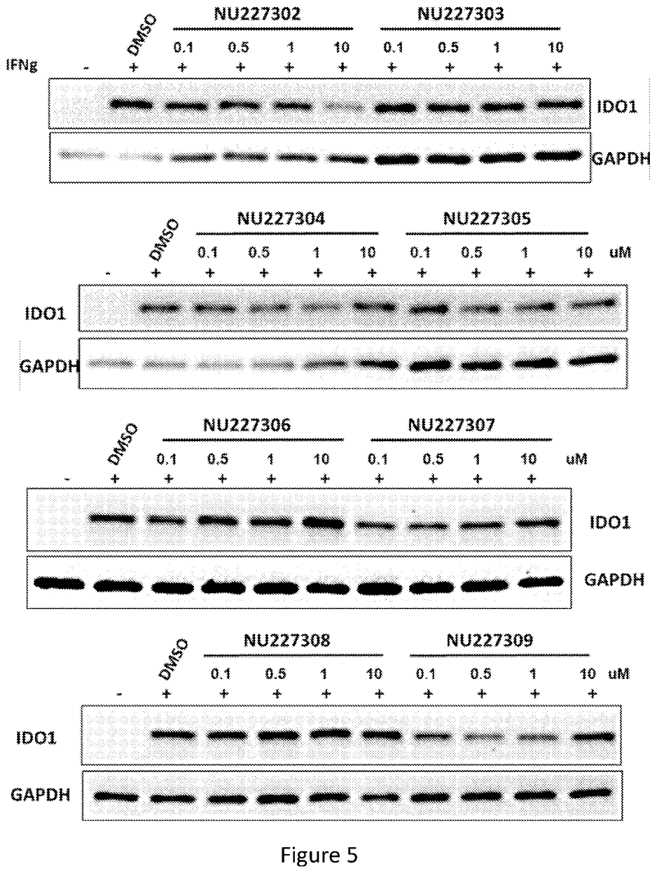

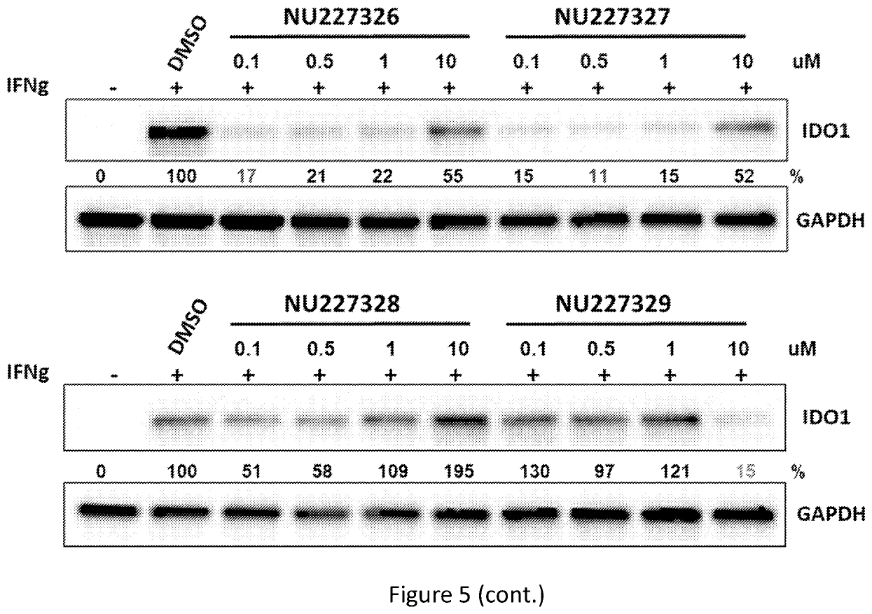

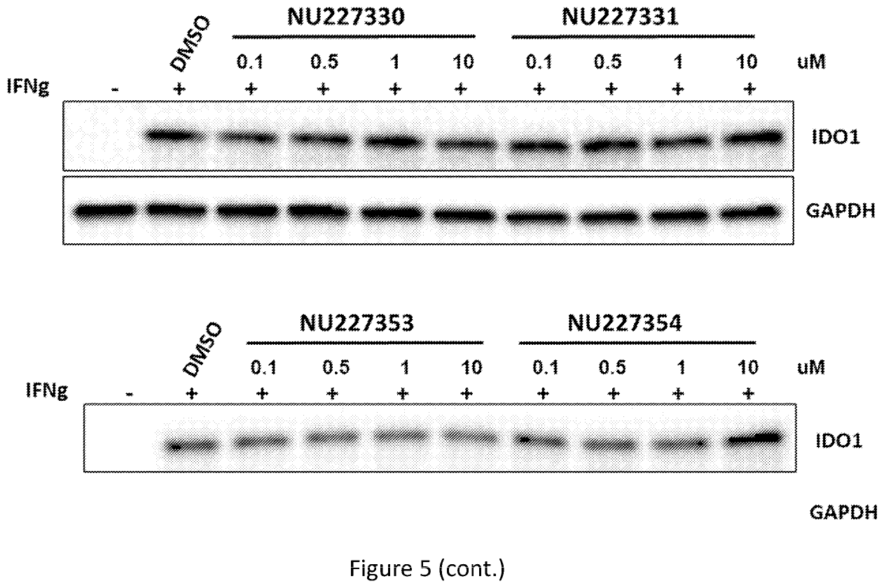

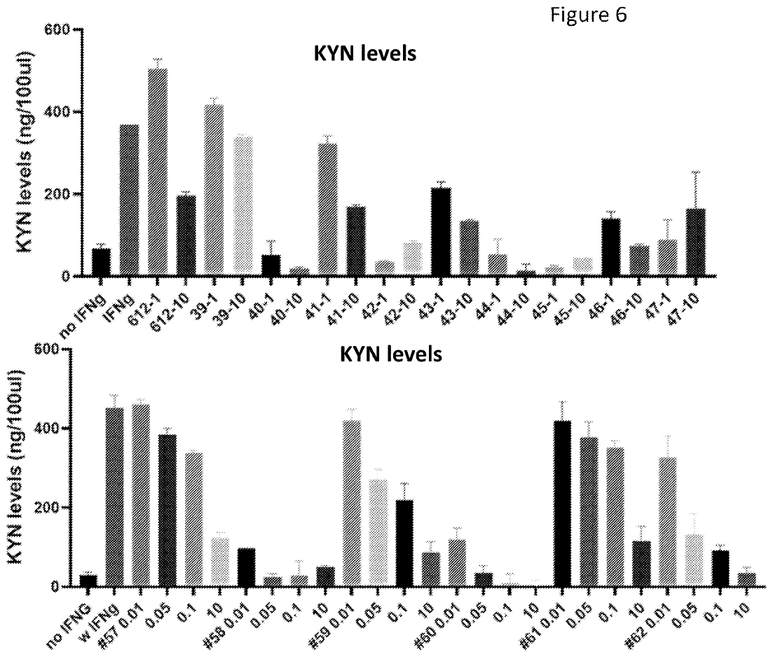

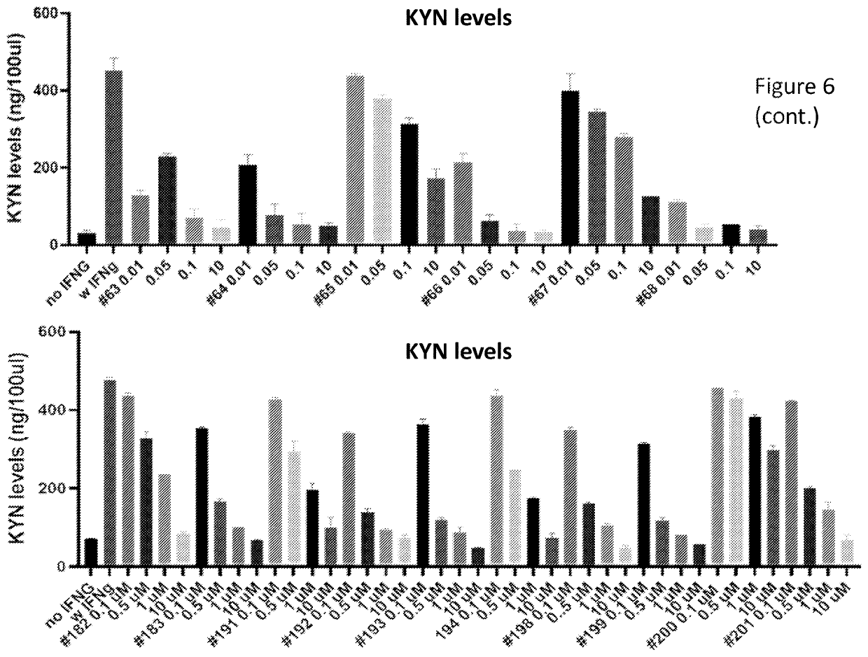

### DETAILED DESCRIPTION OF THE INVENTION

The present invention is described herein using several definitions, as set forth below and throughout the application.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### Molecules

Disclosed herein are proteolytic-targeted chimeric molecules (PROTACs) that induce degradation of IDO1 protein. In some embodiments, the disclosed molecules may be described as having a having a formula: M_{1DO1}-L-M_{E3} or alternatively M_{E3}-L-M_{1DO1}, wherein M_{1DO1} is a moiety that binds to IDO1, L is a linker covalently attaching M_{1DO1} and M_{E3}, and M_{E3} is a moiety that binds to an E3 ubiquitin ligase. Compounds that bind and/or inhibit IDO1 are disclosed in the prior art and may include, but are not limited to compounds disclosed in U.S. Publication Nos. 2020/0179347; 2020/0172492; 2020/0095231; 2020/0093932; 2020/0069695; 2020/0069646; 2020/0024273; 2019/0367465; 2019/0352307; 2019/0292150; 2019/0284184; 2019/0270812; 2019/0247527; 2019/0231776; 2019/0225618; 2019/0152932; 2019/0144417; 2019/0144416; 2019/0135758; 2019/0119216; 2019/0119215; 2019/0040025; 2019/0031665; 2019/0022157; 2019/0002402; 2018/0354908; 2018/0353483; 2018/0333492; 2018/0312497; 2018/0271861; 2018/0186787; 2018/0079712; 2018/0072716; 2018/0072660; 2018/0037553; 2018/0030026; 2017/0319527; 2017/0267668; 2017/0260188; 2017/0231999; 2017/0182156; 2017/0129911; 2017/0107178; 2017/0095473; 2017/0037125; 2017/0009271; 2016/0367564; 2016/0362412; 2016/0361298; 2016/0289171; 2016/0200674; 2016/0143870; 2016/0137595; 2016/0060266; 2016/0060237; 2016/0046596; 2016/0022619; 2015/0352206; 2014/0377307; 2013/0142815; 2011/0136796; 2010/0311804; 2010/0055111; 2009/0081155; and 2004/0234623, the contents of which are incorporated herein by reference in their entireties.

In some embodiments of the disclosed PROTACs, L in the formula M_{1DO1}-L-M_{E3} comprises a piperidinylene moiety, M_{E3} does not comprise

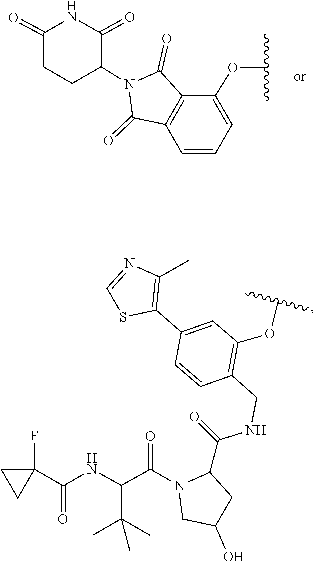 [SMILES: *Oc1cccc2c1C(=O)N(C1CCC(=O)NC1=O)C2=O ; *Oc1cc(-c2scnc2C)ccc1CNC(=O)C1CC(O)CN1C(=O)C(NC(=O)C1(F)CC1)C(C)(C)C]

and the molecule is not (2R)-N-(4-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1 s,4S)-4-(6-fluoroquinolin-4-yl)cyclohexyl)propanamide or (2R)-N-(4-((1-(3-(2-((2-(2,6-dioxopiperidin-3-yl)-1,3-dioxoisoindolin-4-yl)amino)ethoxy)propanoyl)piperidin-4-yl)oxy)phenyl)-2-((1s,4S)-4-(6-fluoroquinolin-4-yl)cyclohexyl)propanamide.

As used herein, the term “piperidinylene” refers to a divalent moiety having a structure of

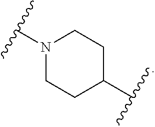 [SMILES: *C1CCN(*)CC1]

As used herein, a wavy line “ [symbol] ” is used to designate the point of attachment for any radical group or substituent group.

In some embodiments of the disclosed PROTACs, M_{1DO1} has a formula I.

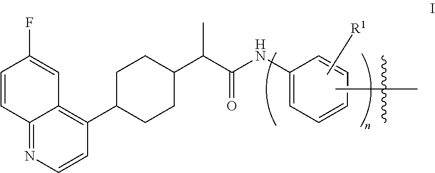

wherein
R¹ is hydrogen or halo; and
n is 0 or 1.

In some embodiments, M_{1DO1} has a formula I(i):

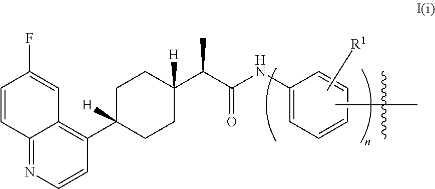

wherein
R¹ is hydrogen or halo; and
n is 0 or 1.

In some embodiments, M_{1DO1} has the formula I(i), wherein R¹ is hydrogen and n is 0. In some embodiments, M_{1DO1} has the formula I(i), wherein R¹ is hydrogen and n is 1. In some embodiments, M_{1DO1} has the formula I(i), wherein R¹ is halo and n is 0. In some embodiments, M_{1DO1} has the formula I(i), wherein R¹ is halo and n is 1.

In some embodiments of the disclosed PROTACs, M_{1DO1} has a formula I(a):

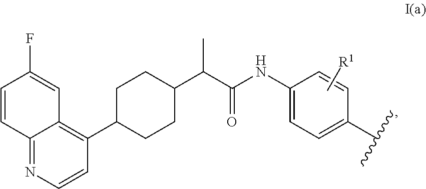

wherein R¹ is hydrogen or halo.

As used herein, the term “halo” refers to a halogen atom or halogen radical (e.g., —F, —Cl, —Br, or —I).

In some embodiments, M_{1DO1} has the formula I(a) and R¹ is hydrogen.

In some embodiments, M_{1DO1} has the formula I(a) and R¹ is halo. In some embodiments, M_{1DO1} has the formula I(a) and R¹ is chloro.

In some embodiments of the disclosed PROTACs, M_{1DO1} has a formula I(ai):

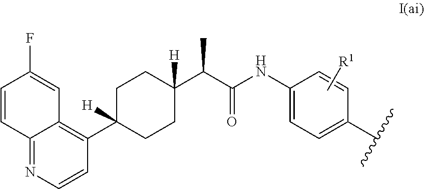

wherein R¹ is hydrogen or halo.

In some embodiments, M_{1DO1} has the formula I(ai) and R¹ is hydrogen.

In some embodiments, M_{1DO1} has the formula I(ai) and R¹ is halo. In some embodiments, M_{1DO1} has the formula I(ai) and R¹ is chloro.

In some embodiments of the disclosed PROTACs, M_{1DO1} has a formula I(b)

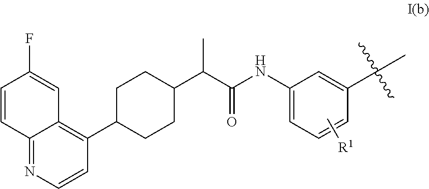

wherein R¹ is hydrogen or halo.

In some embodiments, M_{1DO1} has the formula I(b) and R¹ is hydrogen.

In some embodiments, M_{1DO1} has the formula I(b) and R¹ is halo. In some embodiments, M_{1DO1} has the formula I(b) and R¹ is chloro.

In some embodiments of the disclosed PROTACs, M_{1DO1} has a formula I(bi):

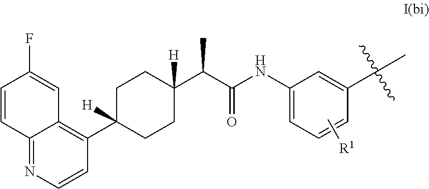

wherein R¹ is hydrogen or halo.

In some embodiments, M_{1DO1} has the formula I(bi) and R¹ is hydrogen.

In some embodiments, M_{1DO1} has the formula I(bi) and R¹ is halo. In some embodiments, M_{1DO1} has the formula I(bi) and R¹ is chloro.

In some embodiments of the disclosed PROTACs, M_{1DO1} has a formula I(c):

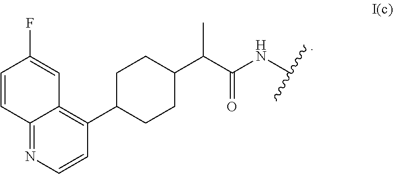 [SMILES: *NC(=O)C(C)C1CCC(c2ccnc3ccc(F)cc23)CC1]

In some embodiments of the disclosed PROTACs, M_{IDO1} has a formula I(ci):

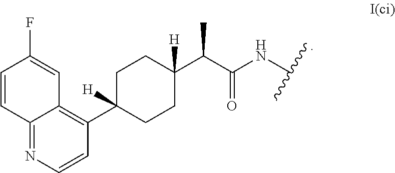 [SMILES: *NC(=O)[C@H](C)[C@H]1CC[C@@H](c2ccnc3ccc(F)cc32)CC1]

The disclosed PROTACs include a linker (L) that conjugates the IDO1 binding moiety (M_{1DO1}) and the E3 ubiquitin ligase binding moiety (M_{E3}). The PROTAC linker connects the functional moieties of a PROTAC, a target protein binder and an E3 ligase recruiter. Linkers used in the development of PROTACs include polyethylene glycol (PEG) linkers, Alkyl-Chain linkers, and Alkyl/ether linkers. Other PROTAC linkers may include those linkers described in one or more of U.S. Publication Nos. 2020/0140456; 2020/0102298; 2020/0085817; 2020/0022966; 2019/0275161; 2019/0263798; 2019/0262502; 2019/0194190; 2019/0151457; 2019/0151295; 2019/0106417; 2019/0076542; 2019/0076541; 2019/0076540; 2019/0076539; 2019/0071415; 2019/0016703; 2018/0327419; 2018/0186785; 2018/0134684; and 2018/0085465; the contents of which are incorporated herein by reference in their entireties.

In some embodiments, the linker L is selected from the group consisting of

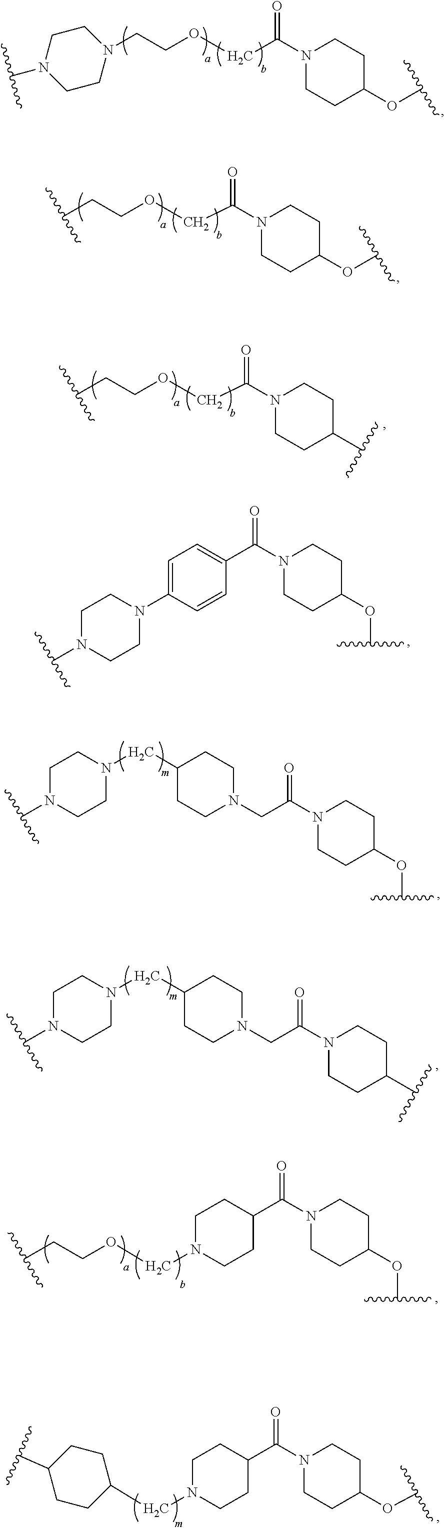

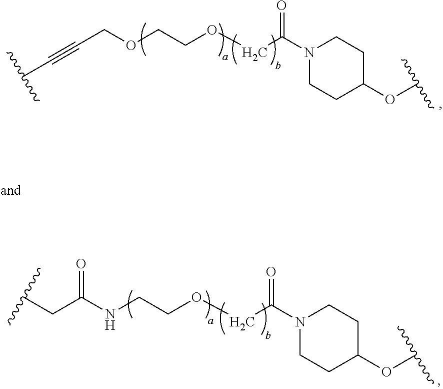

wherein each of a, b, and m is independently an integer selected from 0 to 20. In some such embodiments, each of a, b, and m is independently an integer selected from 0 to 15, from 0 to 12, from 0 to 10, or from 0 to 8. In some embodiments, a is an integer selected from 0 to 3. In some embodiments, b is an integer selected from 1 to 8. In some embodiments, m is an integer selected from 0 to 1. In some embodiments, a, b, and m are different. In some embodiments, a, b, and m are same.

In some embodiments, the linker L is selected from the group consisting of

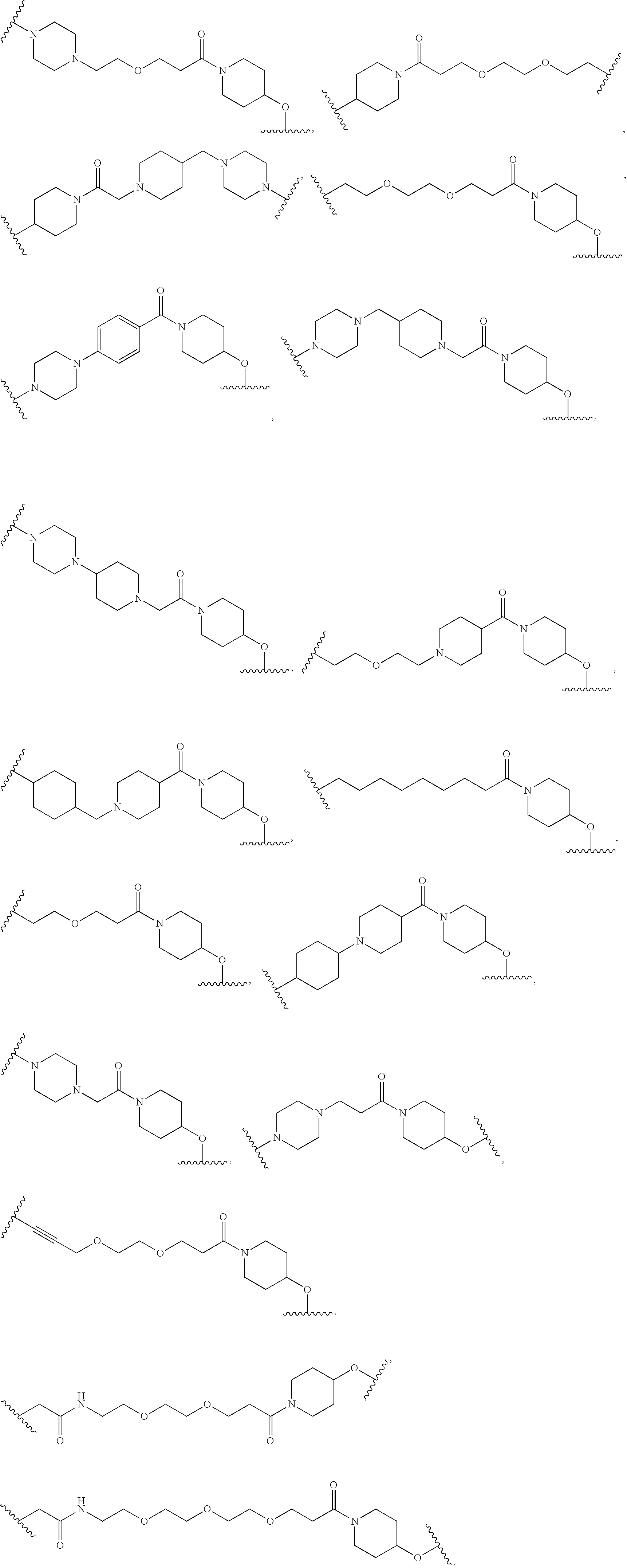

The disclosed PROTACs typically include a moiety that binds to an E3 ubiquitin ligase (M_{E3}), for example, as a ligand for the E3 ubiquitin ligase (M_{E3}). Ligands for E3 ubiquitin ligases for use in preparing PROTACs are known in the art. (See, e.g., An et al., “Small-molecule PROTACs: An emerging and promising approach for the development of targeted therapy drugs,” EBioMedicine. 2018 October; 36: 553-562; and Gu et al., “PROTACs: An Emerging Targeting Technique for Protein Degradation in Drug Discovery,” Bioessays. 2018 April; 40(4):e1700247, the contents of which are incorporated herein by reference in their entireties).

In some embodiments of the disclosed PROTACs, M_{E3} is a moiety that binds to an E3 ubiquitin ligase selected from Von Hippel-Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

In other embodiments of the disclosed PROTACs, M_{E3} is a moiety derived from thalidomide, pomalidomide, lenalidomide, iberdomide, (S,R,S)-AHPC-Me hydrochloride, (S,R,S)-AHPC-Me dihydrochloride, cereblon modulator 1, thalidomide-propargyl, (S,R,S)-AHPC-propargyl, (S,R,S)-AHPC hydrochloride, CC-885, thalidomide-O—COOH, lenalidomide hemihydrate, thalidomide fluoride, thalidomide-OH, lenalidomide-Br, thalidomide D4, lenalidomide hydrochloride, (S,R,S)-AHPC-Me, clAP1 ligand 1, TD-106, E3 ligase Ligand 8, E3 ligase Ligand 9, E3 ligase Ligand 10, E3 ligase Ligand 13, E3 ligase Ligand 14, E3 ligase Ligand 18, BC-1215, VHL ligand 1 (VHL-1), VHL ligand 2 (VHL-2), VHL Ligand 8 (VHL-8), VH032, VH032-cyclopropane-F, VH032 thiol, VH-298, VL-269, VL-285, LCL161, hydroxyproline-based ligands, HIF-1α-derived (R)-hydroxyproline, Nutlin carboxylic acid, (4R,5S)-Nutlin carboxylic acid, (S,R,S)-AHPC-Boc, AR antagonist 1, NV03, (S,R,S)-AHPC TFA, (S,R,S)-AHPC, β-Naphthoflavone-CH2-Br, β-Naphthoflavone-CH2-OH, Bestatin-amido-Me, MV-1-NH-Me, (S,S,S)-AHPC hydrochloride, and clAP1 ligand 2.

In some embodiments, M_{E3} has a formula selected from the group consisting of:

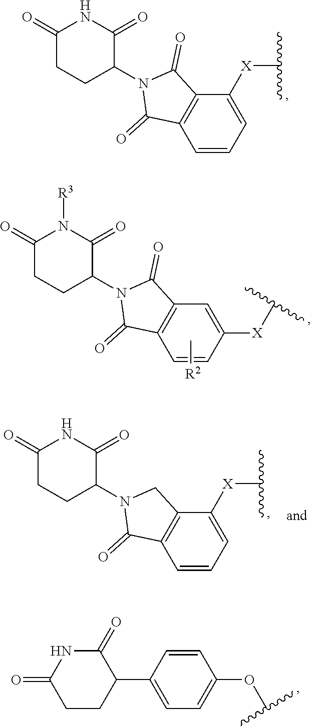

wherein
X is a bond, NH, or NMe;
R² is hydrogen or halo; and
R³ is hydrogen or methyl.

In some embodiments, X is a bond in the formula of M_{E3}. In some embodiments, X is NH in the formula of M_{E3}. In some embodiments, X is NMe in the formula of M_{E3}. In some embodiments, R² is hydrogen in the formula of M_{E3}. In some embodiments, R² is halo in the formula of M_{E3}. In some embodiments, R² is fluoro in the formula of M_{E3}. In some embodiments, R² is fluoro and X is a bond in the formula of M_{E3}.

In some embodiments, M_{E3} has a formula selected from the group consisting of

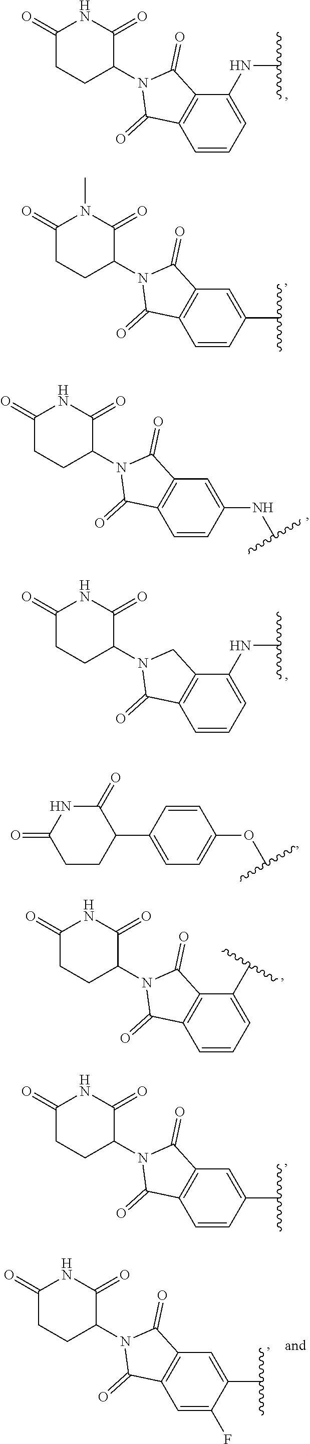

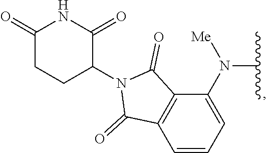 [SMILES: *N(C)c1cccc2c1C(=O)N(C1CCC(=O)NC1=O)C2=O]

In some embodiments, the molecule has a formula selected from the group consisting of

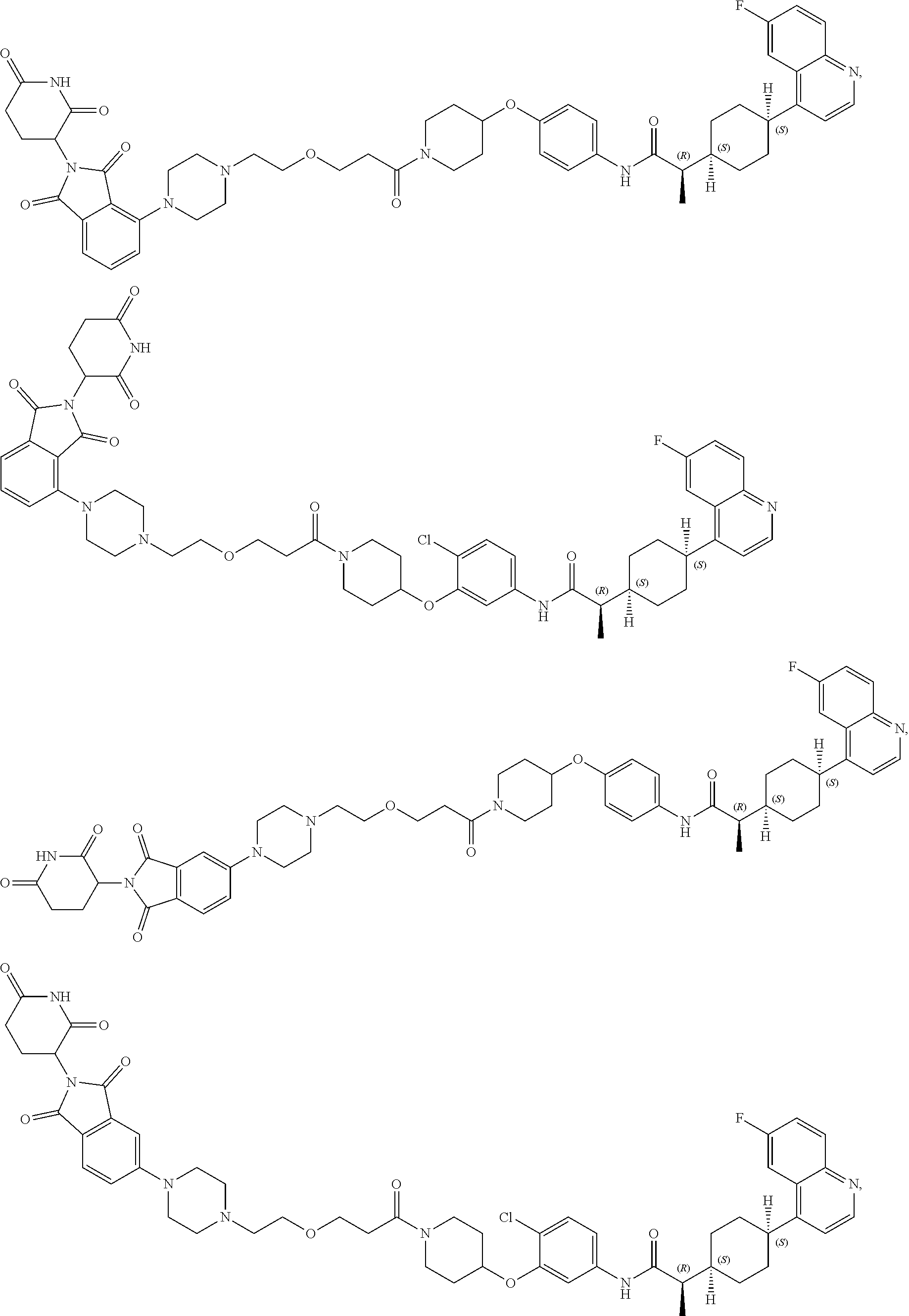

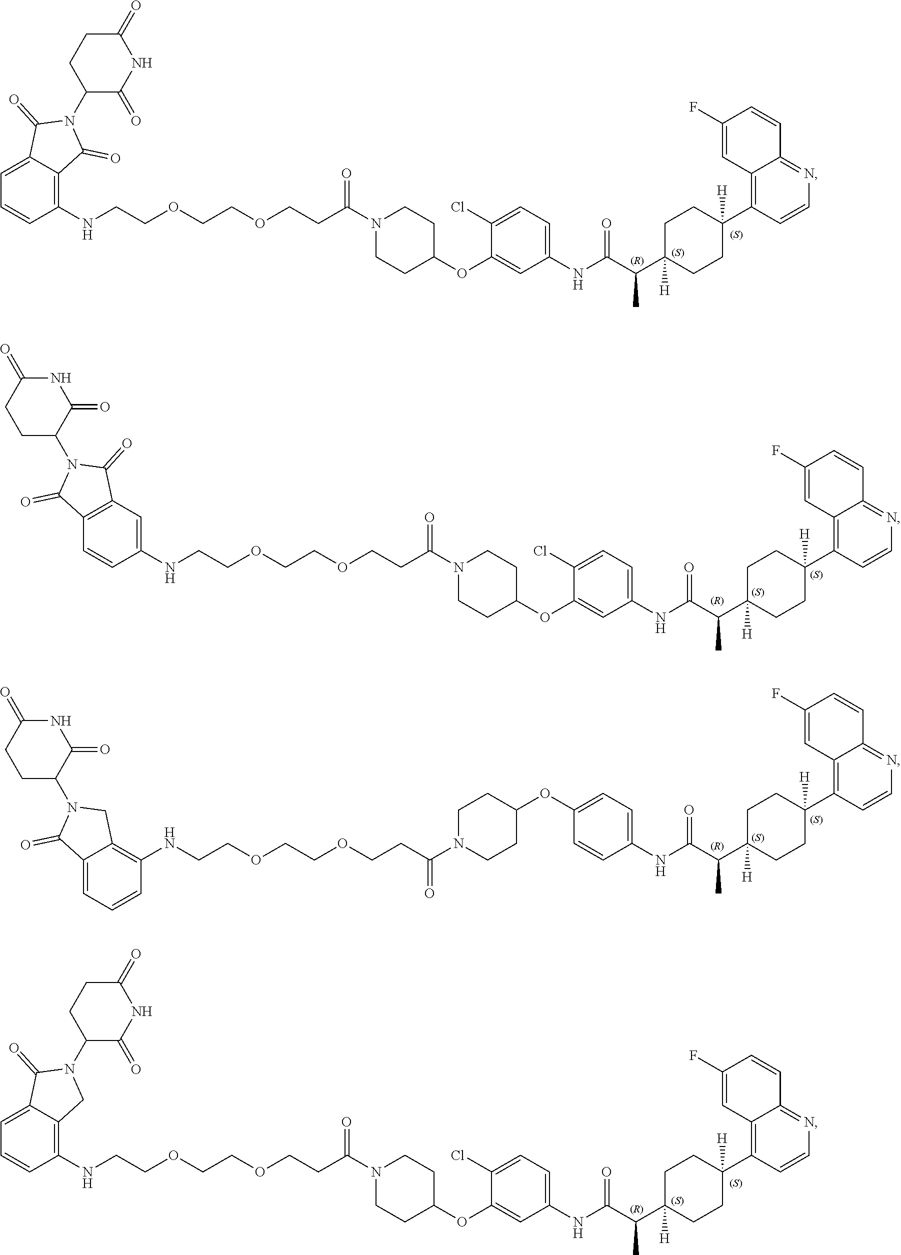

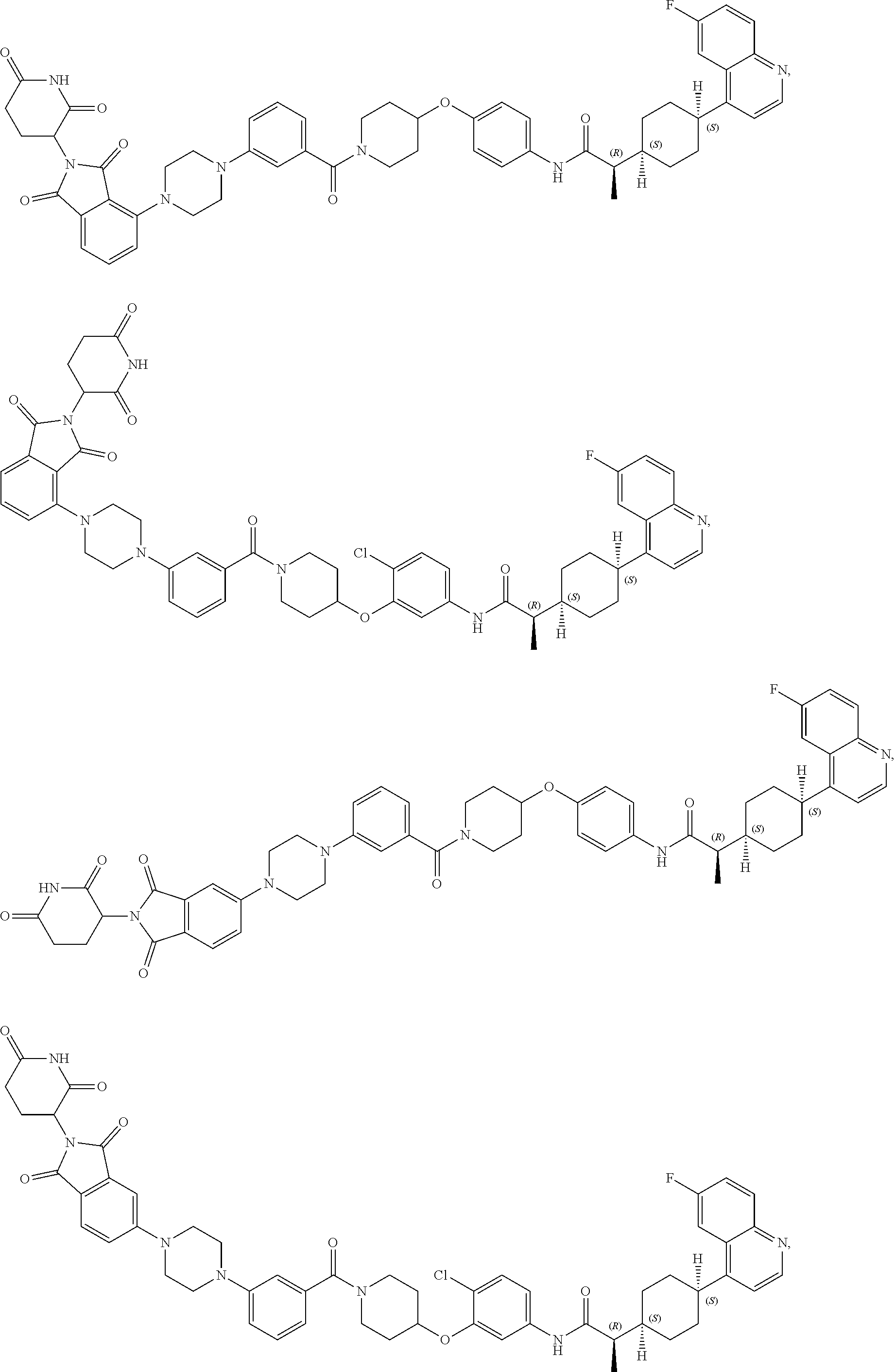

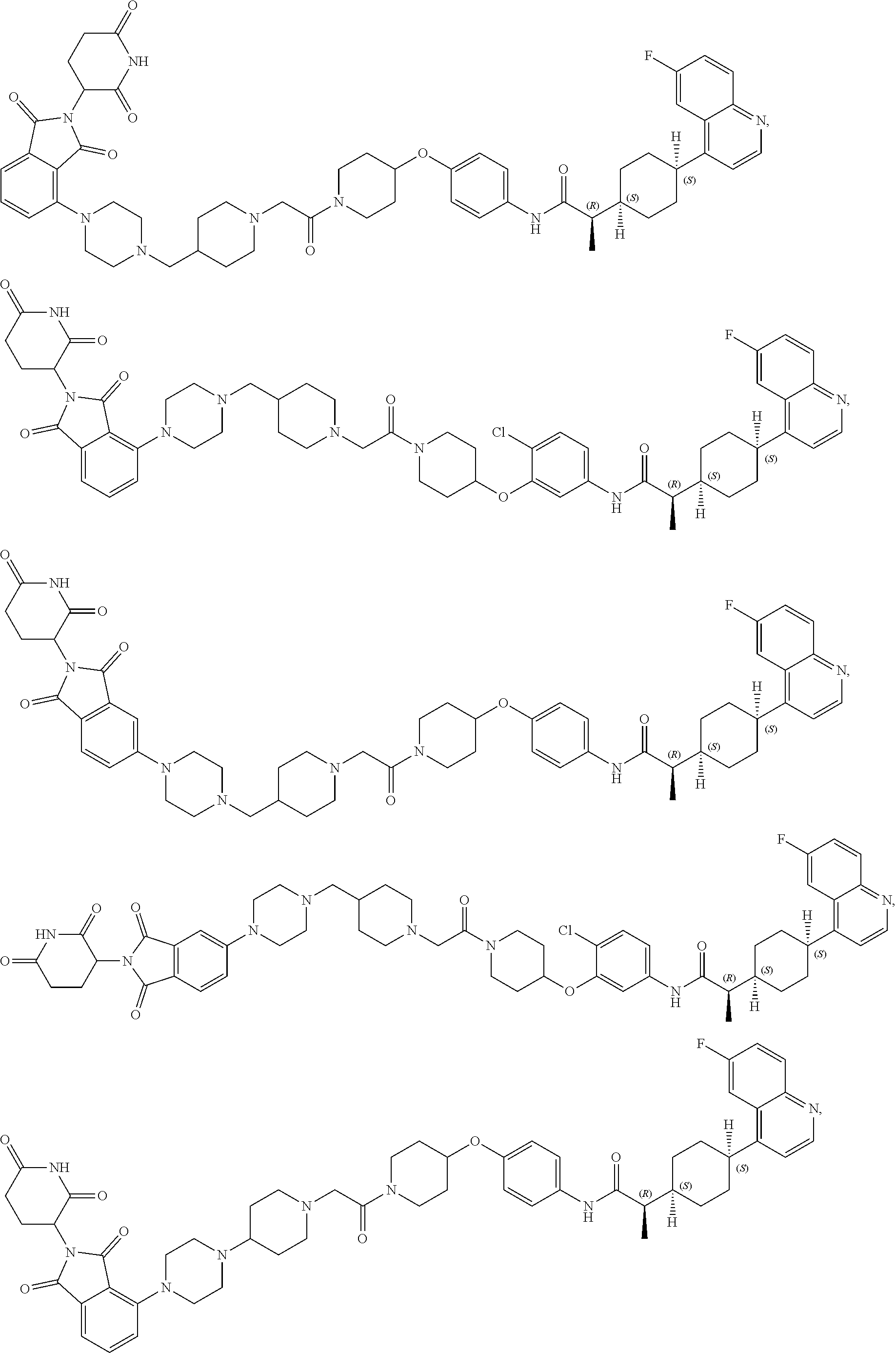

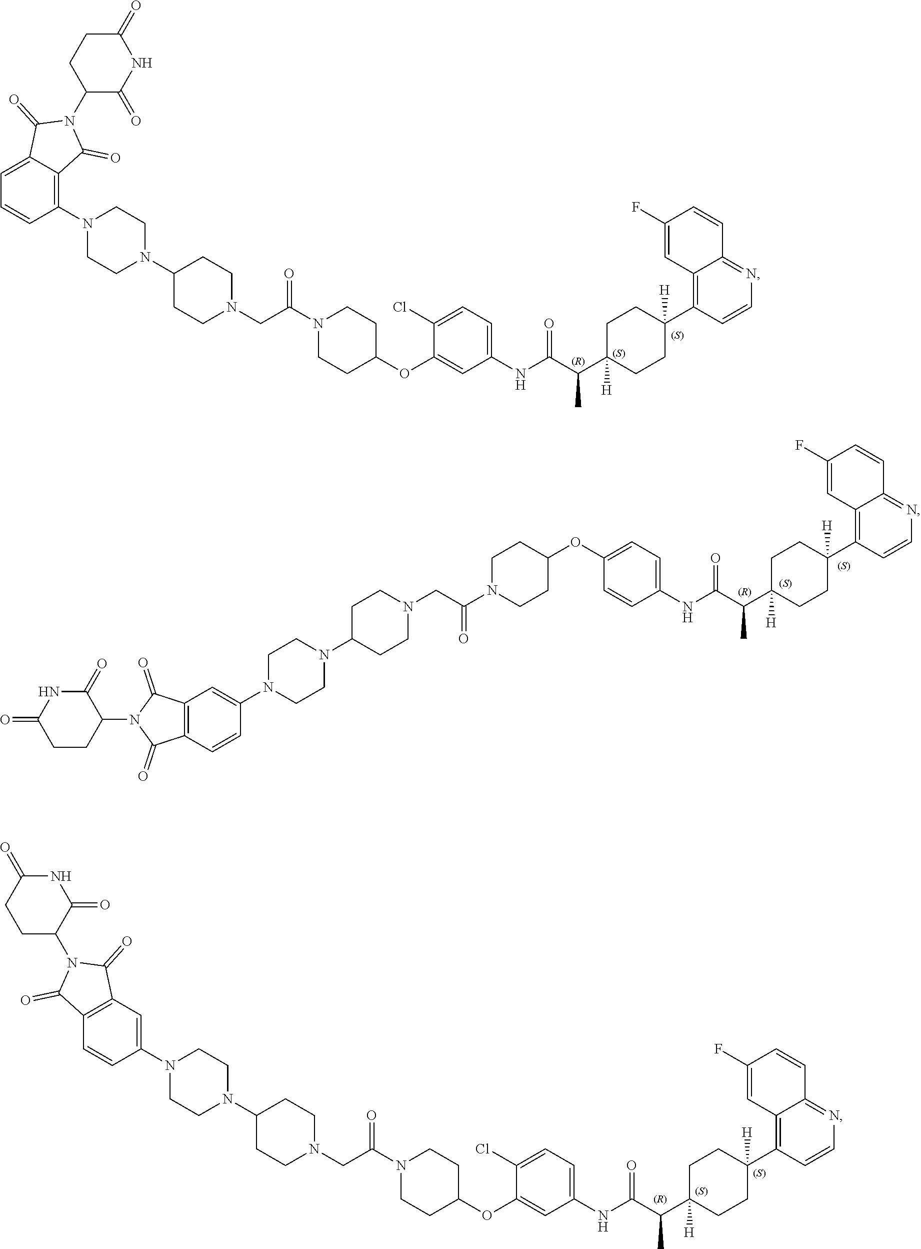

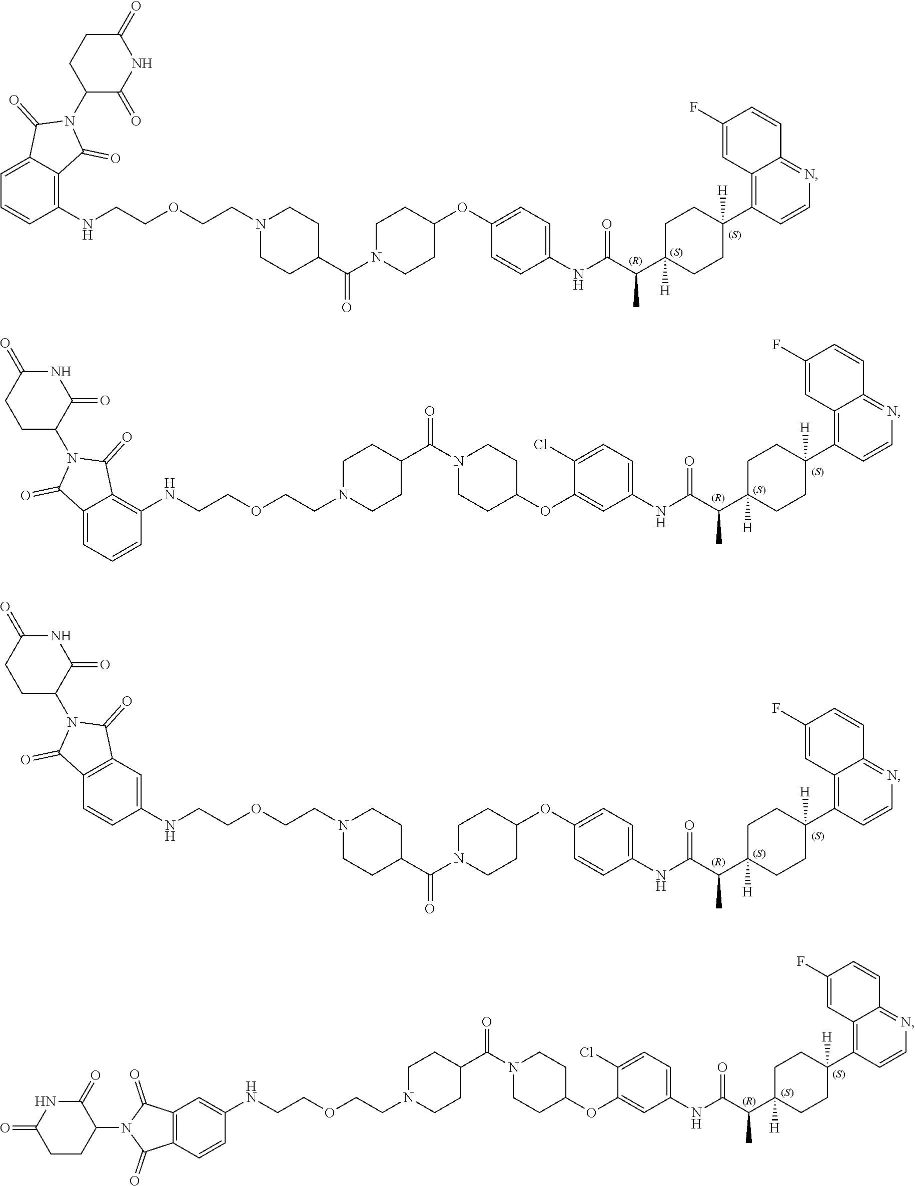

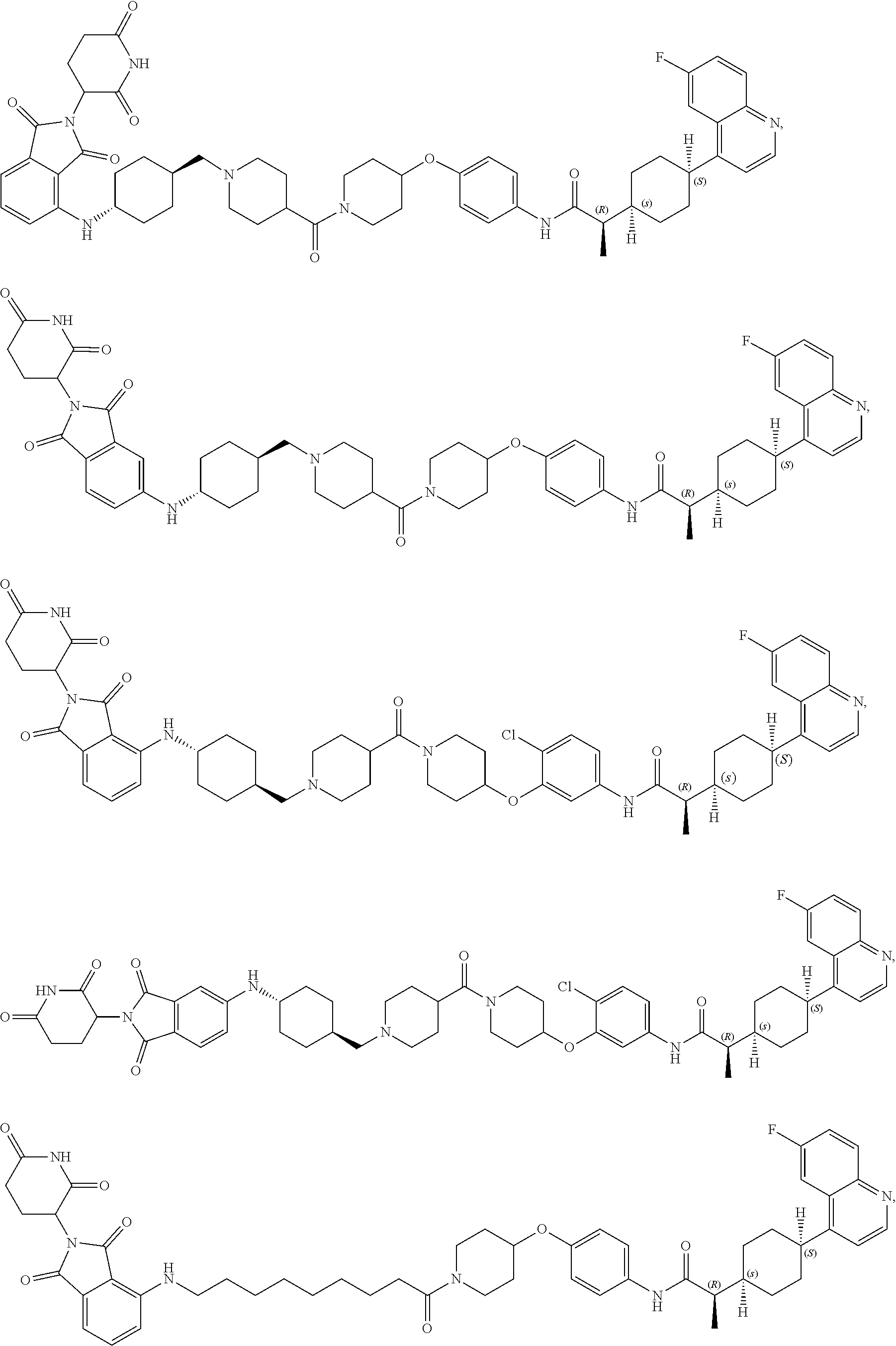

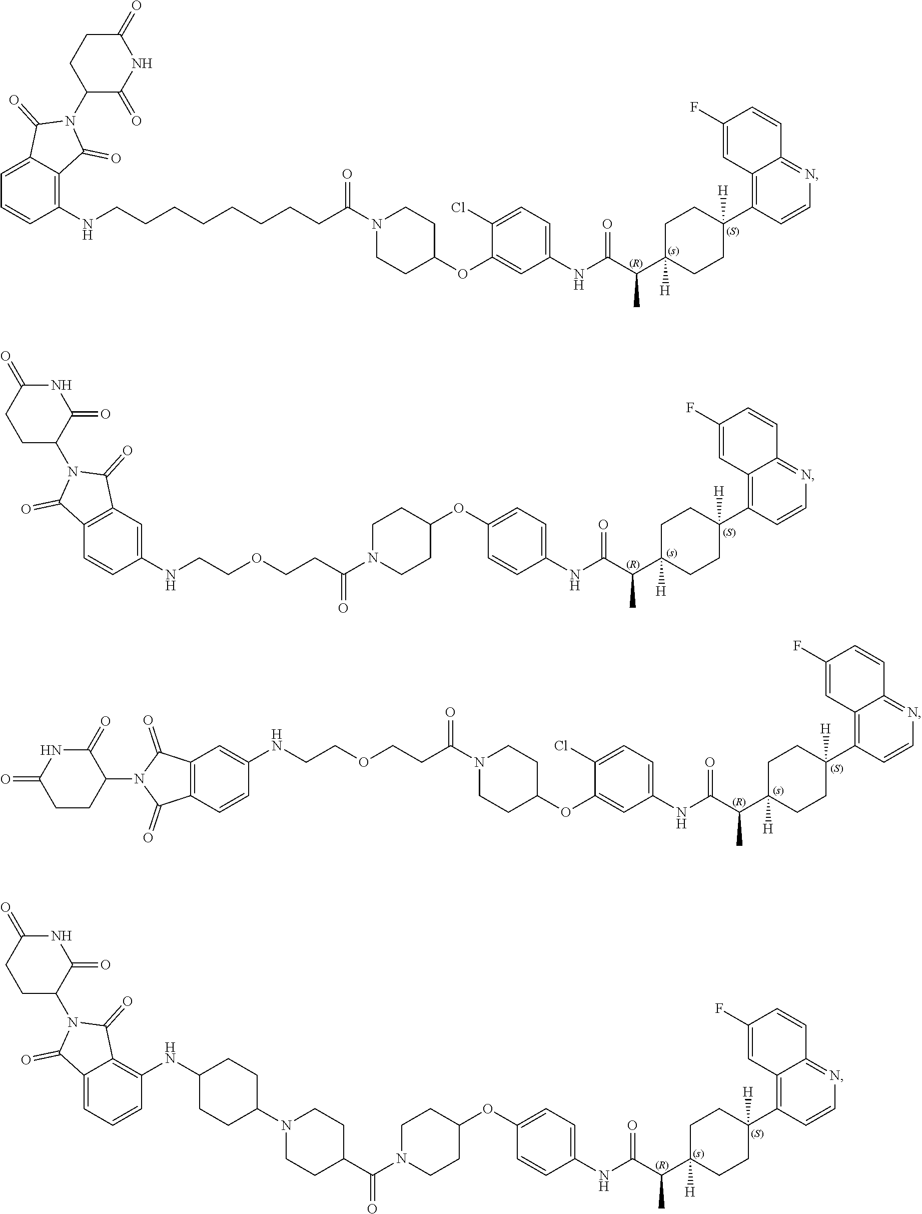

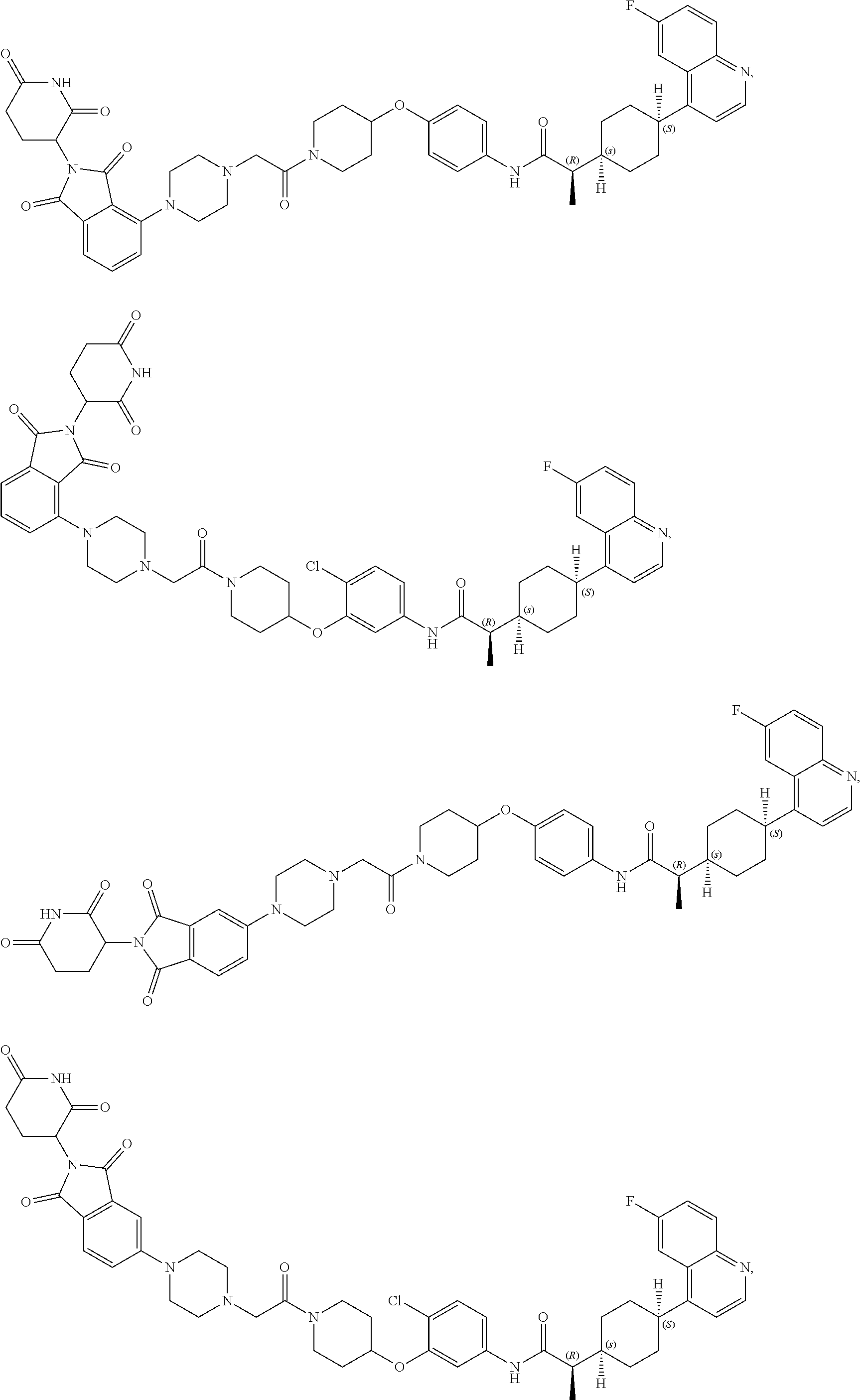

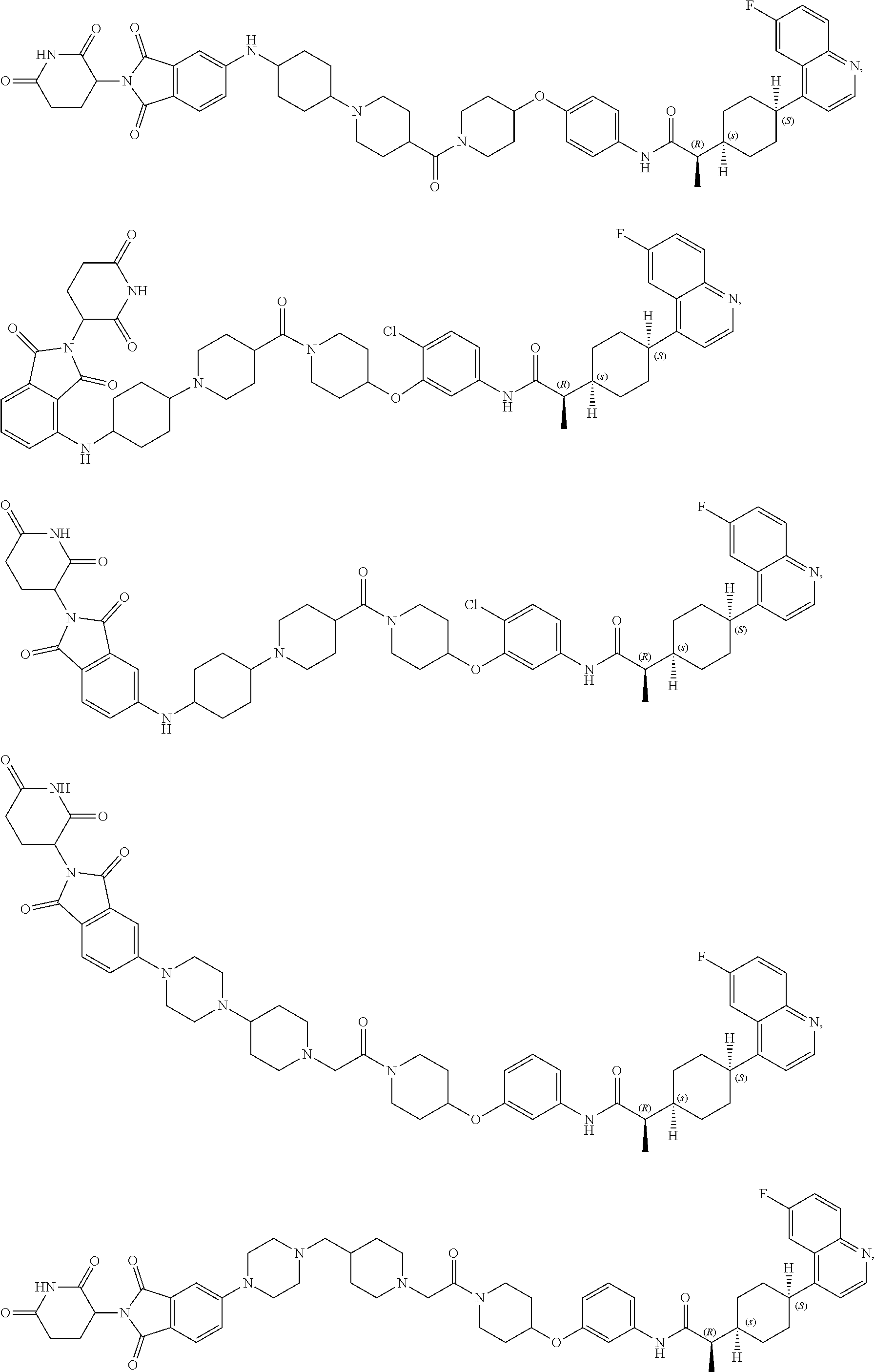

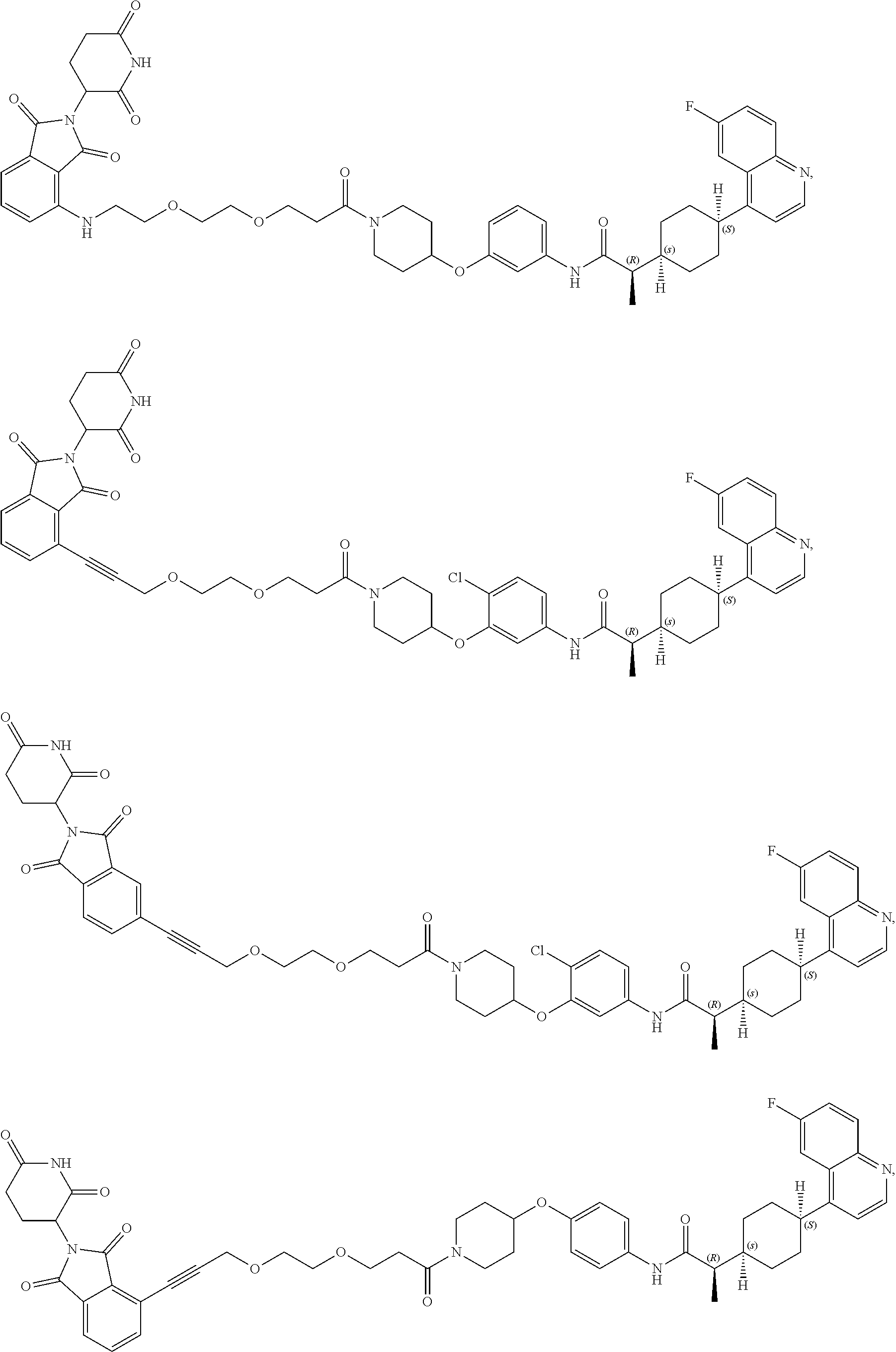

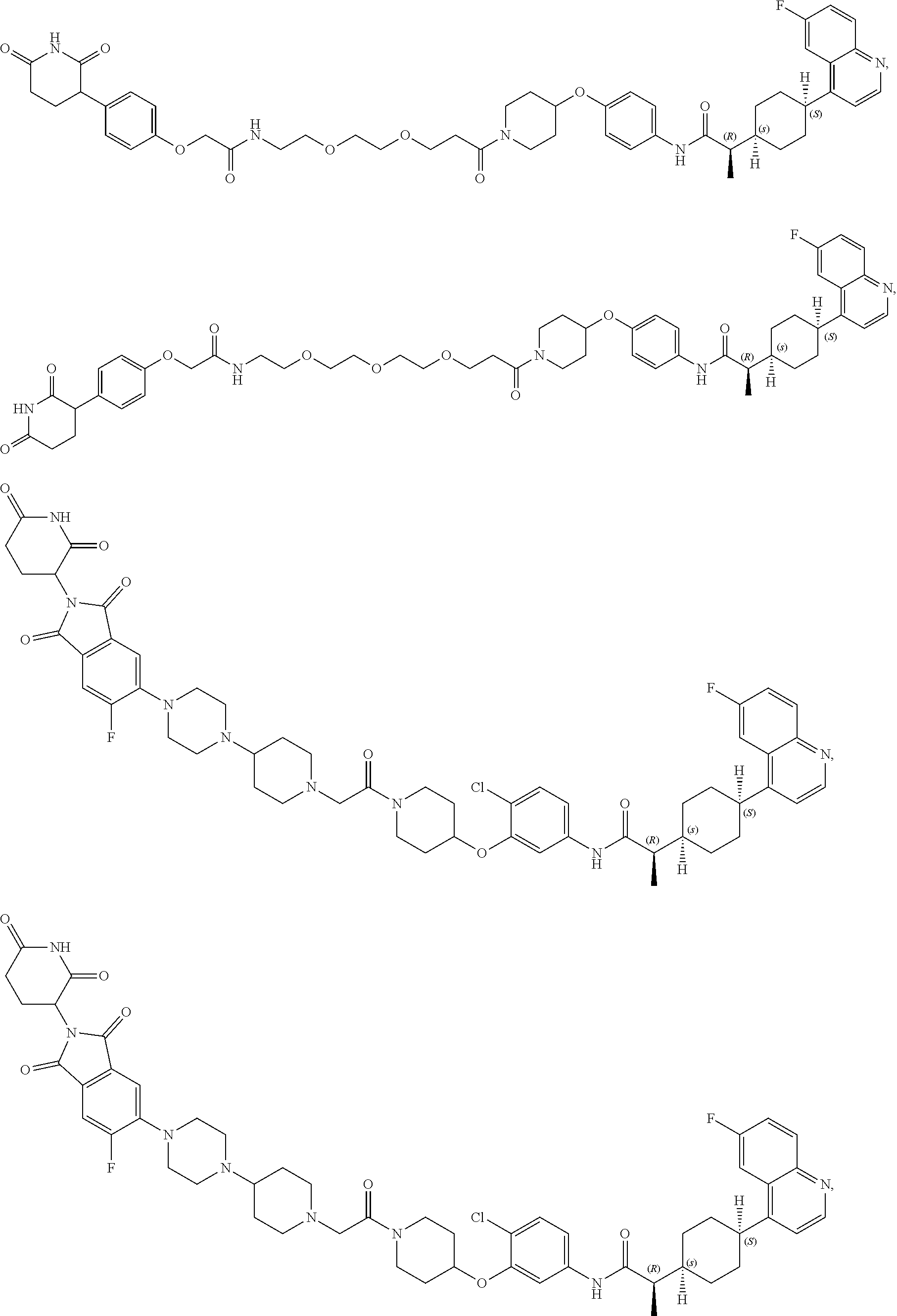

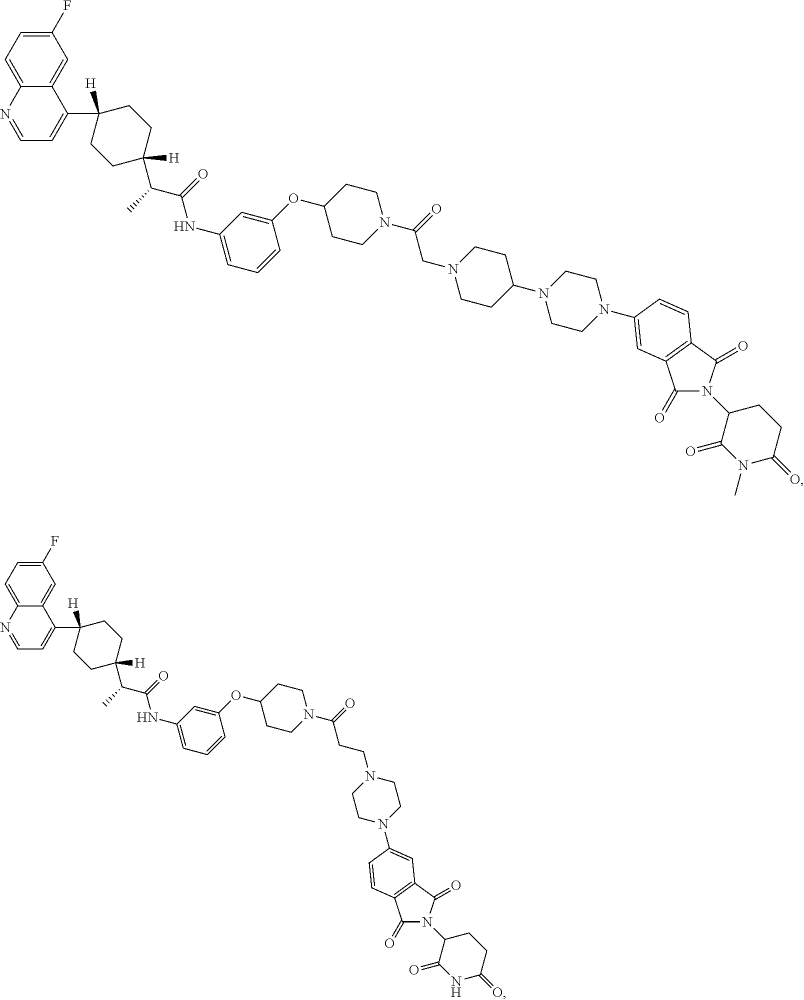 [SMILES: C[C@@H](C(=O)Nc1cccc(OC2CCN(C(=O)CN3CCC(N4CCN(c5ccc6c(c5)C(=O)N(C5CCC(=O)N(C)C5=O)C6=O)CC4)CC3)CC2)c1)[C@H]1CC[C@@H](c2ccnc3ccc(F)cc32)CC1 ; C[C@@H](C(=O)Nc1cccc(OC2CCN(C(=O)CCN3CCN(c4ccc5c(c4)C(=O)N(C4CCC(=O)NC4=O)C5=O)CC3)CC2)c1)[C@H]1CC[C@@H](c2ccnc3ccc(F)cc32)CC1]

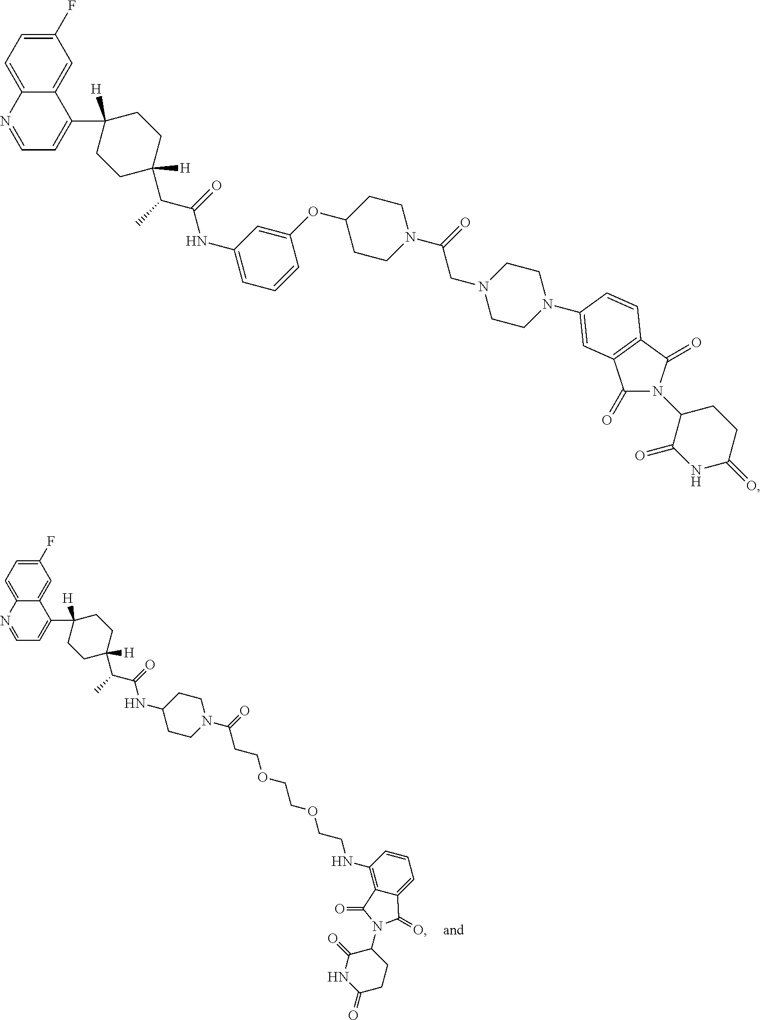 [SMILES: C[C@@H](C(=O)Nc1cccc(OC2CCN(C(=O)CN3CCN(c4ccc5c(c4)C(=O)N(C4CCC(=O)NC4=O)C5=O)CC3)CC2)c1)[C@H]1CC[C@@H](c2ccnc3ccc(F)cc32)CC1 ; C[C@@H](C(=O)NC1CCN(C(=O)CCOCCOCCNc2cccc3c2C(=O)N(C2CCC(=O)NC2=O)C3=O)CC1)[C@H]1CC[C@@H](c2ccnc3ccc(F)cc32)CC1 ; "and"]

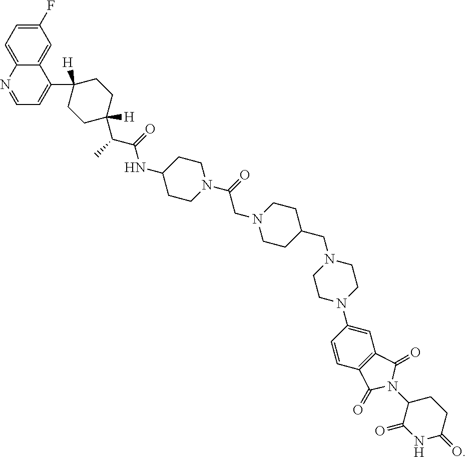 [SMILES: C[C@@H](C(=O)NC1CCN(C(=O)CN2CCC(CN3CCN(c4ccc5c(c4)C(=O)N(C4CCC(=O)NC4=O)C5=O)CC3)CC2)CC1)[C@H]1CC[C@@H](c2ccnc3ccc(F)cc32)CC1]

In some embodiments, the molecule as a formula selected rom the group consisting of

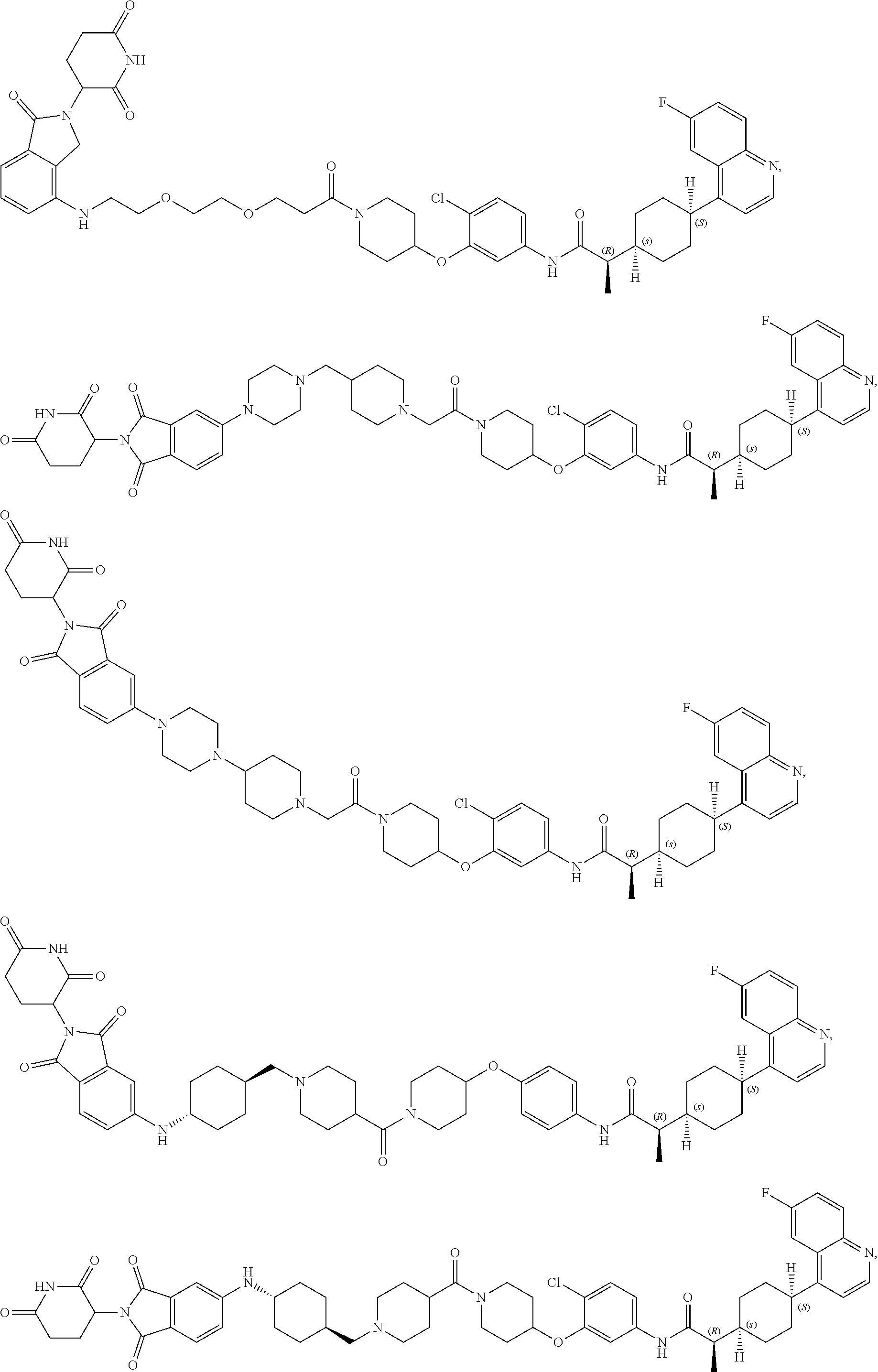

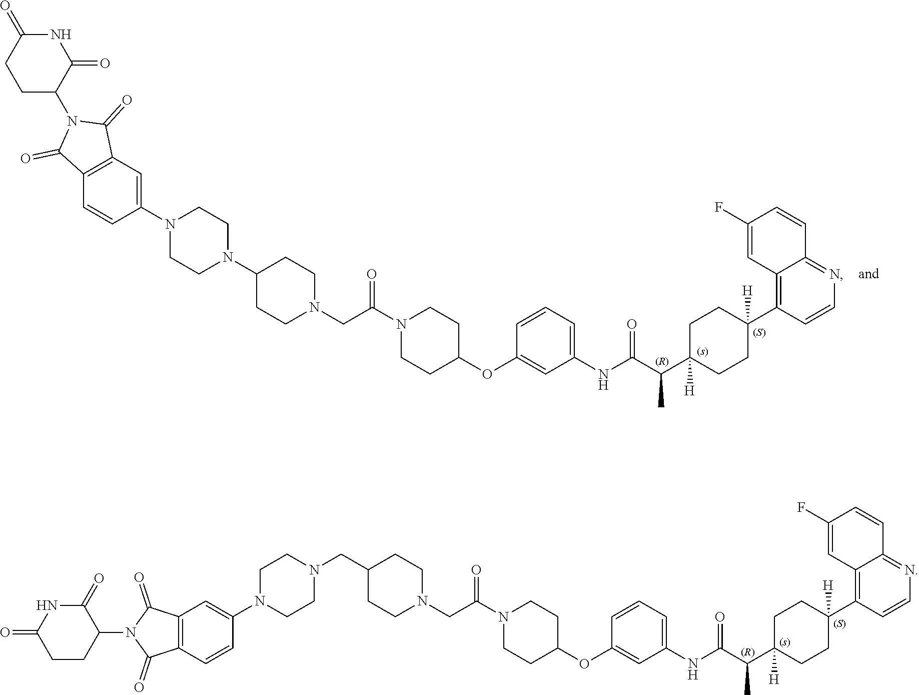

In some embodiments, the molecule has a formula

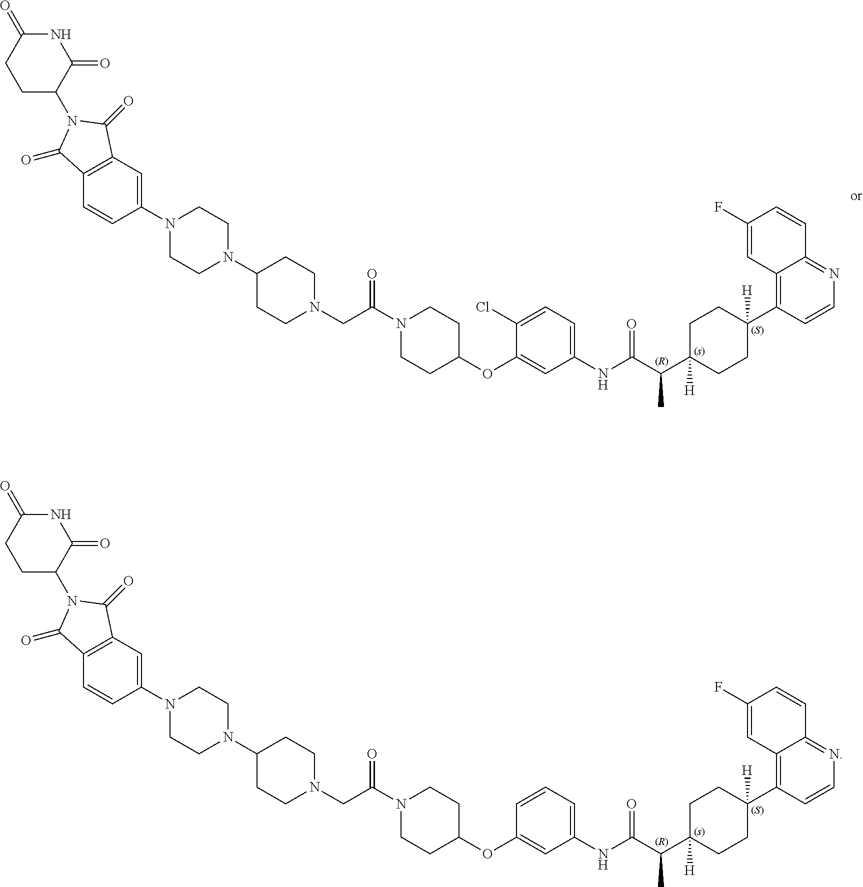

In some embodiments, the molecule has a formula

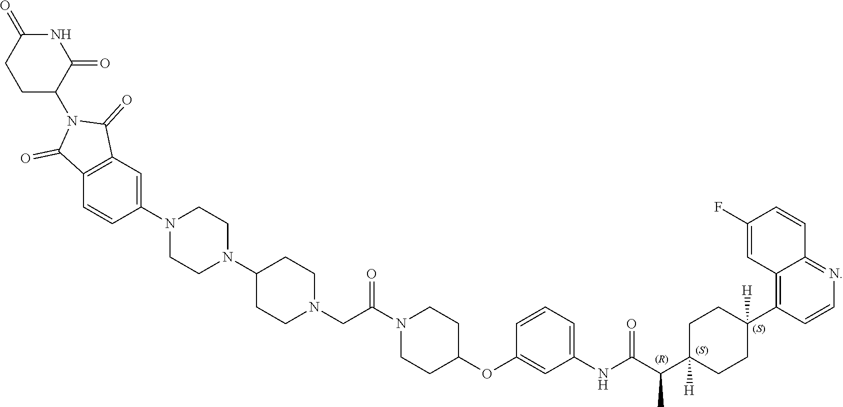

In some embodiments, the molecule is more potent than the compound having a formula

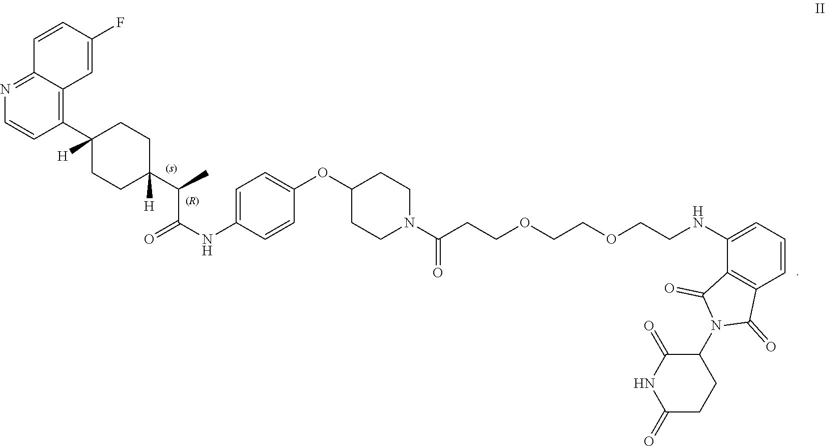

II. As used herein, the term “potent” refers to the ability of a molecule to degrade IDO1 proteins, with the more potent molecule having a better degrading effect on IDO1. The “potency” of molecules, as discussed herein, may be measured and assessed by comparing their DC50 values obtained or amount of IDO1 remaining under the same conditions. “DC50” means the concentration of a molecule at which the IDO1 is degraded by fifty percent (50%). As such, a lower DC50 value indicates that the molecule is more potent than other molecules with higher DC50 values. The compounds disclosed herein may have a DC50 value 95%, 90%, 85%, 80%, 75%, 70%, 65%, 60%, 55%, or 50% less than a reference compound. The compounds disclosed herein may have a value of remaining IDO1 90%, 85%, 80%, 75%, 70%, 65%, 60%, 55%, or 50% less than a reference compound at equivalent concentrations and conditions. The reference compound may be selected from US 2022/0023431, such as NU223612. In some embodiments, the molecule as described herein has a lower DC50 than the reference compound at equivalent conditions and concentrations of IDO1. In some embodiments, a lower concentration of the molecule as described herein than the reference compound is required to degrade the same amount of IDO1 protein. In some embodiments, at equivalent conditions and concentrations (of IDO1 and the tested IDO1 degrader), the amount of IDO1 remaining is lower after IDO1 is contacted with the molecule as described herein than that after IDO1 is contacted with the reference compound.

As used herein, the phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts have been described in S. M. Berge et al. J. Pharmaceutical Sciences, 1977, 66: 1-19.

The molecules disclosed herein may contain either a basic or an acidic functionality, or both, and can be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

The compounds and molecules (e.g., PROTACs) of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds and molecules may be designated by the symbols “R” or “S,” or “+” or “-” depending on the configuration of substituents around the stereogenic carbon atom and or the optical rotation observed. The present invention encompasses various stereo isomers of these compounds and molecules and mixtures thereof. Stereoisomers include enantiomers and diastereomers. Mixtures of enantiomers or diastereomers may be designated (+)” in nomenclature, but the skilled artisan will recognize that a structure may denote a chiral center implicitly. It is understood that graphical depictions of chemical structures, e.g., generic chemical structures, encompass all stereoisomeric forms of the specified compounds and molecules, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

The formulae of the compounds and molecules disclosed herein should be interpreted as encompassing all possible stereoisomers, enantiomers, or epimers of the compounds and molecules unless the formulae indicate a specific stereoisomer, enantiomer, or epimer. The formulae of the compounds and molecules disclosed herein should be interpreted as encompassing salts, esters, amides, or solvates thereof of the compounds and molecules.

### Pharmaceutical Compositions

Also disclosed herein are pharmaceutical composition comprising a therapeutically effective amount of the compounds as disclosed herein and a pharmaceutically acceptable carrier, excipient, or diluent.

As used herein, the phrase “effective amount” shall mean that drug dosage that provides the specific pharmacological response for which the drug is administered in a significant number of subject in need of such treatment. An effective amount of a drug that is administered to a particular subject in a particular instance will not always be effective in treating the conditions/diseases described herein, even though such dosage is deemed to be a therapeutically effective amount by those of skill in the art.

The pharmaceutical composition may include the molecule in a range of about 0.1 to 2000 mg. In some embodiments, the pharmaceutical composition may include the molecule in a range of from about 0.5 to 500 mg. In some embodiments, the pharmaceutical composition may include the molecule in a range of from about 1 to 100 mg. The pharmaceutical composition may be administered to provide the molecule at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The molecule utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The molecules utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™) Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The molecules utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the molecules may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

Optionally, the disclosed molecules or pharmaceutical compositions comprising the disclosed molecules may be administered with additional therapeutic agents, optionally in combination, in order to treat cancers. In some embodiments of the disclosed methods, one or more additional therapeutic agents are administered with the disclosed compounds or with pharmaceutical compositions comprising the disclosed molecules, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed molecules and further to comprise one or more additional therapeutic agents, for example, one or more additional therapeutic agents for treating cell proliferative diseases and disorders.

The disclosed compounds and molecules (e.g., PROTACs) may be formulated as anti-cancer therapeutics, including hematologic malignancies, breast, lung, pancreas and prostate malignancies. The disclosed compounds and molecules also may be formulated as anti-inflammation therapeutics.

### Methods

Disclosed herein are also methods of treating cancer or inhibit cell proliferation of cancer cells in a subject in need thereof. The methods comprise administering to the subject the molecule or the pharmaceutical composition as disclosed herein.

As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with a proteolytic-targeted chimeric molecule (PROTAC), which his targeted to IDO for degradation of IDO. For example, a “subject in need of treatment” may include a subject having a cell proliferative disease, disorder, or condition such as cancer (e.g., cancers such as glioblastoma multiforme (GBM), multiple myeloma, leukemia, non-small cell lung cancer, colon cancer, cancer of the central nervous system, melanoma, ovarian cancer, renal cancer, prostate cancer, colorectal cancer, uterine cancer, pancreatic cancer, and breast cancer). A “subject in need of treatment” may include a subject having a cell proliferative disease, disorder, or condition such as cancer that is associated with IDO activity and/or that may be treated by administering an effective amount of an agent that modulates IDO activity.

As used herein, the term “modulate” means decreasing or inhibiting activity and/or increasing or augmenting activity. For example, modulating IDO activity may mean increasing or augmenting IDO activity and/or decreasing or inhibiting IDO activity. The proteolytic-targeted chimeric molecules (PROTACs) disclosed herein may be administered to modulate IDO activity.

In some embodiments, the disclosed proteolytic-targeted chimeric molecules (PROTACs) may exhibit one or more biological activities. The disclosed PROTACs may inhibit the growth of cells that express IDO (preferably by at least 50%, 60%, 70%, 80%, 90%, 95%, 96%, 97%, 98%, or 99% at a concentration of less than about 100 μM, 50 μM, 10 μM, 1 μM, 0.1 μM, 0.0 μM, 0.01 μM, 0.005 μM, 0.00 μM, or less). The disclosed PROTACs may not inhibit the growth of cells that do not express IDO (preferably by not more than 50%, 40%, 30%, 20%, 10%, 5%, 4%, 3%, 2% or less at a concentration of greater than about 0.001 μM, 0.005 μM, 0.01 μM, 0.5 μM, 0.1 μM, 1.0 μM, 10 μM, and 100 μM or higher). Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 10 μM. Preferably, the disclosed PROTACs may not produce significant DNA damage (e.g., in an rH2AX staining assay at a concentration greater than about 0.001 μM, 0.005 μM, 0.01 μM, 0.1 μM, 1.0 μM, 10 μM, 100 μM, or higher).

In some embodiments, the disclosed PROTACs may be effective in inhibiting cell proliferation of cancer cells, including cancer cells that express IDO and whose proliferation is inhibited by inhibiting the biological activity of IDO. The disclosed PROTACs may be effective in inhibiting cell proliferation of one or more types of cancer cells including: multiple myeloma cells, such as MM.IlS cells; leukemia cells, such as CCRF-CEM, HL-60(TB), MOLT-4, RPMI-8226 and SR; non-small lung cancer cells, such as A549/ATCC, EKVX, HOP-62, HOP-92, NCI-H226, NCI-H23, NCI-H322M, NCI-H460 and NCI-H522; colon cancer cells, such as COLO 205, HCC-2998, HCT-116, HCT-15, HT29, KM12 and SW-620; CNS: SF-268, SF-295, SF-539, SNB-19, SNB-75 and U251; melanoma cancer cells, such as LOX IMVI, MALME-3M, M14, MDA-MB-435, SK-MEL-2, SK-MEL-28, SK-MEL-5, UACC-257 and UACC-62; ovarian cancer cells, such as IGR-OV1, OVCAR-3, OVCAR-4, OVCAR-5, OVCAR-8, NCI/ADR-RES and SK-OV-3; renal cancer cells, such as 786-0, A498, ACHN, CAKI-1, RXF 393, SN12C, TK-10 and UO-31; prostate cancer cells, such as DU-145 and PC-3; pancreatic cancer cells, such as PANC-1, AsPC-1, KP-3, BxPC-3, TCC-PAN2, and MIA PaCa-2. AsPC-1, BxPC-3 and MIA PaCa-2; and breast cancer cells, such as MCF7, MDA-MB-231/ATCC, MDA-MB-468, HS 578T, BT-549 and T-47D.

In some embodiments, the cancer is glioblastoma.

Cell proliferation and inhibition thereof by the presently disclosed PROTACs may be assessed by cell viability methods disclosed in the art including colorimetric assays that utilize dyes such as MTT, XTT, and MTS to assess cell viability. Preferably, the disclosed PROTACs have an IC₅₀ of less than about 10 μM, 5 μM, 1 μM, 0.5 μM, 0.01 μM, 0.005 μM, 0.001 μM or lower in the selected assay.

In some embodiments of the disclosed methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as endpoints any of these disclosed dose levels (e.g., 500-2000 ng/kg body weight of the subject).

In some embodiments, the method further comprises administering a therapy comprising chemotherapy, radiation, checkpoint inhibitors, or immunotherapy to the subject. The therapy is administered before, concurrently with, or after administering the pharmaceutical composition.

The disclosed PROTACs may be administered with additional therapeutic agents. In some embodiments, the disclosed PROTACs are administered with additional immunotherapeutic agents in order to treat cancers, such as GBM. In some embodiments, the disclosed PROTACs may be administered to a subject in need thereof in a treatment method comprising administering the disclosed PROTACs to the subject and further comprising administering an anti-PD1 agent to the subject (e.g., an anti-PD1 antibody), before, concurrently with, or after administering the disclosed PROTACs. In some embodiments, the disclosed PROTACs may be administered to a subject in need thereof in a treatment method comprising administering the disclosed PROTACs to the subject and further comprising administering an anti-PD-L1 agent to the subject (e.g., an anti-PD-L1 antibody), before, concurrently with, or after administering the disclosed PROTACs.

### ILLUSTRATIVE EMBODIMENTS

The following embodiments are illustrative and should not be interpreted to limit the scope of the claimed subject matter.
Embodiment 1. A molecule having a formula: M_{1DO1}-L-M_{E3}, or a pharmaceutically acceptable salt thereof, wherein M_{1DO1} is a moiety that binds to indoleamine 2,3-dioxygenase 1 (IDO1), L is a linker covalently attaching M_{1DO1} and M_{E3}, and M_{E3} is a moiety that binds to an E3 ubiquitin ligase;

wherein
L comprises a piperidinylene moiety;
with the proviso that M_{E3} does not comprise

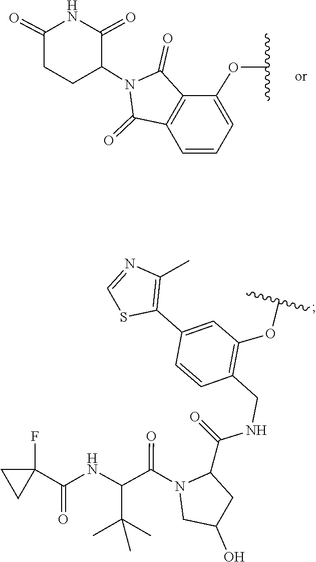 [SMILES: *Oc1cccc2c1C(=O)N(C1CCC(=O)NC1=O)C2=O ; *Oc1cc(-c2scnc2C)ccc1CNC(=O)C1CC(O)CN1C(=O)C(NC(=O)C1(F)CC1)C(C)(C)C]

with the proviso that the molecule is not (2R)-N-(4-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide or (2R)-N-(4-((1-(3-(2-((2-(2,6-dioxopiperidin-3-yl)-1,3-dioxoisoindolin-4-yl)amino)ethoxy)propanoyl)piperidin-4-yl)oxy)phenyl)-2-((1s,4S)-4-(6-fluoroquinolin-4-yl)cyclohexyl)propanamide.
Embodiment 2. The molecule of embodiment 1, wherein M_{1DO1} has a formula I:

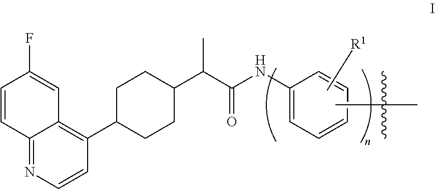

wherein
R¹ is hydrogen or halo; and
   n is 0 or 1.
Embodiment 3. The molecule of embodiment 1 or 2, wherein M_{1DO1} has a formula

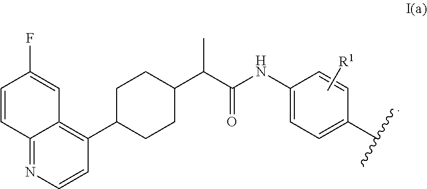

Embodiment 4. The molecule of embodiment, wherein R¹ is hydrogen.
Embodiment 5. The molecule of embodiment 3, wherein R¹ is chloro.
Embodiment 6. The molecule of embodiment 1 or 2, wherein M_{1DO1} has a formula I(b):

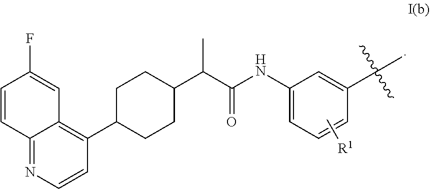

Embodiment 7. The molecule of embodiment 6, wherein R¹ is hydrogen.
Embodiment 8. The molecule of embodiment 6, wherein R¹ is chloro.
Embodiment 9. The molecule of embodiment 1 or 2, wherein M_{1DO1} has a formula

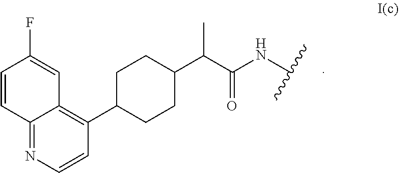 [SMILES: *NC(=O)C(C)C1CCC(c2ccnc3ccc(F)cc23)CC1 ; "."]

Embodiment 10. The molecule of any one of embodiments 1-9, wherein L is selected from the group consisting of

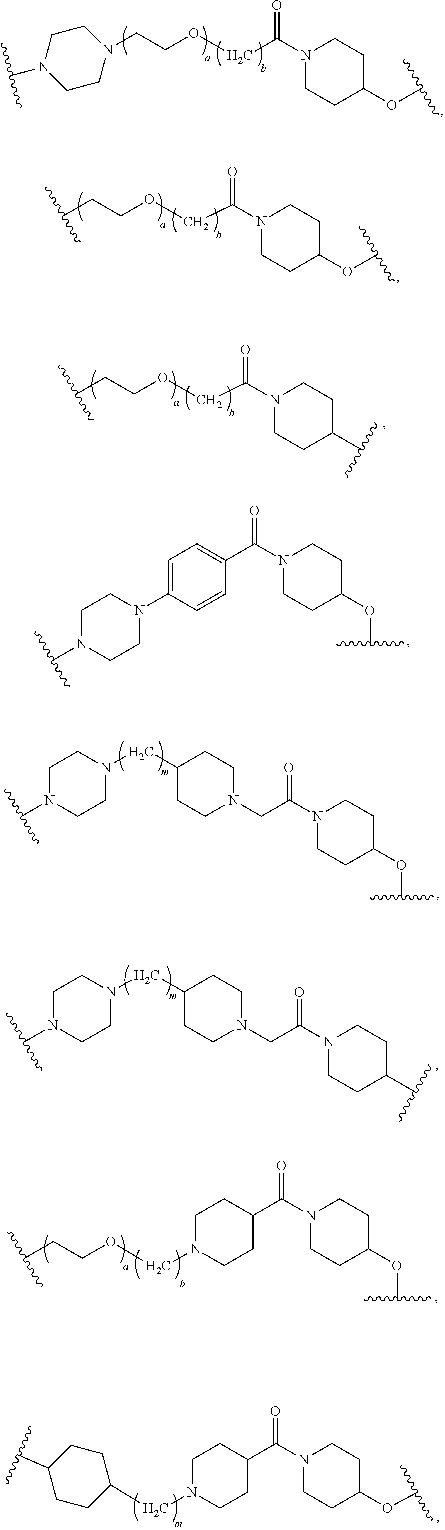

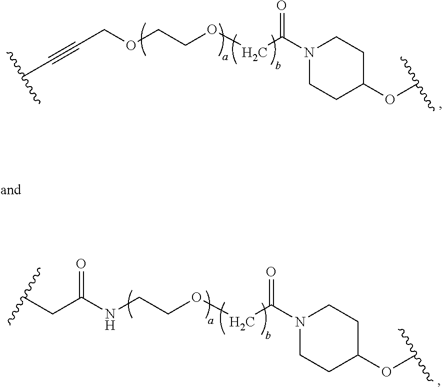

wherein each of a, b, and m is independently an integer selected from 0 to 20.
Embodiment 11. The molecule of any one of embodiments 1-10, wherein L is selected from the group consisting of

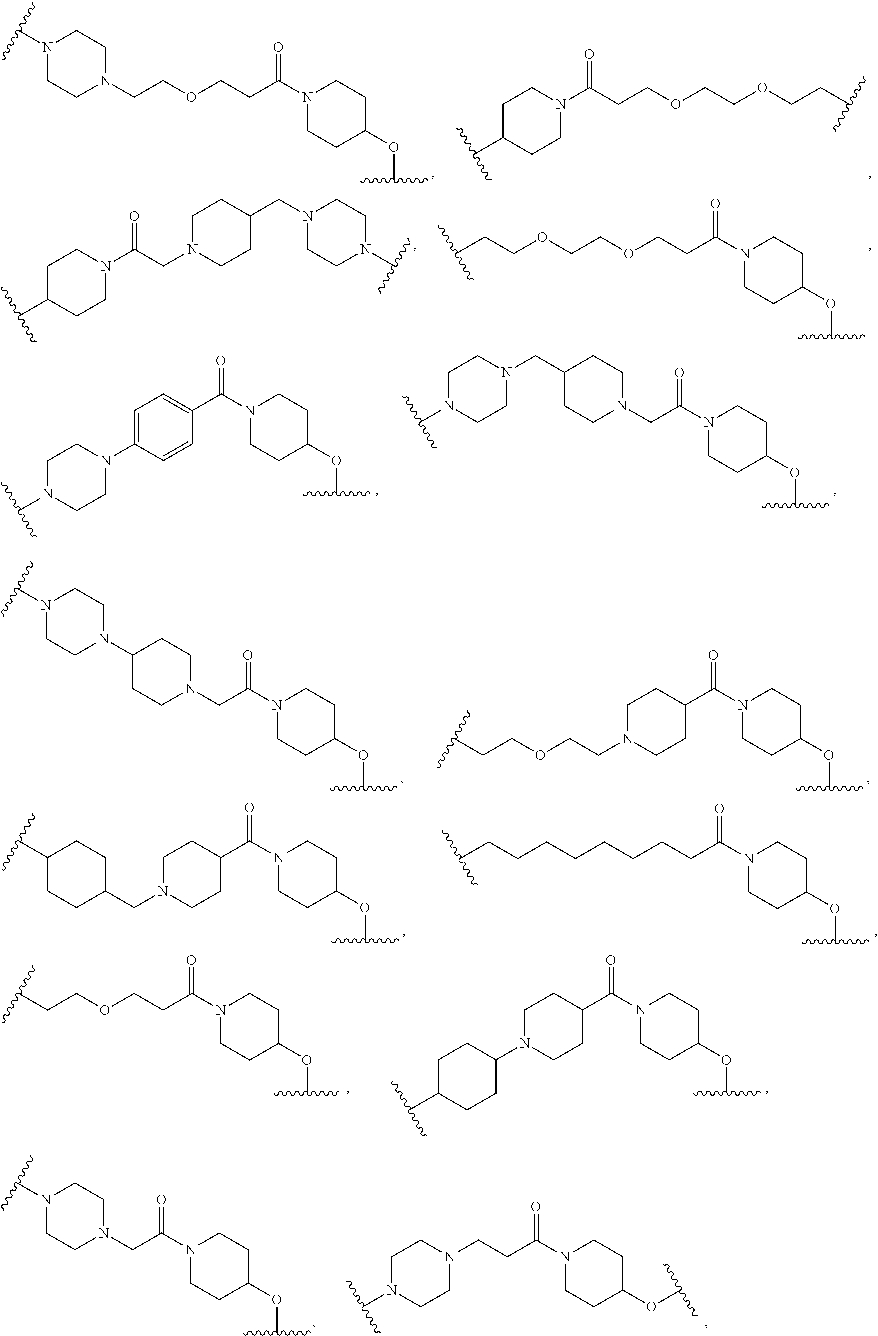

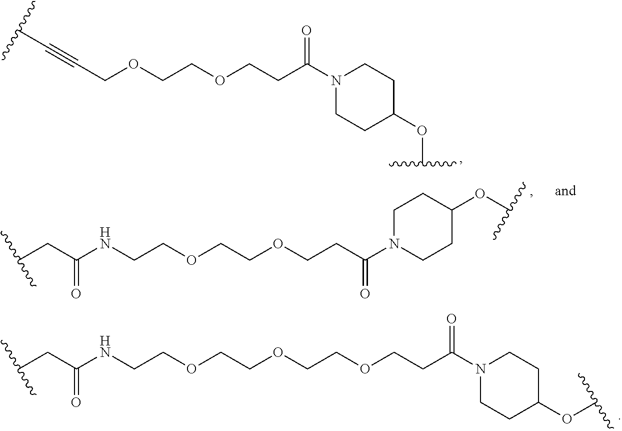

Embodiment 12. The molecule of any one of embodiments 1-11, wherein M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.
Embodiment 13. The molecule of any one of embodiments 1-12, wherein M_{E3} is a moiety derived from thalidomide, pomalidomide, lenalidomide, iberdomide, (S,R,S)-AHPC-Me hydrochloride, (S,R,S)-AHPC-Me dihydrochloride, cereblon modulator 1, thalidomide-propargyl, (S,R,S)-AHPC-propargyl, (S,R,S)-AHPC hydrochloride, CC-885, thalidomide-O—COOH, lenalidomide hemihydrate, thalidomide fluoride, thalidomide-OH, lenalidomide-Br, thalidomide D4, lenalidomide hydrochloride, (S,R,S)-AHPC-Me, clAP1 ligand 1, TD-106, E3 ligase Ligand 8, E3 ligase Ligand 9, E3 ligase Ligand 10, E3 ligase Ligand 13, E3 ligase Ligand 14, E3 ligase Ligand 18, BC-1215, VHL ligand 1 (VHL-1), VHL ligand 2 (VHL-2), VHL Ligand 8 (VHL-8), VH032, VH032-cyclopropane-F, VH032 thiol, VH-298, VL-269, VL-285, LCL161, hydroxyproline-based ligands, HIF-1α-derived (R)-hydroxyproline, Nutlin carboxylic acid, (4R,5S)-Nutlin carboxylic acid, (S,R,S)-AHPC-Boc, AR antagonist 1, NV03, (S,R,S)-AHPC TFA, (S,R,S)-AHPC, P-Naphthoflavone-CH2—Br, β-Naphthoflavone-CH2—OH, Bestatin-amido-Me, MV-1-NH-Me, (S,S,S)-AHPC hydrochloride, and clAP1 ligand 2.
Embodiment 14. The molecule of any one of embodiments 1-13, wherein M_{E3} has a formula selected from the group consisting of:

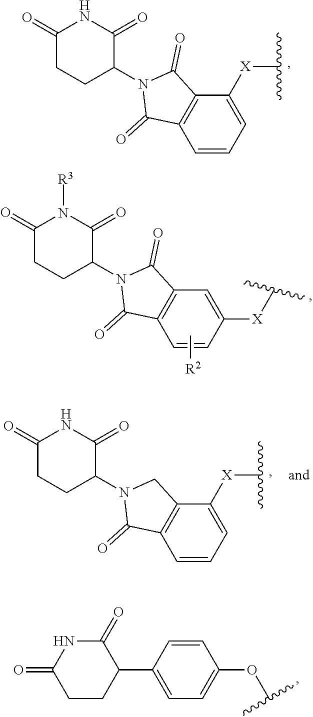

wherein
X is a bond, NH, or NMe;
R² is hydrogen or halo; and
R³ is hydrogen or methyl.
Embodiment 15. The molecule of any one of embodiments 1-14, wherein M_{E3} has a formula selected from the group consisting of

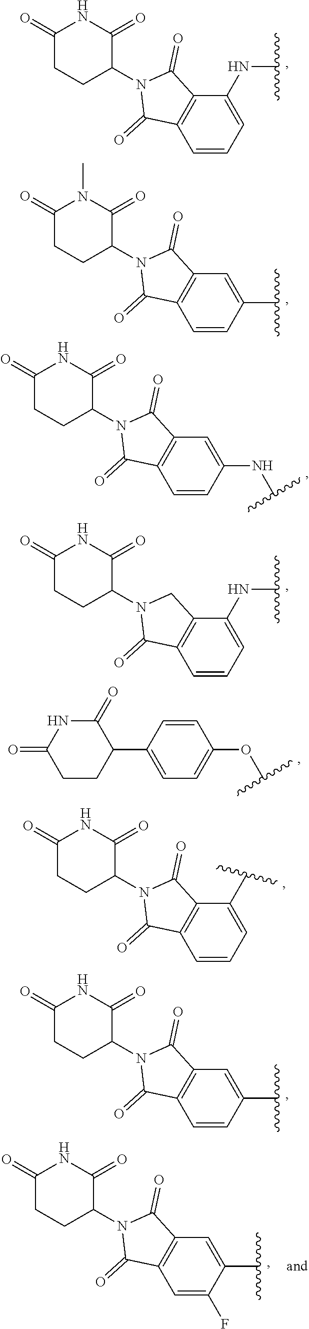

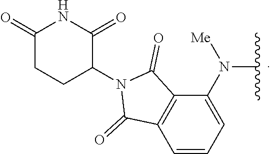 [SMILES: *N(C)c1cccc2c1C(=O)N(C1CCC(=O)NC1=O)C2=O]

Embodiment 16. The molecule of any one of embodiments 1-15, wherein the molecule has a formula selected from the group consisting of

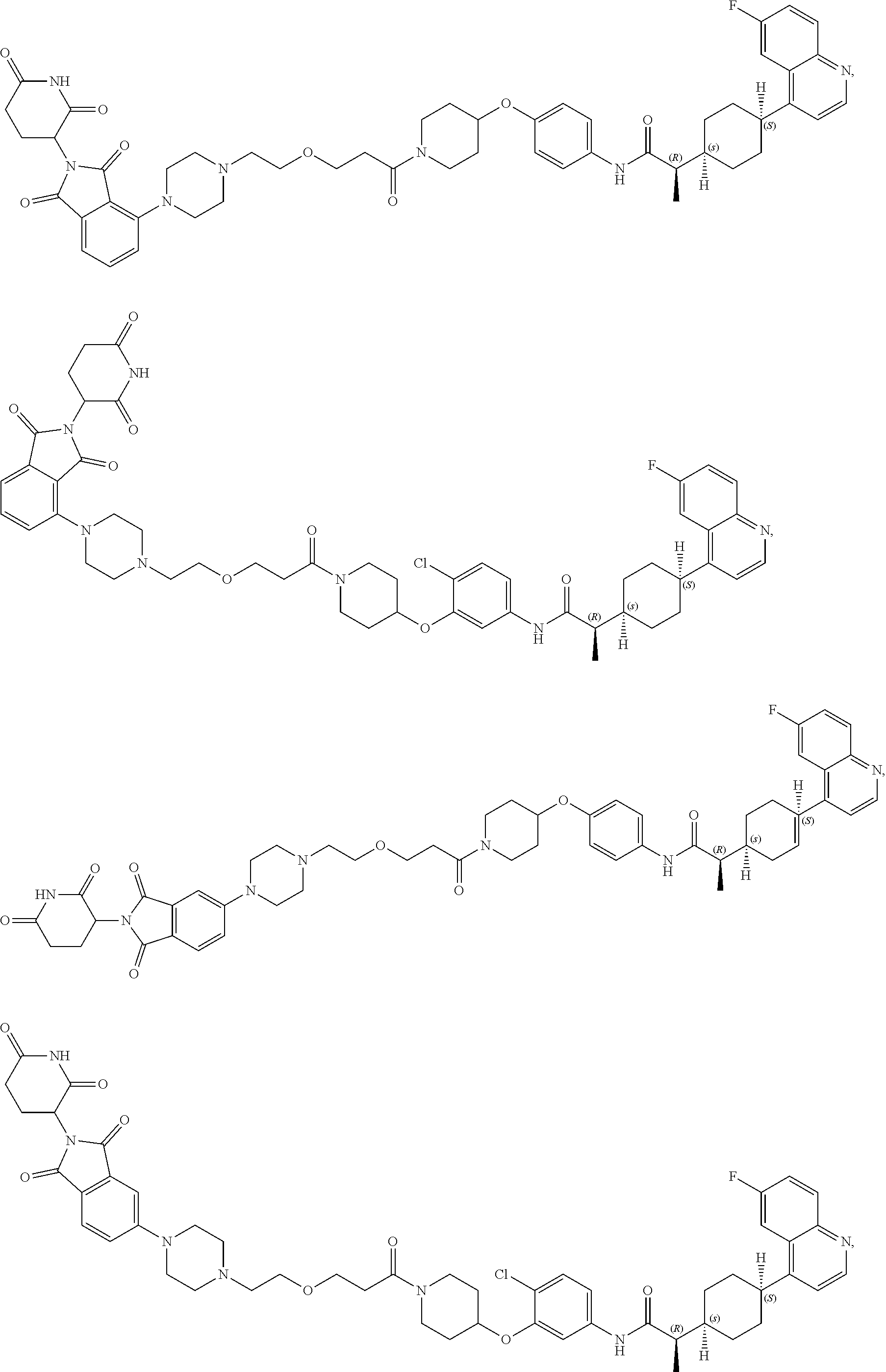

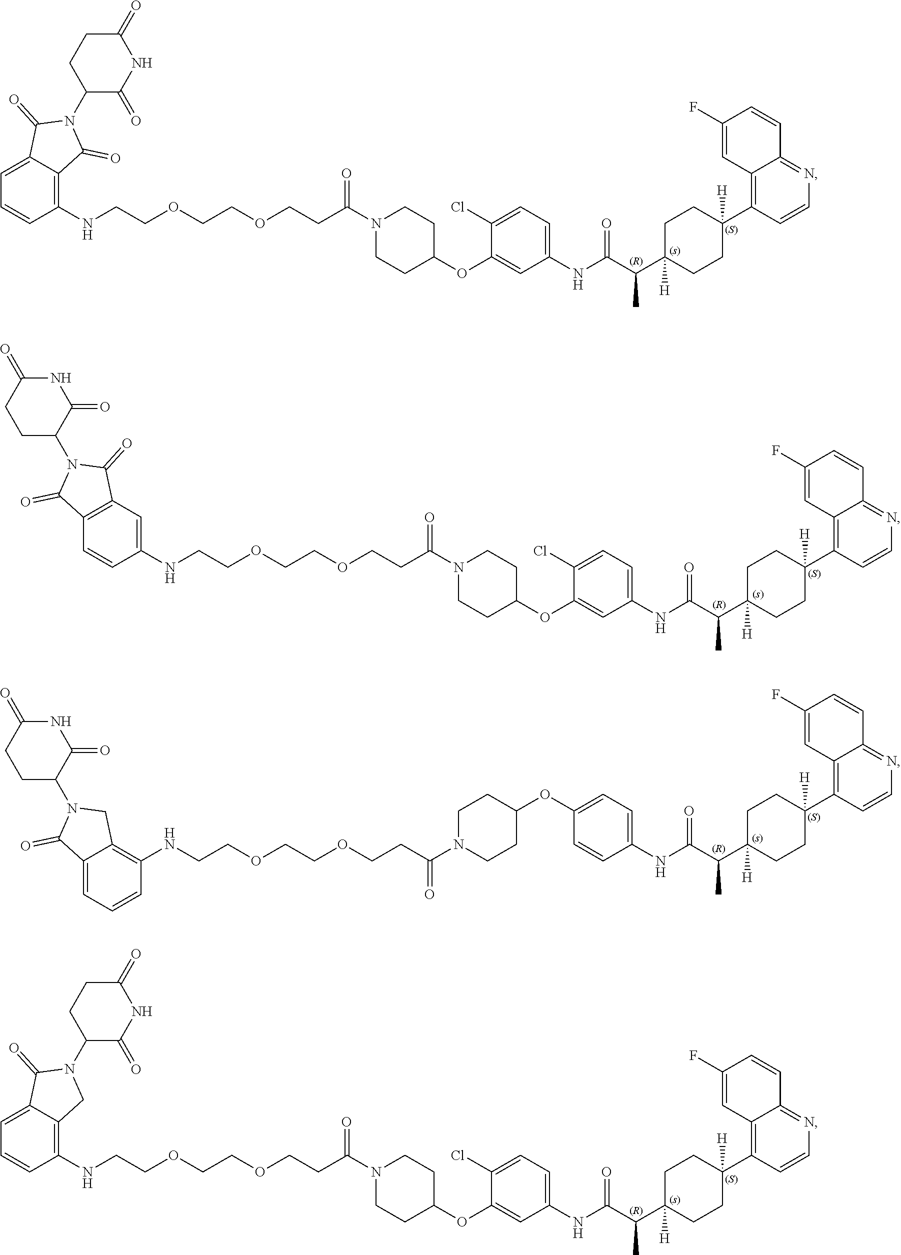

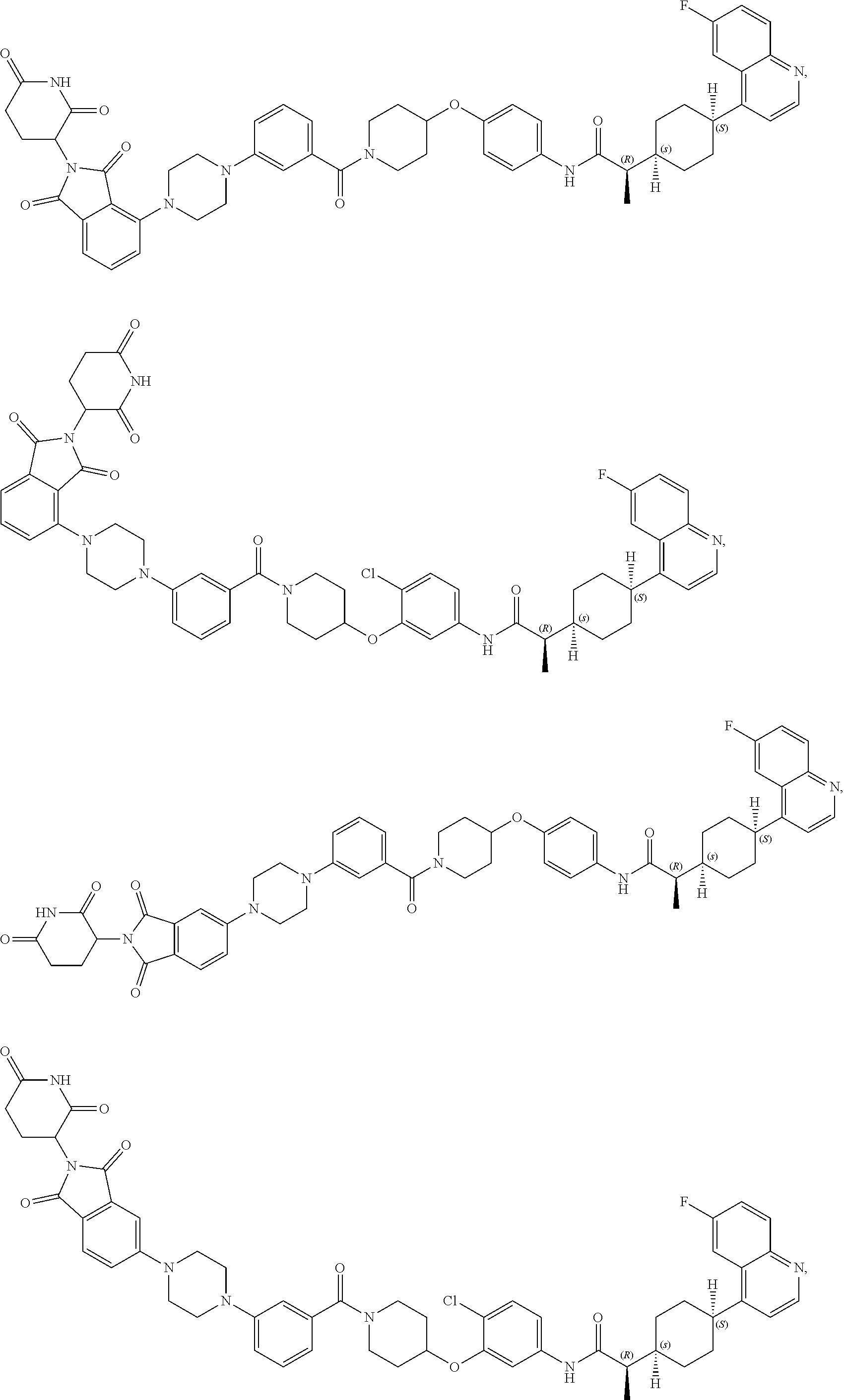

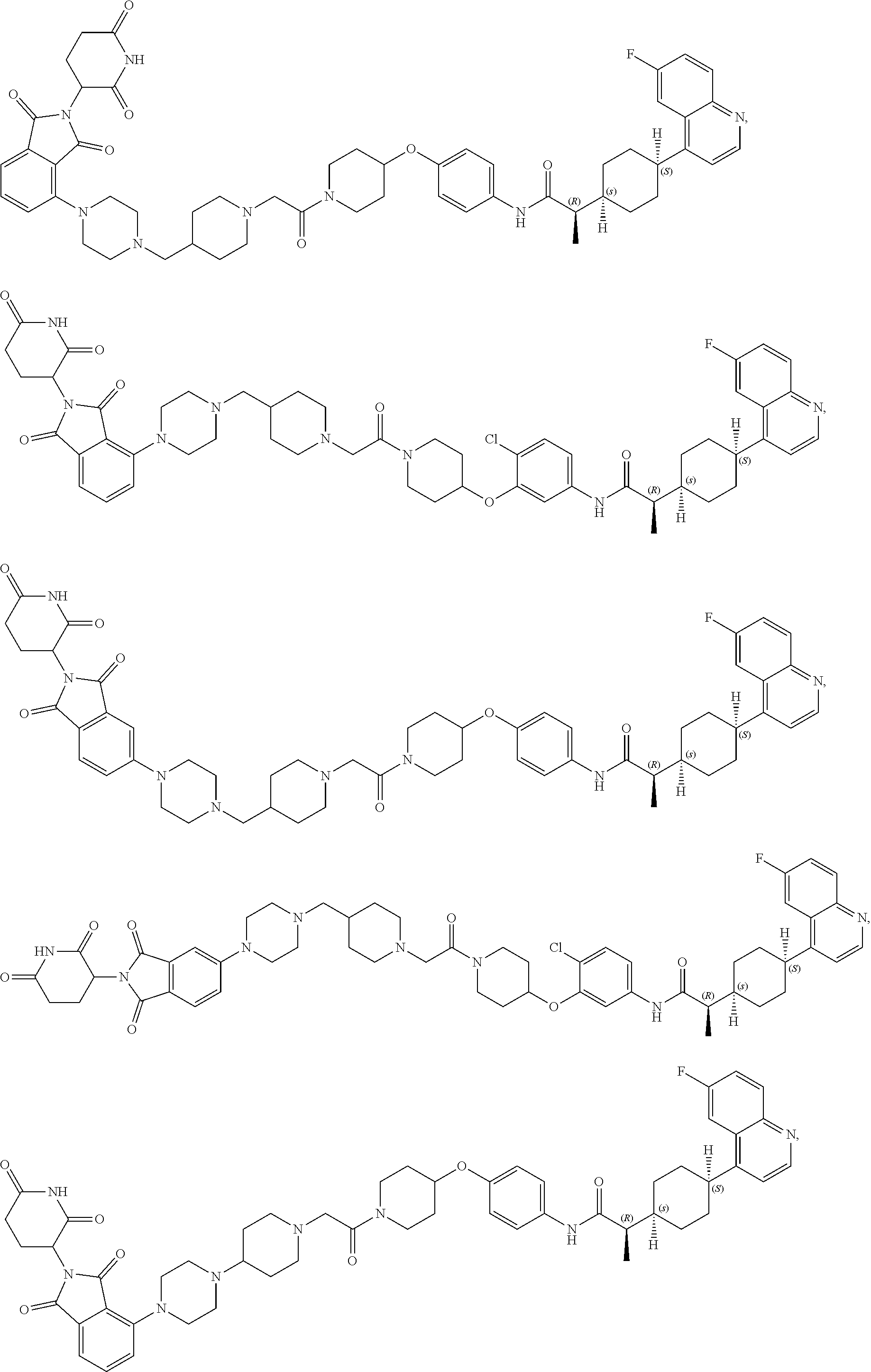

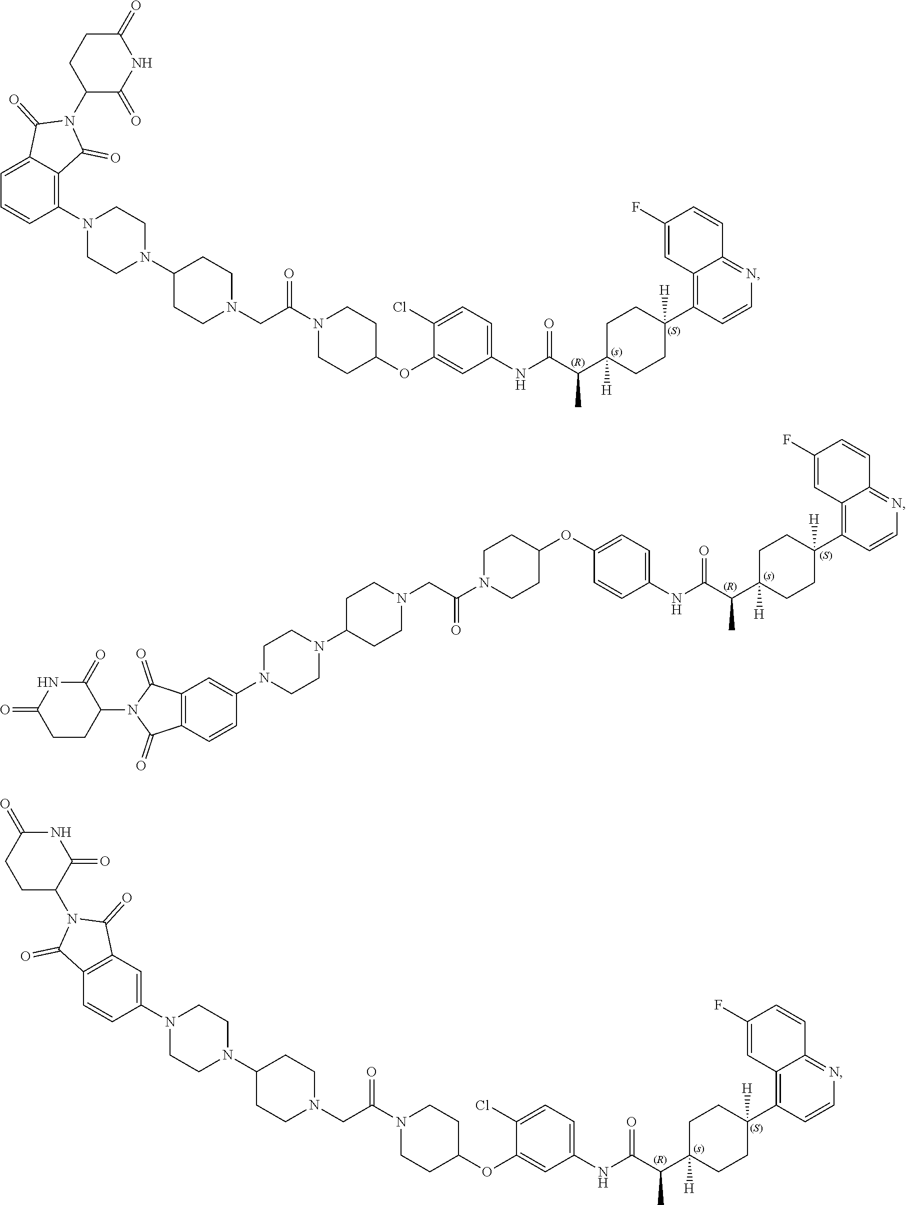

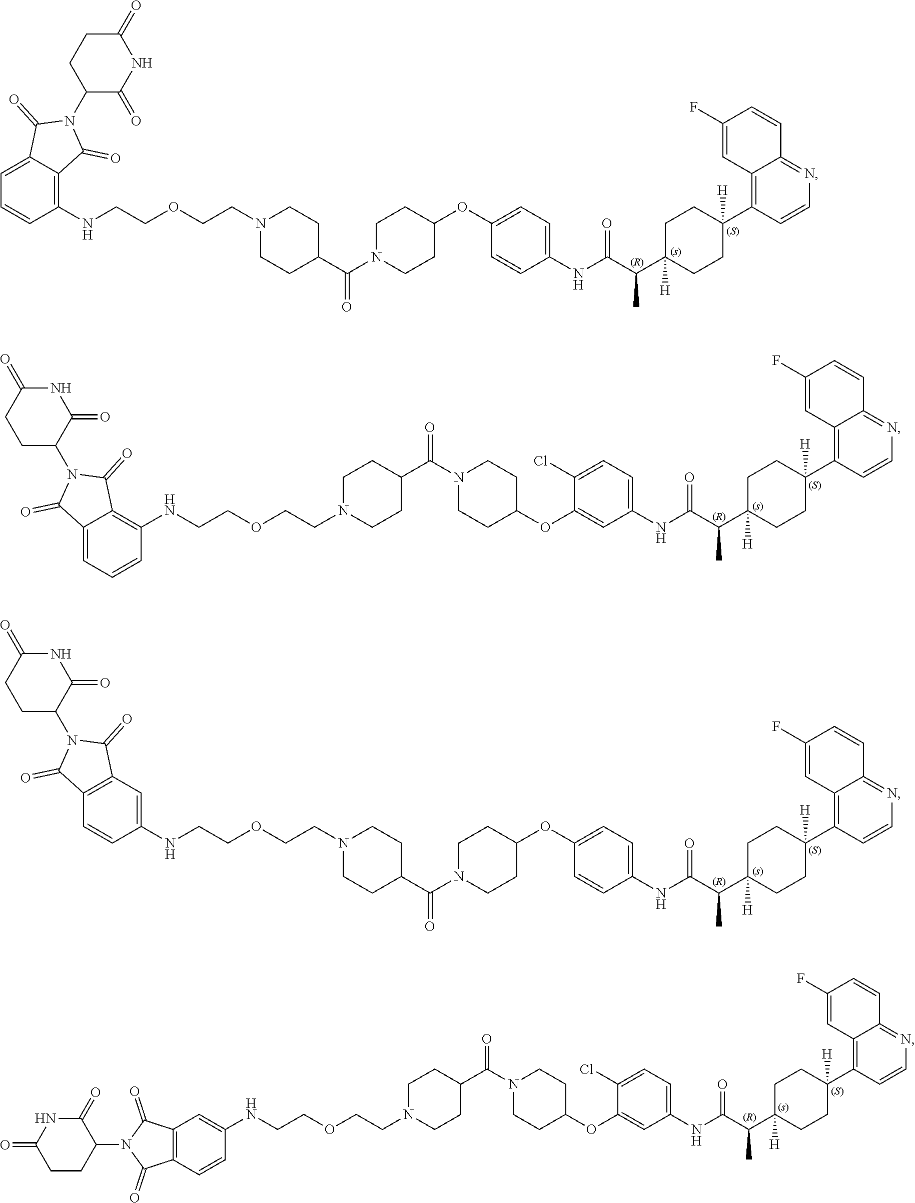

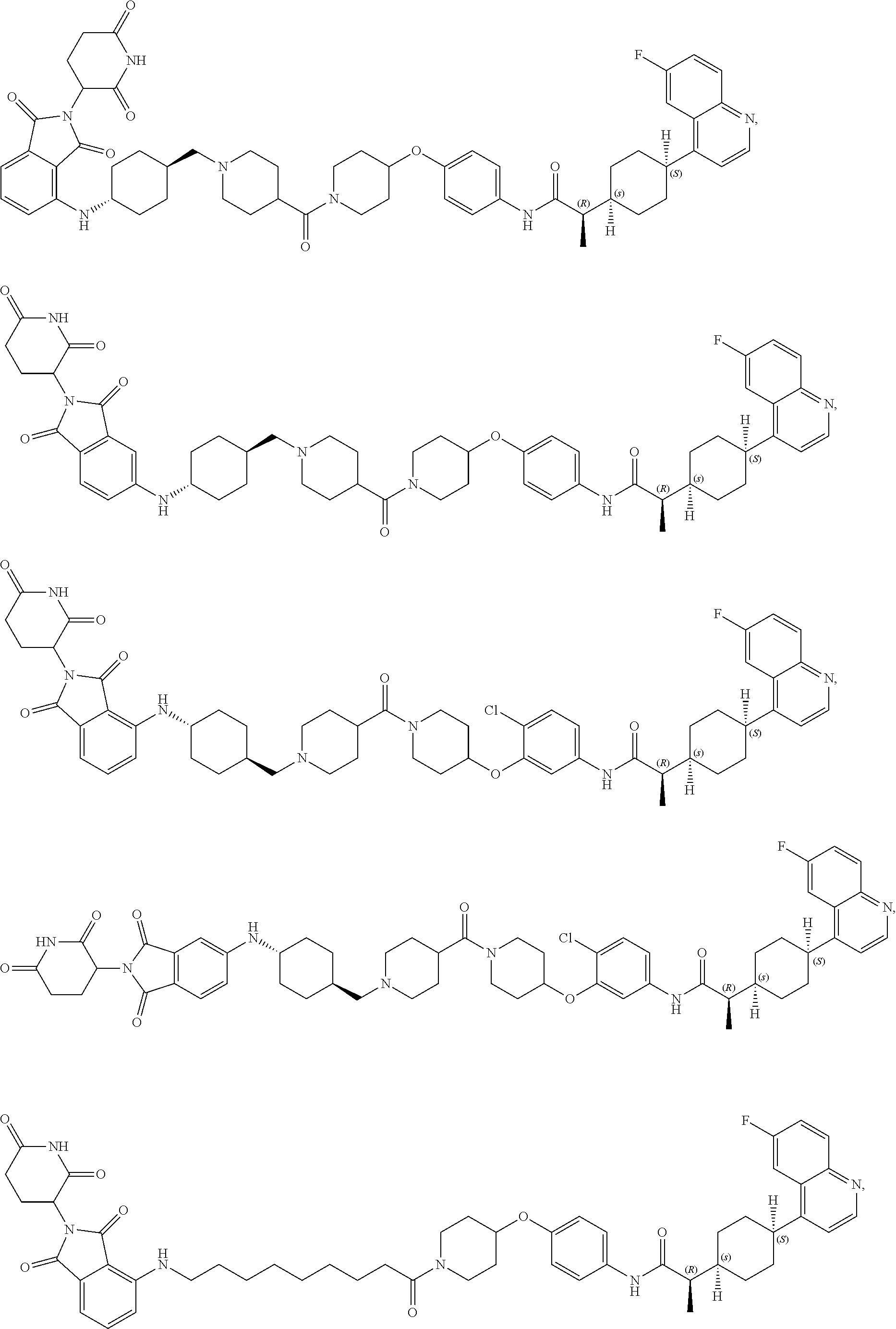

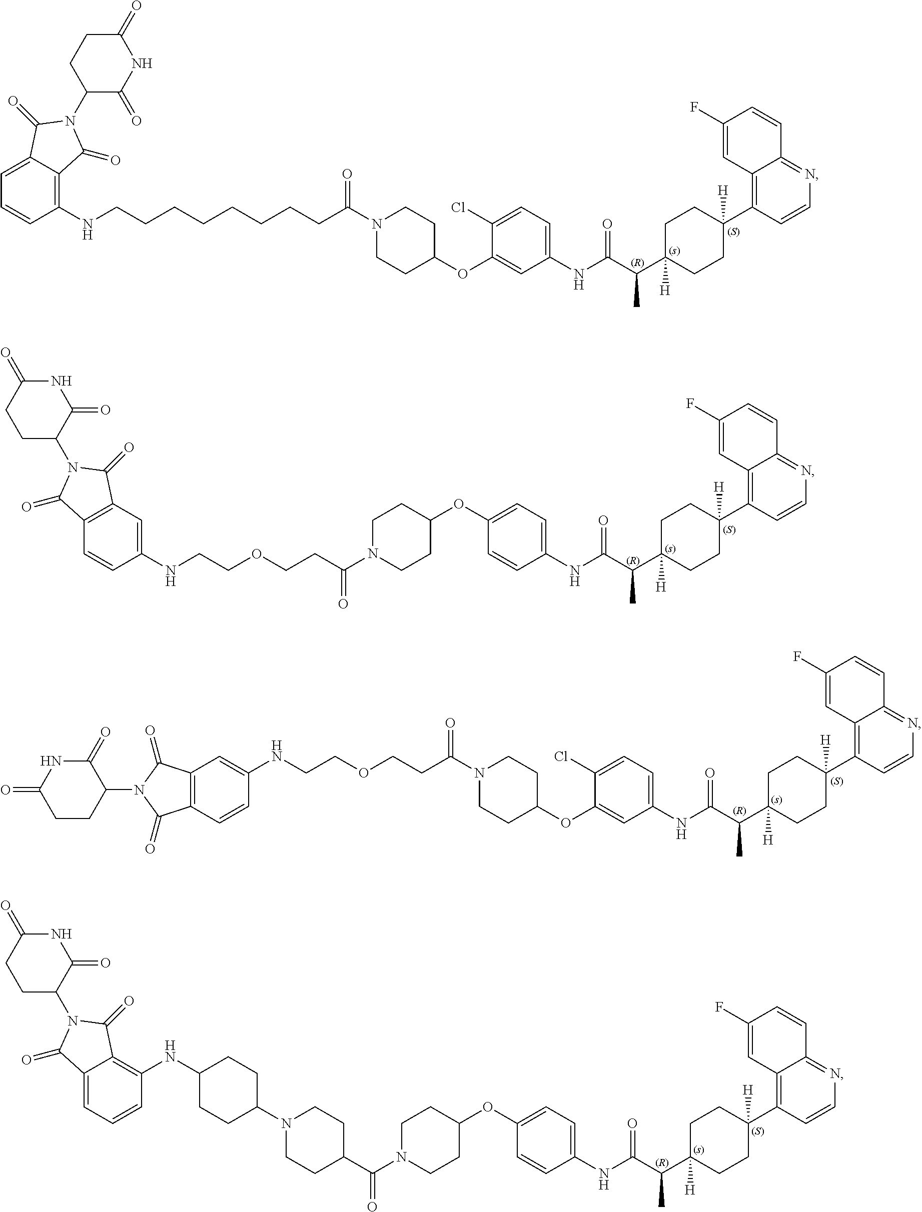

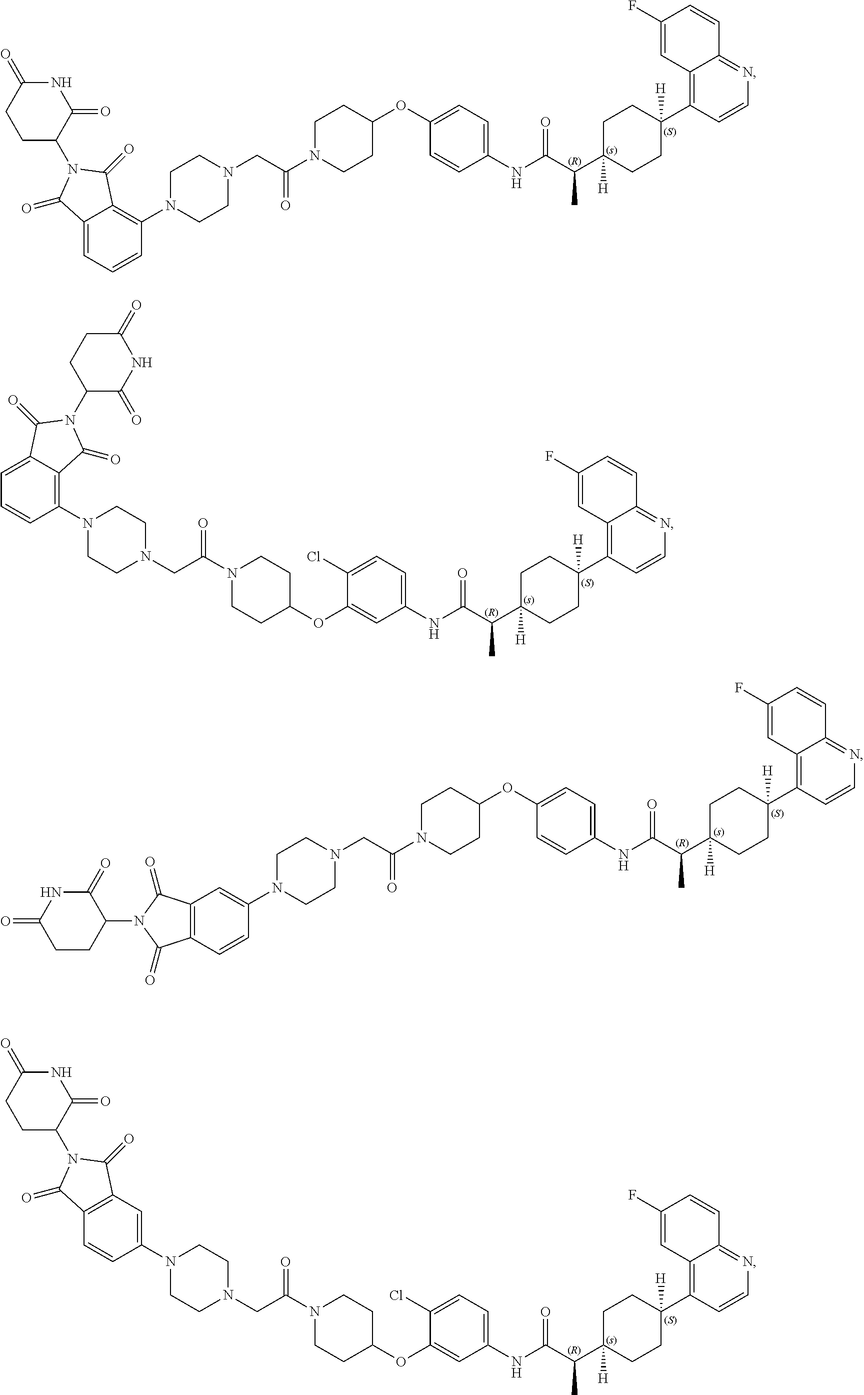

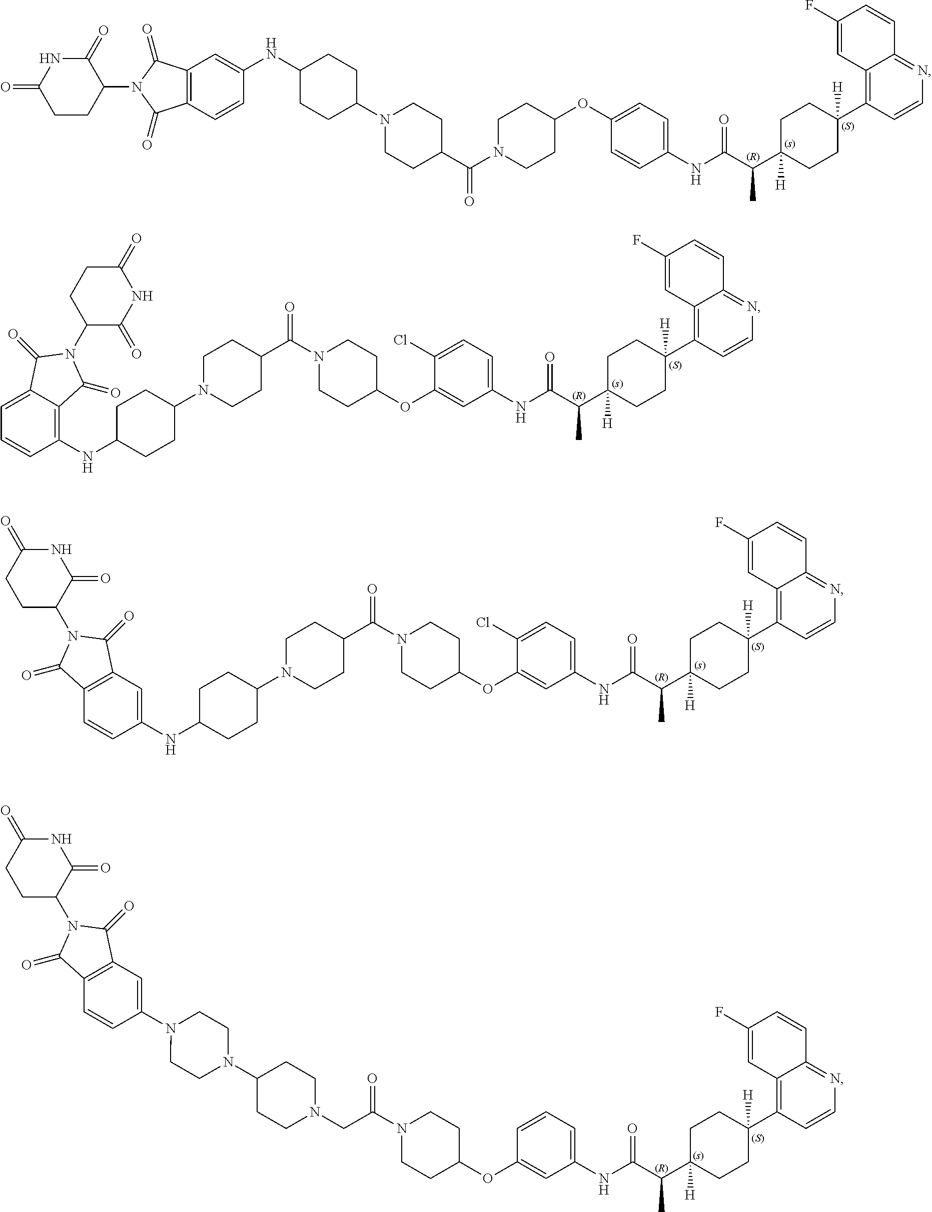

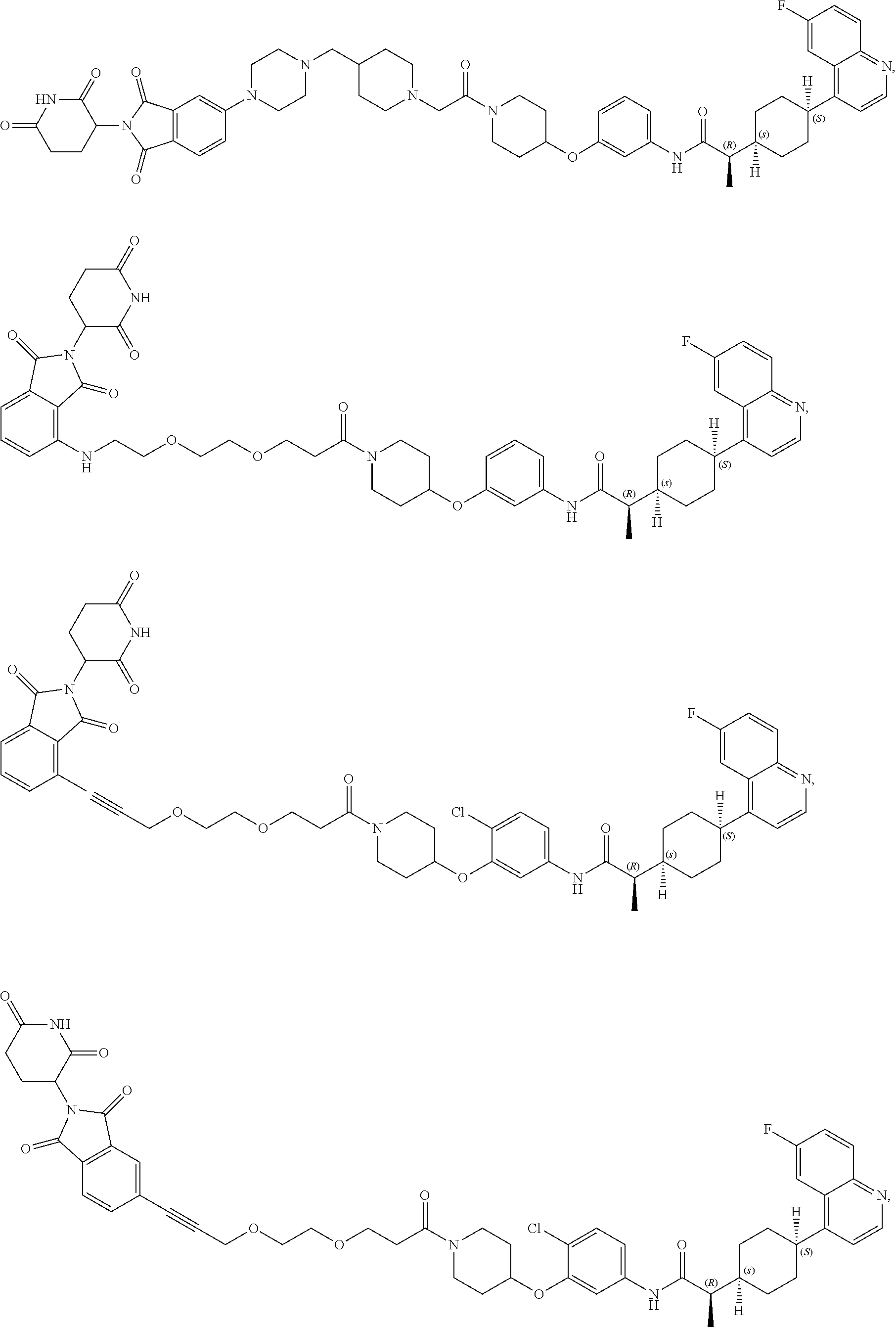

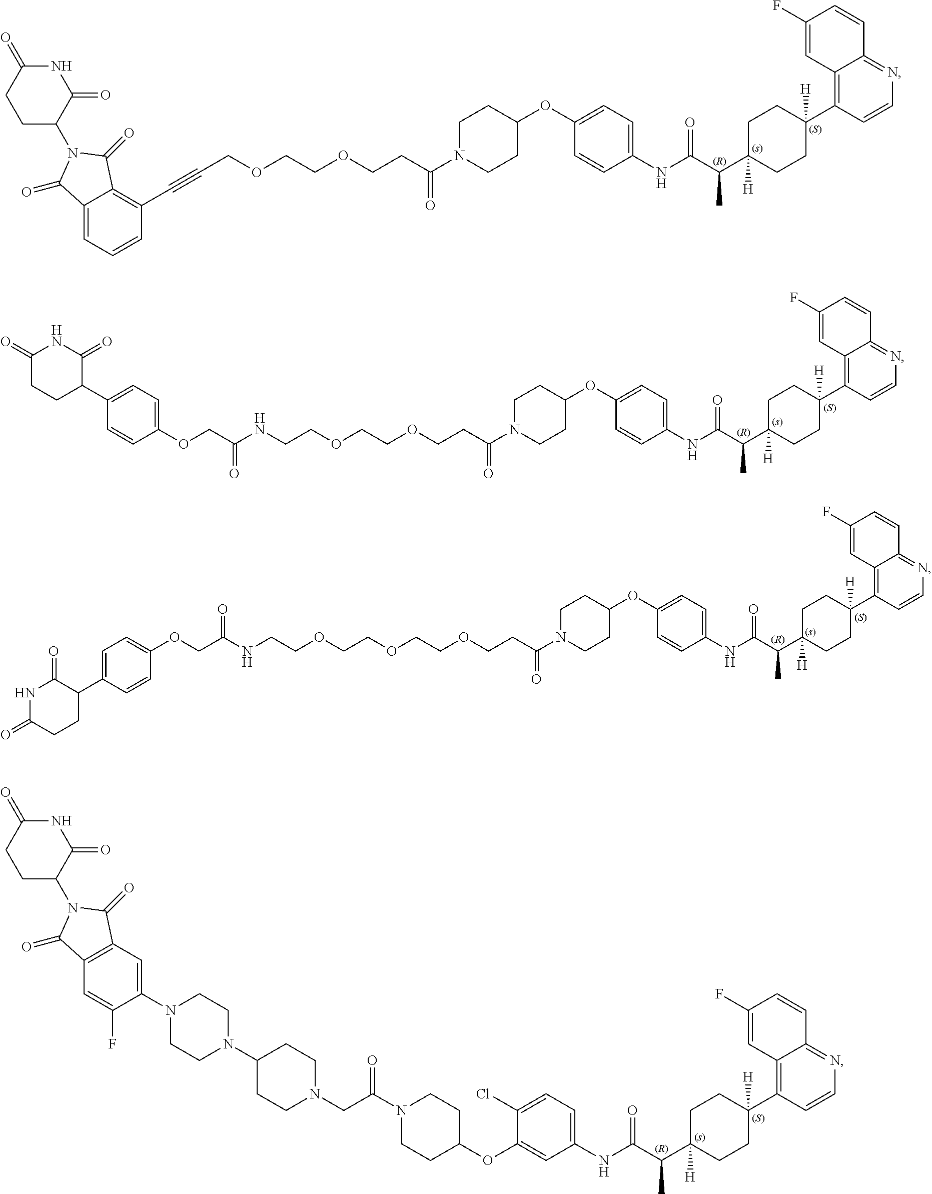

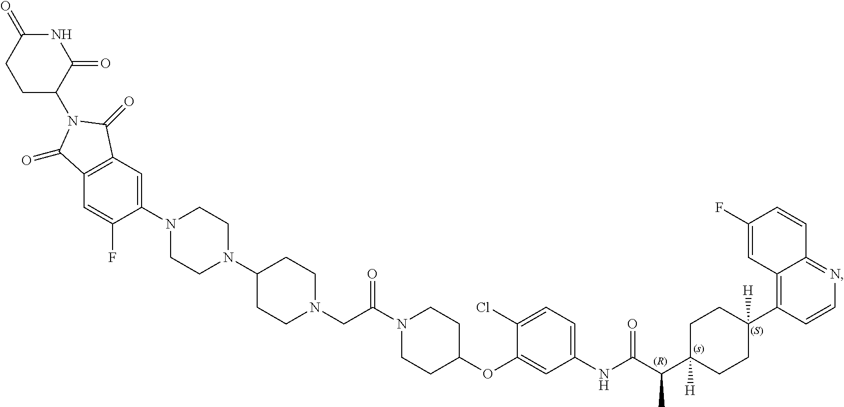

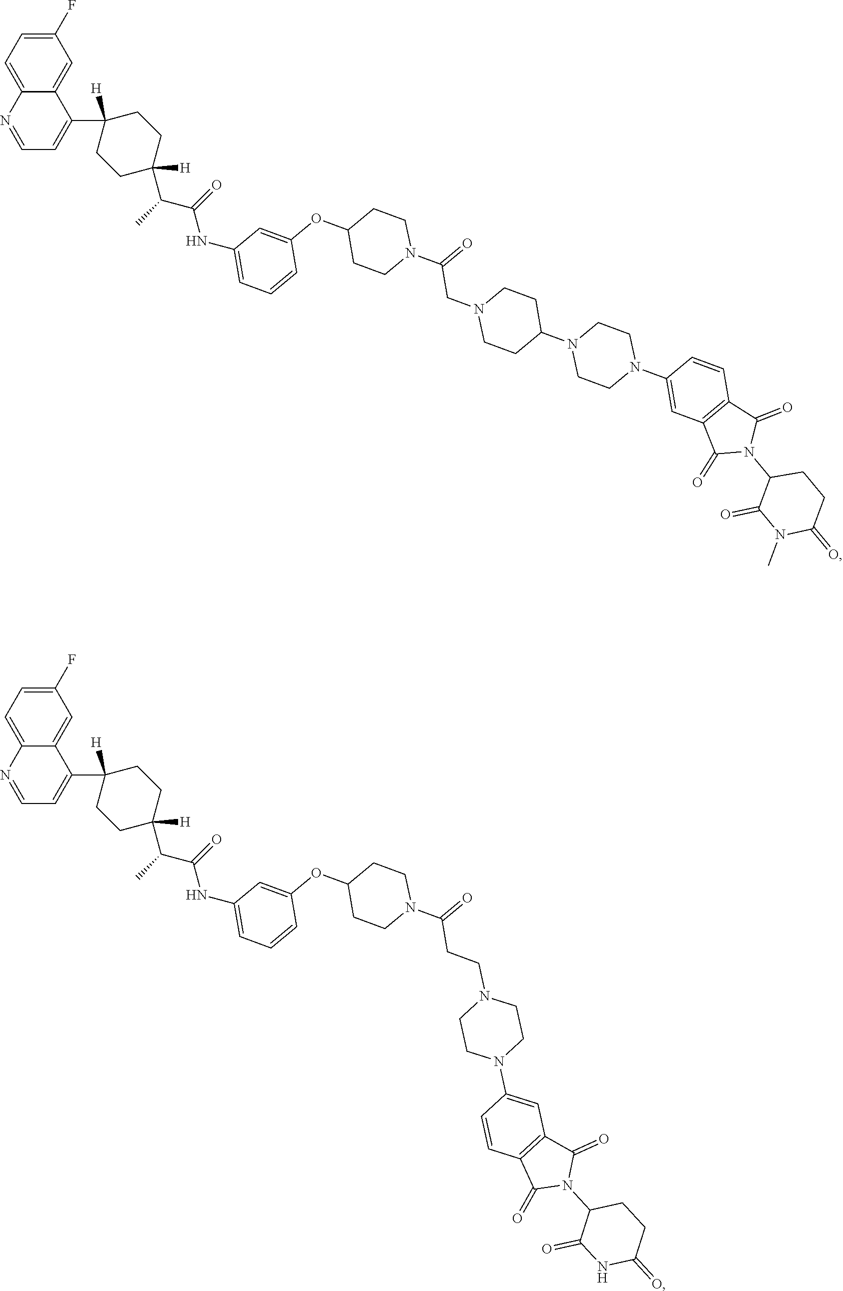 [SMILES: C[C@@H](C(=O)Nc1cccc(OC2CCN(C(=O)CN3CCC(N4CCN(c5ccc6c(c5)C(=O)N(C5CCC(=O)N(C)C5=O)C6=O)CC4)CC3)CC2)c1)[C@H]1CC[C@@H](c2ccnc3ccc(F)cc32)CC1 ; C[C@@H](C(=O)Nc1cccc(OC2CCN(C(=O)CCN3CCN(c4ccc5c(c4)C(=O)N(C4CCC(=O)NC4=O)C5=O)CC3)CC2)c1)[C@H]1CC[C@@H](c2ccnc3ccc(F)cc32)CC1]

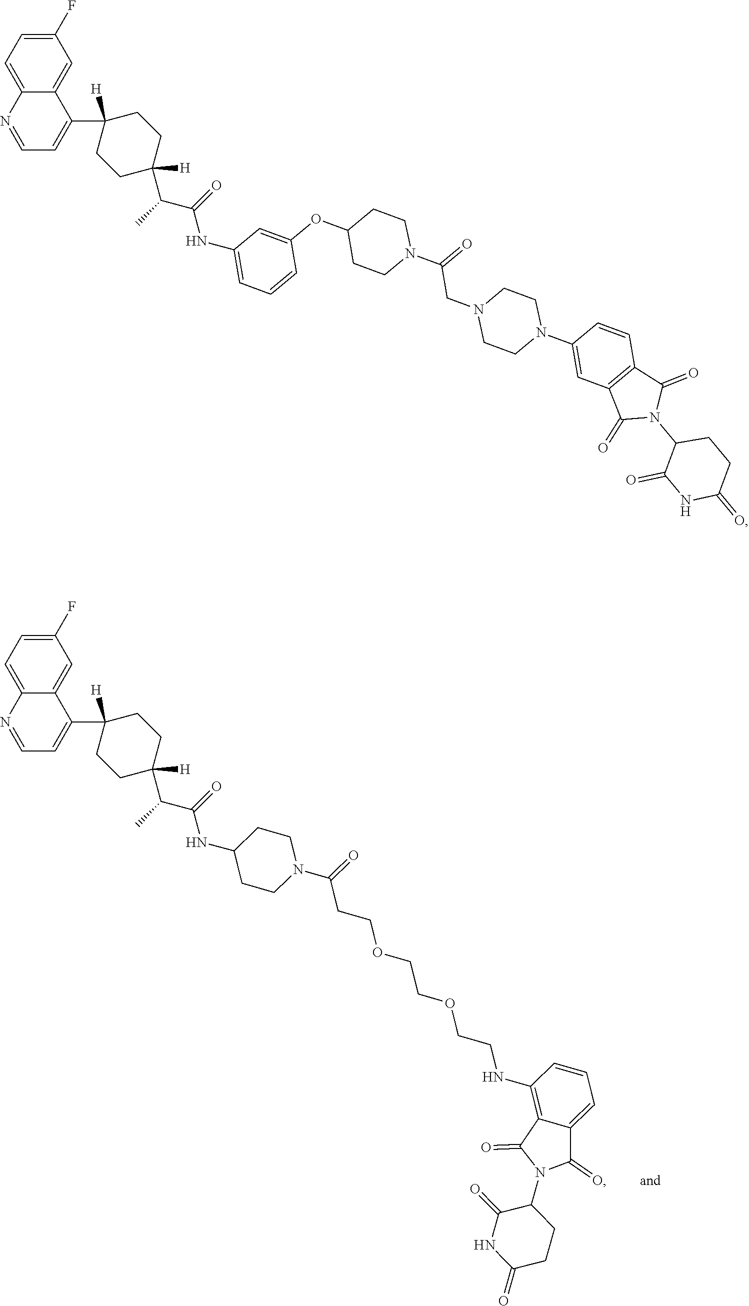 [SMILES: C[C@@H](C(=O)Nc1cccc(OC2CCN(C(=O)CN3CCN(c4ccc5c(c4)C(=O)N(C4CCC(=O)NC4=O)C5=O)CC3)CC2)c1)[C@H]1CC[C@@H](c2ccnc3ccc(F)cc32)CC1 ; C[C@@H](C(=O)NC1CCN(C(=O)CCOCCOCCNc2cccc3c2C(=O)N(C2CCC(=O)NC2=O)C3=O)CC1)[C@H]1CC[C@@H](c2ccnc3ccc(F)cc32)CC1]

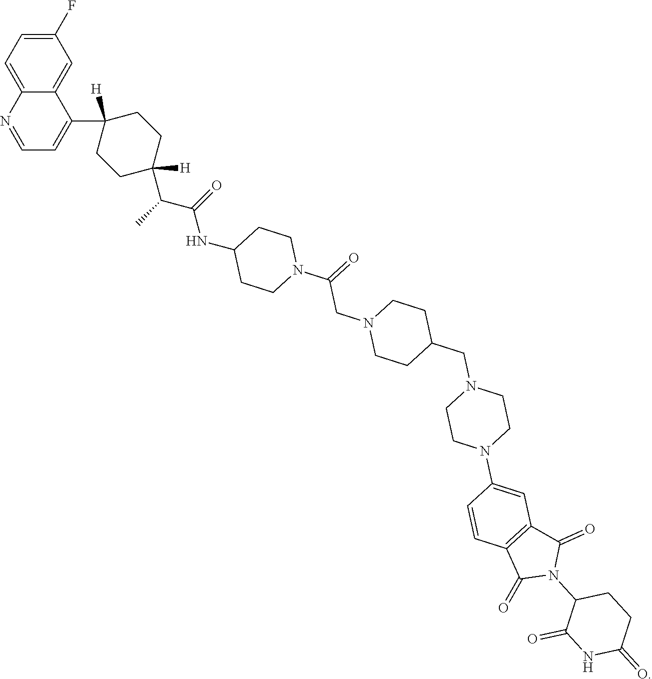 [SMILES: C[C@@H](C(=O)NC1CCN(C(=O)CN2CCC(CN3CCN(c4ccc5c(c4)C(=O)N(C4CCC(=O)NC4=O)C5=O)CC3)CC2)CC1)[C@H]1CC[C@@H](c2ccnc3ccc(F)cc32)CC1]

Embodiment 17. The molecule of any one of embodiments 1-16, wherein the molecule has a formula selected from the group consisting of

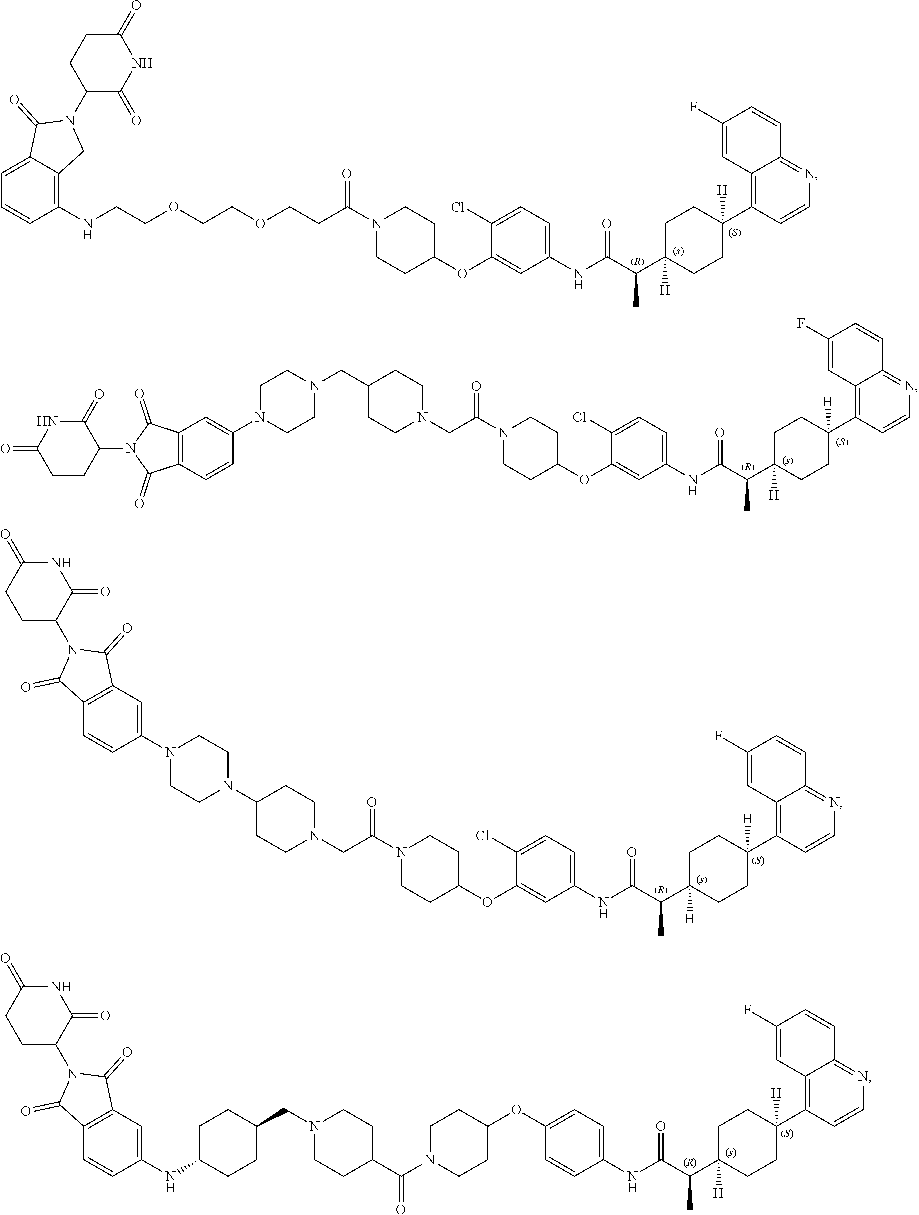

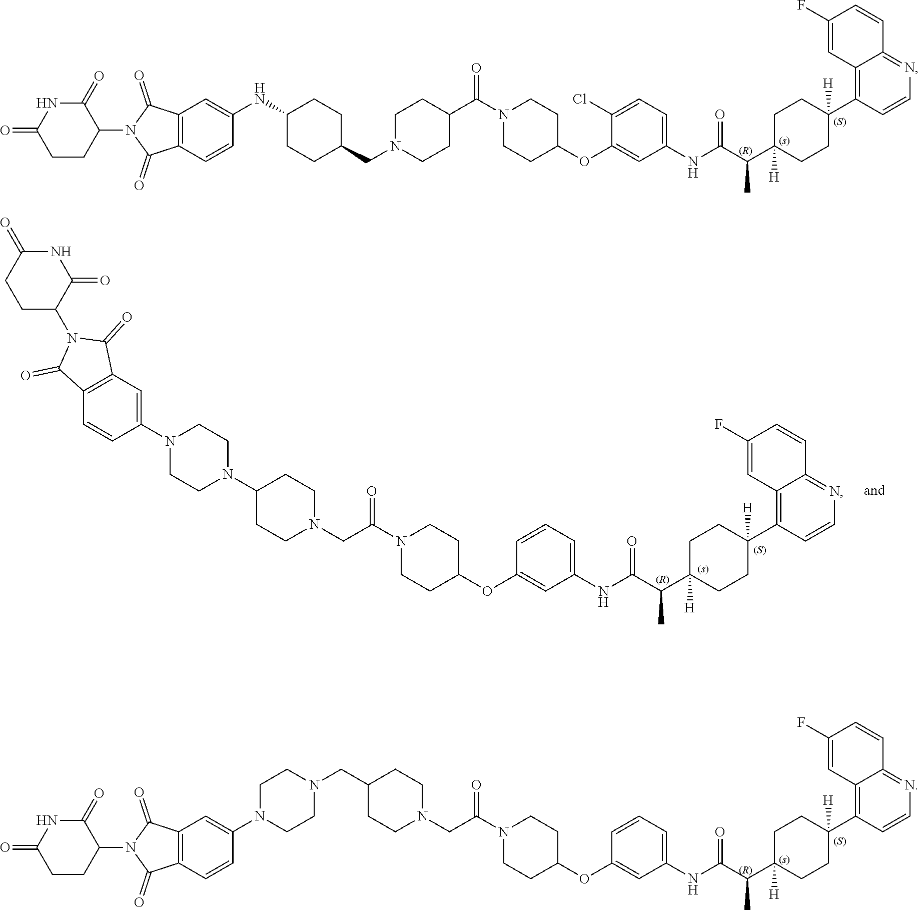

Embodiment 18. The molecule of any one of embodiments 1-17, wherein the molecule has a formula

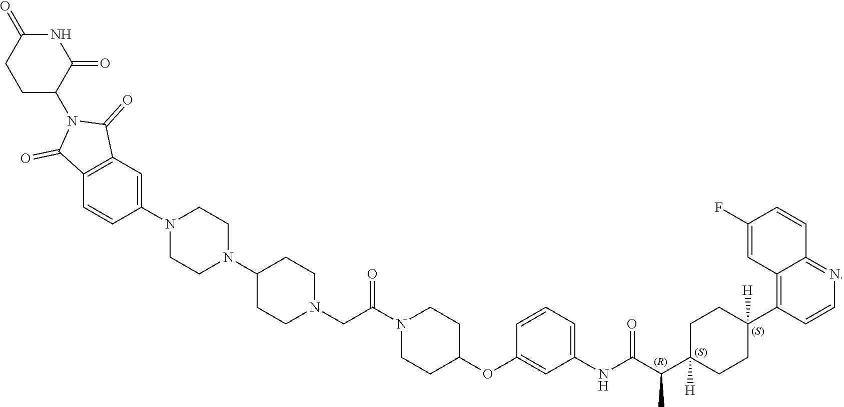

Embodiment 19. The molecule of any one of embodiments 1-18, wherein, under the same conditions, the molecule is more potent than the compound having a formula

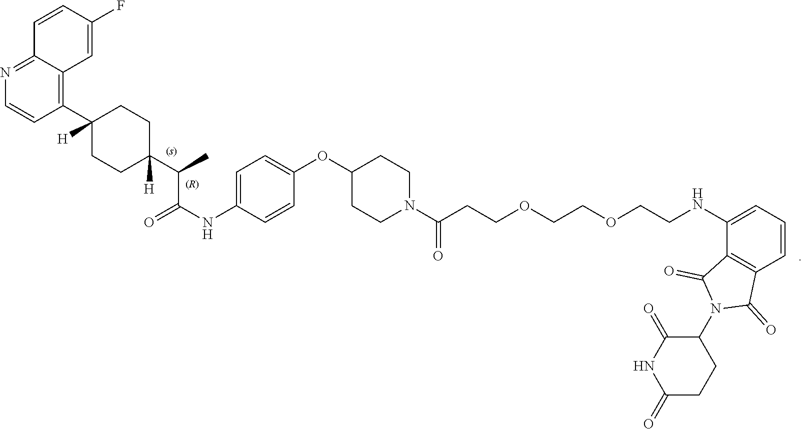

Embodiment 20. A pharmaceutical composition comprising a therapeutically effective amount of the molecule of any one of embodiments 1-19, or a pharmaceutically acceptable salt thereof, and a pharmaceutically acceptable carrier, excipient, or diluent.
Embodiment 21. A method of treating cancer in a subject in need thereof, the method comprising administering the molecule of any one of embodiments 1-19, or a pharmaceutically acceptable salt thereof, or the pharmaceutical composition of embodiment 20 to the subject having the cancer.
Embodiment 22. The method of embodiment 21, wherein the cancer is selected from multiple glioblastoma, colorectal cancer, myeloma, leukemia, non-small cell lung cancer, colon cancer, cancer of the central nervous system, melanoma, ovarian cancer, renal cancer, prostate cancer, uterine cancer, pancreatic cancer, and breast cancer.
Embodiment 23. The method of embodiment 21 or 22, wherein the cancer is glioblastoma.
Embodiment 24. The method of any one of embodiments 21-23, wherein the method further comprises administering a therapy comprising chemotherapy, radiation, checkpoint inhibitors, or immunotherapy to the subject, wherein the therapy is administered before, concurrently with, or after administering the pharmaceutical composition of embodiment 20.
Embodiment 25. The method of any one of embodiments 21-24, wherein the method further comprises administering an anti-PD1 agent to the subject, before, concurrently with, or after administering the pharmaceutical composition of embodiment 20.
Embodiment 26. The method of any one of embodiments 21-25, wherein the method further comprises administering an anti-PD-L1 agent to the subject, before, concurrently with, or after administering the pharmaceutical composition of embodiment 20.

### EXAMPLES

The following Examples are illustrative and are not intended to limit the scope of the claimed subject matter.

### Example 1

TABLE 1
Structures of IDO1-PROTACs

| ID | Structure |
|---|---|
| NU227039 | 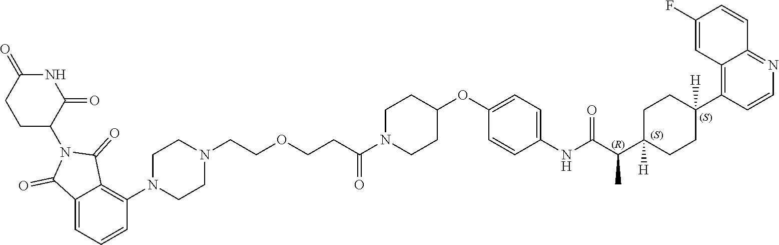 |
| NU227040 | 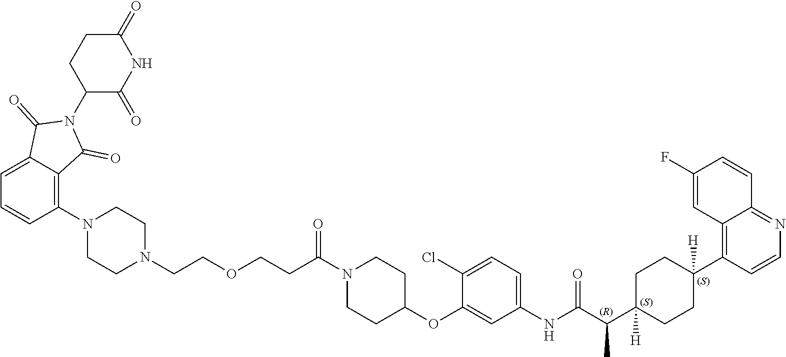 |
| NU227041 | 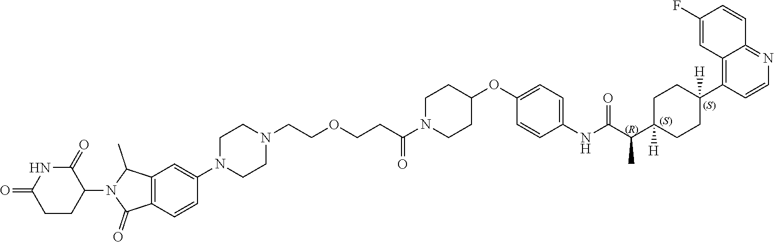 |
| NU227042 | 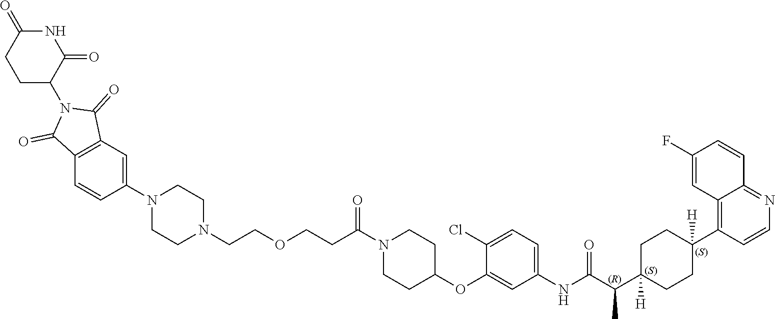 |
| NU227043 | 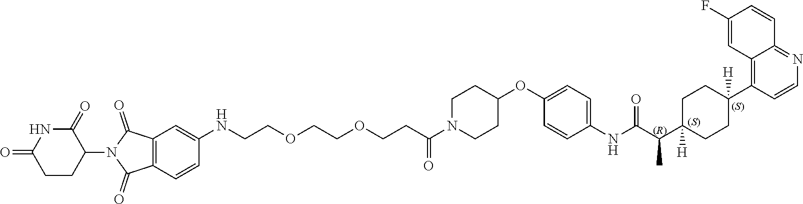 |
| NU227044 | 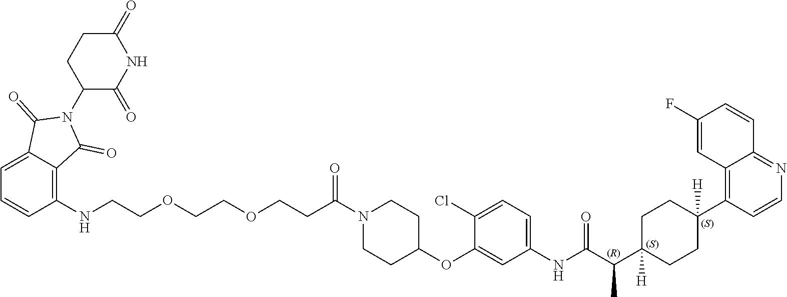 |
| NU227045 | 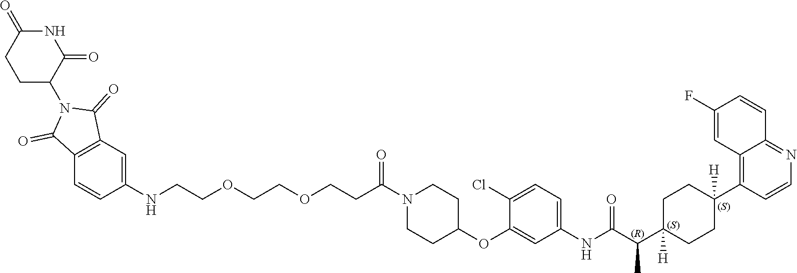 |
| NU227046 | 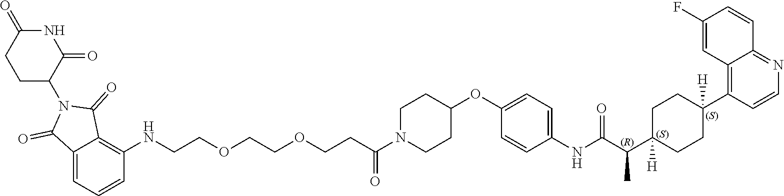 |
| NU227047 | 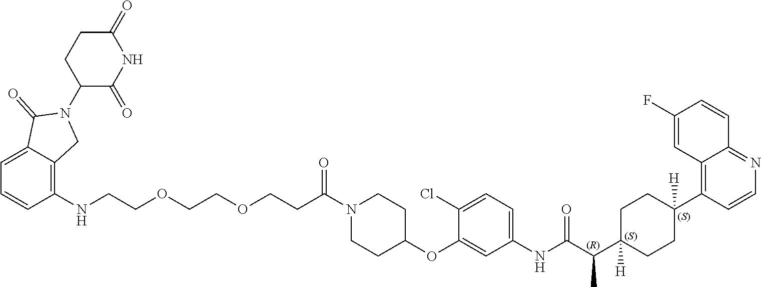 |
| NU227157 | 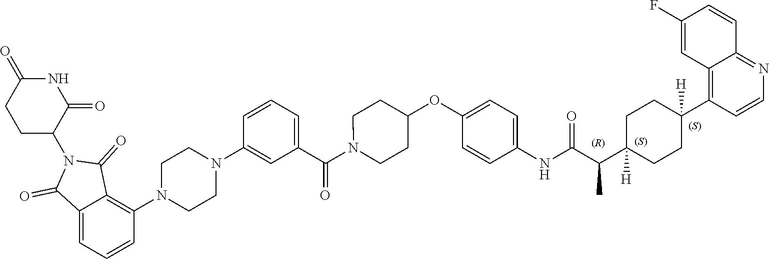 |
| NU227158 | 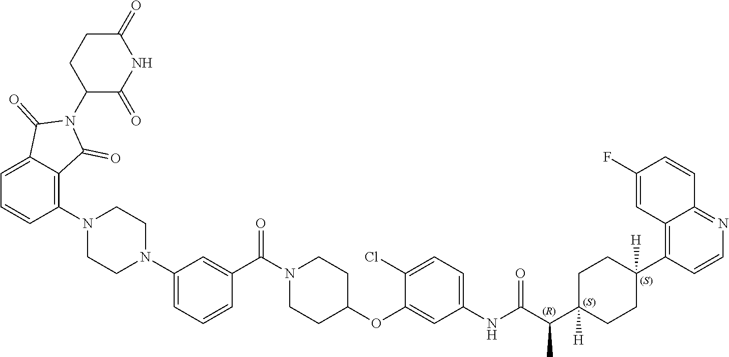 |
| NU227159 | 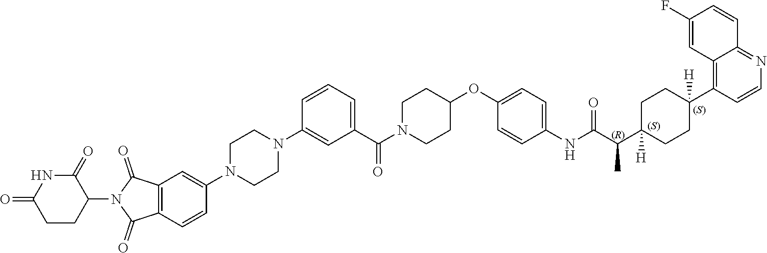 |
| NU227160 | 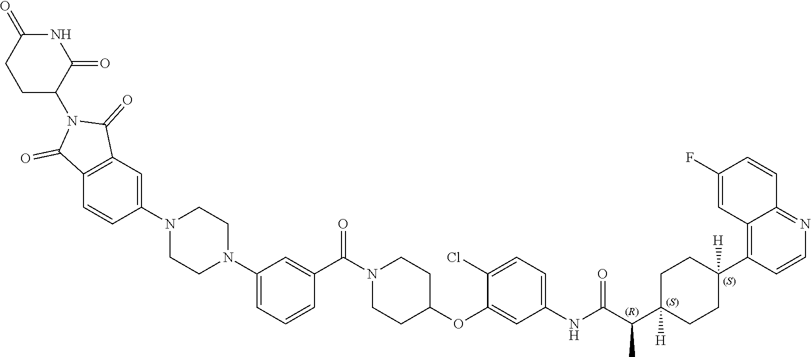 |
| NU227161 | 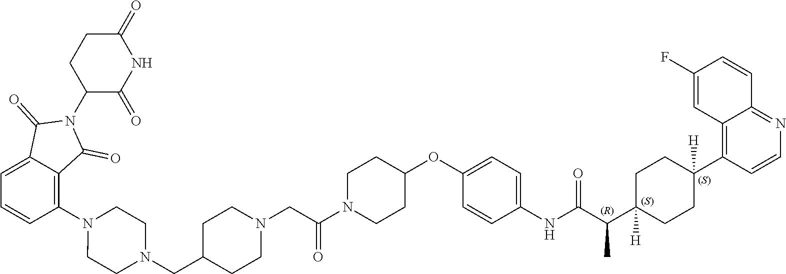 |
| NU227162 | 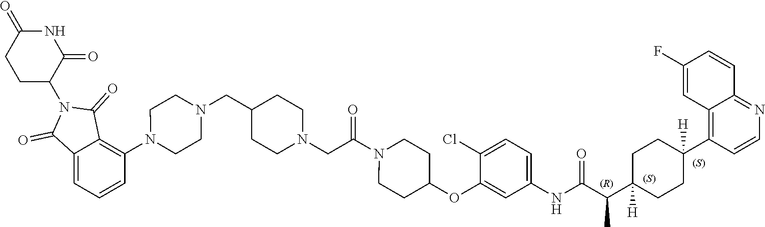 |
| NU227163 | 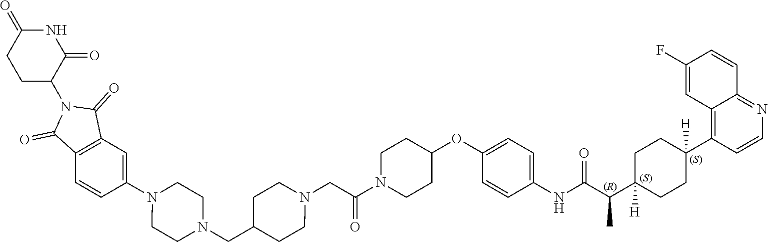 |
| NU227164 | 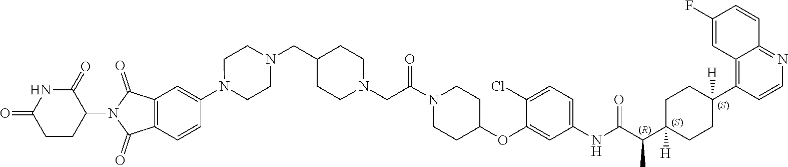 |
| NU227165 | 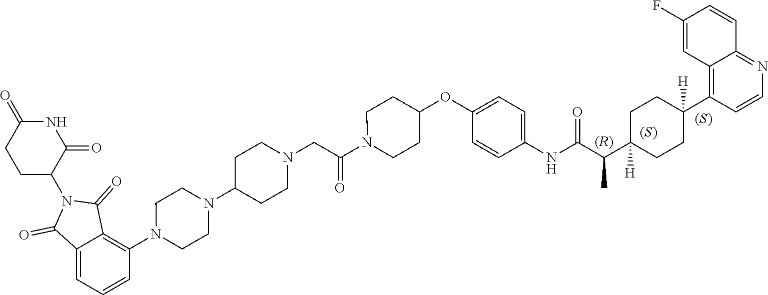 |
| NU227166 | 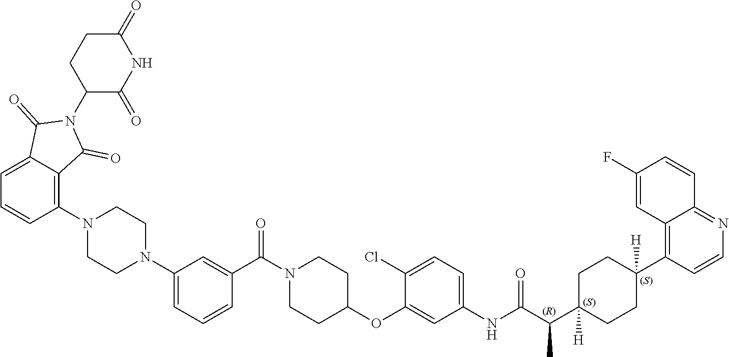 |
| NU227167 | 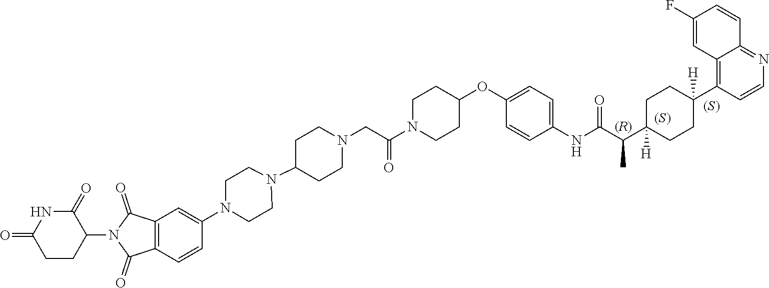 |
| NU227168 | 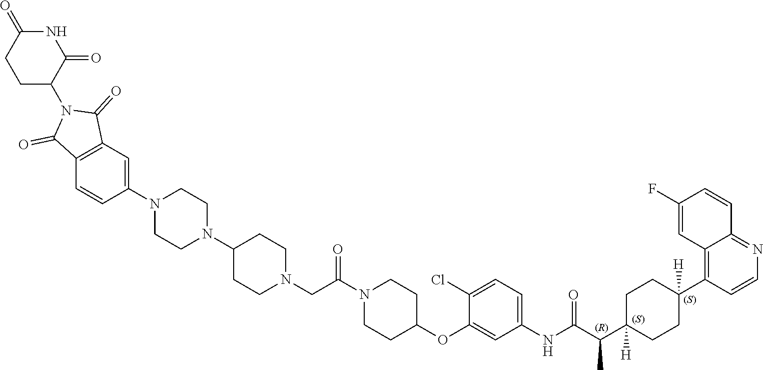 |
| NU227182 | 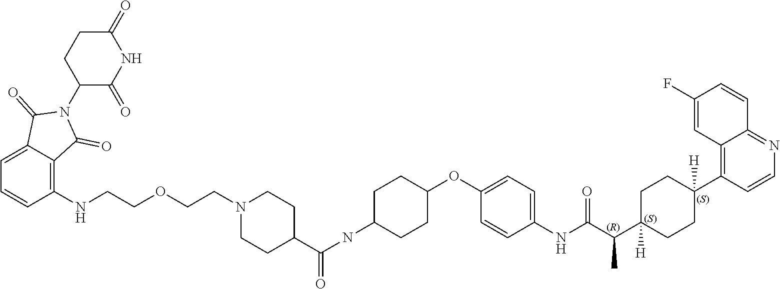 |
| NU227183 | 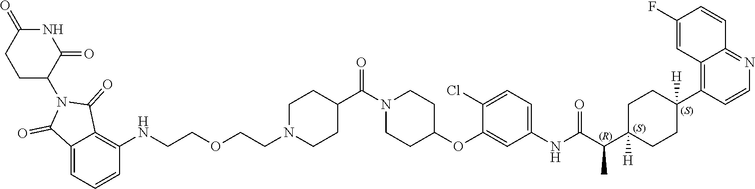 |
| NU227191 | 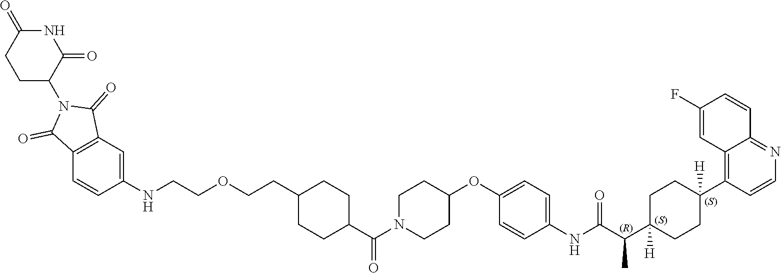 |
| NU227192 | 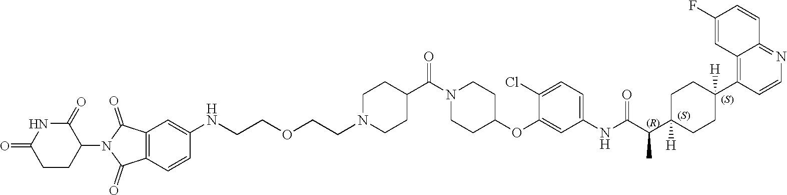 |
| NU227193 | 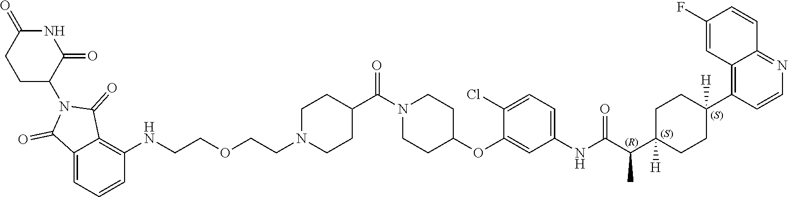 |
| NU227194 | 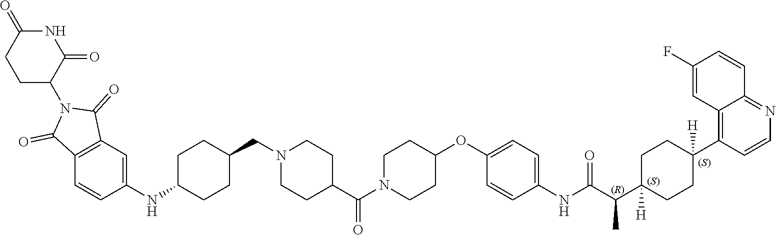 |
| NU227198 | 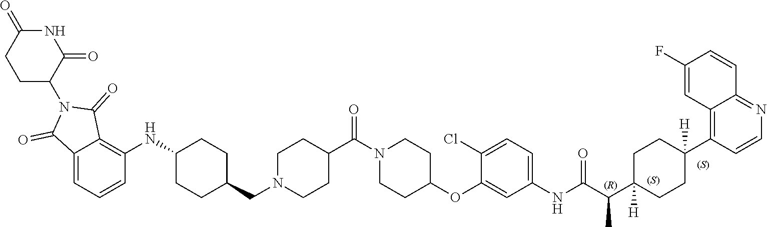 |
| NU227199 | 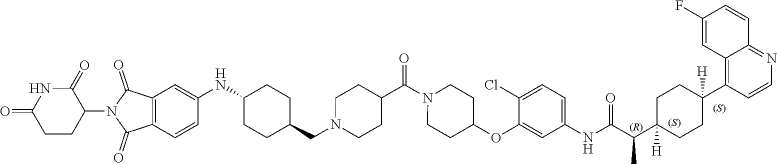 |
| NU227200 | 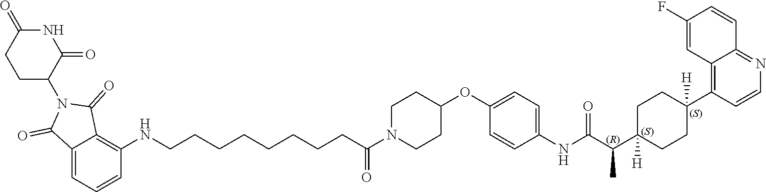 |
| NU227201 | 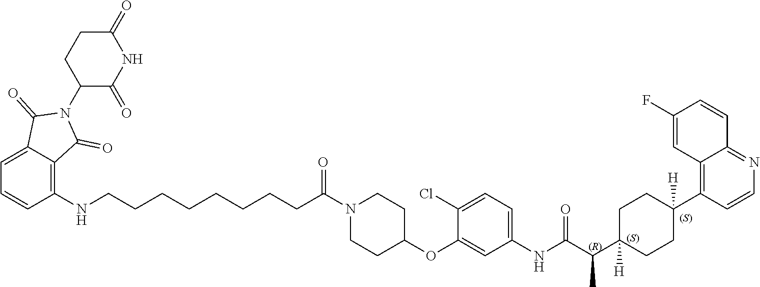 |
| NU227302 | 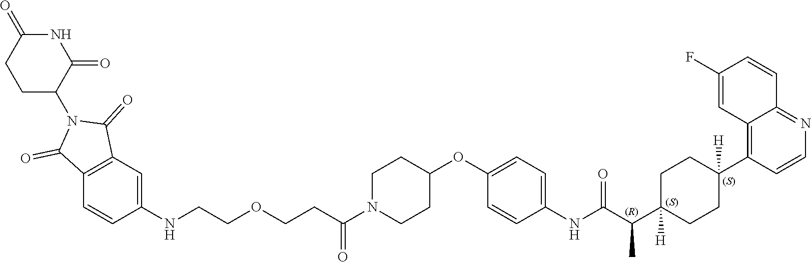 |
| NU227303 | 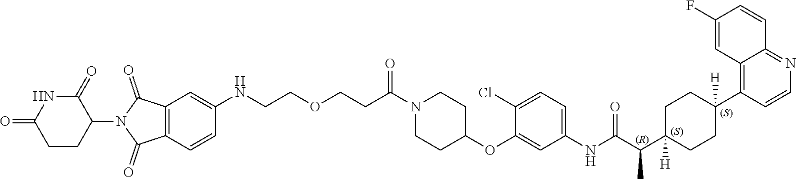 |
| NU227304 | 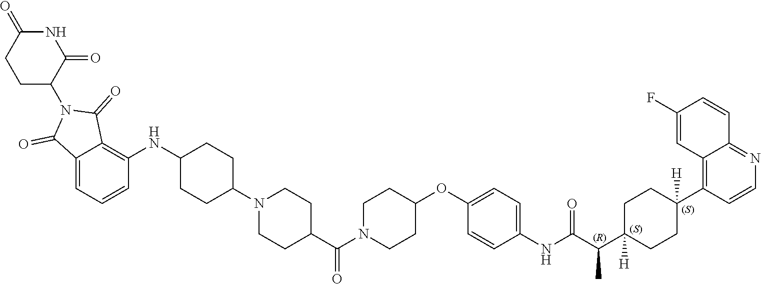 |
| NU227305 | 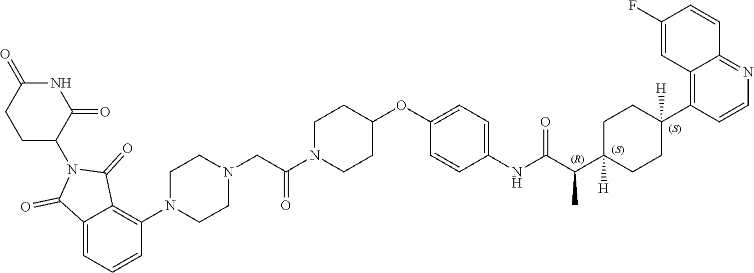 |
| NU227306 | 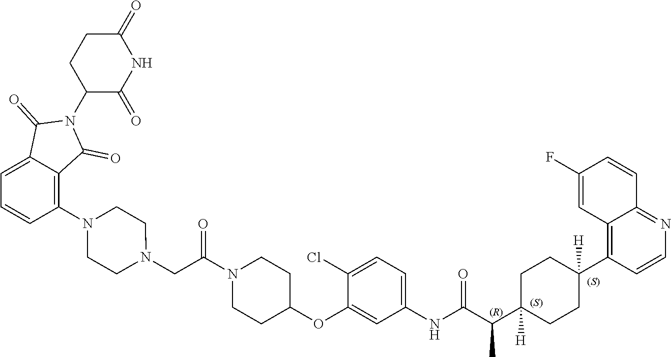 |
| NU227307 | 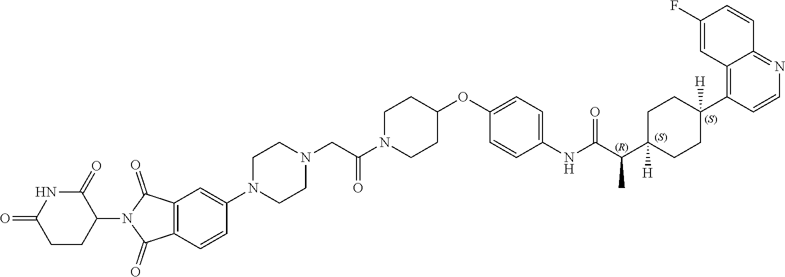 |
| NU227308 | 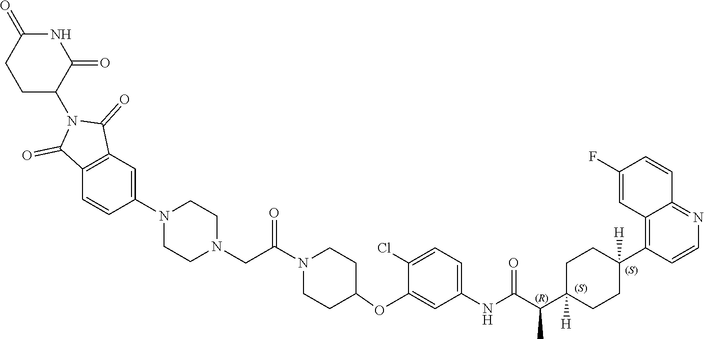 |
| NU227309 | 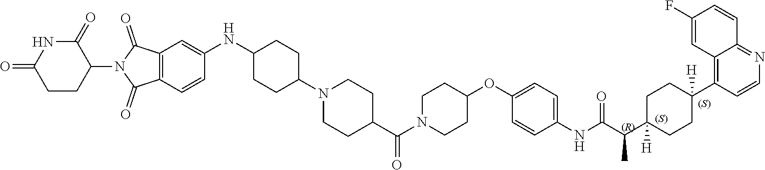 |
| NU227310 | 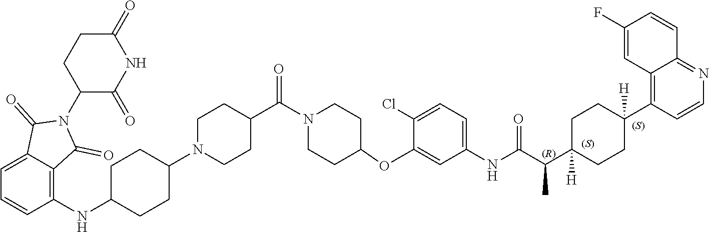 |
| NU227311 | 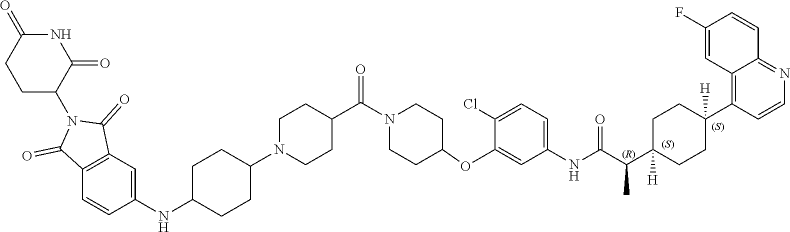 |
| NU227326 | 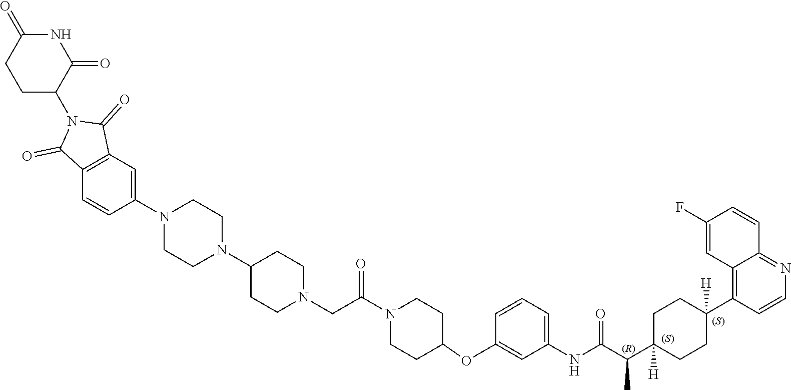 |
| NU227327 | 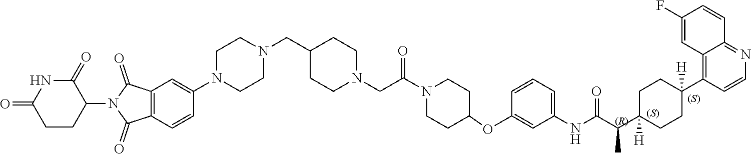 |
| NU227328 | 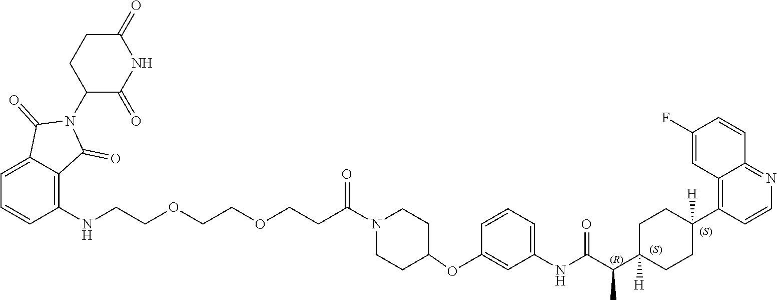 |
| NU227329 | 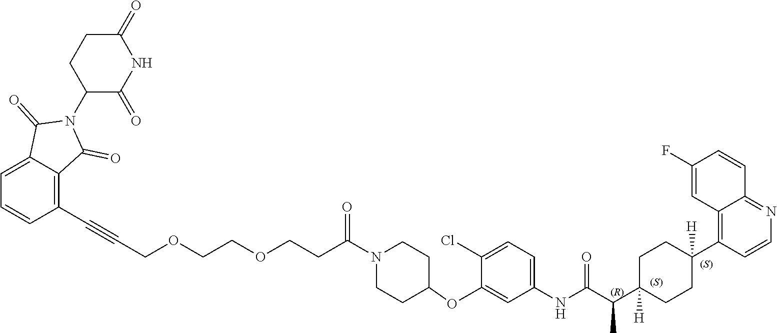 |
| NU227330 | 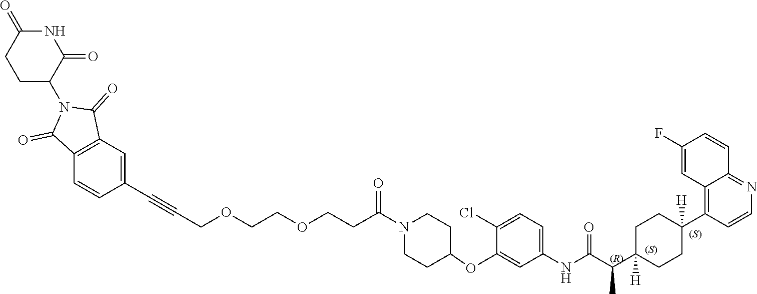 |
| NU227331 | 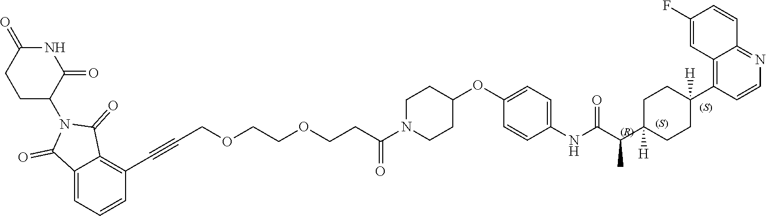 |
| NU227353 | 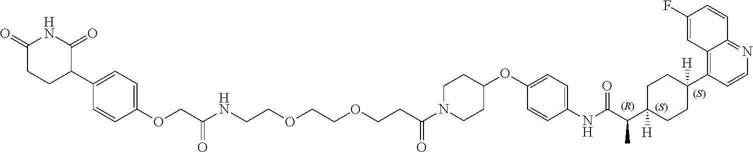 |
| NU227354 | 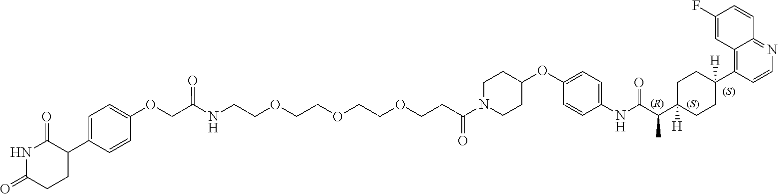 |
| NU227355 | 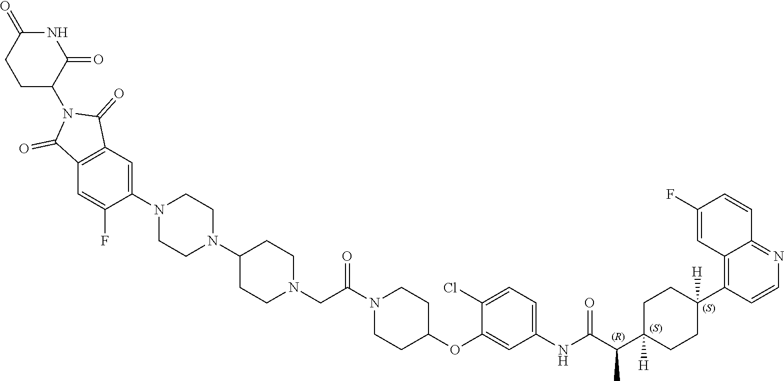 |
| NU227361 | 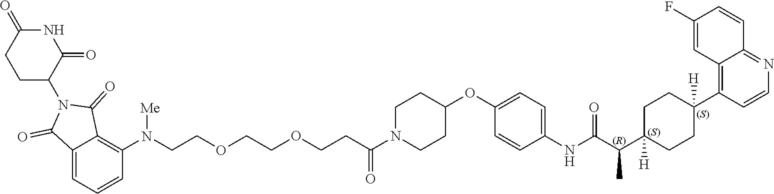 |
| NUCC- 0227428 | 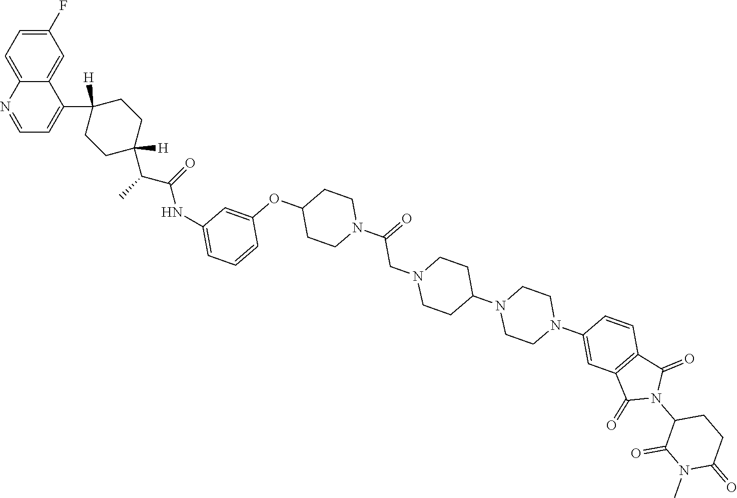 [SMILES: C[C@@H](C(=O)Nc1cccc(OC2CCN(C(=O)CN3CCC(N4CCN(c5ccc6c(c5)C(=O)N(C5CCC(=O)N(C)C5=O)C6=O)CC4)CC3)CC2)c1)[C@H]1CC[C@@H](c2ccnc3ccc(F)cc32)CC1] |
| NUCC- 0227427 | 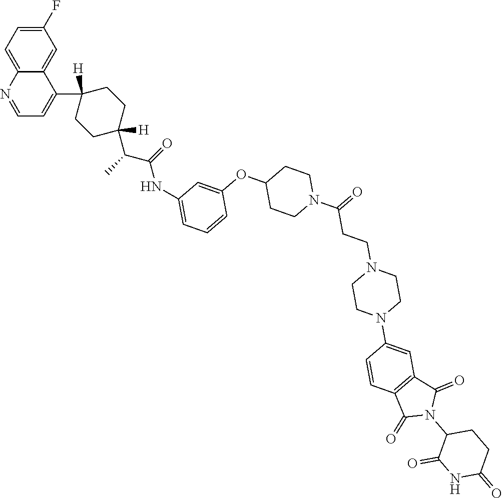 [SMILES: C[C@@H](C(=O)Nc1cccc(OC2CCN(C(=O)CCN3CCN(c4ccc5c(c4)C(=O)N(C4CCC(=O)NC4=O)C5=O)CC3)CC2)c1)[C@H]1CC[C@@H](c2ccnc3ccc(F)cc32)CC1] |
| NUCC- 0227426 | 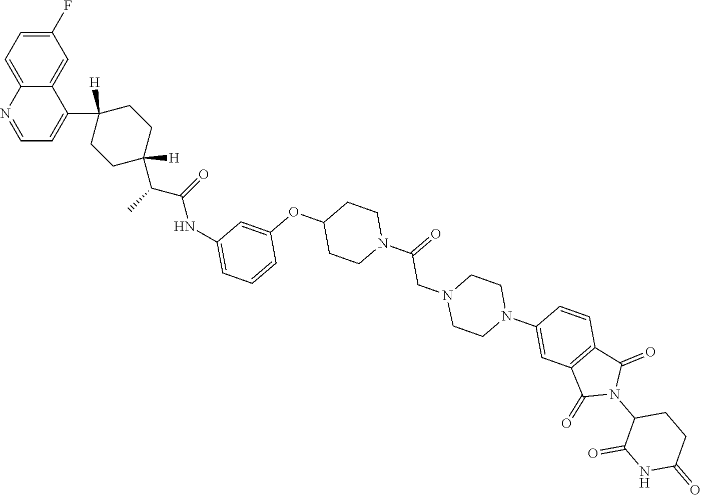 [SMILES: C[C@@H](C(=O)Nc1cccc(OC2CCN(C(=O)CN3CCN(c4ccc5c(c4)C(=O)N(C4CCC(=O)NC4=O)C5=O)CC3)CC2)c1)[C@H]1CC[C@@H](c2ccnc3ccc(F)cc32)CC1] |
| NUCC- 0227425 | 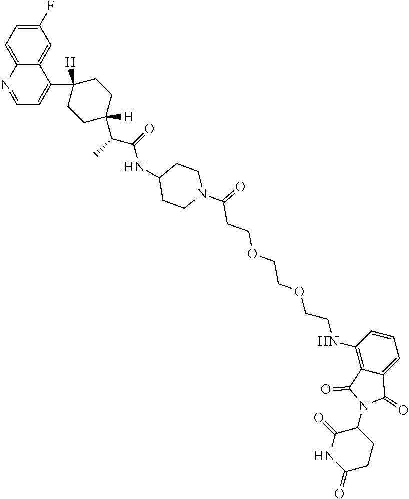 [SMILES: C[C@@H](C(=O)NC1CCN(C(=O)CCOCCOCCNc2cccc3c2C(=O)N(C2CCC(=O)NC2=O)C3=O)CC1)[C@H]1CC[C@@H](c2ccnc3ccc(F)cc32)CC1] |
| NUCC- 0227424 | 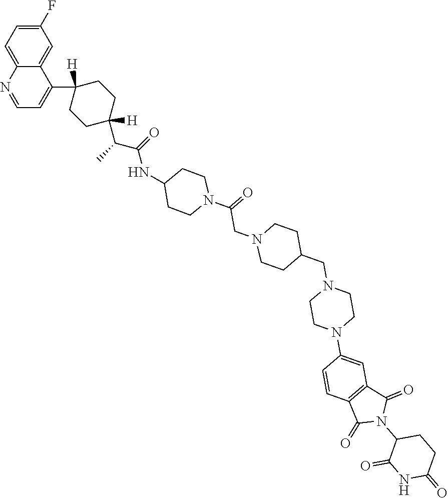 [SMILES: C[C@@H](C(=O)NC1CCN(C(=O)CN2CCC(CN3CCN(c4ccc5c(c4)C(=O)N(C4CCC(=O)NC4=O)C5=O)CC3)CC2)CC1)[C@H]1CC[C@@H](c2ccnc3ccc(F)cc32)CC1] |

TABLE 2
Characterization of IDO1 PROTACs

| ID | HNMR | LCMS |
|---|---|---|
| NU227039 | ¹H NMR (500 MHz, CD3CN) 8 8.83 (s, 1H), 8.66 (d, J = 4.6 | LCMS: t_{R} = |
| | Hz, 1H), 8.18 (s, 1H), 7.95-7.91 (m, 1H), 7.69 (dd, J = 11.0, | 1.20 min, |
| | 2.8 Hz, 1H), 7.49 (dd, J = 8.4, 7.2 Hz, 1H), 7.38 (ddd, J = 9.3, | 100% purity, |
| | 8.2, 2.8 Hz, 1H), 7.34 (d, J = 4.6 Hz, 1H), 7.33-7.29 (m, | m/z = 459.0 |
| | 2H), 7.19 (d, J = 7.2 Hz, 1H), 7.11 (d, J = 8.4 Hz, 1H), 6.76- | (M/2 + H)⁺ |
| | 6.68 (m, 2H), 4.81 (dd, J = 12.5, 5.4 Hz, 1H), 4.34 (dq, J = | |
| | 7.6, 3.8 Hz, 1H), 3.71 (ddd, J = 12.2, 6.8, 3.8 Hz, 1H), 3.50 | |
| | (dt, J = 24.5, 5.8 Hz, 5H), 3.21 (dt, J = 14.4, 5.1 Hz, 6H), 2.70 | |
| | (t, J = 4.9 Hz, 3H), 2.67-2.39 (m, 9H), 1.92 (dtd, J = 12.8, | |
| | 5.1, 2.5 Hz, 1H), 1.84 (dt, J = 14.7, 3.3 Hz, 2H), 1.75-1.47 | |
| | (m, 9H), 1.42 (dp, J = 12.7, 3.9 Hz, 1H), 1.02 (d, J = 6.7 Hz, | |
| | 3H) ppm. | |
| NU227040 | ¹H NMR (500 MHZ, CD3CN) 8 9.02 (s, 1H), 8.82 (d, J = 4.5 | LCMS: t_{R} = |
| | Hz, 1H), 8.53 (s, 1H), 8.09 (dd, J = 9.2, 5.8 Hz, 1H), 7.84 (dd, | 1.30 min, 99% |
| | J = 11.0, 2.8 Hz, 1H), 7.61 (ddt, J = 13.8, 8.0, 2.4 Hz, 2H), | purity, m/z = |
| | 7.57-7.47 (m, 2H), 7.32 (dd, J = 7.2, 3.8 Hz, 1H), 7.26 (t, J = | 950.5 (M + H)⁻. |
| | 8.1 Hz, 2H), 7.10 (tt, J = 7.1, 2.2 Hz, 1H), 4.96 (dd, J = | |
| | 12.5, 5.4 Hz, 1H), 4.66-4.56 (m, 1H), 3.78-3.63 (m, 4H), | |
| | 3.62-3.21 (m, 9H), 2.81-2.51 (m, 11H), 2.11-1.95 (m, | |
| | 5H), 1.91-1.64 (m, 10H), 1.18 (d, J = 6.7 Hz, 3H) ppm. | |
| NU227041 | ¹H NMR (500 MHZ, CD3CN) 8 9.01 (d, J = 15.2 Hz, 1H), | LCMS: t_{R} = |
| | 8.82 (d, J = 4.5 Hz, 1H), 8.35 (s, 1H), 8.17-8.00 (m, 2H), | 1.19 min, |
| | 7.85 (dd, J = 11.0, 2.8 Hz, 1H), 7.67 (d, J = 8.5 Hz, 1H), 7.54 | 100% purity, |
| | (ddd, J = 9.2, 8.1, 2.8 Hz, 1H), 7.51-7.44 (m, 3H), 7.32 (d, | m/z = 916.6 |
| | J = 2.3 Hz, 1H), 7.18 (dd, J = 8.6, 2.4 Hz, 1H), 6.97-6.77 | (M + H)⁺. |
| | (m, 2H), 4.94 (ddd, J = 12.4, 5.3, 1.5 Hz, 1H), 4.50 (tt, J = | |
| | 7.4, 3.6 Hz, 1H), 3.85 (ddd, J = 12.3, 7.2, 3.9 Hz, 1H), 3.68 | |
| | (dt, J = 11.3, 5.7 Hz, 5H), 3.53 (t, J = 5.1 Hz, 4H), 3.36 (ddd, | |
| | J = 13.7, 8.4, 3.6 Hz, 4H), 2.88 (dt, J = 11.1, 5.3 Hz, 6H), | |
| | 2.83-2.58 (m, 7H), 2.12-1.98 (m, 2H), 1.92-1.49 (m, 9H), 1.17 | |
| | (d, J = 6.7 Hz, 3H) ppm. | |
| NU227042 | ¹H NMR (500 MHZ, CD3CN) 8 9.04-8.91 (m, 1H), 8.81 (d, | LCMS: t_{R} = |
| | J = 4.5 Hz, 1H), 8.52 (s, 1H), 8.12-8.04 (m, 2H), 7.84 (dd, | 1.28 min, |
| | J = 11.0, 2.9 Hz, 1H), 7.68-7.58 (m, 2H), 7.58-7.47 (m, | 100% purity, |
| | 2H), 7.31-7.22 (m, 2H), 7.16 (dd, J = 8.6, 2.4 Hz, 1H), 7.08 | m/z = 950.6 |
| | (dt, J = 8.7, 2.2 Hz, 1H), 4.93 (dd, J = 12.4, 5.4 Hz, 1H), 4.61 | (M + H)⁺. |
| | (dt, J = 7.1, 3.5 Hz, 1H), 3.70 (q, J = 6.8 Hz, 4H), 3.63 (td, J = | |
| | 5.6, 1.6 Hz, 2H), 3.58-3.30 (m, 7H), 2.83-2.64 (m, 13H), | |
| | 2.11-1.98 (m, 3H), 1.89-1.68 (m, 9H), 1.18 (d, J = 6.7 Hz, | |
| | 3H) ppm. | |
| NU227043 | ¹H NMR (500 MHZ, CD3CN) & 8.98 (d, J = 6.0 Hz, 2H), | LCMS: tr = |
| | 8.41-8.37 (m, 2H), 8.19 (dd, J = 10.1, 2.7 Hz, 1H), 7.99 (d, J = | 1.29 min, |
| | 5.8 Hz, 1H), 7.91 (ddd, J = 9.4, 7.9, 2.7 Hz, 1H), 7.52-7.42 | 100% purity, |
| | (m, 4H), 6.96 (dd, J = 8.0, 1.8 Hz, 1H), 6.84 (ddd, J = 17.4, | m/z = 446.5 |
| | 6.7, 2.6 Hz, 3H), 4.90 (ddd, J = 12.3, 5.4, 1.7 Hz, 1H), 4.48 | (M/2 + H)⁺. |
| | (tt, J = 7.5, 3.6 Hz, 1H), 3.74-3.55 (m, 10H), 3.35 (dq, J = | |
| | 20.9, 6.2 Hz, 4H), 2.78-2.56 (m, 6H), 2.08-1.97 (m, 3H), | |
| | 1.91-1.51 (m, 12H), 1.20 (d, J = 6.7 Hz, 3H) ppm. | |
| NU227044 | ¹H NMR (500 MHZ, CD3CN) 8 9.08 (s, 1H), 8.99 (h, J = 4.0 | LCMS: t_{R} = |
| | Hz, 1H), 8.58 (s, 1H), 8.41 (dd, J = 9.3, 5.2 Hz, 1H), 8.08 (dd, | 1.43 min, |
| | J = 10.4, 2.8 Hz, 1H), 7.90-7.83 (m, 1H), 7.80 (ddd, J = 9.3, | 100% purity, |
| | 7.9, 2.7 Hz, 1H), 7.58 (ddt, J = 7.4, 5.2, 2.7 Hz, 1H), 7.53-7.45 | m/z = 463.4 |
| | (m, 1H), 7.27 (dd, J = 8.6, 1.0 Hz, 1H), 7.10 (tt, J = 8.8, | (M/2 + H)⁺. |
| | 2.1 Hz, 1H), 7.06-6.96 (m, 2H), 4.92 (dd, J = 12.5, 5.4 Hz, | |
| | 1H), 4.57 (dt, J = 7.0, 3.5 Hz, 1H), 3.74-3.34 (m, 15H), | |
| | 2.80-2.62 (m, 4H), 2.55 (tt, J = 6.3, 3.1 Hz, 2H), 2.13-1.97 (m, | |
| | 3H), 1.93-1.60 (m, 12H), 1.20 (d, J = 6.8 Hz, 3H) ppm. | |
| NU227045 | ¹H NMR (500 MHZ, CD3CN) 8 9.03 (t, J = 11.4 Hz, 1H), 8.83 | LCMS: t_{R} = |
| | (t, J = 4.9 Hz, 1H), 8.51 (d, J = 5.5 Hz, 1H), 8.14 (dd, J = 9.3, | 1.39 min, |
| | 5.6 Hz, 1H), 7.90 (dd, J = 10.8, 2.8 Hz, 1H), 7.64-7.54 (m, | 100% purity, |
| | 3H), 7.54-7.49 (m, 1H), 7.31-7.25 (m, 1H), 7.10 (td, J = | m/z = 925.5 |
| | 8.8, 2.3 Hz, 1H), 7.01 (dd, J = 4.5, 2.2 Hz, 1H), 6.86 (ddd, J = | (M + H)⁺. |
| | 7.4, 4.9, 2.1 Hz, 1H), 4.94-4.88 (m, 1H), 4.61-4.55 (m, | |
| | 1H), 3.79-3.31 (m, 15H), 2.78-2.62 (m, 4H), 2.59-2.53 | |
| | (m, 2H), 2.09-1.97 (m, 3H), 1.93-1.60 (m, 12H), 1.19 (d, | |
| | J = 6.7 Hz, 3H) ppm. | |
| NU227046 | ¹H NMR (500 MHz, CD3CN) & 8.81 (s, 1H), 8.69 (d, J = 4.9 | LCMS: t_{R} = |
| | Hz, 1H), 8.22 (s, 1H), 8.00 (dd, J = 9.3, 5.6 Hz, 1H), 7.78 (dd, | 1.25 min, |
| | J = 10.8, 2.8 Hz, 1H), 7.53-7.43 (m, 2H), 7.38-7.29 (m, | 100% purity, |
| | 2H), 7.15 (td, J = 7.8, 2.5 Hz, 1H), 6.88 (d, J = 7.4 Hz, 1H), | m/z = 439.5 |
| | 6.75-6.62 (m, 3H), 4.91 (ddd, J = 13.4, 5.1, 2.2 Hz, 1H), | (M/2 + H)⁺. |
| | 4.30 (tt, J = 7.5, 3.6 Hz, 1H), 4.13-3.91 (m, 2H), 3.68 (dd, J = | |
| | 13.7, 6.6 Hz, 1H), 3.52 (dt, J = 17.1, 5.9 Hz, 4H), 3.47-3.36 | |
| | (m, J = 2.3 Hz, 4H), 3.27 (td, J = 10.8, 5.5 Hz, 1H), 3.16 | |
| | (dt, J = 32.6, 5.6 Hz, 4H), 2.70-2.52 (m, 4H), 2.39 (q, J = | |
| | 6.6 Hz, 2H), 2.22 (qd, J = 13.2, 4.8 Hz, 1H), 2.01-1.81 (m, | |
| | 3H), 1.79-1.32 (m, 12H), 1.03 (d, J = 6.7 Hz, 3H) ppm. | |
| NU227047 | ¹H NMR (500 MHZ, CD3CN) & 9.06 (s, 1H), 8.89-8.64 (m, | LCMS: tr = |
| | 2H), 8.16 (dq, J = 7.9, 4.1 Hz, 1H), 7.93 (dt, J = 10.8, 2.5 Hz, | 1.37 min, |
| | 1H), 7.69-7.42 (m, 3H), 7.34-7.19 (m, 3H), 7.02 (dd, J = | 100% purity, |
| | 9.5, 7.4 Hz, 1H), 6.83 (dt, J = 8.3, 2.2 Hz, 1H), 5.08 (dq, J = | m/z = 456.5 |
| | 13.2, 4.3 Hz, 1H), 4.48 (d, J = 17.9 Hz, 1H), 4.35-4.14 (m, | (M/2 + H)⁺. |
| | 2H), 3.74-3.67 (m, 5H), 3.59 (h, J = 4.0 Hz, 4H), 3.46-3.32 | |
| | (m, 5H), 2.89-2.67 (m, 4H), 2.62-2.49 (m, 2H), 2.41 (dd, | |
| | J = 13.8, 6.1 Hz, 1H), 2.21-1.99 (m, 3H), 1.92-1.56 (m, | |
| | 12H), 1.22 (d, J = 6.7 Hz, 3H) ppm. | |
| NU227157 | ¹H NMR (500 MHZ, CD3CN) 8 8.96 (s, 1H), 8.82 (d, J = 4.5 | LCMS: t_{R} = |
| | Hz, 1H), 8.32 (s, 1H), 8.08 (dd, J = 9.2, 5.8 Hz, 1H), 7.85 (dd, | 1.38 min, 85% |
| | J = 11.0, 2.8 Hz, 1H), 7.68 (dd, J = 8.5, 7.1 Hz, 1H), 7.57-7.51 | purity, m/z = |
| | (m, 1H), 7.51-7.44 (m, 3H), 7.40-7.27 (m, 3H), 7.06 (dd, | 919.1 (M + H)⁻. |
| | J = 8.0, 2.6 Hz, 1H), 6.98 (dd, J = 2.6, 1.4 Hz, 1H), 6.93-6.87 | |
| | (m, 2H), 6.82 (dt, J = 7.5, 1.1 Hz, 1H), 4.99 (dd, J = | |
| | 12.5, 5.4 Hz, 1H), 4.55 (tt, J = 7.6, 3.7 Hz, 1H), 3.99 (s, 1H), | |
| | 3.58 (s, 1H), 3.51-3.32 (m, 9H), 3.26 (s, 1H), 2.83-2.61 | |
| | (m, 4H), 2.14-2.08 (m, 1H), 2.03-1.96 (m, 2H), 1.93-1.59 | |
| | (m, 12H), 1.17 (d, J = 6.7 Hz, 3H) ppm. | |
| NU227158 | ¹H NMR (500 MHZ, CD3CN) 8 9.06 (d, J = 25.0 Hz, 1H), | LCMS: tr = |
| | 8.84 (d, J = 4.9 Hz, 1H), 8.49 (s, 1H), 8.14 (ddd, J = 9.2, 5.6, | 1.49 min, 94% |
| | 2.2 Hz, 1H), 7.93 (dd, J = 10.8, 2.8 Hz, 1H), 7.72-7.55 (m, | purity, m/z = |
| | 4H), 7.41-7.26 (m, 4H), 7.12 (dd, J = 8.9, 2.2 Hz, 1H), 7.06 | 953.0 (M − H)⁺. |
| | (dd, J = 8.3, 2.5 Hz, 1H), 6.98 (t, J = 1.9 Hz, 1H), 6.83 (d, J = | |
| | 7.4 Hz, 1H), 5.00 (dd, J = 12.5, 5.3 Hz, 1H), 4.65 (dt, J = | |
| | 7.3, 3.8 Hz, 1H), 3.86 (s, 1H), 3.64 (d, J = 49.0 Hz, 2H), | |
| | 3.51-3.26 (m, 9H), 2.86-2.61 (m, 4H), 2.17-1.96 (m, 5H), | |
| | 1.93-1.64 (m, 10H), 1.18 (d, J = 6.7 Hz, 3H) ppm. | |
| NU227159 | ¹H NMR (500 MHZ, CD3CN) § 8.93 (s, 1H), 8.82 (d, J = 4.6 | LCMS: t_{R} = |
| | Hz, 1H), 8.31 (s, 1H), 8.09 (dd, J = 9.2, 5.8 Hz, 1H), 7.85 (dd, | 1.39 min, 73% |
| | J = 11.0, 2.8 Hz, 1H), 7.68 (d, J = 8.5 Hz, 1H), 7.54 (td, J = | purity, m/z = |
| | 8.7, 2.8 Hz, 1H), 7.48 (dd, J = 10.8, 3.8 Hz, 3H), 7.35 (d, J = | 918.4 (M − H)⁺. |
| | 2.3 Hz, 1H), 7.30 (t, J = 7.9 Hz, 1H), 7.22 (dd, J = 8.6, 2.4 | |
| | Hz, 1H), 7.04 (dd, J = 8.3, 2.6 Hz, 1H), 6.96 (t, J = 1.9 Hz, | |
| | 1H), 6.94-6.87 (m, 2H), 6.83 (d, J = 7.4 Hz, 1H), 4.94 (dd, | |
| | J = 12.2, 5.4 Hz, 1H), 4.55 (tt, J = 7.4, 3.6 Hz, 1H), 3.99 (s, | |
| | 1H), 3.62-3.56 (m, 4H), 3.49 (s, 1H), 3.41-3.31 (m, 5H), | |
| | 2.82-2.62 (m, 4H), 2.13-2.04 (m, 2H), 2.04-1.97 (m, 2H), | |
| | 1.93-1.52 (m, 11H), 1.18 (d, J = 6.7 Hz, 3H) ppm. | |
| NU227160 | ¹H NMR (500 MHZ, CD3CN) § 8.94 (s, 1H), 8.84 (d, J = 4.8 | LCMS: t_{R} = |
| | Hz, 1H), 8.49 (s, 1H), 8.13 (dd, J = 9.3, 5.6 Hz, 1H), 7.92 (dd, | 1.49 min, 70% |
| | J = 10.8, 2.8 Hz, 1H), 7.68 (d, J = 8.5 Hz, 1H), 7.66-7.56 | purity, m/z = |
| | (m, 3H), 7.38-7.25 (m, 3H), 7.22 (dd, J = 8.6, 2.4 Hz, 1H), | 952.9 (M + H)⁻. |
| | 7.10 (dd, J = 8.6, 2.2 Hz, 1H), 7.04 (dd, J = 8.4, 2.6 Hz, 1H), | |
| | 6.96 (s, 1H), 6.84 (d, J = 7.4 Hz, 1H), 4.95 (dd, J = 12.2, 5.4 | |
| | Hz, 1H), 4.65 (dt, J = 7.3, 3.7 Hz, 1H), 3.87 (s, 1H), 3.68 (s, | |
| | 1H), 3.59 (t, J = 5.3 Hz, 4H), 3.46-3.27 (m, 6H), 2.79-2.62 | |
| | (m, 4H), 2.12-2.05 (m, 2H), 2.05-1.97 (m, 3H), 1.88-1.65 | |
| | (m, 10H), 1.19 (d, J = 6.7 Hz, 3H) ppm. | |
| NU227161 | ¹H NMR (500 MHZ, CD3CN) 8 8.98 (d, J = 4.1 Hz, 2H), 8.42 | LCMS: t_{R} = |
| | (s, 1H), 8.35 (dd, J = 9.3, 5.4 Hz, 1H), 8.03 (dd, J = 10.6, 2.7 | 1.11 min, |
| | Hz, 1H), 7.80 (d, J = 5.2 Hz, 1H), 7.78-7.67 (m, 2H), 7.53- | 100% purity, |
| | 7.47 (m, 2H), 7.44 (dd, J = 7.3, 0.7 Hz, 1H), 7.34-7.28 (m, | m/z = 956.8 |
| | 1H), 6.98-6.86 (m, 2H), 4.98 (dd, J = 12.8, 5.3 Hz, 1H), 4.55 | (M + H)⁺. |
| | (s, 1H), 3.99 (d, J = 8.9 Hz, 2H), 3.86-2.98 (m, 23H), | |
| | 2.85-2.57 (m, 4H), 2.18-1.97 (m, 5H), 1.92-1.56 (m, 11H), | |
| | 1.19 (d, J = 6.7 Hz, 3H) ppm. | |
| NU227162 | ¹H NMR (500 MHZ, CD3CN) 8 9.10 (s, 1H), 9.06-8.93 (m, | LCMS: t_{R} = |
| | 2H), 8.43 (dd, J = 9.3, 5.2 Hz, 1H), 8.10 (dd, J = 10.3, 2.7 Hz, | 1.18 min, |
| | 1H), 7.95 (d, J = 5.4 Hz, 1H), 7.87-7.77 (m, 1H), 7.74-7.61 | 100% purity, |
| | (m, 2H), 7.43 (d, J = 7.2 Hz, 1H), 7.28 (dd, J = 13.1, 8.5 Hz, | m/z = 990.9 |
| | 2H), 7.15 (dd, J = 8.6, 2.2 Hz, 1H), 4.99 (dd, J = 12.8, 5.4 Hz, | (M + H)⁺. |
| | 1H), 4.65 (s, 1H), 4.02 (d, J = 4.6 Hz, 2H), 3.89-3.00 (m, | |
| | 18H), 2.88-2.57 (m, 4H), 2.26 (s, 1H), 2.18-1.68 (m, 19H), | |
| | 1.18 (d, J = 6.6 Hz, 3H) ppm. | |
| NU227163 | ¹H NMR (500 MHZ, DMSO) 8 11.06 (s, 1H), 10.13 (s, 1H), | LCMS: t_{R} = |
| | 8.92 (d, J = 4.7 Hz, 1H), 8.12 (dd, J = 9.2, 5.8 Hz, 1H), 8.04 | 1.10 min, 98% |
| | (dd, J = 10.9, 2.8 Hz, 1H), 7.77-7.65 (m, 2H), 7.65-7.56 | purity, m/z = |
| | (m, 2H), 7.34 (d, J = 8.7 Hz, 1H), 7.15 (dd, J = 18.4, 8.7 Hz, | 956.9 (M + H)⁻. |
| | 2H), 7.06 (dd, J = 7.1, 2.5 Hz, 1H), 6.63 (s, 1H), 5.10-5.00 | |
| | (m, 1H), 4.61 (s, 1H), 3.73 (dt, J = 50.7, 5.0 Hz, 8H), 3.57-3.39 | |
| | (m, 7H), 3.35-3.23 (m, 3H), 3.12-2.80 (m, 5H), 2.64-2.51 | |
| | (m, 2H), 2.05-1.56 (m, 19H), 1.13 (d, J = 6.6 Hz, 3H) | |
| | ppm. | |
| NU227164 | ¹H NMR (500 MHZ, DMSO) & 11.13 (s, 1H), 10.22 (s, 1H), | LCMS: t_{R} = |
| | 8.92 (d, J = 4.6 Hz, 1H), 8.13 (dd, J = 9.2, 5.8 Hz, 1H), 8.05 | 1.11 min, 99% |
| | (dd, J = 10.9, 2.8 Hz, 1H), 7.83-7.67 (m, 3H), 7.63 (d, J = | purity, m/z = |
| | 4.7 Hz, 1H), 7.56-7.47 (m, 1H), 7.37 (dd, J = 11.2, 8.7 Hz, | |
| | 2H), 7.14 (ddd, J = 8.8, 5.1, 2.2 Hz, 1H), 5.10 (dd, J = 12.8, | 495.7 |
| | 5.5 Hz, 1H), 4.67 (s, 1H), 4.29 (dd, J = 43.3, 8.9 Hz, 4H), | (M/2 + H)⁺. |
| | 3.76-3.76.83 (m, 20H), 2.66-2.55 (m, 1H), 2.07-1.53 (m, 19H), | |
| | 1.13 (d, J = 6.6 Hz, 3H) ppm. | |
| NU227165 | ¹H NMR (500 MHZ, DMSO) 8 11.13 (s, 1H), 9.92 (s, 1H), | LCMS: t_{R} = |
| | 8.95 (d, J = 4.8 Hz, 1H), 8.15 (dd, J = 9.2, 5.7 Hz, 1H), 8.08 | 1.10 min, 98% |
| | (dd, J = 10.8, 2.8 Hz, 1H), 7.77 (ddd, J = 10.7, 8.8, 6.6 Hz, | purity, m/z = |
| | 2H), 7.68 (d, J = 4.8 Hz, 1H), 7.58-7.51 (m, 2H), 7.48 (d, J = | 942.8 (M + H)⁻. |
| | 7.2 Hz, 1H), 7.42 (d, J = 8.4 Hz, 1H), 6.98-6.89 (m, 2H), | |
| | 5.11 (dd, J = 12.8, 5.6 Hz, 1H), 4.59 (tt, J = 7.7, 3.7 Hz, 1H), | |
| | 4.35 (s, 2H), 4.05-3.12 (m, 15H), 3.15-2.77 (m, 4H), 2.60 | |
| | (dt, J = 16.7, 3.6 Hz, 3H), 2.39 (d, J = 11.1 Hz, 2H), 2.30- | |
| | 1.44 (m, 16H), 1.12 (d, J = 6.5 Hz, 3H) ppm. | |
| NU227166 | ¹H NMR (500 MHZ, DMSO) & 11.13 (s, 1H), 10.23 (d, J = | LCMS: t_{R} = |
| | 2.0 Hz, 1H), 8.92 (d, J = 4.7 Hz, 1H), 8.13 (dd, J = 9.2, 5.7 | 1.16 min, 98% |
| | Hz, 1H), 8.06 (dd, J = 10.9, 2.8 Hz, 1H), 7.83-7.70 (m, 3H), | purity, m/z = |
| | 7.64 (d, J = 4.8 Hz, 1H), 7.48 (d, J = 7.2 Hz, 1H), 7.43 (d, J = | 976.8 (M + H)⁻. |
| | 8.4 Hz, 1H), 7.36 (d, J = 8.7 Hz, 1H), 7.15 (ddd, J = 8.7, 5.2, | |
| | 2.2 Hz, 1H), 5.12 (dd, J = 12.8, 5.6 Hz, 1H), 4.68 (s, 1H), 4.36 | |
| | (s, 2H), 3.91 (s, 2H), 3.81-3.17 (m, 15H), 3.03 (s, 2H), 2.97-2.81 | |
| | (m, 2H), 2.65-2.56 (m, 1H), 2.39 (d, J = 11.1 Hz, 2H), | |
| | 2.07-1.57 (m, 16H), 1.14 (d, J = 6.6 Hz, 3H) ppm. | |
| NU227167 | ¹H NMR (500 MHZ, CD3CN) 8 9.00 (d, J = 6.2 Hz, 2H), 8.47 | LCMS: tr = |
| | (s, 1H), 8.40 (dd, J = 9.3, 5.3 Hz, 1H), 8.07 (dd, J = 10.5, 2.7 | 1.08 min, |
| | Hz, 1H), 7.86 (d, J = 5.3 Hz, 1H), 7.84-7.75 (m, 1H), 7.71 | 100% purity, |
| | (d, J = 8.4 Hz, 1H), 7.53-7.46 (m, 2H), 7.37 (d, J = 2.4 Hz, | m/z = 942.8 |
| | 1H), 7.23 (dd, J = 8.5, 2.4 Hz, 1H), 6.96-6.84 (m, 2H), 4.95 | (M+H)1. |
| | (dd, J = 12.5, 5.4 Hz, 1H), 4.55 (tt, J = 7.2, 3.5 Hz, 1H), 4.07 | |
| | (s, 2H), 3.85-3.13 (m, 17H), 2.85-2.61 (m, 5H), 2.40-2.28 | |
| | (m, 4H), 2.15-1.97 (m, 4H), 1.91-1.62 (m, 10H), 1.19 (d, | |
| | J = 6.6 Hz, 3H) ppm. | |
| NU227168 | ¹H NMR (500 MHz, DMSO) § 11.13 (s, 1H), 10.22 (d, J = | LCMS: t_{R} = |
| | 2.0 Hz, 1H), 8.92 (d, J = 4.7 Hz, 1H), 8.13 (dd, J = 9.3, 5.7 | 1.17 min, |
| | Hz, 1H), 8.05 (dd, J = 10.9, 2.8 Hz, 1H), 7.84-7.70 (m, 3H), | 100% purity, |
| | 7.64 (d, J = 4.8 Hz, 1H), 7.51 (d, J = 2.3 Hz, 1H), 7.37 (dd, J = | m/z = 976.9 |
| | 11.9, 8.7 Hz, 2H), 7.15 (ddd, J = 8.0, 5.4, 2.1 Hz, 1H), 5.10 | (M+H)+ |
| | (dd, J = 12.8, 5.4 Hz, 1H), 4.68 (s, 1H), 4.35 (s, 4H), 3.75- | |
| | 3.10 (m, 15H), 3.02 (s, 2H), 2.97-2.82 (m, 2H), 2.70-2.53 | |
| | (m, 1H), 2.32 (d, J = 12.5 Hz, 2H), 2.08-1.56 (m, 16H), 1.14 | |
| | (s, 3H) ppm. | |
| NU227182 | ¹H NMR (500 MHZ, DMSO) 8 11.11 (d, J = 17.1 Hz, 1H), | LCMS: t_{R} = |
| | 9.87 (s, 1H), 8.89 (d, J = 4.6 Hz, 1H), 8.11 (dd, J = 9.2, 5.8 | 1.22 min, |
| | Hz, 1H), 8.02 (dd, J = 11.0, 2.9 Hz, 1H), 7.70 (td, J = 8.7, 2.8 | 100% purity, |
| | Hz, 1H), 7.64-7.58 (m, 2H), 7.58-7.50 (m, 2H), 7.17 (d, J = | m/z = 931.8 |
| | 8.7 Hz, 1H), 7.10-7.04 (m, 1H), 6.93 (dd, J = 9.3, 2.5 Hz, | (M + H)⁺. |
| | 2H), 6.64 (t, J = 5.8 Hz, 1H), 5.12-4.97 (m, 1H), 4.54 (dd, J = | |
| | 8.1, 4.3 Hz, 1H), 3.89-3.18 (m, 12H), 3.02-2.80 (m, 5H), | |
| | 2.67-2.52 (m, 2H), 2.05-1.43 (m, 21H), 1.12 (d, J = 6.6 | |
| | Hz, 3H) ppm. | |
| NU227183 | ¹H NMR (500 MHZ, DMSO) 8 11.10 (s, 1H), 10.17 (d, J = | LCMS: t_{R} = |
| | 3.3 Hz, 1H), 8.93 (d, J = 4.7 Hz, 1H), 8.13 (dd, J = 9.3, 5.7 | 1.39 min, 80% |
| | Hz, 1H), 8.06 (dd, J = 10.9, 2.8 Hz, 1H), 7.81-7.55 (m, 4H), | purity, m/z = |
| | 7.35 (d, J = 8.7 Hz, 1H), 7.20-7.04 (m, 3H), 6.64 (t, J = 5.7 | 965.8 (M + H)⁻. |
| | Hz, 1H), 5.12-5.01 (m, 2H), 4.62 (s, 1H), 3.72 (dt, J = 50.9, | |
| | 4.9 Hz, 6H), 3.55-3.44 (m, 6H), 3.27 (q, J = 5.0 Hz, 2H), | |
| | 2.98 (d, J = 11.6 Hz, 1H), 2.93-2.82 (m, 3H), 2.65-2.51 | |
| | (m, 2H), 2.09-1.51 (m, 20H), 1.13 (d, J = 6.6 Hz, 3H) ppm. | |
| NU227191 | ¹H NMR (500 MHZ, DMSO) 8 11.06 (s, 1H), 9.85 (s, 1H), | LCMS: t_{R} = |
| | 9.25 (d, J = 31.0 Hz, 1H), 8.93 (d, J = 4.7 Hz, 1H), 8.13 (dd, | 1.29 min, 91% |
| | J = 9.2, 5.7 Hz, 1H), 8.05 (dd, J = 10.9, 2.9 Hz, 1H), 7.73 | purity, m/z = |
| | (ddd, J = 9.2, 8.1, 2.8 Hz, 1H), 7.64 (d, J = 4.7 Hz, 1H), 7.58 | 931.7 (M + H)⁻. |
| | (d, J = 8.4 Hz, 1H), 7.56-7.49 (m, 2H), 7.02 (d, J = 2.1 Hz, | |
| | 1H), 6.96-6.88 (m, 3H), 5.03 (dd, J = 12.7, 5.5 Hz, 1H), 4.56- | |
| | 4.53 (m, 1H), 3.87-3.73 (m, 4H), 3.64 (t, J = 5.4 Hz, 2H), | |
| | 3.54-3.39 (m, 5H), 3.27-3.21 (m, 3H), 2.98 (d, J = 10.9 | |
| | Hz, 1H), 2.91-2.81 (m, 3H), 2.56 (dd, J = 18.9, 13.4 Hz, | |
| | 2H), 2.01-1.44 (m, 20H), 1.12 (d, J = 6.6 Hz, 3H) ppm. | |
| NU227192 | ¹H NMR (500 MHZ, DMSO) 8 11.06 (s, 1H), 10.13 (d, J = | LCMS: t_{R} = |
| | 2.4 Hz, 1H), 8.92 (d, J = 4.7 Hz, 1H), 8.13 (dd, J = 9.2, 5.7 | 1.79 min, 86% |
| | Hz, 1H), 8.04 (dd, J = 10.9, 2.8 Hz, 1H), 7.72 (ddd, J = 11.9, | purity, m/z = |
| | 7.0, 2.6 Hz, 2H), 7.60 (dd, J = 18.0, 6.6 Hz, 2H), 7.34 (d, J = | 483.1 |
| | 8.7 Hz, 1H), 7.14 (t, J = 12.4 Hz, 2H), 7.02 (d, J = 2.1 Hz, | (M/2 + H)⁺. |
| | 1H), 6.91 (dd, J = 8.4, 2.2 Hz, 1H), 5.03 (dd, J = 12.7, 5.5 Hz, | |
| | 1H), 4.62 (s, 1H), 3.76 (q, J = 5.8 Hz, 3H), 3.64 (t, J = 5.4 Hz, | |
| | 2H), 3.52 (d, J = 12.2 Hz, 2H), 3.43 (q, J = 4.6 Hz, 5H), 2.99 | |
| | (s, 1H), 2.93-2.82 (m, 3H), 2.60-2.51 (m, 2H), 2.00-1.57 | |
| | (m, 20H), 1.14 (d, J = 6.7 Hz, 3H) ppm. | |
| NU227193 | ¹H NMR (500 MHZ, DMSO) 8 11.13 (s, 1H), 9.87 (s, 1H), | LCMS: t_{R} = |
| | 8.89 (d, J = 4.5 Hz, 1H), 8.10 (dd, J = 9.2, 5.7 Hz, 1H), 8.01 | 1.16 min, 80% |
| | (dd, J = 11.0, 2.9 Hz, 1H), 7.69 (t, J = 9.0 Hz, 1H), 7.59 (q, J = | purity, m/z = |
| | 5.8 Hz, 2H), 7.53 (d, J = 8.6 Hz, 2H), 7.18 (d, J = 8.8 Hz, | 478.0 |
| | 1H), 7.11-7.00 (m, 1H), 6.93 (d, J = 8.7 Hz, 2H), 6.18 (d, J = | (M/2 + H)⁺. |
| | 8.3 Hz, 1H), 5.11-4.97 (m, 1H), 4.55 (s, 1H), 3.60-3.19 | |
| | (m, 9H), 2.97-2.80 (m, 6H), 2.67-2.54 (m, 2H), 2.04-1.43 | |
| | (m, 24H), 1.31-1.10 (m, 6H) ppm. | |
| NU227194 | Both glutarimide isomers present in HNMR | LCMS: t_{R} = |
| | ¹H NMR (500 MHZ, DMSO) § 11.09 (s, 1H), 9.87 (d, J = 3.9 | 1.12 min, 90% |
| | Hz, 2H), 8.88 (d, J = 4.5 Hz, 2H), 8.10 (dd, J = 9.2, 5.8 Hz, | purity, m/z = |
| | 2H), 8.01 (dd, J = 11.0, 2.9 Hz, 2H), 7.73-7.63 (m, 2H), 7.63- | 478.0 |
| | 7.44 (m, 7H), 7.04 (d, J = 7.7 Hz, 1H), 7.01-6.80 (m, 6H), | (M/2 + H)⁺ |
| | 5.03 (dd, J = 12.8, 5.4 Hz, 1H), 4.57-4.52 (m, 2H), 3.01-2.77 | |
| | (m, 13H), 2.75-2.53 (m, 6H), 2.07-1.53 (m, 43H), | |
| | 1.36-1.08 (m, 15H) ppm. | |
| NU227198 | ¹H NMR(DMSO, 500 MHZ): 8 = 11.13 (s, 1H), 10.20-10.12 | LCMS: t_{R} = |
| | (m, 1H), 8.96-8.71 (m, 2H), 8.11 (dd, 1H, J =9.2, 5.8 Hz), | 1.21 min, 93% |
| | 8.02 (dd, 1H, J =11.1, 3.0 Hz), 7.88-7.49 (m, 4H), 7.35 (d, | purity, m/z = |
| | 1H, J =8.6 Hz), 7.22-7.03 (m, 2H), 6.18 (d, 1H, J-8.2 Hz), | 495.1 |
| | 5.05 (dd, 1H, J =12.8, 5.4 Hz), 4.62 (s, 1H), 3.97-3.09 (m, | (M/2 + H)⁺. |
| | 17H), 3.00-2.81 (m, 5H), 2.67-2.52 (m, 2H), 2.08-1.50 | |
| | (m, 18H), 1.37-1.09 (m, 5H) ppm. | |
| NU227199 | ¹H NMR (500 MHZ, DMSO) § 11.08 (s, 1H), 10.16 (s, 1H), | LCMS: t_{R} = |
| | 8.87 (d, J = 4.5 Hz, 1H), 8.09 (dd, J = 9.2, 5.8 Hz, 1H), 7.99 | 1.25 min, 83% |
| | (dd, J = 11.1, 2.8 Hz, 1H), 7.67 (ddd, J = 15.7, 8.8, 4.7 Hz, | purity, m/z = |
| | 2H), 7.55 (dd, J = 11.3, 6.5 Hz, 2H), 7.34 (d, J = 8.6 Hz, 1H), | 495.2 |
| | 7.18 (s, 1H), 7.08-6.80 (m, 3H), 5.02 (dd, J = 12.8, 5.5 Hz, | (M/2 + H)⁺. |
| | 1H), 4.60 (s, 1H), 3.68 (s, 2H), 3.45-3.28 (m, 15H), 2.85 (d, | |
| | J = 11.4 Hz, 5H), 2.65-2.54 (m, 2H), 1.97-1.58 (m, 18H), | |
| | 1.23 (s, 2H), 1.13 (d, J = 6.6 Hz, 3H) ppm. | |
| NU227200 | ¹H NMR (500 MHZ, DMSO) 8 11.12 (s, 1H), 9.87 (s, 1H), | LCMS: t_{R} = |
| | 8.99 (d, J = 4.9 Hz, 1H), 8.14 (ddd, J = 17.2, 10.1, 3.7 Hz, | 1.46 min, 98% |
| | 2H), 7.78 (t, J = 8.0 Hz, 1H), 7.71 (d, J = 4.9 Hz, 1H), 7.57 | purity, m/z = |
| | (dd, J = 8.6, 7.1 Hz, 1H), 7.54-7.50 (m, 2H), 7.09 (d, J = 8.6 | 444.5 |
| | Hz, 1H), 7.01 (d, J = 7.0 Hz, 1H), 6.95-6.89 (m, 2H), 6.54 | (M/2 + H)⁺. |
| | (s, 1H), 5.05 (dd, J = 12.8, 5.4 Hz, 1H), 4.51 (dt, J = 7.9, 4.1 | |
| | Hz, 1H), 3.48 (d, J = 11.3 Hz, 2H), 3.35-3.13 (m, 5H), 2.94- | |
| | 2.79 (m, 2H), 2.67-2.51 (m, 2H), 2.29 (t, J = 7.5 Hz, 2H), | |
| | 2.07-1.41 (m, 18H), 1.28 (t, J = 12.7 Hz, 8H), 1.12 (d, J = | |
| | 6.6 Hz, 3H) ppm. | |
| NU227201 | ¹H NMR (500 MHZ, DMSO) § 11.12 (s, 1H), 10.14 (s, 1H), | LCMS: t_{R} = |
| | 8.97 (d, J = 4.7 Hz, 1H), 8.12 (ddd, J = 23.3, 10.1, 4.3 Hz, | 1.57 min, 98% |
| | 2H), 7.77 (dt, J = 8.7, 4.3 Hz, 1H), 7.70-7.61 (m, 2H), 7.57 | purity, m/z = |
| | (dd, J = 8.5, 7.0 Hz, 1H), 7.33 (d, J = 8.7 Hz, 1H), 7.17 (td, J = | 461.7 |
| | 9.0, 2.2 Hz, 1H), 7.08 (d, J = 8.6 Hz, 1H), 7.01 (d, J = 7.0 | (M/2 + H)⁺ |
| | Hz, 1H), 6.54 (s, 1H), 5.05 (dd, J = 12.8, 5.4 Hz, 1H), 4.58 | |
| | (dd, J = 7.3, 3.9 Hz, 1H), 3.68-3.61 (m, 2H), 3.49-3.39 (m, | |
| | 3H), 3.31-3.24 (m, 2H), 2.93-2.81 (m, 2H), 2.66-2.51 (m, | |
| | 2H), 2.30 (td, J = 7.5, 2.9 Hz, 2H), 2.06-1.43 (m, 18H), 1.28 | |
| | (t, J = 8.6 Hz, 8H), 1.13 (d, J = 6.6 Hz, 3H) ppm. | |
| NU227302 | Both glutarimide isomers present in HNMR | LCMS: tr = |
| | ¹H NMR (500 MHZ, DMSO) 8 11.15 (s, 1H), 11.09 (s, 1H), | 1.31 min, 99% |
| | 9.86 (s, 1H), 8.94 (d, J = 4.7 Hz, 1H), 8.13 (dd, J = 9.3, 5.7 | purity, m/z = |
| | Hz, 1H), 8.07 (dd, J = 10.8, 2.8 Hz, 1H), 7.91 (d, J = 1.2 Hz, | 848.8 (M + H)⁻. |
| | 1H), 7.74 (td, J = 8.7, 2.8 Hz, 2H), 7.65 (d, J = 4.7 Hz, 1H), | |
| | 7.59-7.40 (m, 4H), 7.03-6.81 (m, 5H), 5.03 (dd, J = 12.7, | |
| | 5.6 Hz, 1H), 4.49 (s, 2H), 3.80 (d, J = 7.0 Hz, 4H), 3.46-3.18 | |
| | (m, 13H), 2.94-2.77 (m, 3H), 2.61 (td, J = 12.7, 4.6 Hz, 3H), | |
| | 2.06-1.37 (m, 23H), 1.12 (d, J = 6.6 Hz, 5H) ppm. | |
| NU227303 | Both glutarimide isomers present in HNMR | LCMS: t_{R} = |
| | ¹H NMR (500 MHz, DMSO) 8 11.09 (s, 1H), 10.14 (s, 1H), | 1.30 min, 92% |
| | 8.95 (d, J = 4.6 Hz, 1H), 8.11 (ddd, J = 27.1, 10.2, 4.2 Hz, | purity, m/z = |
| | 3H), 7.81-7.70 (m, 2H), 7.69-7.61 (m, 3H), 7.56 (d, J = | 441.5 |
| | 8.4 Hz, 1H), 7.34 (dd, J = 8.6, 3.3 Hz, 1H), 7.25-7.12 (m, | (M/2 + H)⁺. |
| | 2H), 7.00 (d, J = 2.2 Hz, 1H), 6.89 (dd, J = 8.4, 2.1 Hz, 1H), | |
| | 5.03 (dt, J = 12.4, 6.3 Hz, 1H), 4.59 (s, 1H), 3.53-3.38 (m, | |
| | 8H), 2.87 (ddq, J = 14.3, 10.5, 6.6 Hz, 3H), 2.72-2.55 (m, | |
| | 3H), 2.07-1.46 (m, 21H), 1.13 (d, J = 6.6 Hz, 4H) ppm. | |
| NU227304 | ¹H NMR (500 MHz, DMSO) 8 11.13 (d, J = 6.6 Hz, 1H), 9.88 | LCMS: t_{R} = |
| | (s, 1H), 8.90 (d, J = 4.6 Hz, 1H), 8.11 (dd, J = 9.2, 5.8 Hz, | 1.11 min, 96% |
| | 1H), 8.03 (dd, J = 11.0, 2.9 Hz, 1H), 7.71 (td, J = 8.6, 2.8 Hz, | purity, m/z = |
| | 1H), 7.67-7.57 (m, 2H), 7.53 (d, J = 8.6 Hz, 2H), 7.17 (dd, | 471.1 |
| | J = 12.8, 8.8 Hz, 1H), 7.08 (dd, J = 16.1, 7.0 Hz, 1H), 6.99- | (M/2+H) . |
| | 6.82 (m, 2H), 6.43 (d, J = 7.5 Hz, 1H), 5.06 (dt, J = 12.7, 6.4 | |
| | Hz, 1H), 4.55 (dq, J = 8.0, 3.9 Hz, 1H), 3.97-3.80 (m, 4H), | |
| | 3.28-3.20 (m, 2H), 3.10-2.95 (m, 3H), 2.93-2.81 (m, 2H), | |
| | 2.65-2.52 (m, 2H), 2.12-1.70 (m, 21H), 1.66-1.55 (m, | |
| | 6H), 1.50-1.35 (m, 2H), 1.12 (d, J = 6.6 Hz, 3H) ppm. | |
| NU227305 | ¹H NMR (500 MHZ, DMSO) 8 11.14 (s, 1H), 9.90 (s, 1H), | LCMS: t_{R} = |
| | 8.92 (d, J = 4.7 Hz, 1H), 8.12 (dd, J = 9.2, 5.8 Hz, 1H), 8.05 | 1.06 min, 98% |
| | (dd, J = 10.9, 2.9 Hz, 1H), 7.81-7.69 (m, 2H), 7.63 (d, J = | purity, m/z = |
| | 4.7 Hz, 1H), 7.59-7.50 (m, 2H), 7.45 (dd, J = 23.6, 7.8 Hz, | 430.1 |
| | 2H), 7.00-6.86 (m, 2H), 5.11 (dd, J = 12.8, 5.5 Hz, 1H), 4.63- | (M/2 + H)⁺. |
| | 4.55 (m, 1H), 4.46 (s, 2H), 3.80-3.87 (m, 5H), 3.45-3.26 | |
| | (m, 8H), 2.85 (td, J = 12.7, 4.3 Hz, 2H), 2.68-2.51 (m, 2H), | |
| | 2.05-1.55 (m, 14H), 1.12 (d, J = 6.6 Hz, 3H) ppm. | |
| NU227306 | ¹H NMR (500 MHZ, DMSO) 8 11.14 (d, J = 2.2 Hz, 1H), | LCMS: t_{R} = |
| | 10.19-10.13 (m, 1H), 8.90 (d, J = 4.6 Hz, 1H), 8.11 (dd, J = | 1.27 min, 99% |
| | 9.3, 5.8 Hz, 1H), 8.02 (dd, J = 11.0, 2.8 Hz, 1H), 7.84-7.67 | purity, m/z = |
| | (m, 3H), 7.59 (d, J = 4.8 Hz, 1H), 7.47 (d, J = 7.1 Hz, 1H), | 893.7 (M + H)⁻. |
| | 7.43 (d, J = 8.5 Hz, 1H), 7.36 (dd, J = 8.7, 4.4 Hz, 1H), 7.22- | |
| | 7.07 (m, 1H), 5.11 (d, J = 18.3 Hz, 1H), 4.68 (s, 1H), 4.48 | |
| | (s, 2H), 3.89-3.72 (m, 5H), 3.42-3.29 (m, 7H), 2.88 (td, J = | |
| | 17.6, 4.4 Hz, 3H), 2.68-2.51 (m, 2H), 2.05-1.99 (m, 2H), | |
| | 1.97-1.87 (m, 4H), 1.83-1.56 (m, 8H), 1.14 (d, J = 6.8 Hz, | |
| | 3H) ppm. | |
| NU227307 | ¹H NMR (500 MHZ, DMSO) § 11.13 (s, 1H), 9.90 (s, 1H), | LCMS: tr = |
| | 8.90 (d, J = 4.6 Hz, 1H), 8.11 (dd, J = 9.2, 5.8 Hz, 1H), 8.02 | 1.05 min, 95% |
| | (dd, J = 11.0, 2.9 Hz, 1H), 7.78 (d, J = 8.4 Hz, 1H), 7.70 (td, | purity, m/z = |
| | J = 8.6, 2.8 Hz, 1H), 7.60 (d, J = 4.7 Hz, 1H), 7.58-7.52 (m, | 430.0 |
| | 2H), 7.52-7.47 (m, 1H), 7.34 (dd, J = 8.6, 2.3 Hz, 1H), 6.99- | (M/2 + H)⁺. |
| | 6.83 (m, 2H), 5.10 (dd, J = 12.8, 5.5 Hz, 1H), 4.59 (s, 1H), | |
| | 4.41 (s, 2H), 4.16 (s, 2H), 3.87 (d, J = 7.3 Hz, 2H), 3.37-3.16 | |
| | (m, 8H), 2.95-2.77 (m, 3H), 2.68-2.52 (m, 2H), 2.01-1.53 | |
| | (m, 14H), 1.12 (d, J = 6.6 Hz, 3H) ppm. | |
| NU227308 | ¹H NMR (500 MHz, DMSO) & 11.13 (s, 1H), 10.20 (s, 1H), | LCMS: t_{R} = |
| | 8.90 (d, J = 4.6 Hz, 1H), 8.11 (dd, J = 9.2, 5.8 Hz, 1H), 8.02 | 1.14 min, 93% |
| | (dd, J = 10.9, 2.9 Hz, 1H), 7.83-7.66 (m, 3H), 7.60 (d, J = | purity, m/z = |
| | 4.8 Hz, 1H), 7.49 (d, J = 2.2 Hz, 1H), 7.41-7.31 (m, 2H), | 447.1 |
| | 7.24-7.09 (m, 1H), 5.10 (dd, J = 12.8, 5.4 Hz, 1H), 4.67 (dt, | |
| | J = 10.6, 4.9 Hz, 1H), 4.52-4.08 (m, 3H), 3.79-3.69 (m, | |
| | 2H), 3.32-3.10 (m, 8H), 2.92-2.84 (m, 4H), 2.68-2.52 (m, | (M/2 + H)⁺. |
| | 2H), 2.06-1.97 (m, 2H), 1.98-1.86 (m, 5H), 1.85-1.70 (m, | |
| | 5H), 1.68-1.55 (m, 4H), 1.13 (d, J = 6.6 Hz, 3H) ppm. | |
| NU227309 | Both glutarimide isomers present in HNMR | LCMS: t_{R} = |
| | ¹H NMR (500 MHZ, DMSO) 8 11.09 (s, 1H), 9.88 (s, 1H), | 1.10 min, 81% |
| | 8.92 (d, J = 4.7 Hz, 1H), 8.12 (dd, J = 9.2, 5.7 Hz, 1H), 8.05 | purity, m/z = |
| | (dd, J = 10.9, 2.9 Hz, 1H), 7.72 (td, J = 8.9, 3.1 Hz, 2H), | 471.1 |
| | 7.64-7.56 (m, 2H), 7.55-7.50 (m, 2H), 7.07-6.95 (m, 2H), | (M/2 + H)⁺. |
| | 6.94-6.91 (m, 2H), 5.03 (dd, J = 12.8, 5.4 Hz, 1H), 4.59-4.50 | |
| | (m, 1H), 3.54-2.79 (m, 16H), 2.70-2.52 (m, 2H), 2.15- | |
| | 1.37 (m, 30H), 1.33-1.21 (m, 1H), 1.12 (d, J = 6.5 Hz, 4H) | |
| | ppm. | |
| NU227310 | ¹H NMR (500 MHz, DMSO) 8 11.14 (d, J = 8.1 Hz, 1H), | LCMS: t_{R} = |
| | 10.15 (s, 1H), 8.89 (d, J = 4.6 Hz, 1H), 8.10 (dd, J = 9.3, 5.8 | 1.27 min, 96% |
| | Hz, 1H), 8.04-7.96 (m, 1H), 7.76-7.52 (m, 4H), 7.35 (d, J = | purity, m/z = |
| | 8.7 Hz, 1H), 7.22-7.00 (m, 3H), 6.44 (d, J = 7.4 Hz, 1H), | 488.2 |
| | 5.06 (s, 1H), 4.62 (s, 1H), 3.94 (s, 2H), 3.84-3.22 (m, 12H), | (M/2 + H)⁺ |
| | 3.16-2.83 (m, 5H), 2.62 (d, J = 11.3 Hz, 2H), 2.18-1.40 | |
| | (m, 21H), 1.13 (d, J = 6.5 Hz, 3H) ppm. | |
| NU227311 | ¹H NMR (500 MHz, DMSO) & 11.09 (d, J = 5.2 Hz, 1H), | LCMS: t_{R} = |
| | 10.18 (s, 1H), 8.89 (d, J = 4.6 Hz, 1H), 8.11 (dd, J = 9.3, 5.8 | 1.26 min, 82% |
| | Hz, 1H), 8.01 (d, J = 10.8 Hz, 1H), 7.79-7.49 (m, 4H), 7.35 | purity, m/z = |
| | (d, J = 8.7 Hz, 1H), 7.26-6.79 (m, 4H), 5.12-4.98 (m, 1H), | 488.2 |
| | 4.62 (s, 1H), 3.79-3.63 (m, 4H), 3.54-3.35 (m, 8H), 3.14- | (M/2 + H)⁺ |
| | 2.80 (m, 5H), 2.59 (t, J = 20.2 Hz, 2H), 2.28-1.53 (m, 23H), | |
| | 1.13 (d, J = 6.5 Hz, 3H) ppm. | |
| NU227326 | ¹H NMR (500 MHZ, DMSO) § 11.13 (s, 1H), 10.01 (s, 1H), | LCMS: t_{R} = |
| | 8.89 (d, J = 4.6 Hz, 1H), 8.10 (dd, J = 9.2, 5.8 Hz, 1H), 8.01 | |
| | (dd, J = 10.9, 2.8 Hz, 1H), 7.78 (d, J = 8.3 Hz, 1H), 7.69 (td, | |
| | J = 8.7, 2.8 Hz, 1H), 7.59 (d, J = 4.6 Hz, 1H), 7.56-7.31 (m, | 1.10 min, |
| | 3H), 7.20 (t, J = 8.1 Hz, 1H), 7.06 (d, J = 8.0 Hz, 1H), 6.69 | |
| | (dd, J = 8.4, 2.4 Hz, 1H), 5.10 (dd, J = 12.8, 5.5 Hz, 1H), 4.62 | |
| | (s, 1H), 4.33 (s, 2H), 3.94-2.82 (m, 25H), 2.67-2.54 (m, | 100% purity, |
| | 2H), 2.42-2.19 (m, 2H), 2.17-1.51 (m, 13H), 1.12 (d, J = | m/z = 941.4 |
| | 6.5 Hz, 3H) ppm. | (M+H) *. |
| NU227327 | ¹H NMR (500 MHZ, DMSO) 8 11.13 (s, 1H), 10.03 (s, 1H), | LCMS: t_{R} = |
| | 8.90 (d, J = 4.5 Hz, 1H), 8.11 (dd, J = 9.2, 5.8 Hz, 1H), 8.02 | 1.12 min, |
| | (dd, J = 11.0, 2.8 Hz, 1H), 7.78 (d, J = 8.4 Hz, 1H), 7.70 (td, | 100% purity, |
| | J = 8.6, 2.8 Hz, 1H), 7.60 (d, J = 4.7 Hz, 1H), 7.55-7.42 (m, | m/z = 955.4 |
| | 2H), 7.38 (d, J = 8.7 Hz, 1H), 7.20 (td, J = 8.1, 2.5 Hz, 1H), | (M + H)⁺. |
| | 7.07 (d, J = 7.9 Hz, 1H), 6.69 (dd, J = 8.2, 2.5 Hz, 1H), 5.10 | |
| | (dd, J = 12.8, 5.5 Hz, 1H), 4.62 (s, 1H), 4.34-4.21 (m, 3H), | |
| | 3.90-2.79 (m, 22H), 2.69-2.51 (m, 2H), 2.17-1.44 (m, | |
| | 17H), 1.12 (d, J = 6.6 Hz, 3H) ppm. | |
| NU227328 | ¹H NMR (500 MHZ, DMSO) 8 11.12 (s, 1H), 9.98 (s, 1H), | LCMS: t_{R} = |
| | 8.96 (d, J = 4.8 Hz, 1H), 8.14 (dd, J = 9.2, 5.7 Hz, 1H), 8.09 | |
| | (dd, J = 10.9, 2.9 Hz, 1H), 7.76 (td, J = 8.7, 2.7 Hz, 1H), 7.68 | |
| | (d, J = 4.8 Hz, 1H), 7.57 (dd, J = 8.6, 7.1 Hz, 1H), 7.39 (d, J = | 1.37 min, |
| | 2.3 Hz, 1H), 7.21-7.09 (m, 3H), 7.03 (d, J = 7.0 Hz, 1H), | |
| | 6.68-6.55 (m, 2H), 5.05 (dd, J = 12.8, 5.4 Hz, 1H), 4.52 (tt, | 100% purity, |
| | J = 7.8, 3.7 Hz, 1H), 3.85-3.76 (m, 4H), 3.49-3.41 (m, 7H), | |
| | 3.35-3.16 (m, 4H), 2.95-2.82 (m, 2H), 2.66-2.51 (m, 3H), | m/z = 890.9 |
| | 2.06-1.41 (m, 15H), 1.12 (d, J = 6.7 Hz, 3H) ppm. | (M + H)⁺. |
| NU227329 | ¹H NMR (500 MHZ, DMSO) 8 11.17 (s, 1H), 10.14 (s, 1H), | LCMS: t_{R} = |
| | 8.92 (d, J = 4.7 Hz, 1H), 8.12 (dd, J = 9.2, 5.8 Hz, 1H), 8.05 | 1.46 min, 98% |
| | (dd, J = 10.9, 2.9 Hz, 1H), 7.96-7.80 (m, 3H), 7.73 (td, J = | purity, m/z = |
| | 8.7, 2.8 Hz, 1H), 7.68-7.56 (m, 2H), 7.33 (d, J = 8.7 Hz, | 921.8 (M + H)⁻. |
| | 1H), 7.17 (ddd, J = 8.8, 6.6, 2.3 Hz, 1H), 5.15 (dd, J = 12.9, | |
| | 5.4 Hz, 1H), 4.58 (dt, J = 7.0, 3.8 Hz, 1H), 4.50 (s, 2H), 3.74- | |
| | 3.66 (m, 10H), 2.93-2.82 (m, 2H), 2.68-2.53 (m, 4H), | |
| | (72.09-2.02 (m, 1H), 1.98-1.54 (m, 14H), 1.13 (d, J = 6.6 | |
| | Hz, 3H) ppm. | |
| NU227330 | ¹H NMR (500 MHZ, DMSO) § 11.18 (d, J = 7.5 Hz, 1H), | LCMS: t_{R} = |
| | 10.14 (s, 1H), 8.91 (d, J = 4.6 Hz, 1H), 8.15-8.09 (m, 1H), | 1.45 min, 98% |
| | 8.03 (dd, J = 11.0, 2.8 Hz, 1H), 8.01-7.87 (m, 3H), 7.71 (td, | purity, m/z = |
| | J = 8.7, 2.8 Hz, 1H), 7.64 (dd, J = 9.0, 2.3 Hz, 1H), 7.60 (d, J = | 921.8 (M + H)⁻. |
| | 4.7 Hz, 1H), 7.36-7.26 (m, 1H), 7.18 (td, J = 8.1, 2.3 Hz, | |
| | 1H), 5.24-5.13 (m, 1H), 4.60 (s, 1H), 4.48 (s, 2H), 3.76- | |
| | 3.54 (m, 10H), 2.94-2.81 (m, 2H), 2.68-2.55 (m, 4H), 2.10- | |
| | 2.01 (m, 1H), 2.00-1.52 (m, 14H), 1.14 (d, J = 6.6 Hz, 3H) | |
| | ppm. | |
| NU227331 | ¹H NMR (500 MHZ, DMSO) § 11.16 (s, 1H), 9.86 (s, 1H), | LCMS: tr = |
| | 8.94 (d, J = 4.6 Hz, 1H), 8.13 (dd, J = 9.2, 5.7 Hz, 1H), 8.10- | 1.32 min, 82% |
| | 8.02 (m, 1H), 7.96-7.78 (m, 2H), 7.78-7.69 (m, 1H), 7.66 | purity, m/z = |
| | (d, J = 4.9 Hz, 1H), 7.51 (td, J = 6.4, 3.0 Hz, 2H), 6.91 (td, J = | 885.4 (M)+. |
| | 8.4, 5.8 Hz, 2H), 5.15 (dd, J = 12.9, 5.5 Hz, 1H), 4.49 (s, | |
| | 3H), 3.73-3.58 (m, 6H), 3.49-3.37 (m, 2H), 3.37-3.16 (m, | |
| | 3H), 2.94-2.76 (m, 2H), 2.66-2.51 (m, 3H), 2.13-1.36 (m, | |
| | 15H), 1.12 (d, J = 6.7 Hz, 3H) ppm. | |
| NU227353 | ¹H NMR (500 MHZ, DMSO) § 10.82 (s, 1H), 9.86 (s, 1H), | LCMS: t_{R} = |
| | 8.94 (d, J = 4.7 Hz, 1H), 8.20-8.01 (m, 3H), 7.81-7.71 (m, | 1.28 min, 97% |
| | 1H), 7.65 (d, J = 4.7 Hz, 1H), 7.59-7.47 (m, 2H), 7.19-7.08 | purity, m/z = |
| | (m, 2H), 6.98-6.82 (m, 4H), 4.51 (td, J = 7.6, 3.7 Hz, 1H), | 880.7 (M + H)⁻. |
| | 4.46 (s, 2H), 3.79 (td, J = 13.8, 6.4 Hz, 2H), 3.68 (d, J = 14.4 | |
| | Hz, 1H), 3.61 (t, J = 6.7 Hz, 2H), 3.43 (t, J = 5.9 Hz, 3H), | |
| | 3.35-3.17 (m, 5H), 2.83 (dq, J = 13.2, 6.8 Hz, 1H), 2.70- | |
| | 2.54 (m, 4H), 2.48-2.41 (m, 1H), 2.15 (qd, J = 12.4, 4.4 Hz, | |
| | 1H), 2.03-1.40 (m, 16H), 1.12 (d, J = 6.6 Hz, 3H) ppm. | |
| NU227354 | ¹H NMR (500 MHz, DMSO) 8 10.82 (s, 1H), 9.86 (s, 1H), | LCMS: t_{R} = |
| | 9.02 (d, J = 4.9 Hz, 1H), 8.17 (td, J = 9.6, 4.2 Hz, 2H), 8.09 | 1.30 min, 99% |
| | (t, J = 5.8 Hz, 1H), 7.82 (ddd, J = 10.7, 8.3, 2.8 Hz, 1H), 7.75 | purity, m/z = |
| | (d, J = 4.9 Hz, 1H), 7.55-7.48 (m, 2H), 7.17-7.11 (m, 2H), | 924.5 (M + H)⁻. |
| | 6.91 (t, J = 8.3 Hz, 4H), 4.51 (tt, J = 7.5, 3.5 Hz, 1H), 4.46 (s, | |
| | 2H), 3.80 (ddd, J = 20.8, 12.1, 5.5 Hz, 2H), 3.68 (dd, J = 12.4, | |
| | 6.5 Hz, 1H), 3.60 (t, J = 6.7 Hz, 2H), 3.53-3.50 (m, 5H), | |
| | 3.43 (t, J = 5.9 Hz, 3H), 3.35-3.16 (m, 4H), 2.84 (dq, J = | |
| | 13.6, 6.7 Hz, 1H), 2.70-2.51 (m, 4H), 2.49-2.42 (m, 2H), | |
| | 2.15 (qd, J = 12.1, 4.4 Hz, 1H), 2.08-1.51 (m, 14H), 1.45 | |
| | (dt, J = 12.8, 4.5 Hz, 1H), 1.12 (d, J = 6.5 Hz, 3H) ppm. | |
| NU223755 | ¹H NMR (500 MHZ, DMSO) 8 11.12 (s, 1H), 10.17 (s, 1H), | LCMS: t_{R} = |
| | 8.89 (d, J = 4.5 Hz, 1H), 8.11 (dd, J = 9.2, 5.8 Hz, 1H), 8.01 | 1.17 min, 98% |
| | (dd, J = 10.8, 3.0 Hz, 1H), 7.91-7.62 (m, 3H), 7.59 (d, J = | purity, m/z = |
| | 5.5 Hz, 2H), 7.35 (d, J = 8.7 Hz, 1H), 7.20-7.06 (m, 1H), | 991.9 (M + H)⁻. |
| | 5.17-5.09 (m, 1H), 4.67 (s, 1H), 4.33 (s, 2H), 3.78-2.81 | |
| | (m, 21H), 2.65-2.53 (m, 1H), 2.34 (d, J = 23.1 Hz, 2H), 2.07- | |
| | 1.57 (m, 16H), 1.14 (d, J = 6.5 Hz, 3H). | |
| NU227361 | ¹H NMR (500 MHZ, DMSO) 8 11.11 (d, J = 11.9 Hz, 1H), | LCMS: t_{R} = |
| | 9.87 (s, 2H), 8.89 (d, J = 4.6 Hz, 1H), 8.10 (dd, J = 9.2, 5.8 | 1.23 min, 74% |
| | Hz, 1H), 8.02 (dd, J = 11.0, 3.0 Hz, 1H), 7.73-7.67 (m, 1H), | purity, m/z = |
| | 7.61 (dd, J = 8.8, 6.4 Hz, 2H), 7.52 (d, J = 8.7 Hz, 2H), 7.28 | 453.4 |
| | (d, J = 8.6 Hz, 1H), 7.22 (d, J = 7.0 Hz, 1H), 6.90 (dd, J = 9.1, | (M/2 + H)⁺. |
| | 3.9 Hz, 2H), 5.08 (dd, J = 13.0, 5.5 Hz, 1H), 4.51 (s, 1H), | |
| | 3.86-3.17 (m, 14H), 3.03 (s, 3H), 2.95-2.77 (m, 4H), 2.67-2.53 | |
| | (m, 3H), 2.02-1.70 (m, 9H), 1.68-1.52 (m, 4H), 1.44 (d, J = | |
| | 9.8 Hz, 1H), 1.11 (d, J = 6.6 Hz, 3H). | |
| NUCC- | ¹H NMR (500 MHz, DMSO) & 9.98 (s, 1H), 8.89 (d, J = 4.6 | m/z = 953.6 |
| 0227428 | Hz, 1H), 8.11 (dd, J = 9.2, 5.8 Hz, 1H), 8.00 (dd, J = 11.0, 2.9 | (M + H)+ |
| | Hz, 1H), 7.78 (d, J = 8.4 Hz, 1H), 7.69 (td, J = 8.7, 2.8 Hz, | |
| | 1H), 7.59 (d, J = 4.6 Hz, 1H), 7.50 (t, J = 2.9 Hz, 2H), 7.37 | |
| | (d, J = 8.3 Hz, 1H), 7.20 (t, J = 8.1 Hz, 1H), 7.06 (d, J = 8.1 | |
| | Hz, 1H), 6.69 (dd, J = 8.3, 2.5 Hz, 1H), 5.17 (dd, J = 13.1, 5.4 | |
| | Hz, 1H), 4.62 (s, 2H), 4.32 (s, 4H), 3.88-3.15 (m, 8H | |
| | covered by the water peak), 3.01 (s, 7H), 2.87 (dd, J = 11.0, | |
| | 6.6 Hz, 1H), 2.81-2.74 (m, 1H), 2.64-2.53 (m, 2H), 2.38- | |
| | 2.23 (m, 2H), 2.10-1.53 (m, 19H), 1.13 (d, J = 6.6 Hz, 3H) | |
| | ppm. | |
| NUCC- | ¹H NMR (500 MHZ, DMSO) 8 11.09 (s, 1H), 9.96 (s, 1H), | m/z = 436.6 |
| 0227427 | 8.89 (d, J = 4.6 Hz, 1H), 8.10 (dd, J = 9.2, 5.8 Hz, 1H), 8.00 | (M/2 + H)⁺ |
| | (dd, J = 10.9, 3.1 Hz, 1H), 7.77 (d, J = 8.5 Hz, 1H), 7.69 (td, | |
| | J = 8.6, 2.8 Hz, 1H), 7.59 (d, J = 4.7 Hz, 1H), 7.52-7.46 (m, | |
| | 2H), 7.37 (dd, J = 8.6, 2.3 Hz, 1H), 7.22-7.17 (m, 1H), 7.07 | |
| | (d, J = 8.7 Hz, 1H), 6.68 (dt, J = 7.8, 3.8 Hz, 1H), 5.09 (dd, J = | |
| | 12.8, 5.4 Hz, 1H), 4.58 (s, 1H), 4.23 (d, J = 13.8 Hz, 1H), | |
| | 3.83 (s, 1H), 3.76-3.14 (m, 7H, hidden under water peak), | |
| | 2.94-2.81 (m, 4H), 2.63-2.53 (m, 2H), 2.15-1.44 (m, | |
| | 19H), 1.13 (d, J = 6.6 Hz, 3H). | |
| NUCC- | ¹H NMR (500 MHz, DMSO) 8 11.09 (s, 1H), 9.97 (s, 1H), | m/z = 429.8 |
| 0227426 | 8.89 (d, J = 4.6 Hz, 1H), 8.11 (dd, J = 9.2, 5.8 Hz, 1H), | (M/2 + H)+ |
| | 8.03-7.97 (m, 1H), 7.78 (d, J = 8.5 Hz, 1H), 7.73-7.66 (m, 1H), | |
| | 7.59 (d, J = 4.7 Hz, 1H), 7.48 (s, 2H), 7.38-7.31 (m, 1H), | |
| | 7.22-7.18 (m, 1H), 7.08 (d, J = 7.8 Hz, 1H), 6.72-6.66 (m, | |
| | 1H), 5.10 (dd, J = 12.8, 5.5 Hz, 1H), 4.61 (s, 1H), 4.43-3.17 | |
| | (m, 7H, hidden under water peak), 2.88 (q, J = 11.7 Hz, 3H), | |
| | 2.65-2.53 (m, 3H), 2.36 (s, 1H), 2.06-1.55 (m, 19H), 1.13 | |
| | (d, J = 6.6 Hz, 3H). | |
| NUCC- | ¹H NMR (500 MHz, DMSO) 8 11.09 (s, 1H), 8.90 (d, J = 4.6 | m/z = 447.0 |
| 0227425 | Hz, 1H), 8.13 (dd, J = 9.6, 5.4 Hz, 1H), 8.03 (d, J = 10.4 Hz, | (M/2 + H)+ |
| | 1H), 7.91-7.85 (m, 1H), 7.74-7.68 (m, 1H), 7.64-7.52 (m, | |
| | 2H), 7.16-7.06 (m, 1H), 7.02 (dd, J = 8.9, 6.9 Hz, 1H), 6.58 | |
| | (d, J = 14.7 Hz, 1H), 5.05 (dt, J = 12.8, 4.4 Hz, 1H), 4.16 (dd, | |
| | J = 39.1, 13.1 Hz, 1H), 3.85-3.56 (m, 9H under water peak), | |
| | 3.07 (q, J = 12.3 Hz, 2H), 2.93-2.52 (m, 7H), 2.07-1.53 | |
| | (m, 14H), 1.23 (td, J = 43.3, 11.2 Hz, 3H), 1.01 (d, J = 6.6 Hz, | |
| | 3H). | |
| NUCC- | ¹H NMR (500 MHz, DMSO) 8 11.09 (s, 1H), 8.86 (t, J = 3.3 | m/z = 432.3 |
| 0227424 | Hz, 1H), 8.10 (dd, J = 9.1, 5.7 Hz, 1H), 8.02-7.92 (m, 2H), | (M/2 + H)+ |
| | 7.77 (d, J = 8.4 Hz, 1H), 7.68 (td, J = 8.7, 2.7 Hz, 1H), 7.55 | |
| | (t, J = 4.2 Hz, 1H), 7.50 (s, 1H), 7.37 (s, 1H), 5.09 (dd, J = | |
| | 12.8, 5.5 Hz, 1H), 4.34-4.07 (m, 6H), 3.15 (dd, J = 9.2, 5.0 | |
| | Hz, 5H), 2.91 (td, J = 19.9, 8.3 Hz, 5H), 2.66-2.54 (m, 3H), | |
| | 2.09-1.47 (m, 21H), 1.34-1.18 (m, 5H), 1.03 (d, J = 6.5 | |
| | Hz, 3H). | |

### Synthetic schemes for the preparation of IDO1 PROTACs

### Preparation of (R)-2-((1s,4S)-4-(6-fluoroquinolin-4-yl)cyclohexyl)-N-(4-(piperidin-4-yloxy)phenyl)propanamide

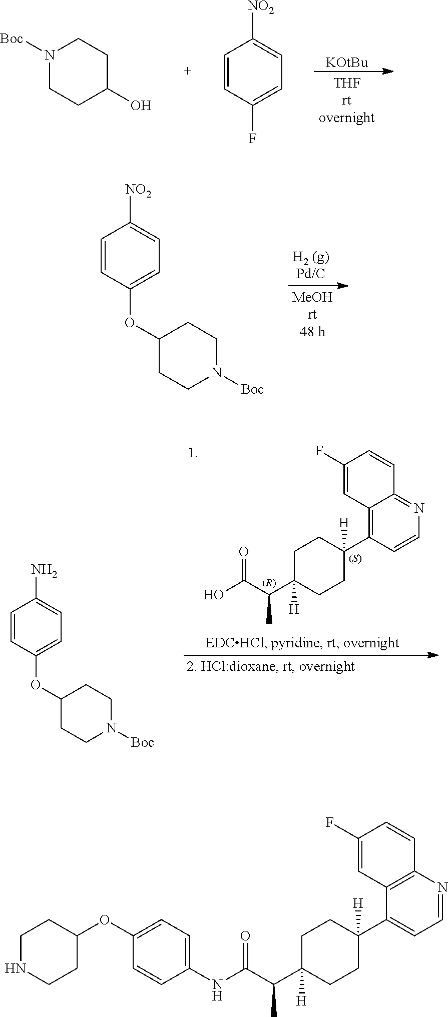

### Preparation of (R)-N-(4-chloro-3-(piperidin-4-yloxy)phenyl)-2-((1s,4S)-4-(6-fluoroquinolin-4-yl)cyclohexyl)propanamide

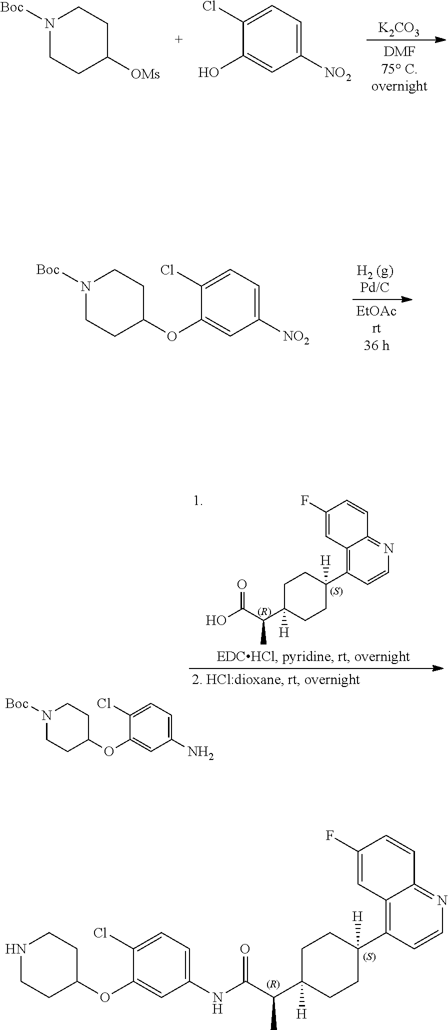

### Preparation of (R)-2-((1s,4S)-4-(6-fluoroquinolin-4-yl)cyclohexyl)-N-(3-(piperidin-4-yloxy)phenyl)propanamide

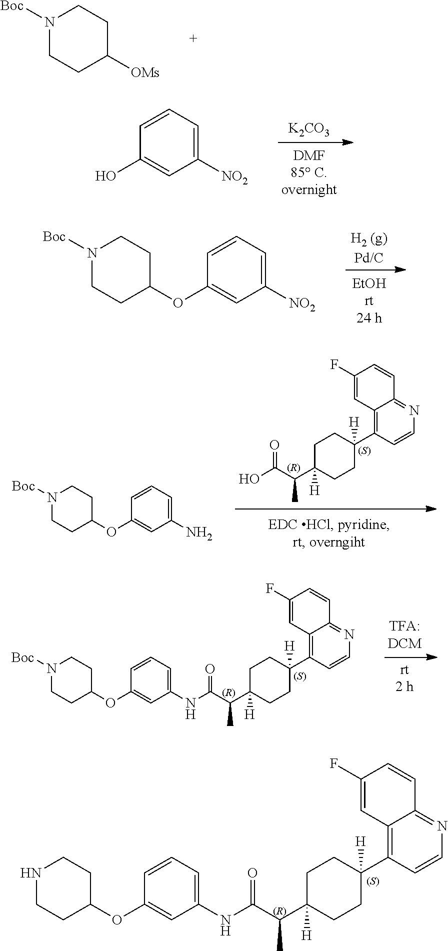

### Preparation of (2R)-N-(4-((1-(3-(2-(4-(2-(2,6-dioxopiperidin-3-yl)-1,3-dioxoisoindolin-4-yl)piperazin-1-yl)ethoxy)propanoyl)piperidin-4-yl)oxy)phenyl)-2-((1s,4S′)-4-(6-fluoroquinolin-4-yl)cyclohexyl)propanamide (NU227039) and (2R)-N-(4-chloro-3-((1-(3-(2-(4-(2-(2,6-dioxopiperidin-3-yl)-1,3-dioxoisoindolin-4-yl)piperazin-1-yl)ethoxy)propanoyl)piperidin-4-yl)oxy)phenyl)-2-((1s,4,S)-4-(6-fluoroquinolin-4-yl)cyclohexyl)propanamide (NU227040)

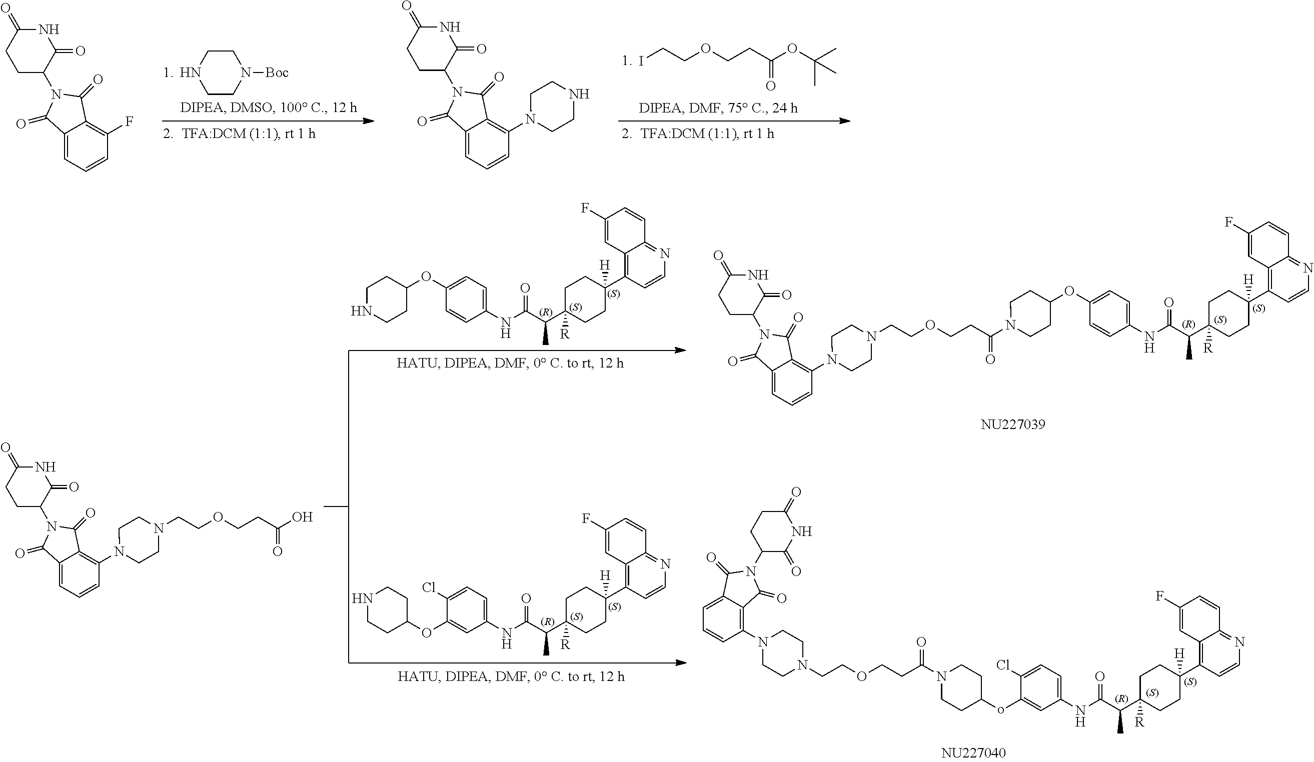

### Preparation of (2R)-N-(4-((1-(3-(2-(4-(2-(2,6-dioxopiperidin-3-yl)-1,3-dioxoisoindolin-5-yl)piperazin-1-yl)ethoxy)propanoyl)piperidin-4-yl)oxy)phenyl)-2-((1s,4S)-4-(6-fluoroquinolin-4-yl)cyclohexyl)propanamide (NU227041) and (2R)-N-(4-chloro-3-((1-(3-(2-(4-(2-(2,6-dioxopiperidin-3-yl)-1,3-dioxoisoindolin-5-yl)piperazin-1-yl)ethoxy)propanoyl)piperidin-4-yl)oxy)phenyl)-2-((1s,4S)-4-(6-fluoroquinolin-4-yl)cyclohexyl)propanamide (NU227042)

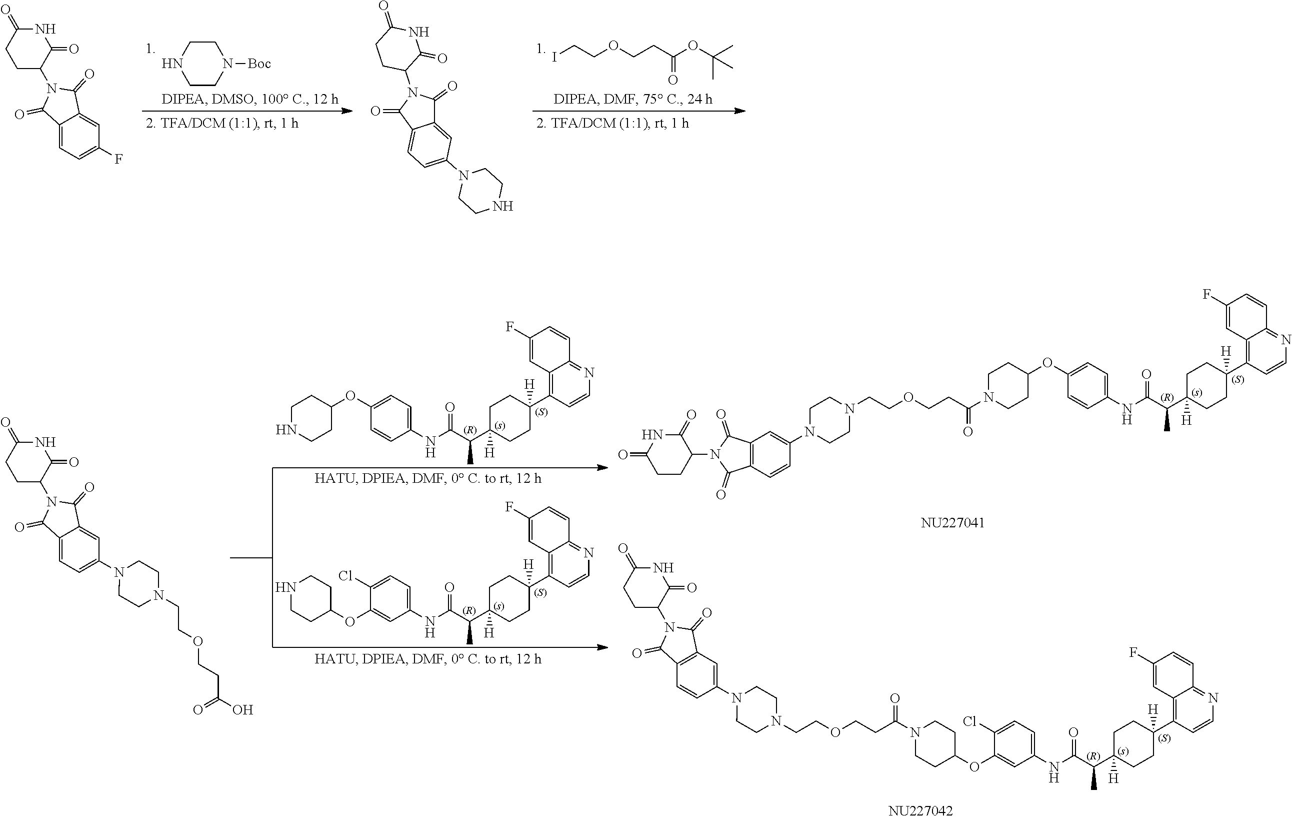

### Preparation of (2R)-N-(4-((1-(3-(2-(2-((2-(2,6-dioxopiperidin-3-yl)-1,3-dioxoisoindolin-5-yl)amino)ethoxy)ethoxy)propanoyl)piperidin-4-yl)oxy)phenyl)-2-((1s,4S)-4-(6-fluoroquinolin-4-yl)cyclohexyl)propanamide (NU227043) and (2R)-N-(4-chloro-3-((1-(3-(2-(2-((2-(2,6-dioxopiperidin-3-yl)-1,3-dioxoisoindolin-5-yl)amino)ethoxy)ethoxy)propanoyl)piperidin-4-yl)oxy)phenyl)-2-((1s,4,S)-4-(6-fluoroquinolin-4-yl)cyclohexyl)propanamide (NU227045)

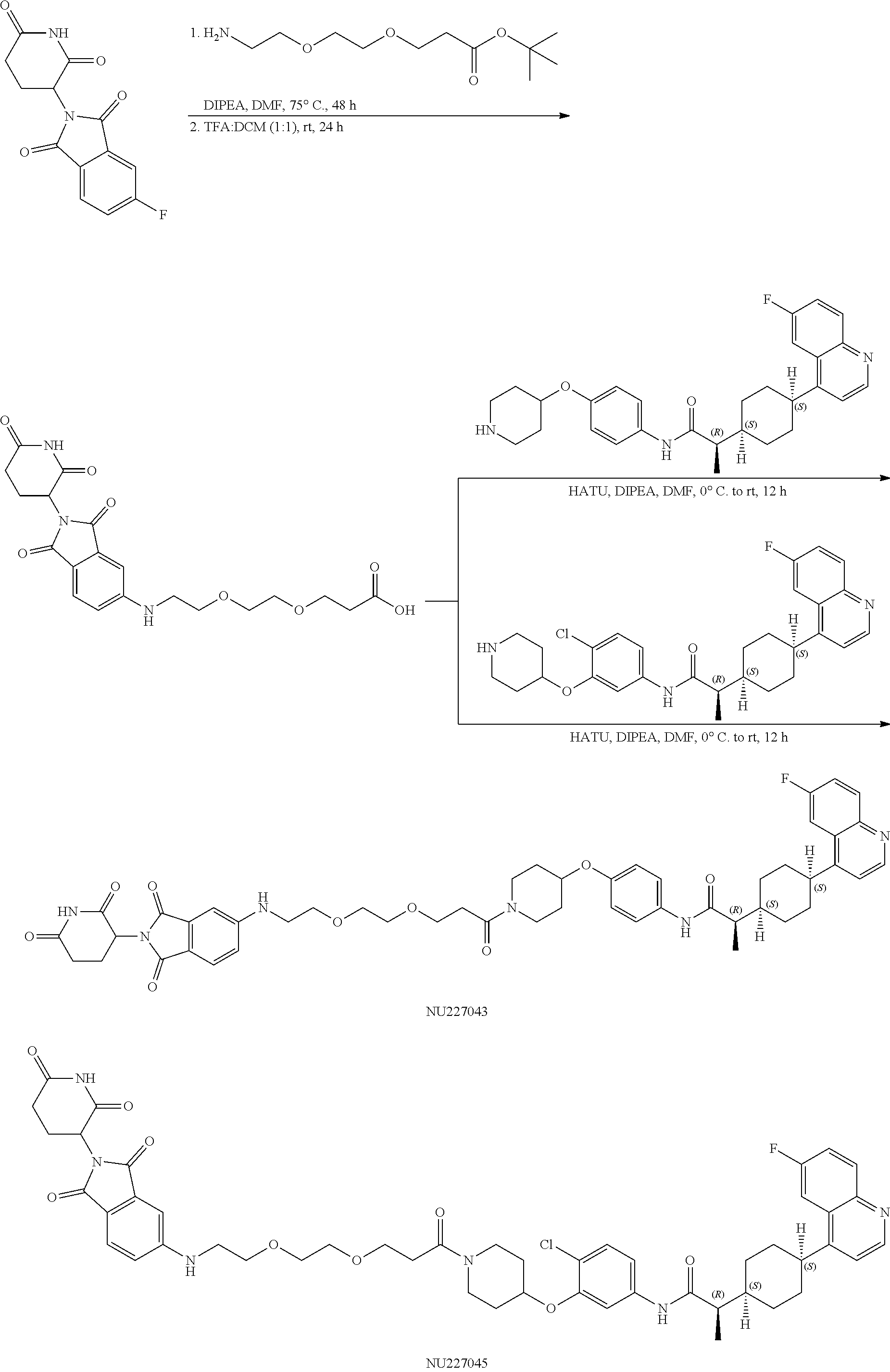

### Preparation of (2R)-N-(4-chloro-3-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide (NU227044) and (2R)-N-(3-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1s,4,S)-4-(6-fluoroquinolin-4-yl)cyclohexyl)propanamide (NU227328)

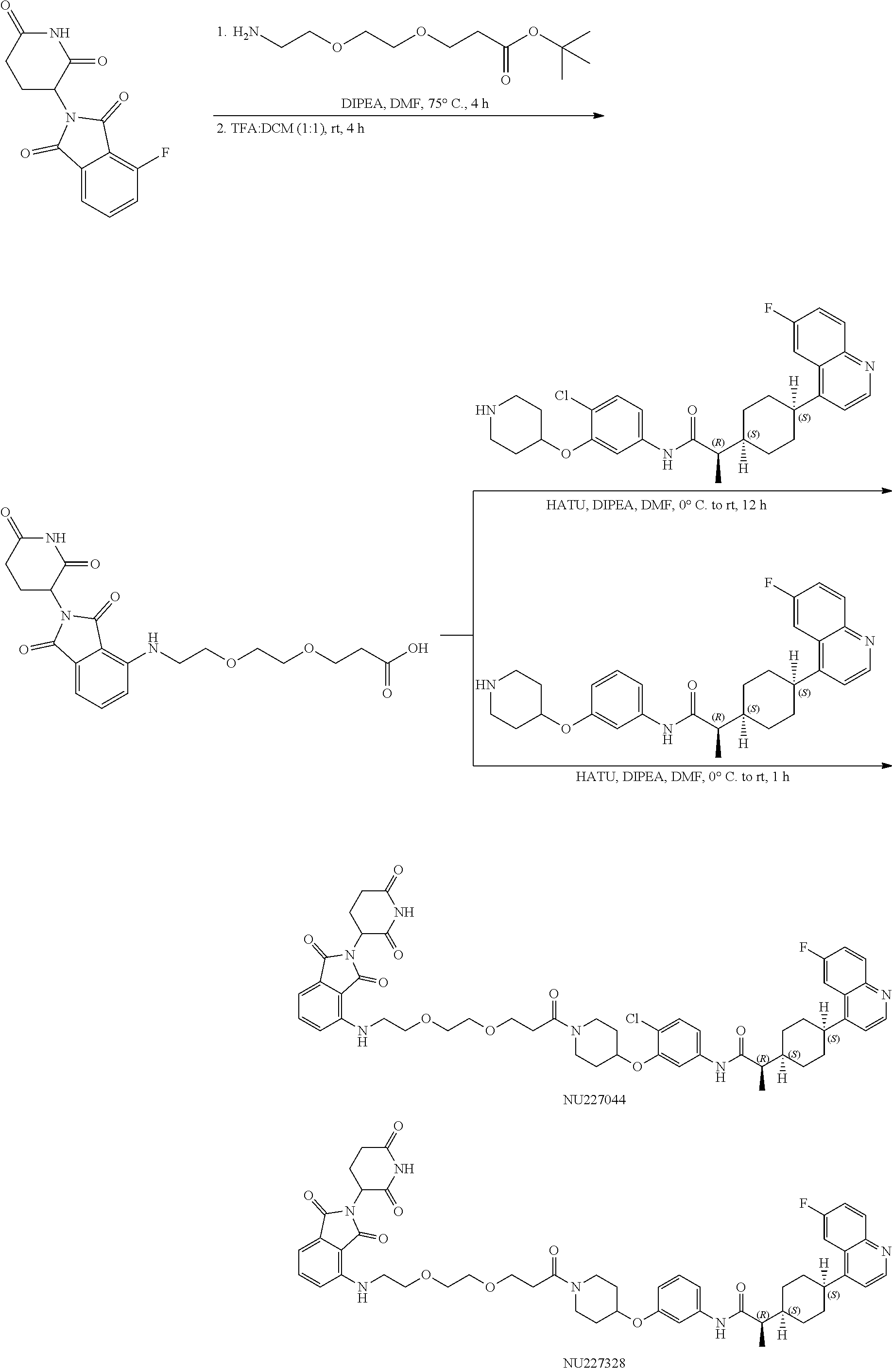

### Preparation of (2R)-N-(4-((1-(3-(2-(2-((2-(2,6-dioxopiperidin-3-yl)-1-oxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide (NU227046) and (2R)-N-(4-chloro-3-((1-(3-(2-(2-((2-(2,6-dioxopiperidin-3-yl)-1-oxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide (NU227047)

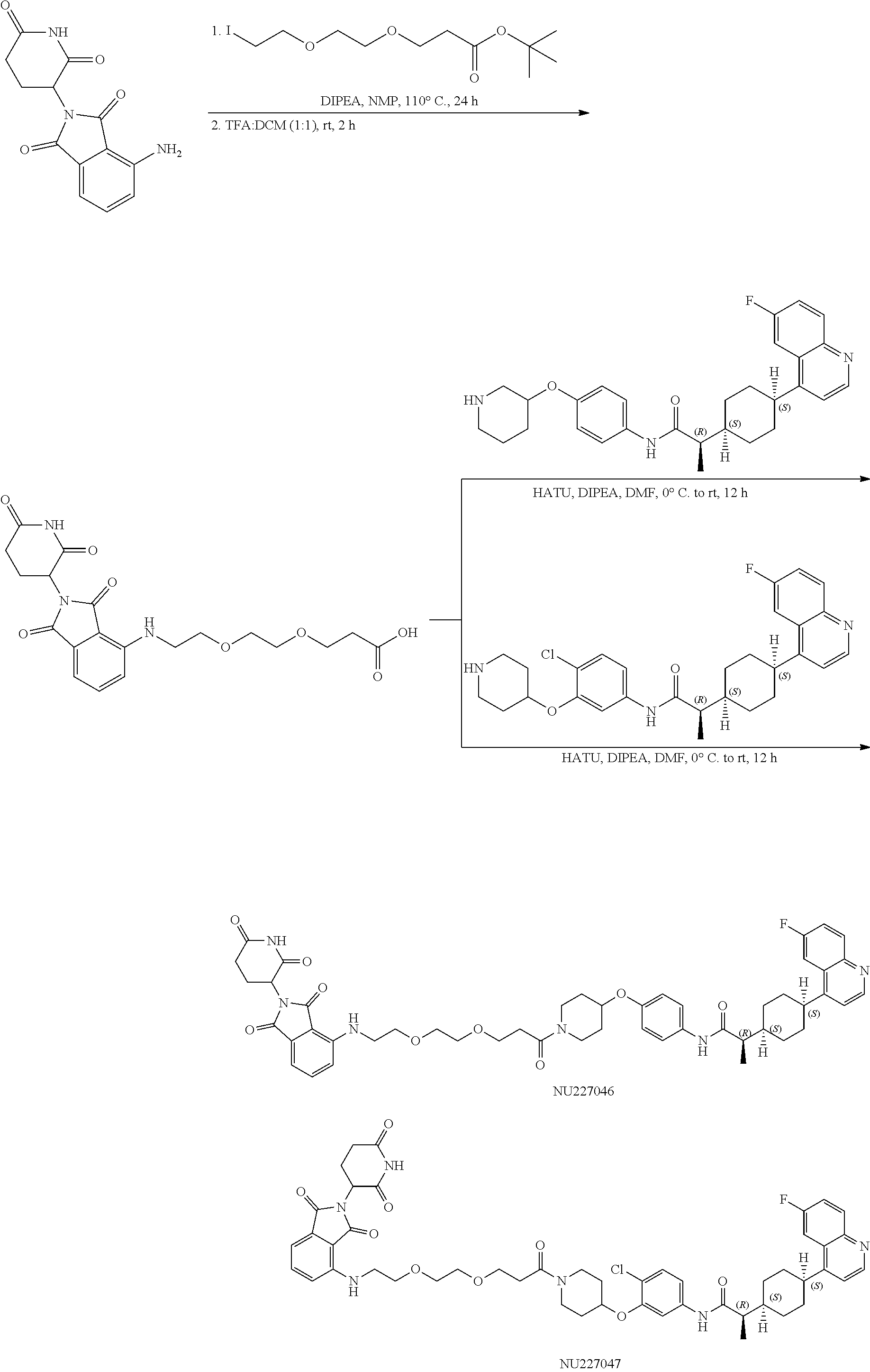

### Preparation of (2R)-N-(4-((1-(3-(4-(2-(2,6-dioxopiperidin-3-yl)-1,3-dioxoisoindolin-4-yl)piperazin-1-yl)benzoyl)piperidin-4-yl)oxy)phenyl)-2-((1s,4S)-4-(6-fluoroquinolin-4-yl)cyclohexyl)propanamide (NU227157) and (2R)-N-(4-((1-(3-(4-(2-(2,6-dioxopiperidin-3-yl)-1,3-dioxoisoindolin-5-yl)piperazin-1-yl)benzoyl)piperidin-4-yl)oxy)phenyl)-2-((1s,4,S)-4-(6-fluoroquinolin-4-yl)cyclohexyl)propanamide (NU227159)

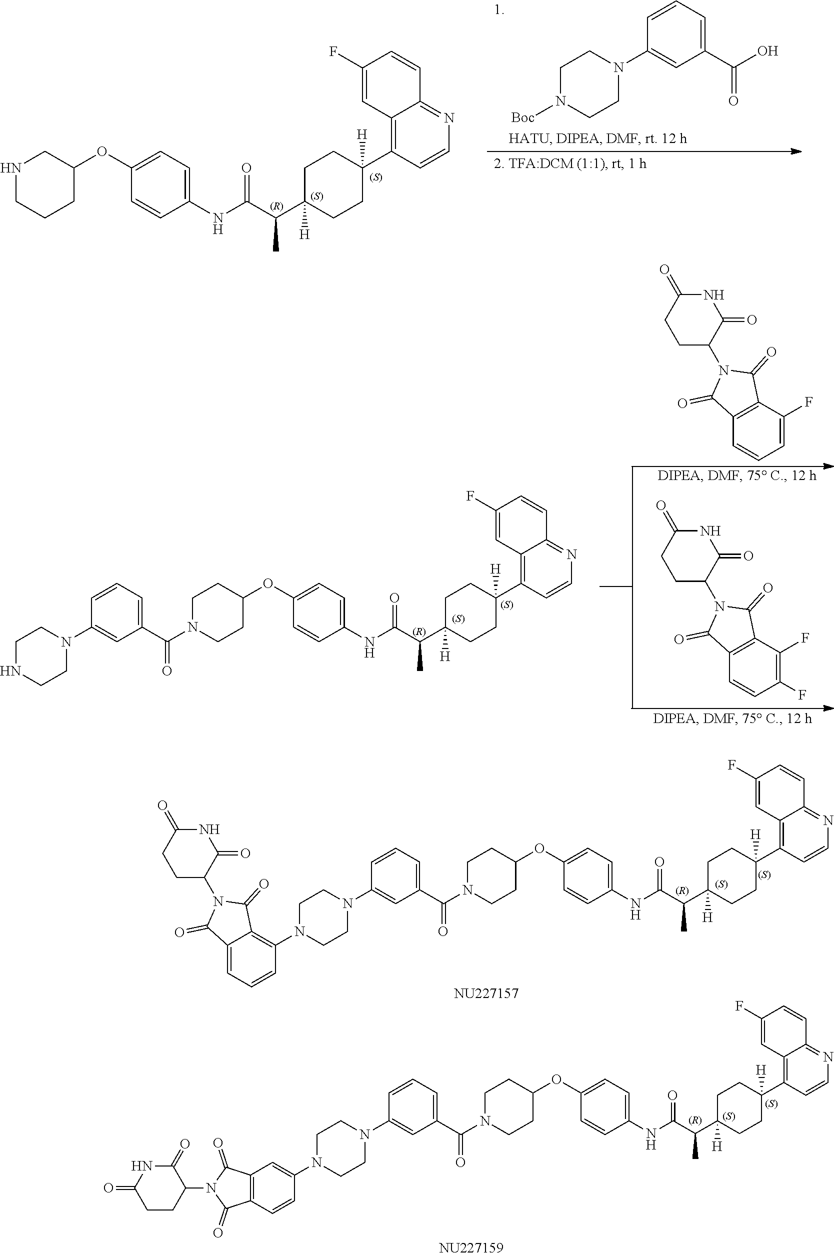

### Preparation of (2R)-N-(4-chloro-3-((1-(3-(4-(2-(2,6-dioxopiperidin-3-yl)-1,3-dioxoisoindolin-4-yl)piperazin-1-yl)benzoyl)piperidin-4-yl)oxy)phenyl)-2-((1s,4S)-4-(6-fluoroquinolin-4-yl)cyclohexyl)propanamide (NU227158) and (2R)-N-(4-chloro-3-((1-(3-(4-(2-(2,6-dioxopiperidin-3-yl)-1,3-dioxoisoindolin-5-yl)piperazin-1-yl)benzoyl)piperidin-4-yl)oxy)phenyl)-2-((1s,4S)-4-(6-fluoroquinolin-4-yl)cyclohexyl)propanamide (NU227160)

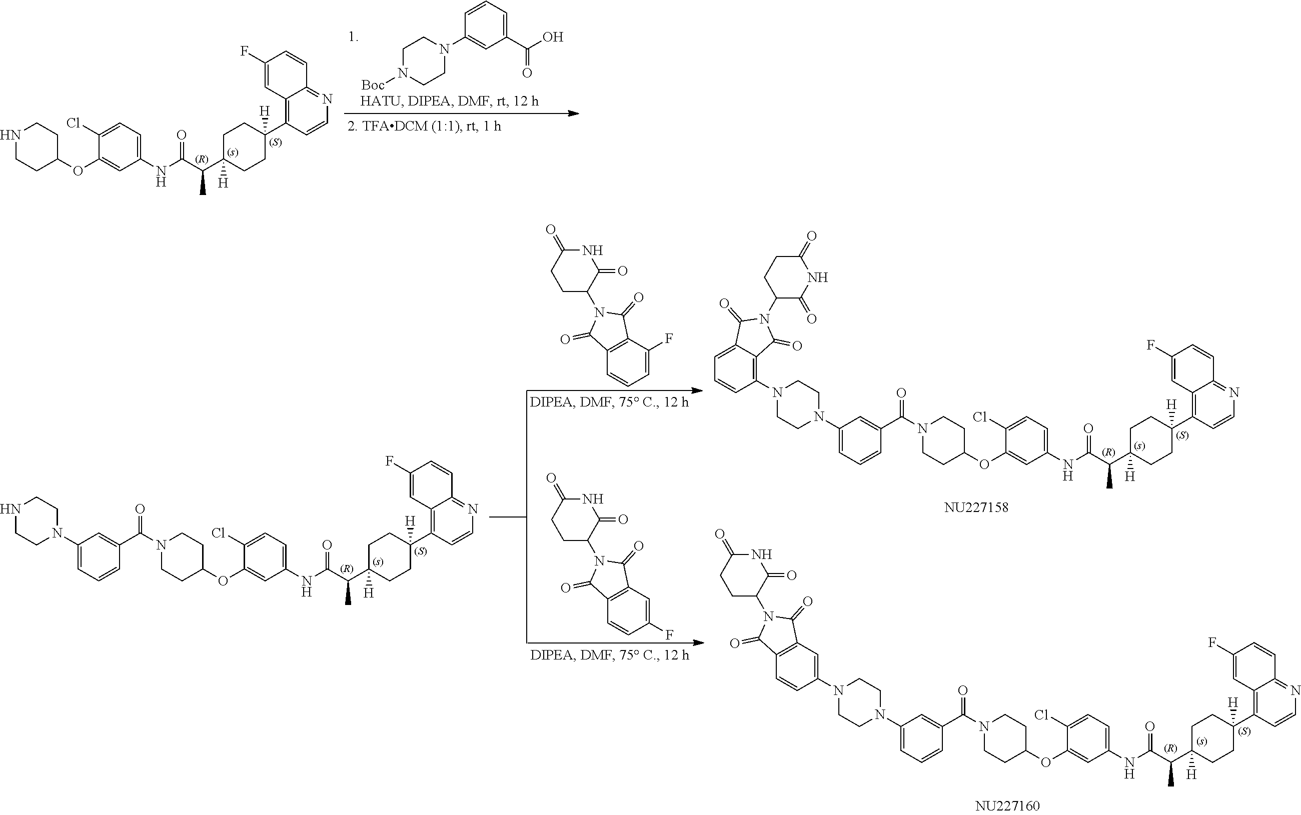

### Preparation of (2R)-N-(4-((1-(2-(4-((4-(2-(2,6-dioxopiperidin-3-yl)-1,3-dioxoisoindolin-4-yl)piperazin-1-yl)methyl)piperidin-1-yl)acetyl)piperidin-4-yl)oxy)phenyl)-2-((1s,4S)-4-(6-fluoroquinolin-4-yl)cyclohexyl)propanamide (NU227161) and (2R)-N-(4-chloro-3-((1-(2-(4-((4-(2-(2,6-dioxopiperidin-3-yl)-1,3-dioxoisoindolin-4-yl)piperazin-1-yl)methyl)piperidin-1-yl)acetyl)piperidin-4-yl)oxy)phenyl)-2-((1s,4S)-4-(6-fluoroquinolin-4-yl)cyclohexyl)propanamide (NU227162)

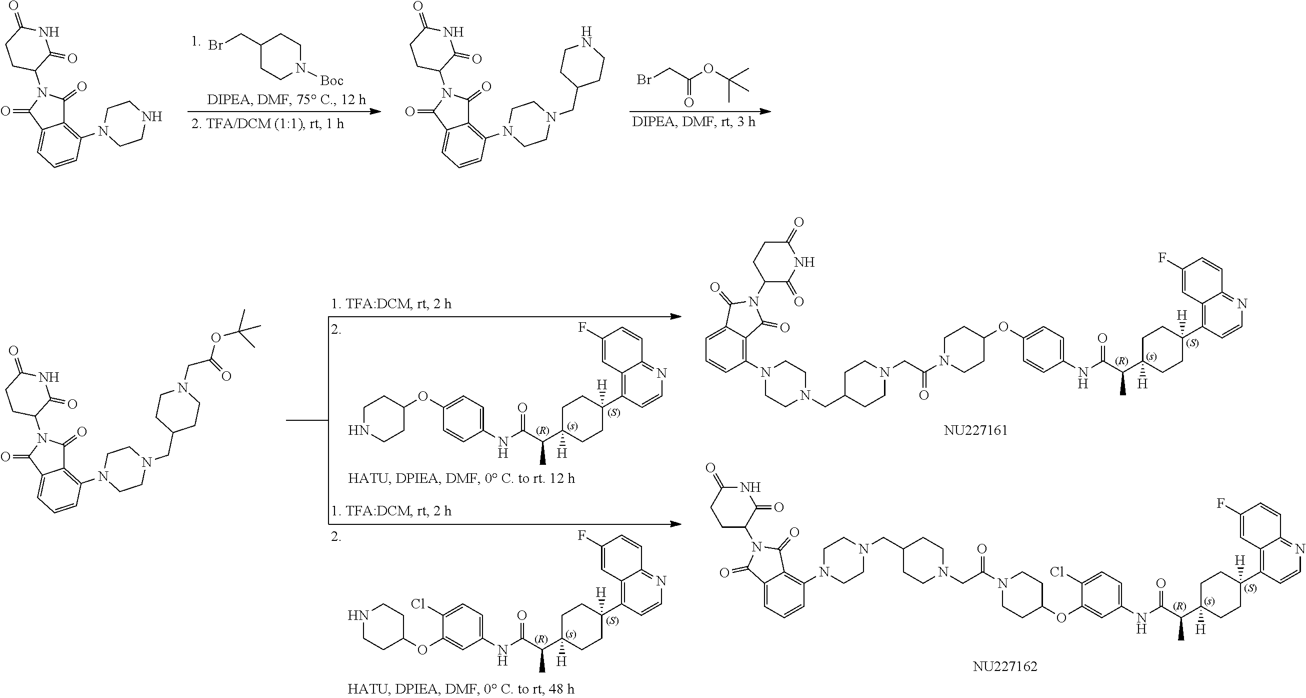

### Preparation of (2R)-N-(4-((1-(2-(4-((4-(2-(2,6-dioxopiperidin-3-yl)-1,3-dioxoisoindolin-5-yl)piperazin-1-yl)methyl)piperidin-1l-yl)acetyl)piperidin-4-yl)oxy)phenyl)-2-((1 s,4,S)-4-(6-fluoroquinolin-4-yl)cyclohexyl)propanamide (NU227163), (2R)-N-(4-chloro-3-((1-(2-(4-((4-(2-(2,6-dioxopiperidin-3-yl)-1,3-dioxoisoindolin-5-yl)piperazin-1-yl)methyl)piperidin-1l-yl)acetyl)piperidin-4-yl)oxy)phenyl)-2-((1s,4S)-4-(6-fluoroquinolin-4-yl)cyclohexyl)propanamide (NU227164) and (2R)-N-(3-((1-(2-(4-((4-(2-(2,6-dioxopiperidin-3-yl)-1,3-dioxoisoindolin-5-yl)piperazin-1-yl)methyl)piperidin-1-yl)acetyl)piperidin-4-yl)oxy)phenyl)-2-((1s,4S)-4-(6-fluoroquinolin-4-yl)cyclohexyl)propanamide (NU227327)

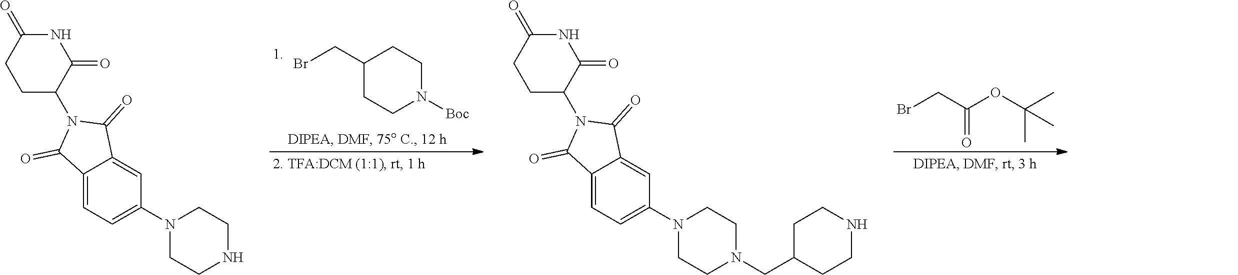

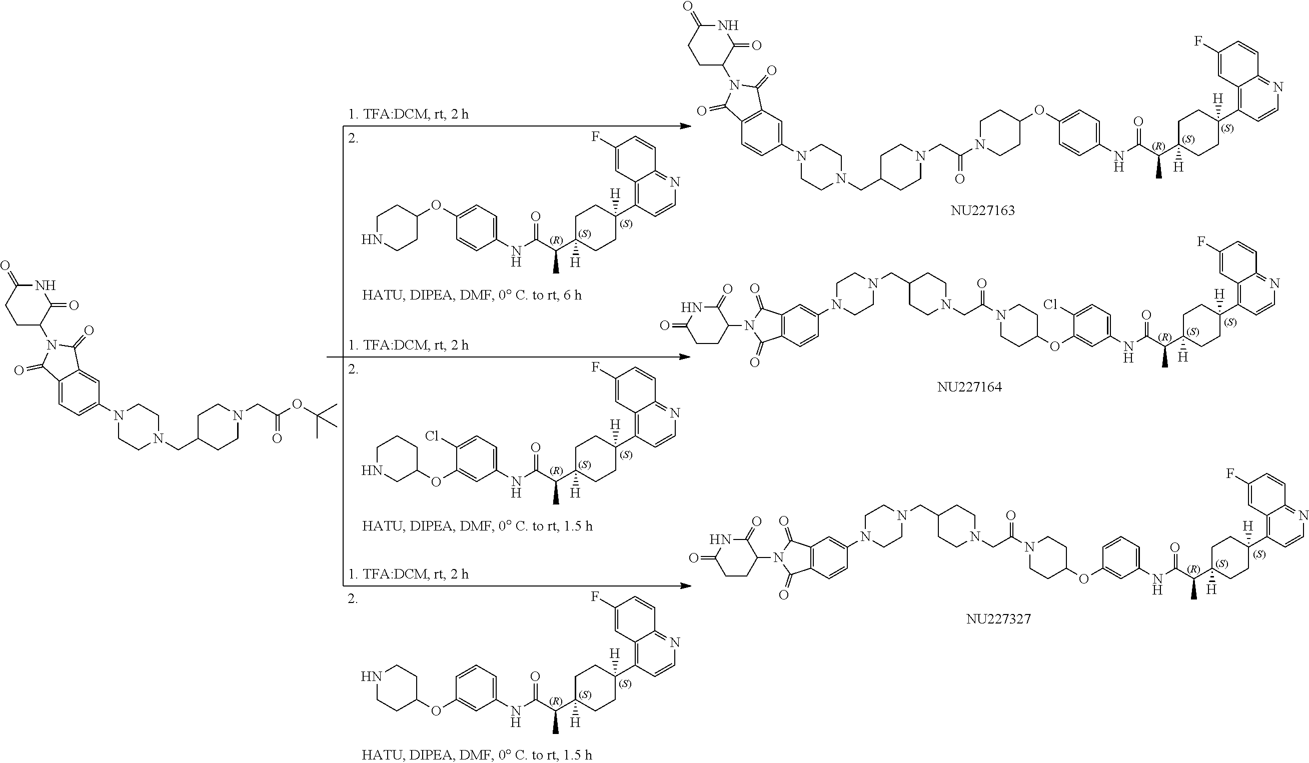

### Preparation of (2R)-N-(4-((1-(2-(4-(4-(2-(2,6-dioxopiperidin-3-yl)-1,3-dioxoisoindolin-4-yl)piperazin-1-yl)piperidin-1-yl)acetyl)piperidin-4-yl)oxy)phenyl)-2-((1s,4S)-4-(6-fluoroquinolin-4-yl)cyclohexyl)propanamide (NU227165) and (2R)-N-(4-chloro-3-((1-(2-(4-(4-(2-(2,6-dioxopiperidin-3-yl)-1,3-dioxoisoindolin-4-yl)piperazin-1-yl)piperidin-1-yl)acetyl)piperidin-4-yl)oxy)phenyl)-2-((1s,4S)-4-(6-fluoroquinolin-4-yl)cyclohexyl)propanamide (N U227166)

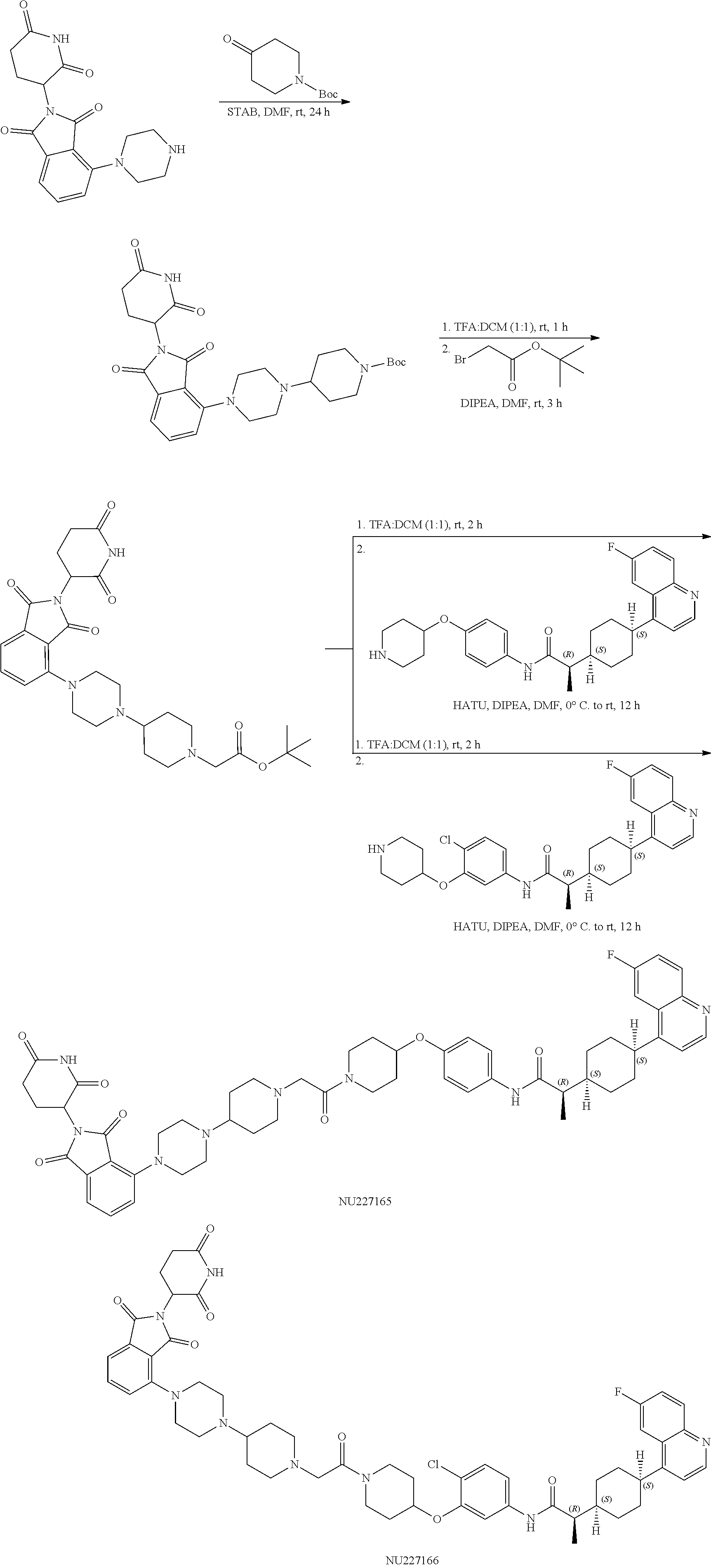

### Preparation of (2R)-N-(4-((1-(2-(4-(4-(2-(2,6-dioxopiperidin-3-yl)-1,3-dioxoisoindolin-5-yl)acetyl)piperidin-4-yl)oxy)phenyl)-2-((1s,4S′)-4-(6-fluoroquinolin-4-yl)cyclohexyl)propanamide (NU227168) and (2R)-N-(3-((1-(2-(4-(4-(2-(2,6-dioxopiperidin-3-yl)-1,3-dioxoisoindolin-5-yl)piperazin-1-yl)piperidin-1l-yl)acetyl)piperidin-4-yl)oxy)phenyl)-2-((1s,4S)-4-(6-fluoroquinolin-4-yl)cyclohexyl)propanamide (NU227326)

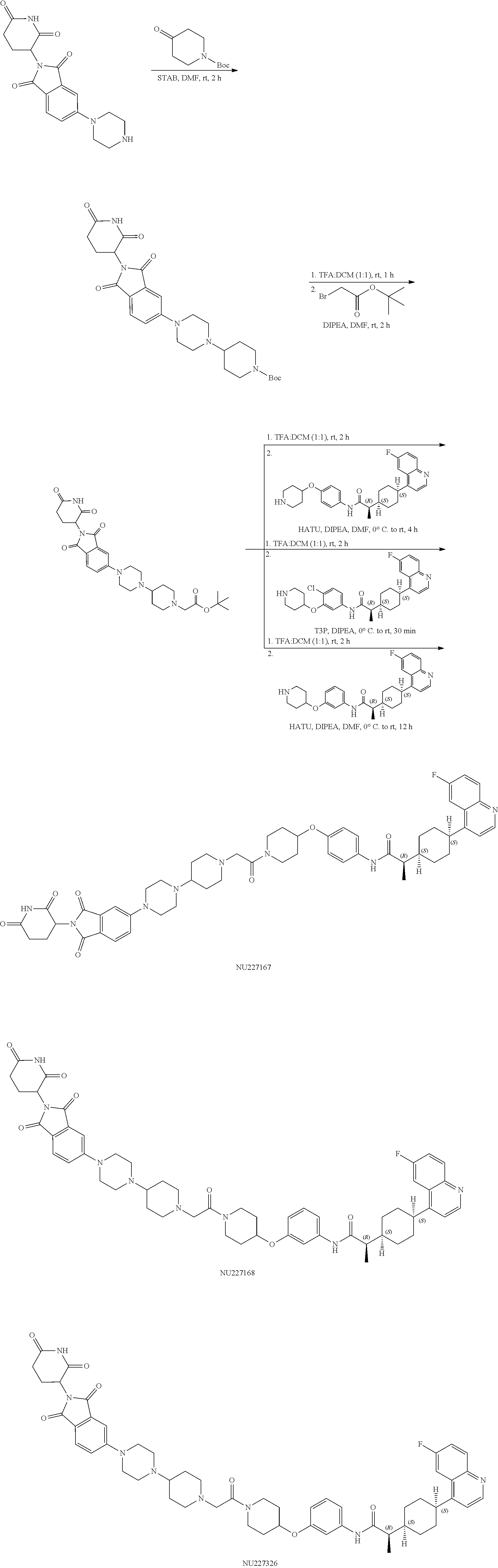

### Preparation of 2-(2,6-dioxopiperidin-3-yl)-4-((2-(2-iodoethoxy)ethyl)amino)isoindoline-1,3-dione

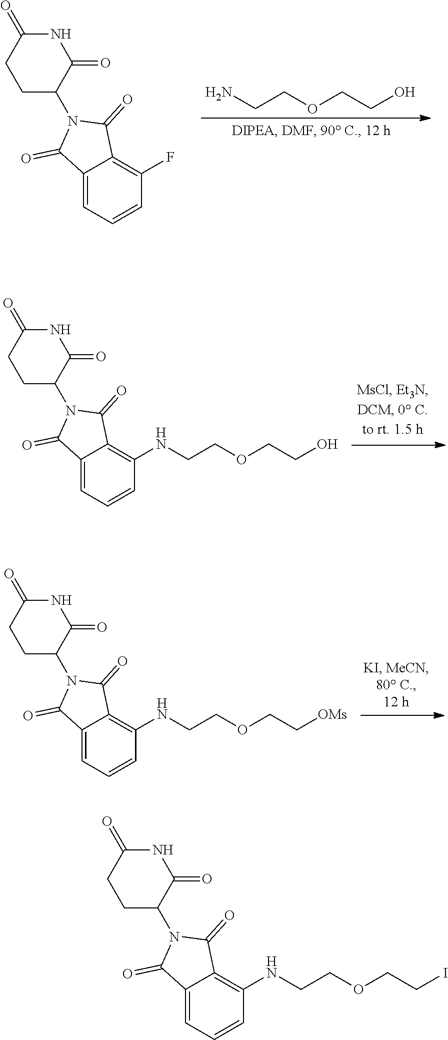

### Preparation of 2-(2,6-dioxopiperidin-3-yl)-5-((2-(2-iodoethoxy)ethyl)amino)isoindoline-1 3-dione

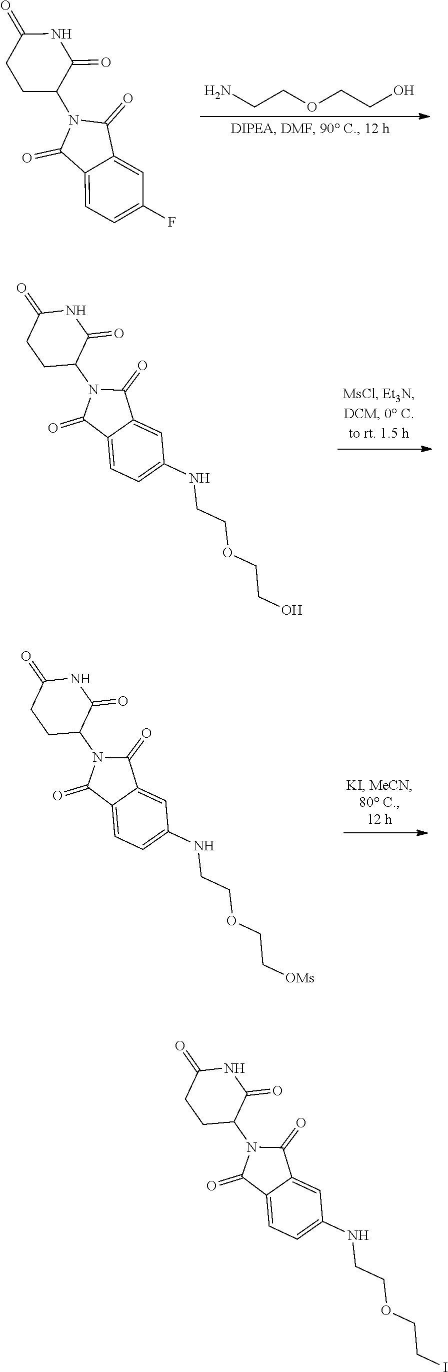 [SMILES: O=C1CCC(N2C(=O)c3ccc(F)cc3C2=O)C(=O)N1 ; NCCOCCO ; O=C1CCC(N2C(=O)c3ccc(NCCOCCO)cc3C2=O)C(=O)N1 ; CS(=O)(=O)OCCOCCNc1ccc2c(c1)C(=O)N(C1CCC(=O)NC1=O)C2=O ; O=C1CCC(N2C(=O)c3ccc(NCCOCCI)cc3C2=O)C(=O)N1]

### Preparation of (2R)-N(4-((1-(2((2-(2,6-dioxopiperidin-3-yl)-1,3-dioxoisoindolin-4-yl)amino)ethyl)piperidine-4-carbonyl)piperidin-4-yl)oxy)phenyl)-2-((1s,4S)-4-(6-fluoroquinolin-4-yl)cyclohexyl)propanamine (NU227182) and (2R)-N-(4-((1-(1-(2-((2-(2,6-dioxopiperidin-3-yl)-1,3-dioxoisoindolin-5-yl)amino)ethoxy)ethyl)piperidine-4-carbonyl)piperidin-4-yl)oxy)phenyl)-2-((1s,4S)-4-(6-fluoroquinolin-4-yl)cyclohexyl)propanamide (NU227191)

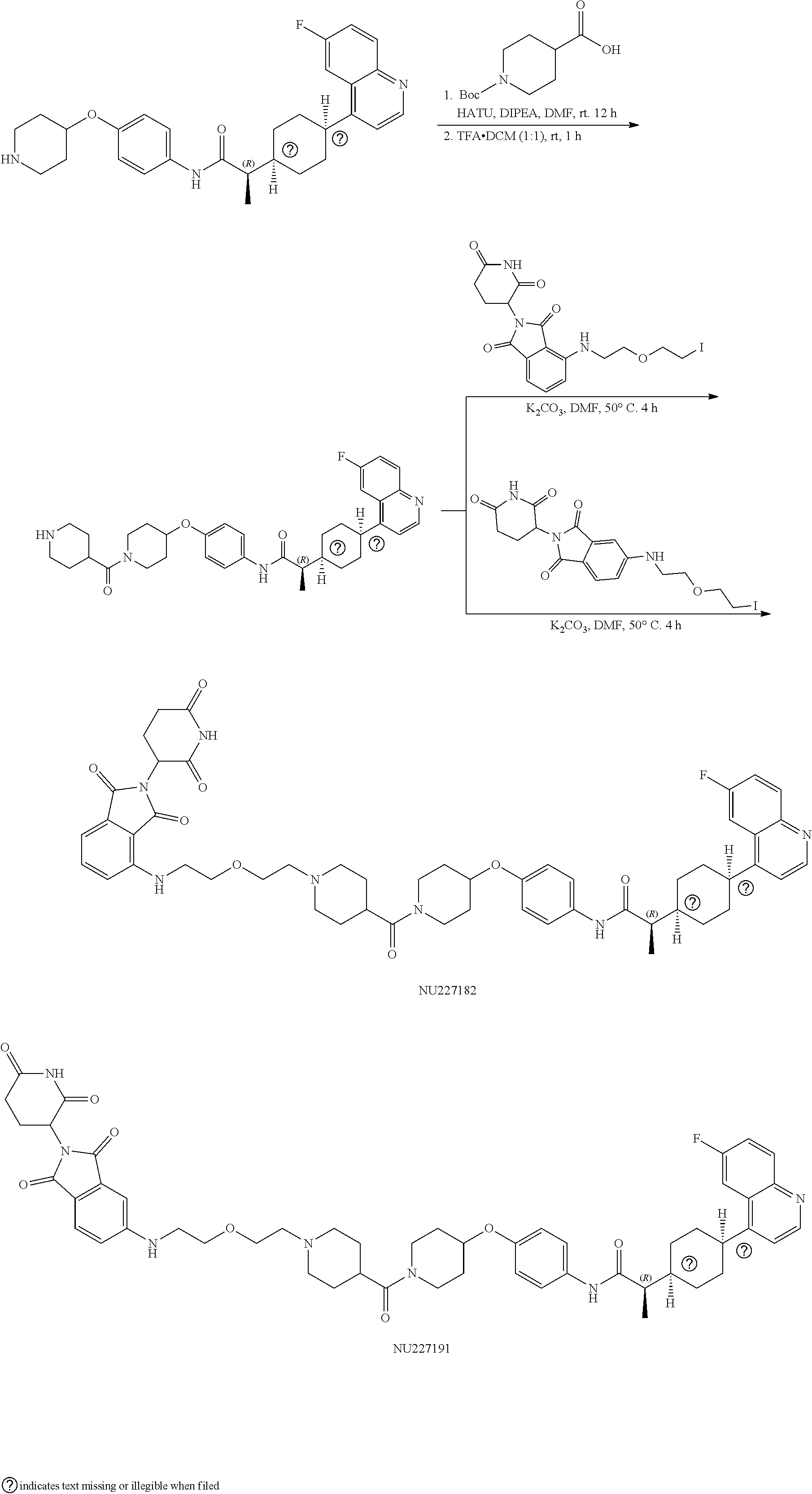

### Preparation of (2R)-N-(4-chloro-3-((1-(1-(2-(2-((2-(2,6-dioxopiperidin-3-yl)-1,3-dioxoisoindolin-4-yl)amino)ethoxy)ethyl)piperidine-4-carbonyl)piperidin-4-yl)oxy)phenyl)-2-((1s,4S)-4-(6-fluoroquinolin-4-yl)cyclohexyl)propanamide (NU227183) and (2R)-N-(4-chloro-3-((1-(1-(2-(2-((2-(2,6-dioxopiperidin-3-yl)-1,3-dioxoisoindolin-5-yl)amino)ethoxy)ethyl)piperidine-4-carbonyl)piperidin-4-yl)oxy)phenyl)-2-((1s,4S)-4-(6-fluoroquinolin-4-yl)cyclohexyl)propanamide (NU227192)

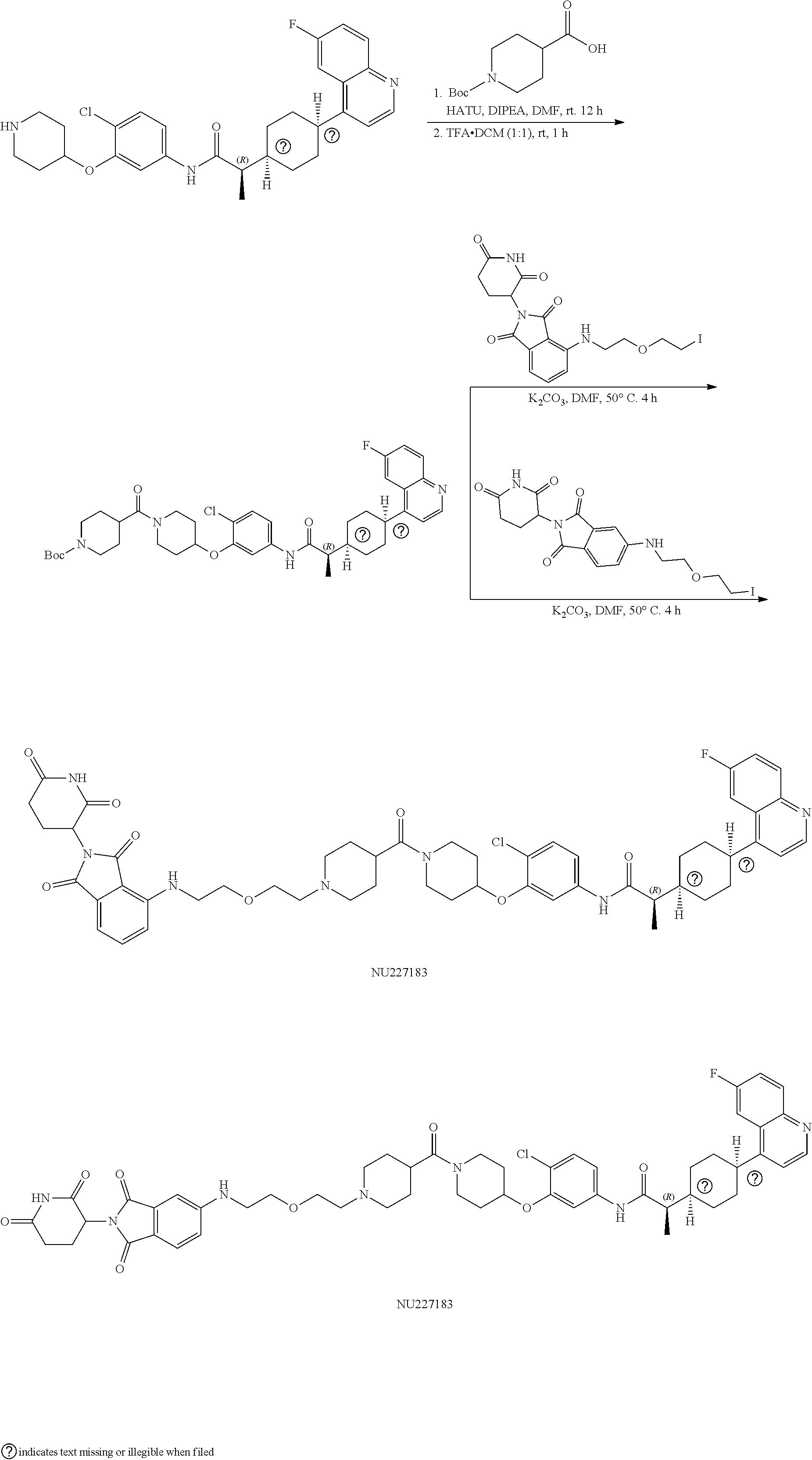

### Preparation i of (2R)-N-(4-((1-(1-(((1r,4r)-4-((2-(2,6-dioxopiperidin-3-yl)-1,3-dioxoisoindolin-4-yl)amino)cyclohexyl)methyl)piperidine-4-carbonyl)piperidin-4-yl)oxy)phenyl)-2-((1s,4&S)-4-(6-fluoroquinolin-4-yl)cyclohexyl)propanamide (NUJ227193) and (2R)-N-(4-((1-(1-(((1r,4r)-4-((2-(2,6-dioxopiperidin-3-yl)-1, 3-dioxoisoindolin-5-yl)amino)cyclohexyl)methyl)piperidine-4-carbonyl)piperidin-4-yl)oxy)phenyl)-2-((1s,4S)-4-(6-fluoroquinolin-4-yl)cyclohexyl) propanamide (NUJ227194)

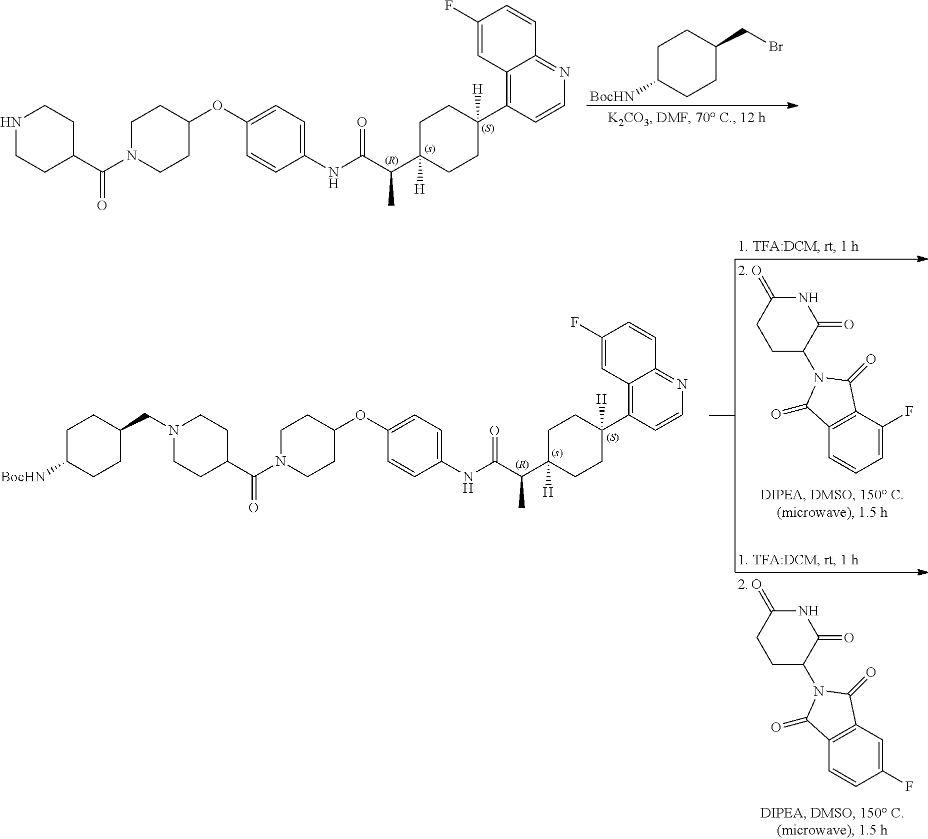

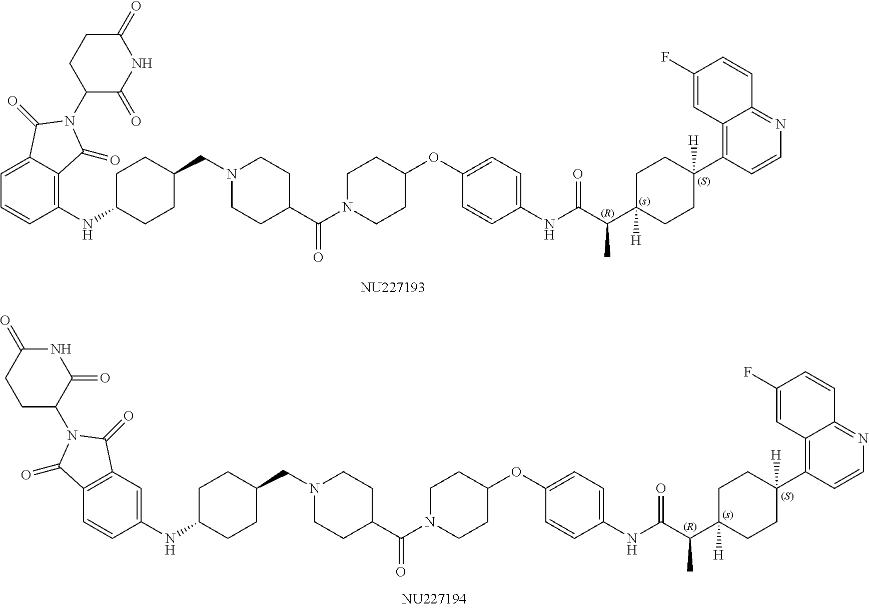

### Preparation of (2R)-N-(4-chloro-3-((1-(1-(((1r,4r)-4-((2-(2,6-dioxopiperidin-3-yl)-1,3-dioxoisoindolin-4-yl)amino)cyclohexyl)methyl)piperidine-4-carbonyl)piperidin-4-yl)oxy)phenyl)-2-((1s,4,S)-4-(6-fluoroquinolin-4-yl)cyclohexyl)propanamide (NU227198) and (2R)-N-(4-chloro-3-((1-(1-(((1r,4r)-4-((2-(2,6-dioxopiperidin-3-yl)-1,3-dioxoisoindolin-5-yl)amino)cyclohexyl)methyl)piperidine-4-carbonyl)piperidin-4-yl)oxy)phenyl)-2-((1s,4S)-4-(6-fluoroquinolin-4-yl)cyclohexyl)propanamide (NU227199)

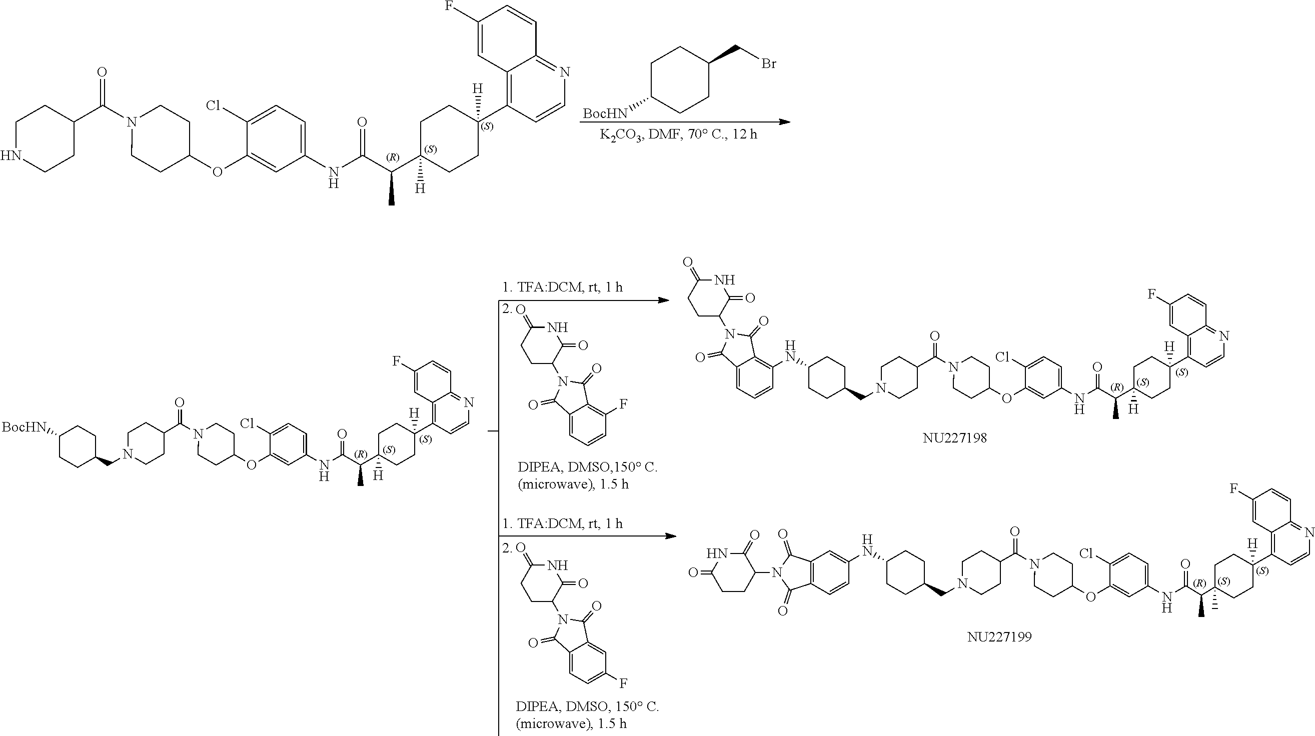

### Preparation of (2R)-N-(4-((1-(9-((2-(2,6-dioxopiperidin-3-yl)-1,3-dioxoisoindolin-4-yl)amino)nonanoyl)piperdine-4-yl)oxy)phenyl)-2-((1s,4S)-4-(6-fluoroquinolin-4-yl)cyclohexyl)propanamide (NU227200) and (NU227201)

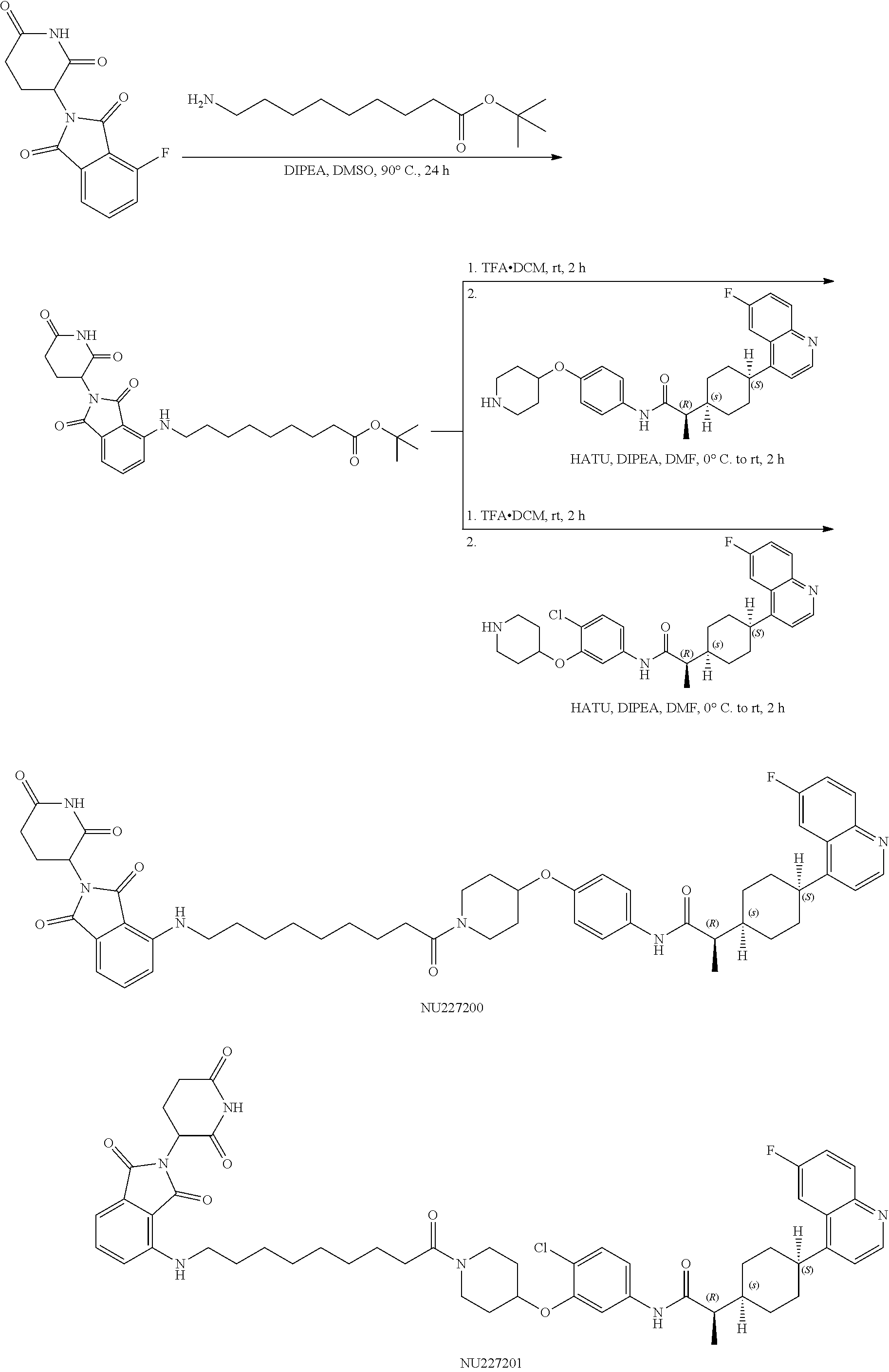

### Preparation of (2R)-N-(4-((1-(3-(2-((2-(2,6-dioxopiperidin-3-yl)-1,3-dioxoisoindolin-5-yl)amino)ethoxy)propanoyl)piperidin-4-yl)oxy)phenyl)-2-((1s,4S)-4-(6-fluoroquinolin-4-yl)cyclohexyl)propanamide (NU227302) and (2R)-N-(4-chloro-3-((1-(3-(2-((2-(2,6-dioxopiperidin-3-yl)-1,3-dioxoisoindolin-5-yl)amino)ethoxy)propanoyl)piperidin-4-yl)oxy)phenyl)-2-((1s,4S)-4-(6-fluoroquinolin-4-yl)cyclohexyl)propanamide (NU227303)

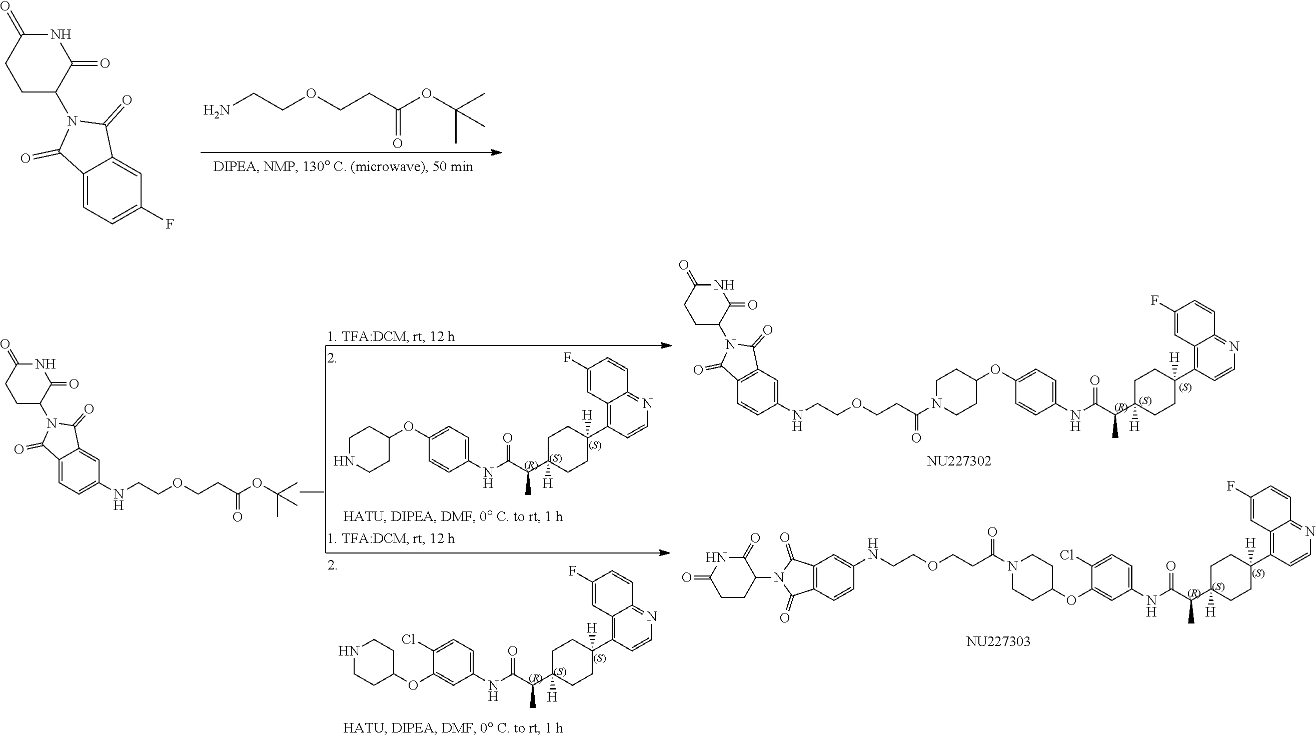

### Preparation of (2R)-N-(4-((1-(1-(4-((2-(2,6-dioxopiperidin-3-yl)-1,3-dioxoisoindolin-4-yl)amino)cyclohexyl)piperidine-4-carbonyl)piperidin-4-yl)oxy)phenyl)-2-((1s,4S)-4-(6-fluoroquinolin-4-yl)cyclohexyl)propanamide (NU227304) and (2R)-N-(4-((1-(1-(4-((2-(2,6-dioxopiperidin-3-yl)-1,3-dioxoisoindolin-5-yl)amino)cyclohexyl)piperidine-4-carbonyl)piperidin-4-yl)oxy)phenyl)-2-((1s,4S)-4-(6-fluoroquinolin-4-yl)cyclohexyl)propanamide (NU227309)

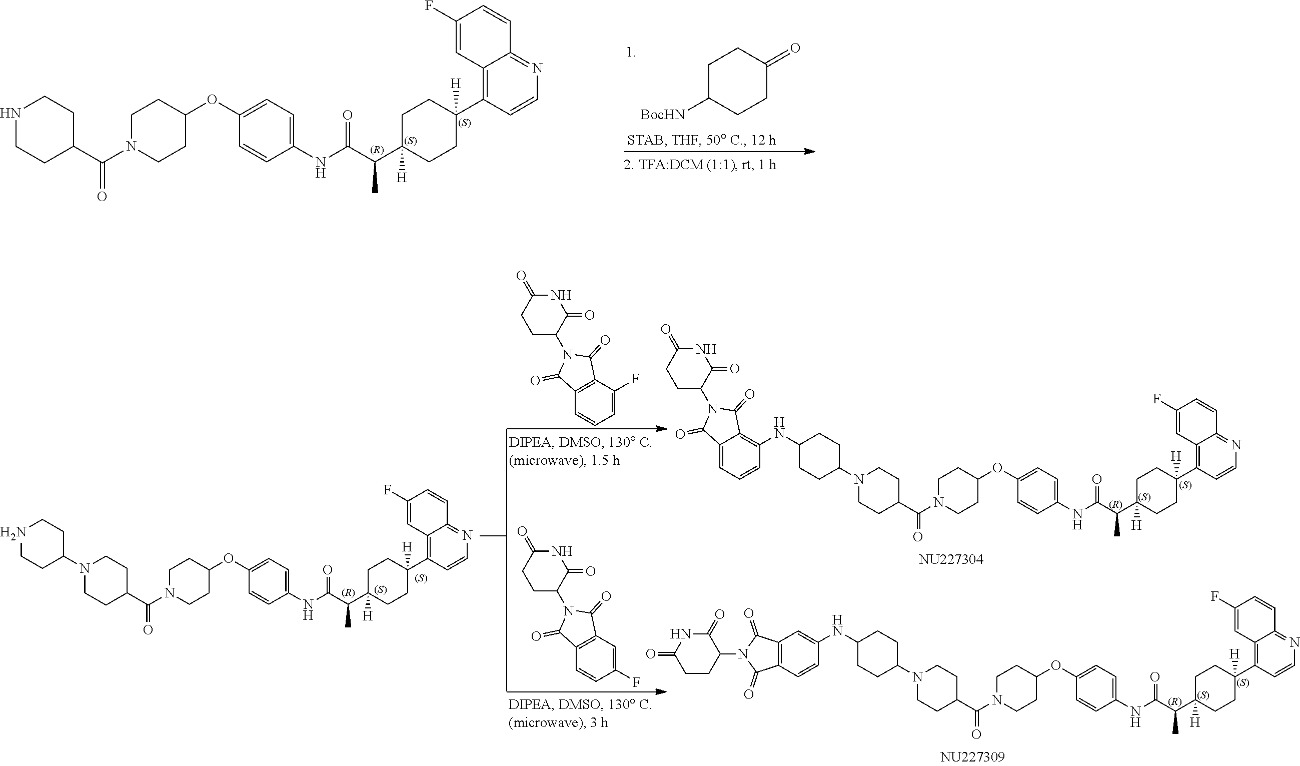

### Preparation of (2R)-N-(4-chloro-3-((1-(1(4-((2-(2,6-dioxopiperidin-3-yl)-1,3-dioxoisoindolin-4-yl)amino)cyclohexyl)piperidine-4-carbonyl)piperidin-4-yl)oxy)phenyl)-2-((1s,4S)-4-(6-fluoroquinolin-4-yl)cyclohexyl)propanamide (NU227310) and (2R)-N-(4-chloro-3-((1-(1-(4-((2-(2,6-dioxopiperidin-3-yl)-1,3-dioxoisoindolin-5-yl)amino)cyclohexyl)piperidine-4-carbonyl)piperidin-4-yl)oxy)phenyl)-2-((1 s,4S)-4-(6-fluoroquinolin-4

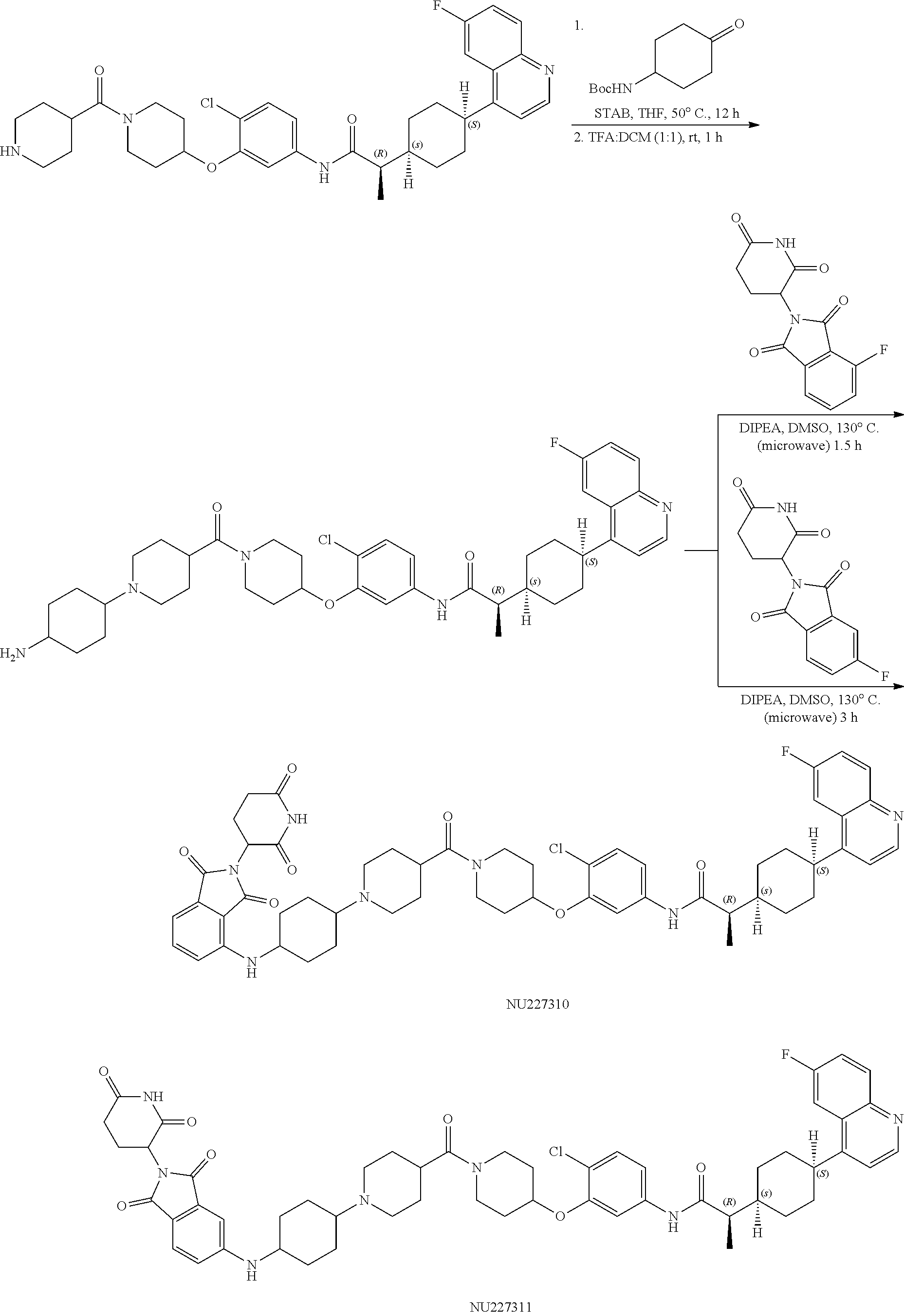

### Preparation of (2R)-N-(4-((1-(2-(4-(2-(2,6-dioxopiperidin-3-yl)-1,3-dioxoisoindolin-4-yl)piperazin-1-yl)acetyl)piperidin-4-yl)oxy)phenyl)-2-((1s,4S)-4-(6-fluoroquinolin-4-yl)cyclohexyl)propanamide (NU227305) and (2R)-N-(4-chloro-3-((1-(2-(4-(2-(2,6-dioxopiperidin-3-yl)-1,3-dioxoisoindolin-4-yl)piperazin-1-yl)acetyl)piperidin-4-yl)oxy)phenyl)-2-((1s,4S)-4-(6-fluoroquinolin-4-yl)cyclohexyl)propanamide (NU227306)

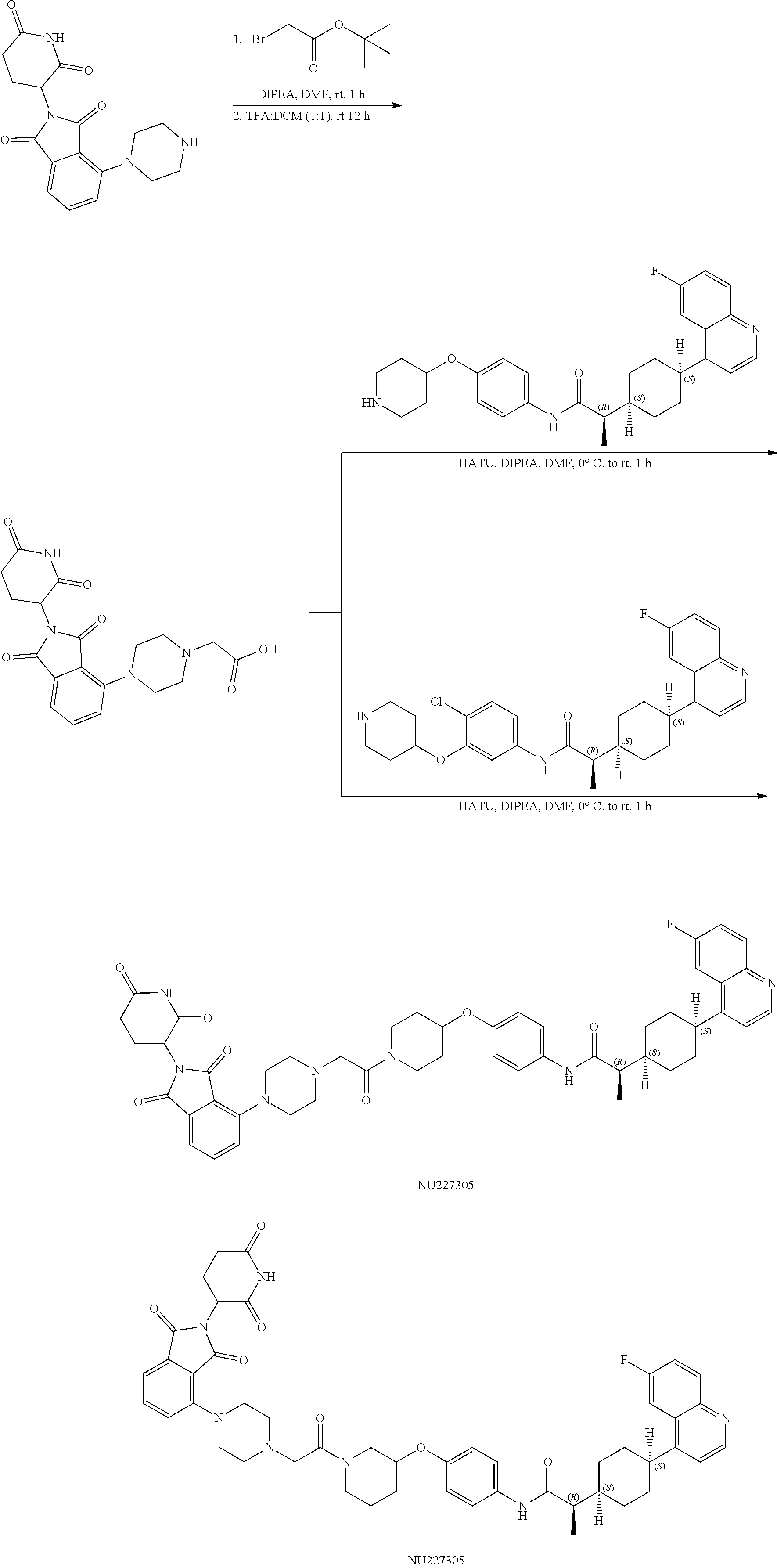

### Preparation of (2R)-N-(4-((1-(2-(4-(2-(2,6-dioxopiperidin-3-yl)-1,3-dioxoisoindolin-5-yl)piperazin-1-yl)acetyl)piperidin-4-yl)oxy)phenyl)-2-((1s,4S)-4-(6-fluoroquinolin-4-yl)cyclohexyl)propanamide (NV227307) and (2R)-NV-(4-chloro-3-((1-(2-(4-(2-(2,6-dioxopiperidin-3-yl)-1, 3-dioxoisoindolin-5-yl)piperazin-1-yl)acetyl)piperidin-4-yl)oxy)phenyl)-2-((1s,4$)-4-(6-fluoroquinolin-4-yl)cyclohexyl)propanamide (NU227308)

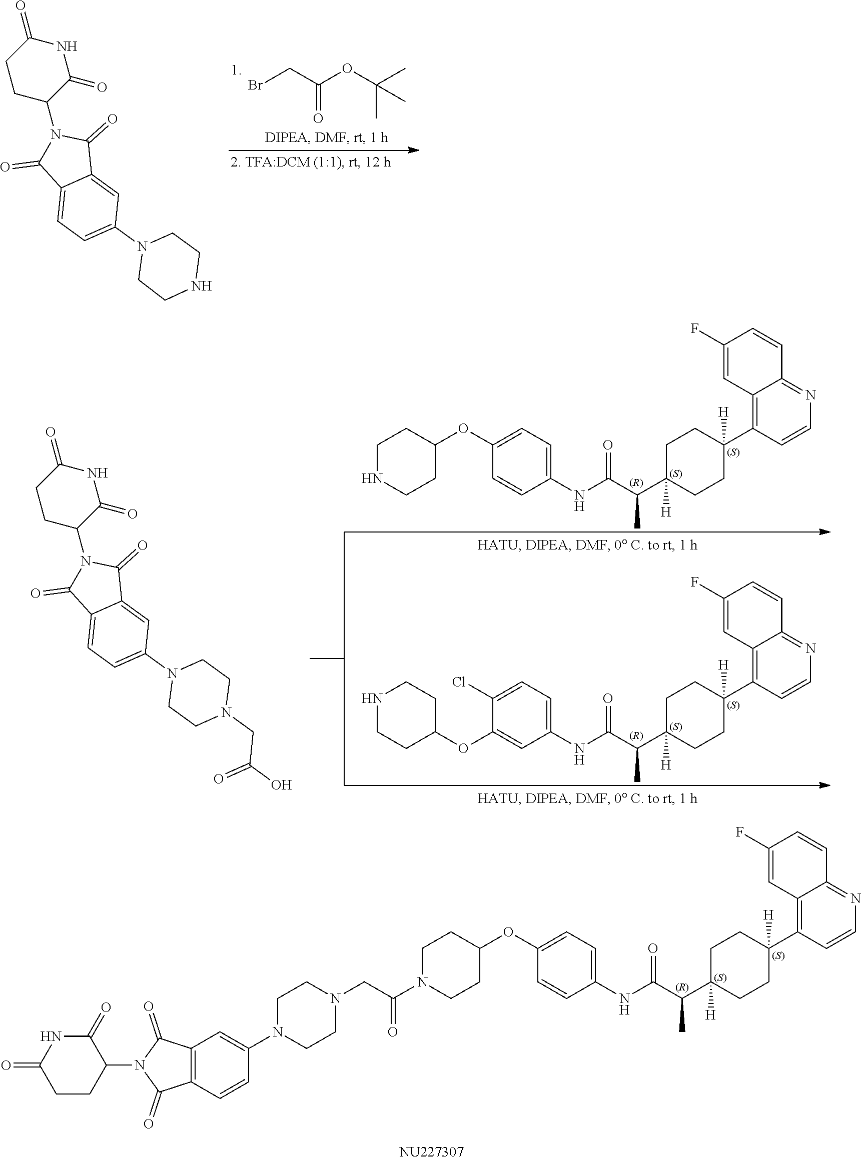

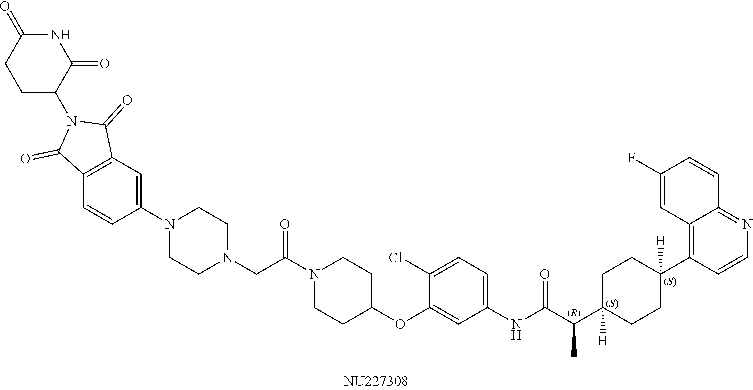

### Preparation of (2R)-N-(4-((1-(3-(2-((3-(2-(2,6-dioxopiperidin-3-yl)-1,3-dioxoisoindolin-4-yl)prop-2-yn-1-yl)oxy)ethoxy)propanoyl)piperidin-4-yl)oxy)phenyl)-2-((1s,4,S)-4-(6-fluoroquinolin-4-yl)cyclohexyl)propanamide (NU227331) and (2R)-N-(4-chloro-3-((1-(3-(2-((3-(2-(2,6-dioxopiperidin-3-yl)-1,3-dioxoisoindolin-4-yl)prop-2-yn-1-yl)oxy)ethoxy)propanoyl)piperidin-4-yl)oxy)phenyl)-2-((1s,4S)-4-(6-fluoroquinolin-4-yl)cyclohexyl)propanamide (NU227329)

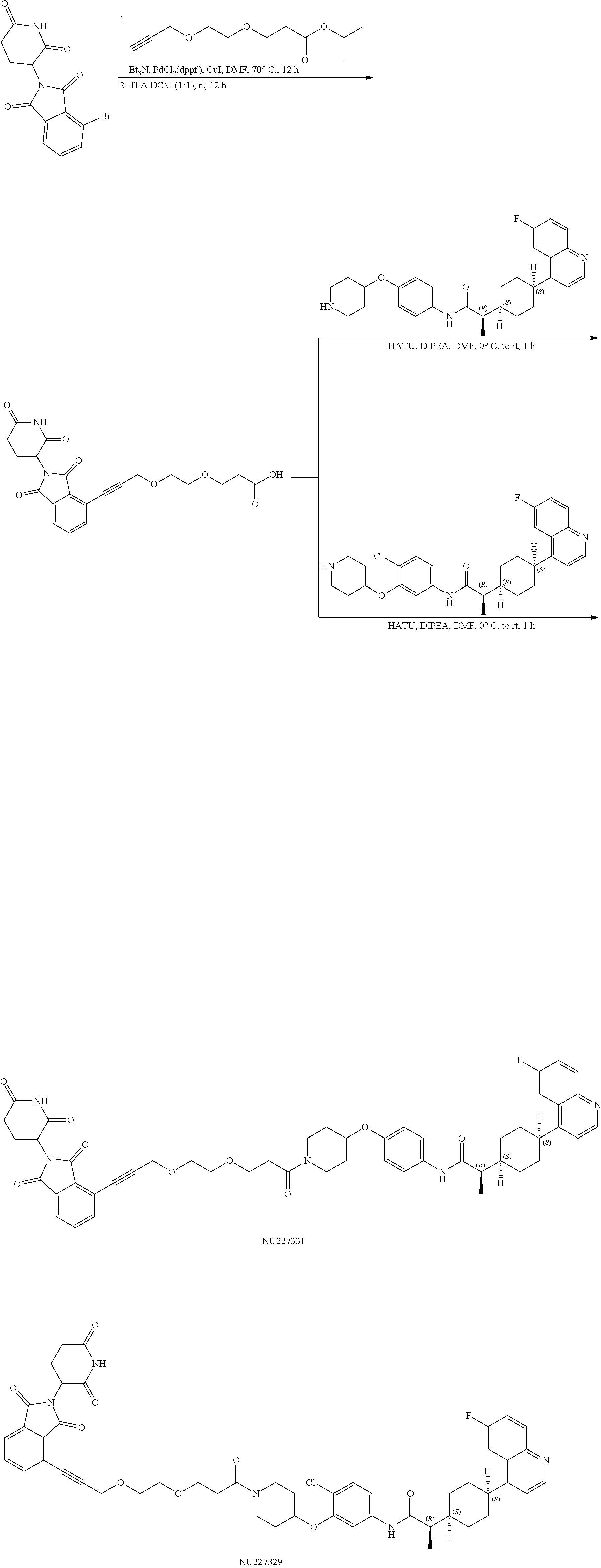

### Preparation of (2R)-N-(4-chloro-3-((1-(3-(2-((3-(2-(2,6-dioxopiperidin-3-yl)-1, 3-dioxoisoindolin-5-yl)prop-2-yn-1-yl)oxy)ethoxy)propanoyl)piperidin-4-yl)oxy)phenyl)-2-((1s,4S)-4-(6-fluoroquinolin-4-yl)cyclohexyl)propanamide (NU227330)

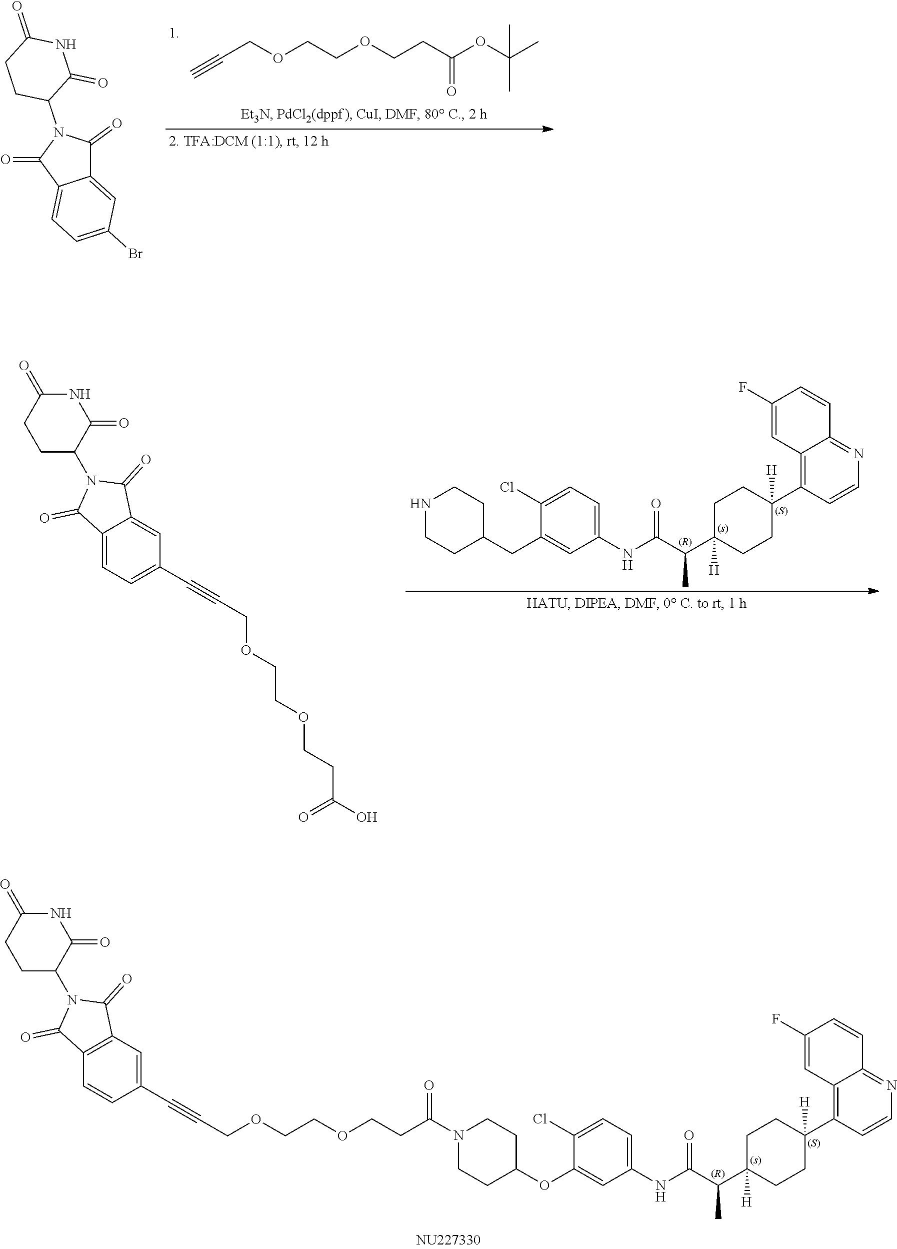

### Preparation of (2R)-N-(4-chloro-3-((1-(2-(4-(4-(2-(2,6-dioxopiperidin-3-yl)-6-fluoro-1,3-dioxoisoindolin-5-yl)piperazin-1-yl)piperidin-1-yl)acetyl)piperidin-4-yl)oxy)phenyl)-2-((1s,4S)-4-(6-fluoroquinolin-4-yl)cyclohexyl)propanamide (NU227355)

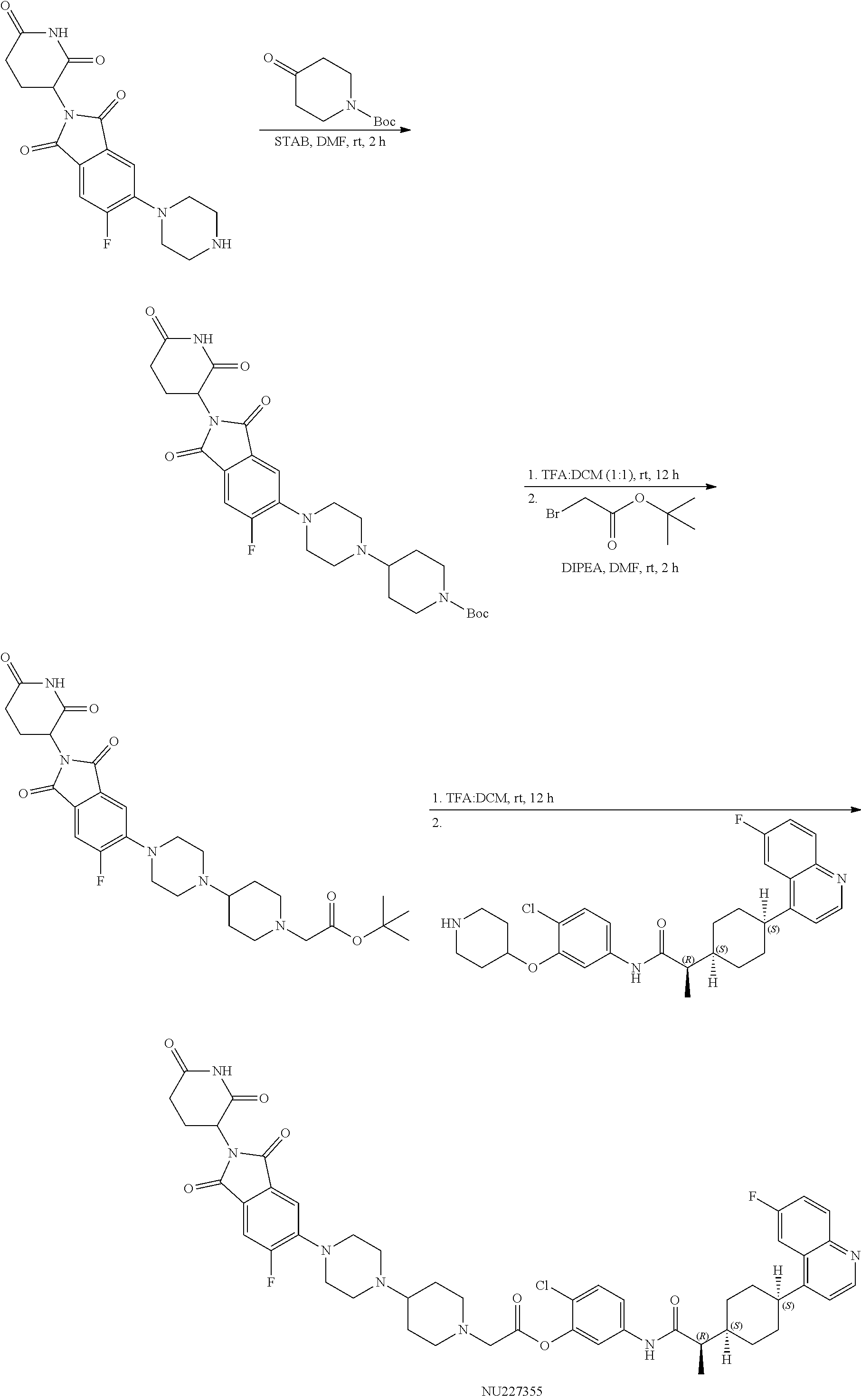

### Preparation of (2R)-N-(4-((1-(3-(2-(2-((2-(2,6-dioxopiperidin-3-yl)-1,3-dioxoisoindolin-4-yl)(methyl)amino)ethoxy)ethoxy)propanoyl)piperidin-4-yl)oxy)phenyl)-2-((1s,4S)-4-(6-fluoroquinolin-4-yl)cyclohexyl)propanamide (NU227361)

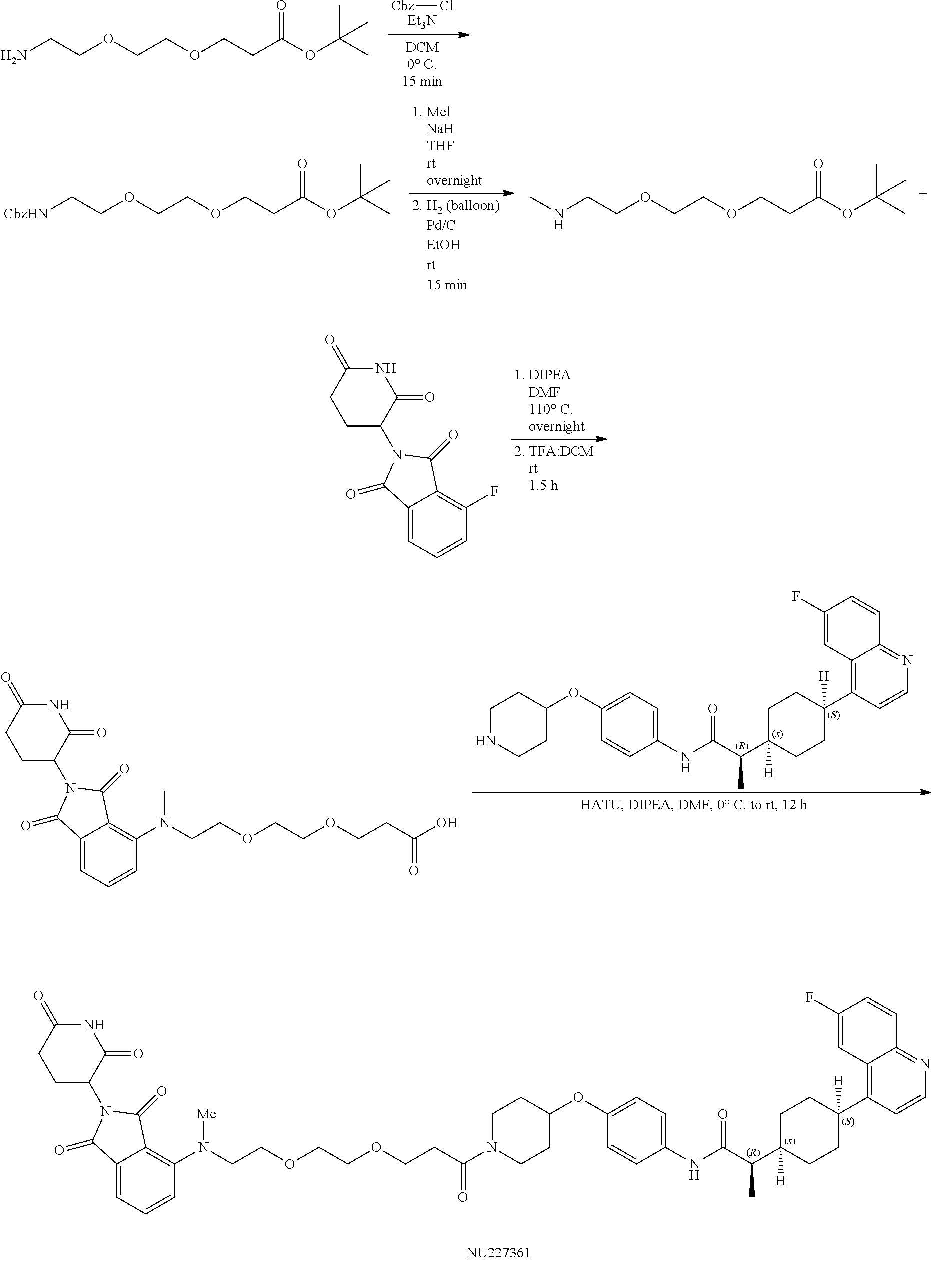

### Preparation of 2-(4-(2,6-dioxopiperidin-3-yl)phenoxy)acetic acid

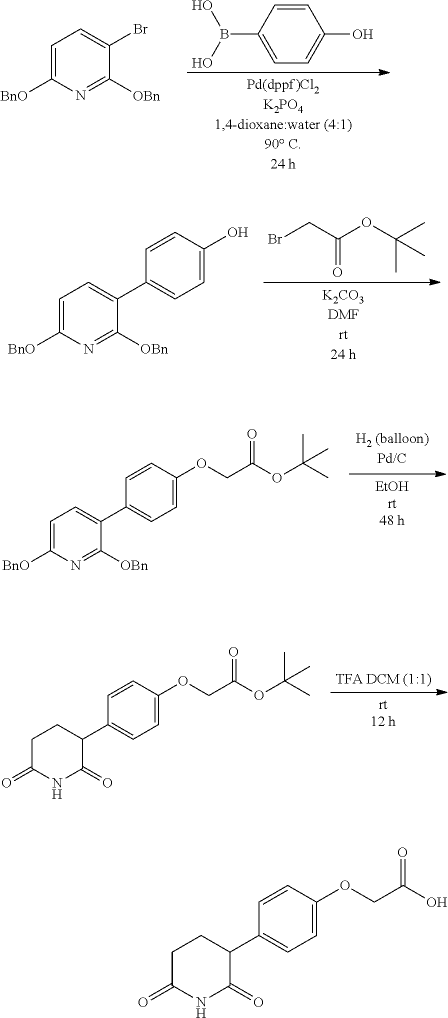

### Preparation of (2R)-N-(4-((1-(3-(2-(2-(2-(4-(2,6-dioxopiperidin-3-yl)phenoxy)acetamido)ethoxy)ethoxy)propanoyl)piperidin-4-yl)oxy)phenyl)-2-((1s,4S)-4-(6-fluoroquinolin-4-yl)cyclohexyl)propanamide (NU227353)

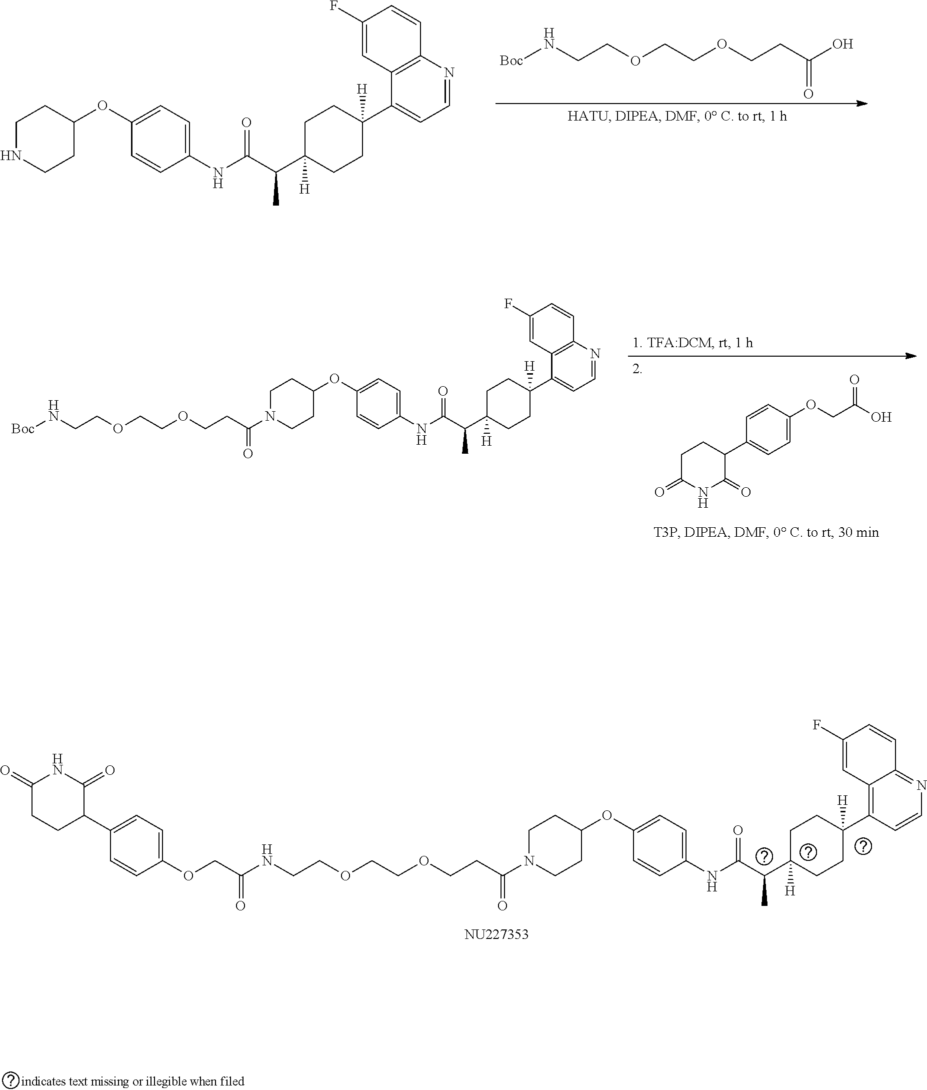 [SMILES: C[C@@H](C(=O)Nc1ccc(OC2CCNCC2)cc1)[C@H]1CC[C@@H](c2ccnc3ccc(F)cc32)CC1 ; CC(C)(C)OC(=O)NCCOCCOCCC(=O)O ; C[C@@H](C(=O)Nc1ccc(OC2CCN(C(=O)CCOCCOCCNC(=O)OC(C)(C)C)CC2)cc1)[C@H]1CC[C@@H](c2ccnc3ccc(F)cc32)CC1 ; O=C(O)COc1ccc(C2CCC(=O)NC2=O)cc1 ; C[C@@H](C(=O)Nc1ccc(OC2CCN(C(=O)CCOCCOCCNC(=O)COc3ccc(C4CCC(=O)NC4=O)cc3)CC2)cc1)[C@H]1CC[C@@H](c2ccnc3ccc(F)cc32)CC1]

### Preparation of (2R)-N-(4-((1-(1-(4-(2,6-dioxopiperidin-3-yl)phenoxy)-2-oxo-6,9,12-trioxa-3-azapentadecan-15-oyl)piperidin-4-yl)oxy)phenyl)-2-((1s,4S)-4-(6-fluoroquinolin-4-yl)cyclohexyl)propanamide (NU227354)

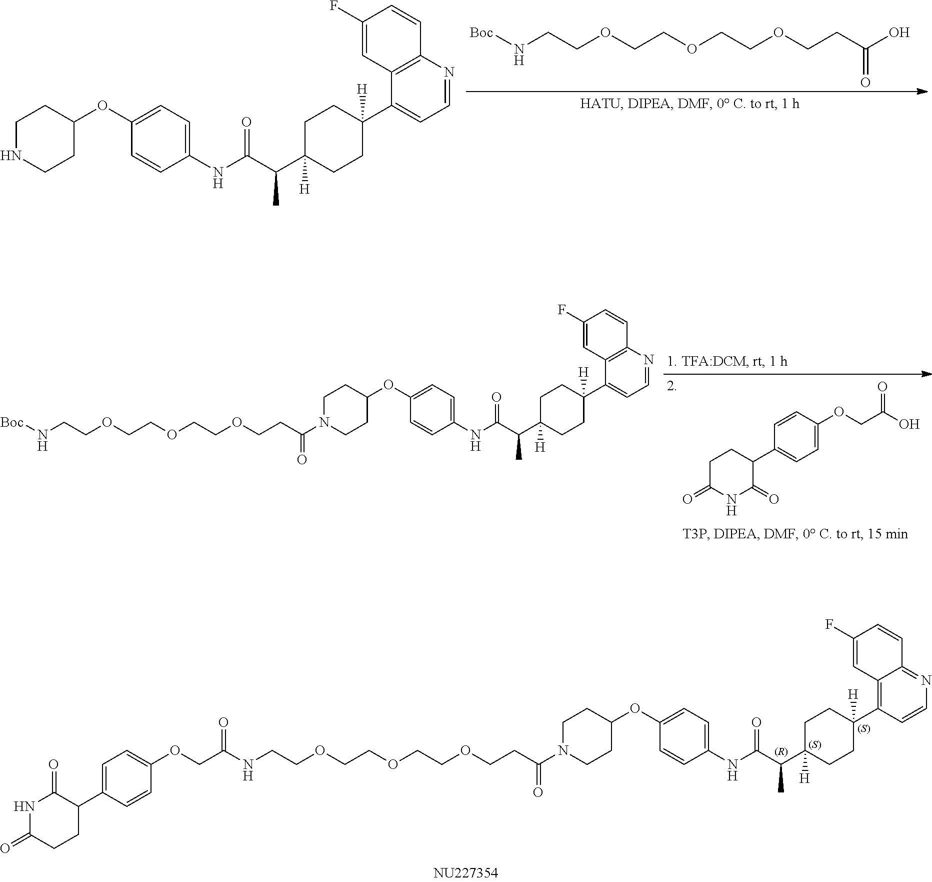

### Experimental Procedures for Selected Structures

General Chemical Methods: All chemical reagents were obtained from commercial suppliers and used without further purification, unless otherwise stated. Reactions were run without taking precautions to exclude air or moisture, unless otherwise noted. Purification of reaction products were carried out by flash chromatography on Biotage Isolera 4 systems with Ultra-grade silica cartridges. Monitoring of reactions and purity were assessed via UPLC-MS (WATERS Acquity-H UPLC-MS with an SQD). All microwave-assisted reactions were carried out in a Biotage initiator. ¹H NMR spectroscopy was recorded on a Bruker Avance III 500 MHz spectrometer. The chemical shifts for ¹H NMR are reported to the second decimal place in parts per million (ppm). Proton coupling constants are expressed in hertz (Hz). Standard abbreviations were used to denote spin multiplicity for ¹H-NMR data. The corresponding residual solvent peak (CDCl₃ ¹H δ=7.27 ppm; CD₃OD-d₄, ¹H δ=3.31 ppm; DMSO-d₆, ¹H δ=2.50 ppm) were used as an internal standard. Reverse phase preparative HPLC was performed with the following conditions: Phenomenex Gemini-NX C18, 110 Å, 150×21.2 mm; 5 μm. Eluting with a gradient of acetonitrile:water specified for each PROTAC with 0.1% formic acid or 0.1% TFA over 25 min, then 1 min at 100% acetonitrile.

### Synthesis of (R)-2-((1s,4S)-4-(6-fluoroquinolin-4-yl)cyclohexyl)-N-(4-(piperidin-4-yloxy)phenyl)propanamide

### Step 1: Preparation of tert-butyl 4-(4-nitrophenoxy)piperidine-1-carboxylate

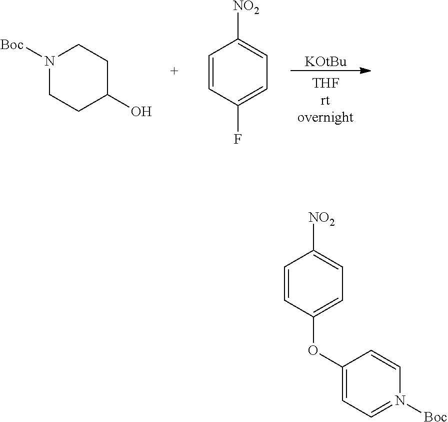

To a solution of 1-fluoro-4-nitrobenzene (15.8 g, 111.8 mmol, 1.5 Eq) and tert-butyl 4-hydroxypiperidine-1-carboxylate (15.0 g, 74.5 mmol, 1.0 Eq) in THF (400 mL) was added potassium tert-butoxide (16.7 g, 149.1 mmol, 2.0 Eq) portion-wise at 0° C. under nitrogen and the mixture was stirred at rt for 5 min, during which time the mixture maintained as a brown solution. TLC (petroleum ether/EtOAc, 3:1) showed the starting material (Rf=0.1) was consumed, a new main spot (Rf=0.5) generated. The mixture was poured into H₂O (50 mL) and concentrated under reduced pressure. The aqueous phase was extracted with EtOAc (3×40 mL). The organic phase was washed with brine (100 mL), dried over Na₂SO₄, filtered, and concentrated under reduced pressure. The residue was purified by column chromatography (SiO₂, petroleum ether/EtOAc, 100:1 to 3:2) to give tert-butyl 4-(4-nitrophenoxy)piperidine-1-carboxylate (23.0 g, 86%) as a brown solid. ¹H NMR (400 MHz, CD₃OD): δ 8.25-8.14 (m, 2H), 7.15-7.05 (m, 2H), 4.74 (tt, J=7.5, 3.7 Hz, 1H), 3.80-3.68 (m, 2H), 3.36 (br t, J=9.1 Hz, 2H), 2.04-1.95 (m, 2H), 1.77-1.62 (m, 2H), 1.47 (s, 9H) ppm.

### Step 2: Preparation of tert-butyl 4-(4-aminophenoxy)piperidine-1-carboxylate

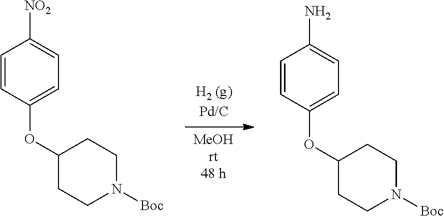 [SMILES: CC(C)(C)OC(=O)N1CCC(Oc2ccc([N+](=O)[O-])cc2)CC1 ; CC(C)(C)OC(=O)N1CCC(Oc2ccc(N)cc2)CC1]

To a solution of tert-butyl 4-(4-nitrophenoxy)piperidine-1-carboxylate (17.0 g, 52.7 mmol, 1.0 Eq) in methanol (MeOH, 340 mL) was added Pd/C (2.8 g, 10% Pd on charcoal, wet, containing 50% H₂O) at rt. The mixture was stirred at rt for 6 h under H₂ atmosphere (15 Psi). TLC (petroleum ether/EtOAc, 1:1) showed the starting material (Rf=0.9) was consumed, a new main spot (R_{f}=0.4) generated. The reaction mixture was filtered. The filtrate was concentrated to give tert-butyl 4-(4-aminophenoxy)piperidine-1-carboxylate (15.0 g, 88%) as brown solid, which was used to next step without further purification. ¹H NMR (400 MHz, CD₃OD): δ 6.82-6.75 (m, 2H), 6.74-6.66 (m, 2H), 4.35 (tt, J=7.4, 3.6 Hz, 1H), 3.76-3.66 (m, 2H), 3.35-3.24 (m, 3H), 1.93-1.84 (m, 2H), 1.67-1.58 (m, 2H), 1.51-1.46 (m, 9H) ppm.

### Steps 3 and 4: Preparation of (R)-2-((1s,4S)-4-(6-fluoroquinolin-4-yl)cyclohexyl)-N-(4-(piperidin-4-yloxy)phenyl)propanamide

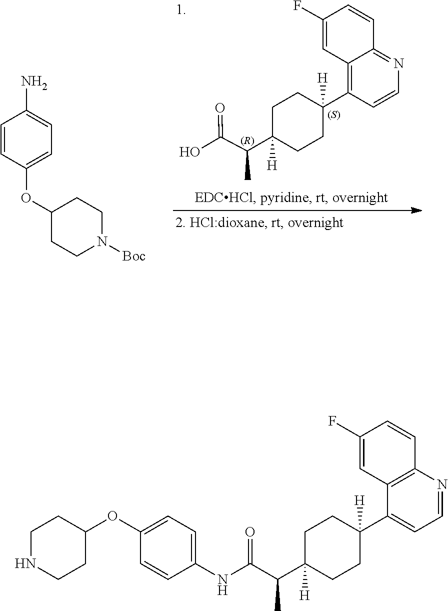

To a solution of (R)-2-((1s,4S)-4-(6-fluoroquinolin-4-yl)cyclohexyl)propanoic acid (2.5 g, 8.3 mmol, 1.0 Eq) and tert-butyl 4-(4-aminophenoxy)piperidine-1-carboxylate (2.8 g, 9.5 mmol, 1.0 Eq) in pyridine (25 mL) was added EDCI (2.4 g, 12.4 mmol, 1.5 Eq) at 0° C. and the mixture was stirred at rt for 12 h, during which time the mixture maintained as a yellow solution. TLC (petroleum ether/THF, 1:1) showed the starting material (R_{f}=0.4) was consumed, a new main spot (R_{f}=0.2) generated. Additional three reactions were set up as described above and combined for purification. The combined reaction mixture was poured into 1 N HCl (120 mL) and EtOAc (200 mL). The two phases were separated, and the aqueous phase was extracted with EtOAc (2×50 mL). The combined organic layers were washed with 1 N HCl (2×30 mL), saturated Na₂CO₃ solution (3×100 mL), then dried over anhydrous Na₂SO₄, filtered, and concentrated under reduced pressure to give boc-protected intermediate (19.0 g, 95% yield) as a yellow solid which was taken forward without further purification. To a solution of boc-protected intermediate (6.0 g, 10.4 mmol, 1.0 E) in dioxane (10 mL) was added HCl/dioxane (4 μM, 40 mL) at rt. The reaction mixture was stirred at rt for 12 h. TLC (petroleum ether/THF, 1:1) showed the starting material (R_{f}=0.2) was consumed, a new main spot (R_{f}=0.01) generated. Additional two reactions were set up as described above and combined for purification. The combined reaction mixtures were concentrated under reduced pressure to give (R)-2-((1s,4S)-4-(6-fluoroquinolin-4-yl)cyclohexyl)-N-(4-(piperidin-4-yloxy)phenyl)propanamide (16.0 g, HCl salt, 97%) as a yellow solid. ¹H NMR (500 MHz, DMSO-d₆): δ 10.27 (s, 1H), 9.31 (d, J=28.1 Hz, 2H), 8.88 (d, J=4.7 Hz, 1H), 8.13 (dd, J=9.2, 5.7 Hz, 1H), 8.02 (dd, J=10.9, 2.8 Hz, 1H), 7.78 (d, J=4.7 Hz, 1H), 7.74-7.66 (m, 1H), 7.64-7.57 (m, 2H), 6.98-6.88 (m, 2H), 4.56 (tt, J=7.3, 3.3 Hz, 1H), 3.40 (td, J=11.1, 5.6 Hz, 2H), 3.120-3.16 (m, 2H), 3.05-2.97 (m, 3H), 2.15-1.51 (m, 13H), 1.09 (d, J=6.6 Hz, 3H) ppm.

### Synthesis of (R)-N-(4-chloro-3-(piperidin-4-yloxy)phenyl)-2-((1s,4S)-4-(6-fluoroquinolin-4-yl)cyclohexyl)propanamide: Step 1: Preparation of tert-butyl 4-(2-chloro-5-nitrophenoxy)piperidine-1-carboxylate

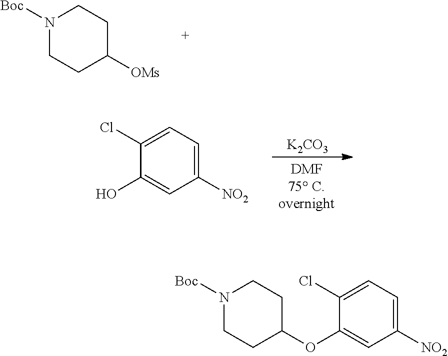

To a solution of tert-butyl 4-((methylsulfonyl)oxy)piperidine-1-carboxylate (1.45 g, 2.0 Eq, 5.19 mmol) and 2-chloro-5-nitrophenol (0.450 g, 1.0 Eq, 2.59 mmol) in DMF (10 mL) was added potassium carbonate (717 mg, 2.0 Eq, 5.19 mmol). The resulting mixture was stirred at 75° C. overnight. The reaction was allowed to cool to rt and then diluted with DI water (20 mL). The resulting mixture was extracted with EtOAc (3×15 mL), and the organic layers were combined, washed with brine (1×30 mL), dried over anhydrous magnesium sulfate, filtered and concentrated under reduced pressure. The crude product was taken forward without further purification.

### Step 2: Preparation of tert-butyl 4-(5-amino-2-chlorophenoxy)piperidine-1-carboxylate

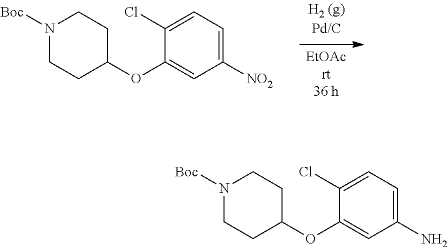 [SMILES: CC(C)(C)OC(=O)N1CCC(Oc2cc([N+](=O)[O-])ccc2Cl)CC1 ; CC(C)(C)OC(=O)N1CCC(Oc2cc(N)ccc2Cl)CC1]

To a solution of tert-butyl 4-(2-chloro-5-nitrophenoxy)piperidine-1-carboxylate (850 mg, 1.0 Eq, 2.38 mmol) in EtOAc (3 mL) was added palladium on carbon (12.7 mg, 0.05 Eq, 119 μmol) (catalytic). The reaction was fitted with a hydrogen balloon and allowed to stir at rt for 36h. The reaction was filtered through a celite pad with EtOAc rinses and concentrated to yield tert-butyl 4-(5-amino-2-chlorophenoxy)piperidine-1-carboxylate (720 mg, 2.20 mmol, 92.5%) as a yellow oil. LCMS (m z): 271.2 [M−t-butyl+1]⁺.

### Steps 3 and 4: Preparation of (R)-N-(4-chloro-3-(piperidin-4-yloxy)phenyl)-2-((1s,4S)-4-(6-fluoroquinolin-4-yl)cyclohexyl)propanamide

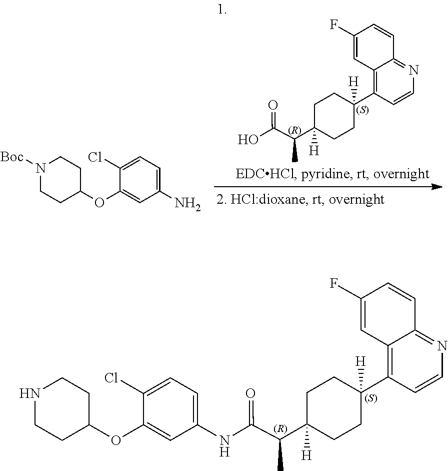

To a solution of tert-butyl 4-(5-amino-2-chlorophenoxy)piperidine-1-carboxylate (656 mg, 1.0 Eq, 2.01 mmol) and (R)-2-((1s,4S)-4-(6-fluoroquinolin-4-yl)cyclohexyl)propanoic acid (550 mg, 1.0 Eq, 1.83 mmol) in Pyridine (5 mL) was added 1-(3-dimethylaminopropyl)-3-ethylcarbodiimide hydrochloride (525 mg, 1.5 Eq, 2.74 mmol). The resulting mixture was allowed to stir at rt overnight. The reaction was poured into 1N HCl (100 mL) and extracted with EtOAc (3×75 mL). the organic layers were combined and washed with 1N HCl (3×30 mL), Sat Na₂CO₃ (3×50 mL), dried over anhydrous magnesium sulfate, filtered and concentrated under reduced pressure to provide tert-butyl 4-(2-chloro-5-((R)-2-((1s,4S)-4-(6-fluoroquinolin-4-yl)cyclohexyl)propanamido)phenoxy)piperidine-1-carboxylate as a light orange/brown residue.

The resulting product was taken forward without additional purification. To a solution of tert-butyl 4-(2-chloro-5-((R)-2-((1s,4S)-4-(6-fluoroquinolin-4-yl)cyclohexyl)propanamido)phenoxy)piperidine-1-carboxylate (1.11 g, 1.0 Eq, 1.82 mmol) in Dioxane (5 mL) was added a solution HCl/Dioxane (7 g, 5 mL, 4 molar, 1e+1.0 Eq, 0.02 mol). The resulting mixture was allowed to stir at rt overnight. To the solution was added cold Et₂O and the solid precipitate was filtered with vacuum to yield (R)-N-(4-chloro-3-(piperidin-4-yloxy)phenyl)-2-((1s,4S)-4-(6-fluoroquinolin-4-yl)cyclohexyl)propanamide, HCl (950 mg, 1.74 mmol, 95.6%) as a dark yellow solid. LCMS (m z): 510.3 [M+1]⁺. ¹H NMR (DMSO, 500 MHz): S=10.77 (s, 1H), 9.22 (d, 1H, J=4.4 Hz), 9.08 (s, 1H), 8.53-8.17 (m, 3H), 8.04 (dt, 1H, J=26.6, 7.8 Hz), 7.85 (t, 1H, J=1.4 Hz), 7.45-7.14 (m, 2H), 4.62 (tt, 1H, J=6.9, 3.2 Hz), 3.68-3.57 (m, 1H), 3.23-3.17 (m, 2H), 3.11 (d, 3H, J=11.8 Hz), 2.29-2.08 (m, 3H), 1.98-1.57 (m, 10H), 1.12 (d, 3H, J=6.6 Hz) ppm.

### Synthesis of (R)-2-((1s,4S)-4-(6-fluoroquinolin-4-yl)cyclohexyl)-N-(3-(piperidin-4-yloxy)phenyl)propanamide: Step 1: Preparation of tert-butyl 4-(3-nitrophenoxy)piperidine-1-carboxylate

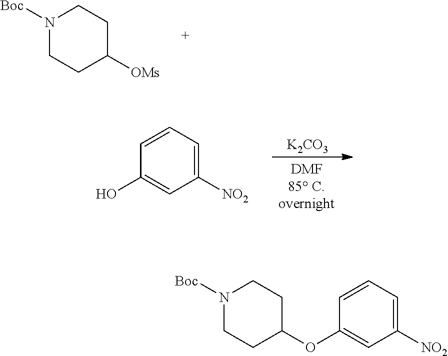

To a solution of tert-butyl 4-((methylsulfonyl)oxy)piperidine-1-carboxylate (4 g, 2.0 Eq, 0.01 mol) and 3-nitrophenol (1 g, 1.0 Eq, 7 mmol) in DMF (20 mL) was added potassium carbonate (2 g, 2.0 Eq, 0.01 mol). The resulting mixture was stirred at 85° C. with stirring overnight. The reaction mixture was then allowed to cool to rt, diluted with DI water, and extracted with EtOAc (3×15 mL). The organic layers were combined, washed with brine (1×30 mL), dried over anhydrous magnesium sulfate, and concentrated in vacuo. The crude residue was purified with flash column chromatography with hexanes:EtOAc (100:0 to 50:50) to yield tert-butyl 4-(3-nitrophenoxy)piperidine-1-carboxylate (1.5 g, 4.7 mmol, 60%) as a yellow oil.

### Step 2: Preparation of tert-butyl 4-(3-aminophenoxy)piperidine-1-carboxylate

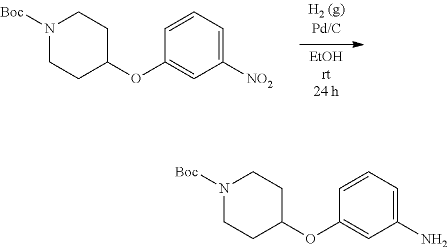 [SMILES: CC(C)(C)OC(=O)N1CCC(Oc2cccc([N+](=O)[O-])c2)CC1 ; CC(C)(C)OC(=O)N1CCC(Oc2cccc(N)c2)CC1]

To a solution of tert-butyl 4-(3-nitrophenoxy)piperidine-1-carboxylate (1.5 g, 1.0 Eq, 4.7 mmol) in EtOH (4 mL) was added palladium on carbon (50 mg, 0.1.0 Eq, 0.47 mmol). The reaction mixture was fitted with a hydrogen gas balloon and allowed to stir at rt for 24 h. The reaction mixture was filtered through a celite pad with EtOH rinse, and the filtrate was concentrated under reduce pressure to provide tert-butyl 4-(3-aminophenoxy)piperidine-1-carboxylate (0.7 g, 2 mmol, 50%) as a yellow oil. The product was taken forward without further purification.

### Step 3: Preparation of tert-butyl 4-(3-((R)-2-((1s,4S)-4-(6-fluoroquinolin-4-yl)cyclohexyl)propanamido)phenoxy)piperidine-1-carboxylate

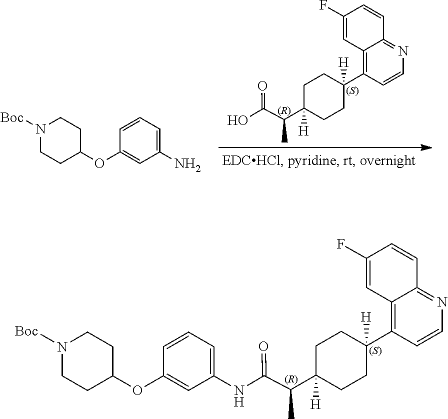

To a solution of tert-butyl 4-(3-aminophenoxy)piperidine-1-carboxylate (1 g, 1.0 Eq, 4 mmol) and (R)-2-((1s,4S)-4-(6-fluoroquinolin-4-yl)cyclohexyl)propanoic acid (1 g, 1.0 Eq, 3 mmol) in Pyridine (15 mL) was added 3-(((ethylimino)methylene)amino)-N,N-dimethylpropan-1-amine hydrochloride (1 g, 1.5 Eq, 5 mmol). The resulting mixture was allowed to stir at rt overnight. The reaction was poured into 1N HCl (75 mL) and extracted with EtOAc (3×50 mL). the organic layers were combined, washed with 1N HCl (3×30 mL), Sat Na₂CO₃ (3×50 mL), dried over anhydrous magnesium sulfate, filtered, and concentrated in vacuo to a brown residue. The crude mixture was purified with flash column chromatography (hexanes:EtOAc, 100:0 to 50:50) to provide tert-butyl 4-(3-((R)-2-((1s,4S)-4-(6-fluoroquinolin-4-yl)cyclohexyl)propanamido)phenoxy)piperidine-1-carboxylate (700 mg, 1.22 mmol, 40%) as a yellow solid. LCMS (m, z): 577.5 [M+2]⁺. ¹H NMR (500 MHz, CDCl₃) δ 8.76 (d, J=4.6 Hz, 1H), 8.10 (dd, J=9.2, 5.6 Hz, 1H), 8.04 (s, 1H), 7.63 (dd, J=10.5, 2.8 Hz, 1H), 7.51-7.40 (m, 2H), 7.28 (d, J=4.6 Hz, 1H), 7.17 (t, J=8.1 Hz, 1H), 7.01 (d, J=8.0 Hz, 1H), 6.68-6.58 (m, 1H), 4.45 (dt, J=7.0, 3.6 Hz, 1H), 3.64 (ddd, J=13.4, 7.7, 3.7 Hz, 2H), 3.31 (ddt, J=16.0, 8.0, 3.9 Hz, 3H), 2.63 (dd, J=10.9, 6.7 Hz, 1H), 2.18-2.09 (m, 1H), 1.92-1.58 (m, 12H), 1.46 (s, 9H), 1.29-1.24 (m, 3H) ppm.

### Step 4: Preparation of (R)-2-((1s,4S)-4-(6-fluoroquinolin-4-yl)cyclohexyl)-N-(3-(piperidin-4-yloxy)phenyl)propanamide

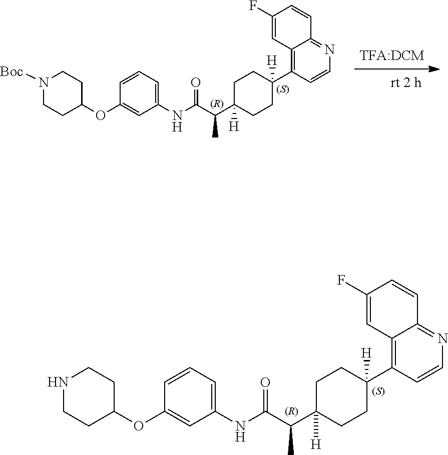

To tert-butyl 4-(3-((R)-2-((1s,4S)-4-(6-fluoroquinolin-4-yl)cyclohexyl)propanamido)phenoxy)piperidine-1-carboxylate (700 mg, 1.0 Eq, 1.22 mmol) was added TFA:DCM (1:1, 3 mL). The resulting mixture was stirred at rt for 2 h. The reaction mixture was concentrated under nitrogen atmosphere and the crude residue was triturated with cold Et₂O (2×15 mL) to provide pure (R)-2-((1s,4S)-4-(6-fluoroquinolin-4-yl)cyclohexyl)-N-(3-(piperidin-4-yloxy)phenyl)propanamide, Trifluoracetate (650 mg, 1.10 mmol, 90.8%) as an orange solid. LCMS (m z): 477.4 [M+2]⁺.

### Synthesis of NU227047

### Steps 1 and 2: Preparation of 3-(2-(2-((2-(2,6-dioxopiperidin-3-yl)-1-oxoisoindolin-4-yl)amino)ethoxy)ethoxy)propanoic acid

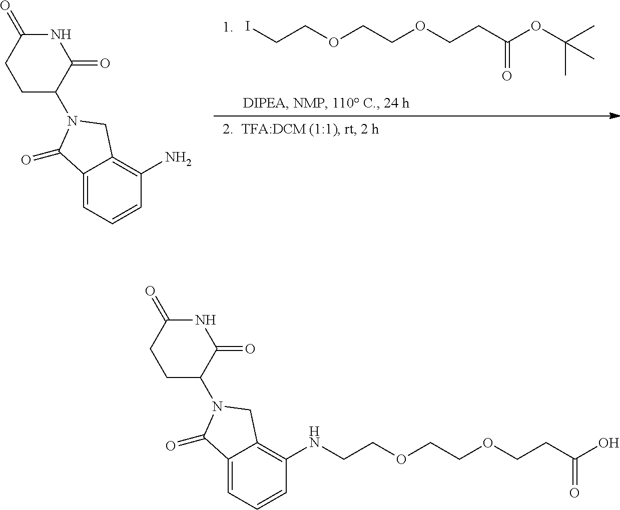 [SMILES: Nc1cccc2c1CN(C1CCC(=O)NC1=O)C2=O ; CC(C)(C)OC(=O)CCOCCOCCI ; O=C(O)CCOCCOCCNc1cccc2c1CN(C1CCC(=O)NC1=O)C2=O]

To a solution of tert-butyl 3-(2-(2-iodoethoxy)ethoxy)propanoate (50 mg, 1.0 Eq, 0.15 mmol) and NMP (1 mL) was added DIPEA (56 mg, 76 μL, 3.0 Eq, 0.44 mmol) and 3-(4-amino-1-oxoisoindolin-2-yl)piperidine-2,6-dione (30 mg, 0.8 Eq, 0.12 mmol). The resulting mixture was allowed to warm with stirring to 110° C. After 24 h the reaction was allowed to cool to rt, quenched with DI water (3 mL) and extracted with EtOAc (3×3 mL). The organic layers were combined, washed with water (2×10 mL), brine (1×15 mL), dried via filtration through a phase separator and concentrated in vacuo to provide the intermediate which was taken forward without further purification. To the intermediate was added DCM:TFA (1:1, 1 mL) and the reaction was allowed to stir at rt for 2 h. The reaction mixture was concentrated under nitrogen atmosphere and the resulting residue was triturated with Et₂O (2×5 mL) to provide 3-(2-(2-((2-(2,6-dioxopiperidin-3-yl)-1-oxoisoindolin-4-yl)amino)ethoxy)ethoxy)propanoic acid, Trifluoroacetic acid (45 mg, 84 μmol, 58%) as a light orange solid. LCMS (m/z): 420.3 [M+1]⁺. ¹H NMR (500 MHz, DMSO) δ 11.03 (s, 1H), 7.29 (t, J=7.7 Hz, 1H), 6.94 (d, J=7.6 Hz, 1H), 6.80 (dd, J=8.0, 2.0 Hz, 1H), 5.11 (dd, J=13.3, 5.1 Hz, 1H), 4.25-4.06 (m, 2H), 3.62-3.56 (m, 4H), 3.54-3.38 (m, 4H), 3.34-3.27 (m, 2H), 2.92 (ddd, J=17.4, 13.6, 5.4 Hz, 1H), 2.65-2.56 (m, 1H), 2.43 (t, J=6.3 Hz, 2H), 2.29 (td, J=13.3, 4.5 Hz, 1H), 2.02 (dtd, J=10.8, 5.3, 2.3 Hz, 1H) ppm.

### Step 3: Preparation of (2R)-N-(4-chloro-3-((1-(3-(2-(2-((2-(2,6-dioxopiperidin-3-yl)-1-oxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide, NU227047

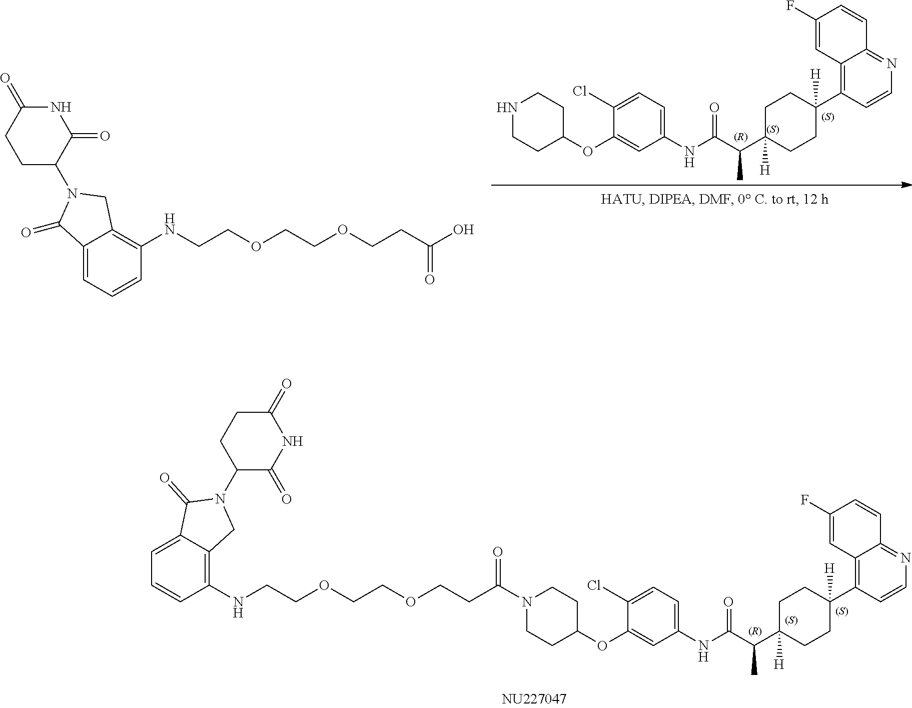

To a solution of 3-(2-(2-((2-(2,6-dioxopiperidin-3-yl)-1-oxoisoindolin-4-yl)amino)ethoxy)ethoxy)propanoic acid, Trifluoroacetic acid (70 mg, 1.0 Eq, 0.13 mmol) in DMF (2 mL) at 0° C. was added DIPEA (51 mg, 69 μL, 3.0 Eq, 0.39 mmol) and HATU (60 mg, 1.2 Eq, 0.16 mmol). The resulting mixture was flushed with nitrogen and allowed to stir at 0° C. for 30 min. (R)-N-(4-chloro-3-(piperidin-4-yloxy)phenyl)-2-((1s,4S)-4-(6-fluoroquinolin-4-yl)cyclohexyl)propanamide, HCl (72 mg, 1.0 Eq, 0.13 mmol) was then added and the reaction mixture was allowed to stir at rt for 12 h. The resulting mixture was directly filtered and purified with reverse phase preparative HPLC eluting from 10 to 90% ACN in water (0.1% FA). The desired fractions were lyophilized (3d) to provide (2R)-N-(4-chloro-3-((1-(3-(2-(2-((2-(2,6-dioxopiperidin-3-yl)-1-oxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide, Formic Acid (54.3 mg, 56.7 μmol, 43%) as a dark orange solid.

### Synthesis of NU227164

### Steps 1 and 2: Preparation of 2-(2,6-dioxopiperidin-3-yl)-5-(piperazin-1-yl)isoindoline-1,3-dione

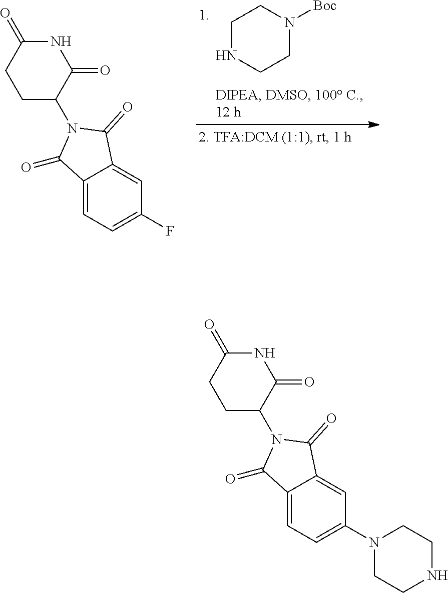 [SMILES: O=C1CCC(N2C(=O)c3ccc(F)cc3C2=O)C(=O)N1 ; CC(C)(C)OC(=O)N1CCNCC1 ; O=C1CCC(N2C(=O)c3ccc(N4CCNCC4)cc3C2=O)C(=O)N1]

To a solution of 2-(2,6-dioxopiperidin-3-yl)-5-fluoroisoindoline-1,3-dione (500 mg, 1.0 Eq, 1.81 mmol) in DMSO (5 mL) was added tert-butyl piperazine-1-carboxylate (539 mg, 1.6 Eq, 2.90 mmol) followed by DIPEA (702 mg, 946 μL, 3.0 Eq, 5.43 mmol). The resulting mixture was warmed with stirring to 100° C. and allowed to stir for 12 h. After cooling to rt the reaction was quenched with DI water and the resulting suspended solid was filtered and washed with cold DI water to afford the intermediate product as a yellow solid. To the intermediate product was added DCM:TFA (1:1, 4 mL) and the resulting reaction mixture was allowed to stir for 1 h at rt. The mixture was concentrated under nitrogen atmosphere and the crude residue was triturated with cold Et₂O (2×10 mL) to yield pure 2-(2,6-dioxopiperidin-3-yl)-5-(piperazin-1-yl)isoindoline-1,3-dione, Trifluoracetate (700 mg, 1.54 mmol, 84.9%) as a yellow solid. LCMS (n/z): 686.5 [2M+1]⁺. ¹H NMR (500 MHz, DMSO) δ 11.11 (s, 1H), 8.94 (s, 2H), 7.75 (d, J=8.5 Hz, 1H), 7.46 (d, J=2.3 Hz, 1H), 7.33 (dd, J=8.6, 2.4 Hz, 1H), 5.09 (dd, J=12.8, 5.4 Hz, 1H), 3.67 (dd, J=6.7, 3.9 Hz, 4H), 3.28-3.18 (m, 4H), 2.89 (ddd, J=16.7, 13.7, 5.4 Hz, 1H), 2.63-2.51 (m, 2H), 2.02 (dtd, J=12.9, 5.1, 2.1 Hz, 1H) ppm.

### Steps 3 and 4: Preparation of 2-(2,6-dioxopiperidin-3-yl)-5-(4-(piperidin-4-ylmethyl)piperazin-1-yl)isoindoline-1,3-dione

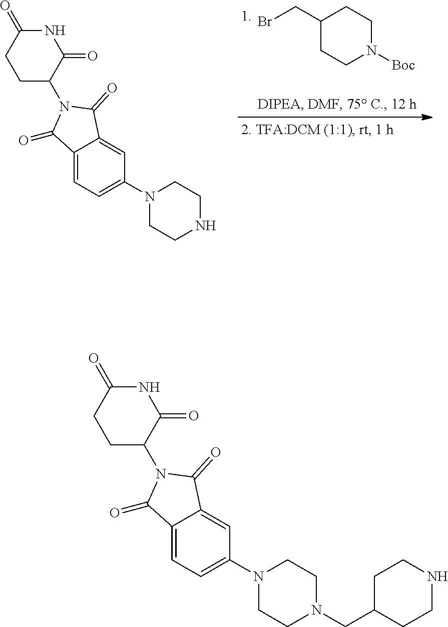 [SMILES: O=C1CCC(N2C(=O)c3ccc(N4CCNCC4)cc3C2=O)C(=O)N1 ; CC(C)(C)OC(=O)N1CCC(CBr)CC1 ; O=C1CCC(N2C(=O)c3ccc(N4CCN(CC5CCNCC5)CC4)cc3C2=O)C(=O)N1]

To a solution of 2-(2,6-dioxopiperidin-3-yl)-5-(piperazin-1-yl)isoindoline-1,3-dione (200 mg, 1.0 Eq, 584 μmol) and tert-butyl 4-(bromomethyl)piperidine-1-carboxylate (195 mg, 1.2 Eq, 701 μmol) in DMF (2.5 mL) was added DIPEA (227 mg, 305 VL, 3.0 Eq, 1.75 mmol). The resulting mixture was stirred at 75° C. for 12 h. The reaction was allowed to cool to rt and then quenched with DI water and extracted with DCM (3×5 mL). The organic layers were combined and washed with water (2×10 mL) and brine (1×20 mL). The organic phase was dried using a phase separator and the solvent was removed under vacuum. The crude residue was purified by flash column chromatography (DCM:acetone, 100:0 to 0:100) to afford tert-butyl 4-((4-(2-(2,6-dioxopiperidin-3-yl)-1,3-dioxoisoindolin-5-yl)piperazin-1-yl)methyl)piperidine-1-carboxylate (300 mg, 556 μmol, 95.2%) as a yellow solid. To tert-butyl 4-((4-(2-(2,6-dioxopiperidin-3-yl)-1,3-dioxoisoindolin-5-yl)piperazin-1-yl)methyl)piperidine-1-carboxylate (300 mg, 1.0 Eq, 556 mol) was added a TFA:DCM (1:1, 2.0 mL). The reaction was stirred at rt for 1 h. The mixture was concentrated to dryness under nitrogen atmosphere. The resulting residue was triturated with Et₂O (2×5 mL) to yield 2-(2,6-dioxopiperidin-3-yl)-5-(4-(piperidin-4-ylmethyl)piperazin-1-yl)isoindoline-1,3-dione, Trifluoroacetic acid (300 mg, 542 μmol, 97.5%) as a yellow solid. LCMS (m/z): 440.1 [M+1]⁺. ¹H NMR (500 MHz, DMSO) δ 11.12 (s, 1H), 7.76 (d, J=8.4 Hz, 1H), 7.49 (s, 1H), 7.36 (d, J=8.5 Hz, 1H), 5.10 (dd, J=12.8, 5.4 Hz, 1H), 4.23 (s, 1H), 3.63 (s, 2H), 3.47-3.26 (m, 7H), 3.14 (d, J=22.1 Hz, 3H), 2.88 (ddt, J=20.6, 9.1, 4.3 Hz, 3H), 2.65-2.51 (m, 2H), 2.23-1.82 (m, 4H), 1.33 (p, J=9.5 Hz, 2H) ppm.

### Step 5: Preparation of tert-butyl 2-(4-((4-(2-(2,6-dioxopiperidin-3-yl)-1,3-dioxoisoindolin-5-yl)piperazin-1-yl)methyl)piperidin-1-yl)acetate

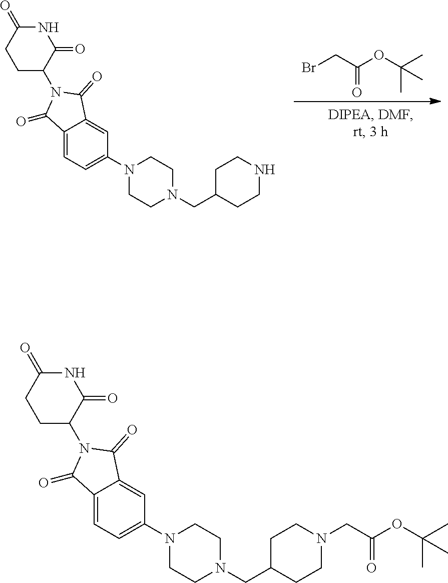

To a solution of 2-(2,6-dioxopiperidin-3-yl)-5-(4-(piperidin-4-ylmethyl)piperazin-1-yl)isoindoline-1,3-dione, Trifluoracetate (300 mg, 1.0 Eq, 543 μmol) and tert-butyl 2-bromoacetate (127 mg, 84.2 μL, 1.2 Eq, 652 mol) in DMF (3.0 mL) was added DIPEA (84.2 mg, 113 μL, 1.2 Eq, 652 mol). The resulting mixture was stirred at rt for 3 h. The reaction mixture was diluted with DI water (5 mL) and extracted with DCM (3×10 mL). The organic layers were combined, washed with brine (1×20 mL), filtered through a phase separator, and concentrated to dryness. The crude mixture was purified with flash column chromatography (DCM:acetone, 100:0 to 0:100) to afford tert-butyl 2-(4-((4-(2-(2,6-dioxopiperidin-3-yl)-1,3-dioxoisoindolin-5-yl)piperazin-1-yl)methyl)piperidin-1-yl)acetate (250 mg, 452 mol, 83.2%) as a yellow solid. LCMS (m/z): 555.3 [M+2]⁺. ¹H NMR (500 MHz, CDCl₃) δ 8.06 (s, 1H), 7.68 (d, J=8.5 Hz, 1H), 7.27 (d, J=2.3 Hz, 1H), 7.05 (dd, J=8.6, 2.3 Hz, 1H), 4.94 (dd, J=12.3, 5.4 Hz, 1H), 3.41 (t, J=5.1 Hz, 4H), 3.16 (s, 2H), 2.99 (s, 2H), 2.93-2.67 (m, 4H), 2.55 (t, J=5.1 Hz, 4H), 2.25 (d, J=7.1 Hz, 3H), 2.13 (dtd, J=11.9, 4.5, 1.9 Hz, 1H), 1.77 (d, J=12.9 Hz, 2H), 1.71-1.51 (m, 3H), 1.46 (s, 9H) ppm.

### Steps 6 and 7: Preparation of (2R)-N-(4-chloro-3-((1-(2-(4-((4-(2-(2,6-dioxopiperidin-3-yl)-1,3-dioxoisoindolin-5-yl)piperazin-1-yl)methyl)piperidin-1-yl)acetyl)piperidin-4-yl)oxy)phenyl)-2-((1s,4,S)-4-(6-fluoroquinolin-4-yl)cyclohexyl)propanamide, NU227164

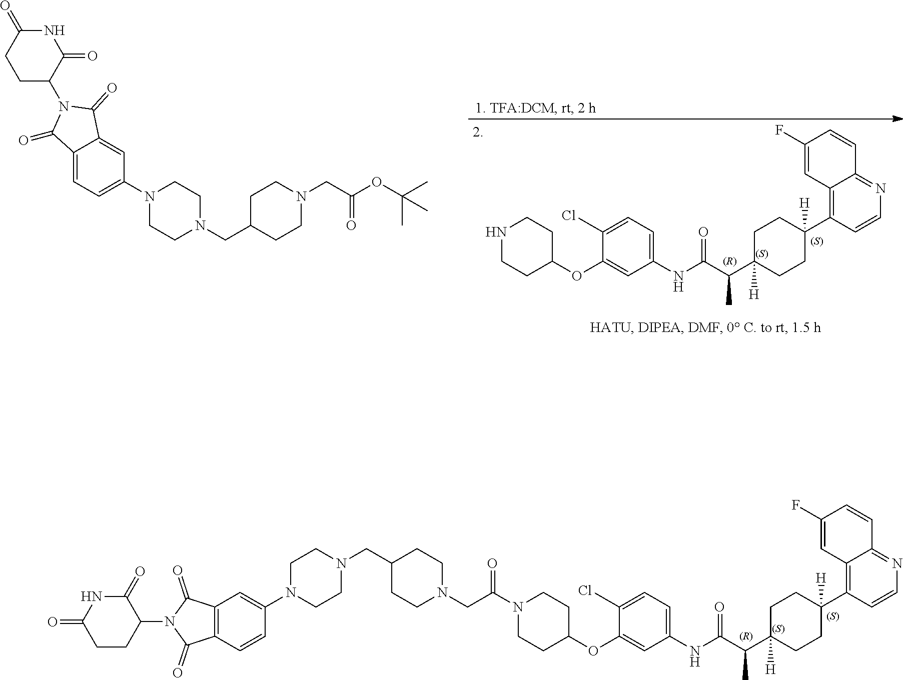

To tert-butyl 2-(4-((4-(2-(2,6-dioxopiperidin-3-yl)-1,3-dioxoisoindolin-5-yl)piperazin-1-yl)methyl)piperidin-1-yl)acetate (300 mg, 1.0 Eq, 542 mol) was added TFA:DCM (1:1, 2.0 mL). The resulting mixture was stirred at rt for 2 h. The reaction mixture was concentrated to dryness under nitrogen atmosphere. The crude oil was triturated with Et₂O (2×10 mL) to yield 2-(4-((4-(2-(2,6-dioxopiperidin-3-yl)-1,3-dioxoisoindolin-5-yl)piperazin-1-yl)methyl)piperidin-1-yl)acetic acid, Trifluoracetate (293 mg, 480 μmol, 88.6%) as a yellow solid. The product was taken forward without further purification. To a solution of intermediate acid (60 mg, 1.0 Eq, 98 mol) and (R)-N-(4-chloro-3-(piperidin-4-yloxy)phenyl)-2-((1s,4S)-4-(6-fluoroquinolin-4-yl) cyclohexyl)propanamide, HCl (54 mg, 1.0 Eq, 98 μmol) in DMF (1 mL) was added DIPEA (42 mg, 56 μL, 3.3 Eq, 0.32 mmol) and HATU (56 mg, 1.5 Eq, 0.15 mmol) at 0° C. under nitrogen atmosphere. The reaction mixture was allowed to stir at 0° C. for 30 min and allowed to warm to rt. After 1 h the reaction was complete, and the resulting mixture was directly filtered and purified with reverse phase preparative HPLC eluting from 10 to 90% ACN in water (0.1% TFA). The desired fractions were lyophilized (3d) to provide (2R)-N-(4-chloro-3-((1-(2-(4-((4-(2-(2,6-dioxopiperidin-3-yl)-1,3-dioxoisoindolin-5-yl)piperazin-1-yl)methyl)piperidin-1-yl)acetyl)piperidin-4-yl)oxy)phenyl)-2-((1s,4S)-4-(6-fluoroquinolin-4-yl)cyclohexyl) propanamide, 2 Trifluoracetate (63 mg, 52 μmol, 53%) as a light green solid.

### Synthesis of NU227168

### Step 1: Preparation of tert-butyl 4-(4-(2-(2,6-dioxopiperidin-3-yl)-1,3-dioxoisoindolin-5-yl)piperazin-1-yl)piperidine-1-carboxylate

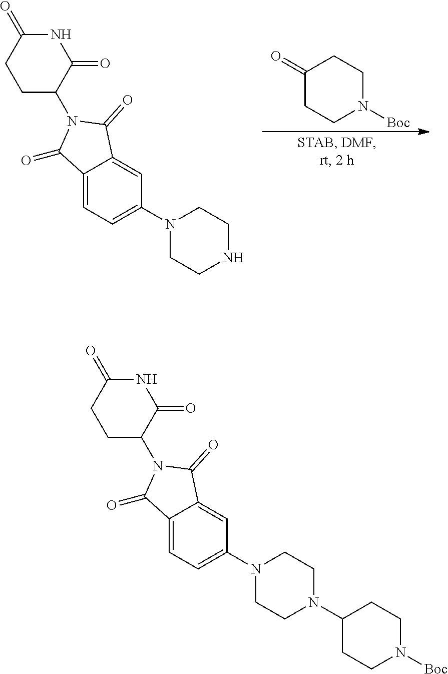 [SMILES: O=C1CCC(N2C(=O)c3ccc(N4CCNCC4)cc3C2=O)C(=O)N1 ; CC(C)(C)OC(=O)N1CCC(=O)CC1 ; CC(C)(C)OC(=O)N1CCC(N2CCN(c3ccc4c(c3)C(=O)N(C3CCC(=O)NC3=O)C4=O)CC2)CC1]

To a solution of 2-(2,6-dioxopiperidin-3-yl)-5-(piperazin-1-yl)isoindoline-1,3-dione, Trifluoracetate (150 mg, 1.0 Eq, 329 mol) and tert-butyl 4-oxopiperidine-1-carboxylate (78.8 mg, 1.2 Eq, 395 μmol) in DMF (2 mL) was added sodium triacetoxyhydroborate (209 mg, 3.0 Eq, 988 mol). The reaction mixture was allowed to stir at rt. After 2 h the reaction was quenched with DI water and extracted with EtOAc (3×5 mL). The organic layers were combined and washed with brine (1×15 mL). The organic layer was dried by filtering through phase separator and concentrated to a yellow oil to provide pure tert-butyl 4-(4-(2-(2,6-dioxopiperidin-3-yl)-1,3-dioxoisoindolin-5-yl)piperazin-1-yl)piperidine-1-carboxylate (160 mg, 304 μmol, 92.4%). LCMS (m z): 527.4 [M+2]. ¹H NMR (500 MHz, CDCl₃) δ 8.10 (s, 1H), 7.69 (d, J=8.5 Hz, 1H), 7.28 (d, J=2.3 Hz, 1H), 7.06 (dd, J=8.6, 2.3 Hz, 1H), 4.94 (dd, J=12.3, 5.4 Hz, 1H), 4.17 (s, 2H), 3.43 (s, 4H), 2.93-2.62 (m, 9H), 2.45 (s, 1H), 2.21-2.06 (m, 2H), 1.82 (s, 2H), 1.62 (s, 2H), 1.46 (s, 9H) ppm.

### Step 2: Preparation of 2-(2,6-dioxopiperidin-3-yl)-5-(4-(piperidin-4-yl)piperazin-1-yl)isoindoline-1,3-dione

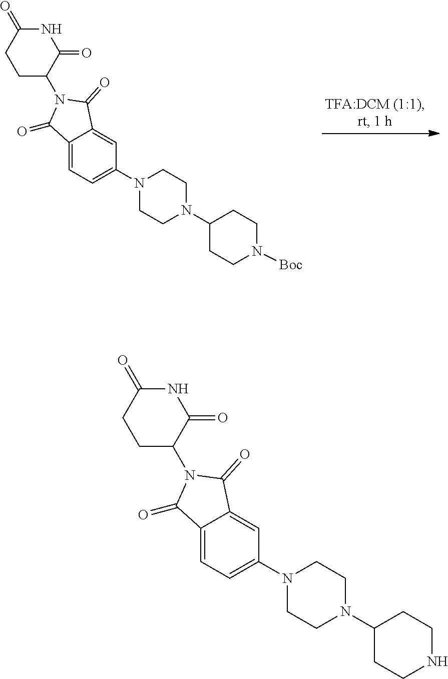 [SMILES: CC(C)(C)OC(=O)N1CCC(N2CCN(c3ccc4c(c3)C(=O)N(C3CCC(=O)NC3=O)C4=O)CC2)CC1 ; O=C1CCC(N2C(=O)c3ccc(N4CCN(C5CCNCC5)CC4)cc3C2=O)C(=O)N1]

To tert-butyl 4-(4-(2-(2,6-dioxopiperidin-3-yl)-1,3-dioxoisoindolin-5-yl)piperazin-1-yl)piperidine-1-carboxylate (250 mg, 1.0 Eq, 476 mol) was added DCM:TFA (1:1, 3.0 mL). The resulting mixture was allowed to stir at rt. After 1 h the reaction was concentrated to dryness under nitrogen atmosphere. The crude residue was triturated with Et₂O and then concentrated to yield 2-(2,6-dioxopiperidin-3-yl)-5-(4-(piperidin-4-yl)piperazin-1-yl)isoindoline-1,3-dione, Trifluoracetate (245 mg, 455 μmol, 95.7%) as a dark yellow solid. The product was taken forward without further purification. LCMS (m z): 427.1 [M+2]⁺.

### Step 3: Preparation of tert-butyl 2-(4-(4-(2-(2,6-dioxopiperidin-3-yl)-1,3-dioxoisoindolin-5-yl)piperazin-1-yl)piperidin-1-yl)acetate

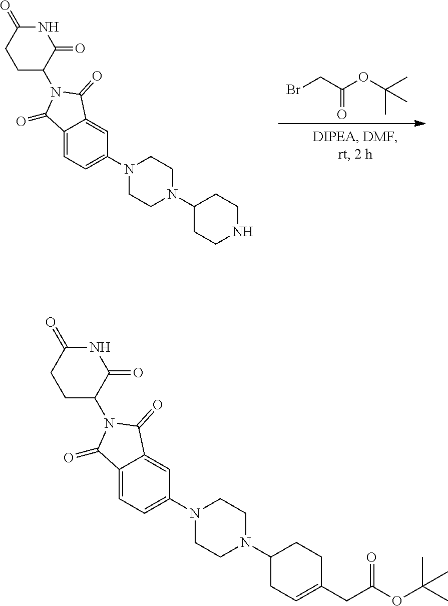

To a solution of 2-(2,6-dioxopiperidin-3-yl)-5-(4-(piperidin-4-yl)piperazin-1-yl)isoindoline-1,3-dione, Trifluoracetate (200 mg, 1.0 Eq, 371 μmol) in DMF (2.5 mL) was added DIPEA (144 mg, 194 μL, 3.0 Eq, 111 mmol) followed by tert-butyl 2-bromoacetate (86.9 mg, 57.6 μL, 1.2 Eq, 446 mol). The resulting mixture was stirred for 2 h at rt. The resulting mixture was diluted with water (5 mL) and extracted with DCM (3×5 mL). The organic layers were combined and washed with brine (1×15 mL), dried via filtration through a phase separator, and the filtrate was concentrated to a yellow oil. Cold Et₂O was added to initiate precipitation of product and the solid was filtered and washed with cold Et₂O to yield tert-butyl 2-(4-(4-(2-(2,6-dioxopiperidin-3-yl)-1,3-dioxoisoindolin-5-yl)piperazin-1-yl)piperidin-1-yl)acetate (190 mg, 352 μmol, 94.8%) as a yellow solid. LCMS (mi/z): 540.3 [M+1]⁺. ¹H NMR (500 MHz, CDCl₃) δ 8.30 (s, 1H), 7.69 (d, J=8.5 Hz, 1H), 7.28 (d, J=2.3 Hz, 1H), 7.05 (dd, J=8.6, 2.4 Hz, 1H), 4.93 (dd, J=12.3, 5.4 Hz, 1H), 3.46 (s, 4H), 3.13 (s, 2H), 3.05 (d, J=10.9 Hz, 2H), 2.94-2.67 (m, 7H), 2.22 (t, J=11.4 Hz, 2H), 2.15-2.03 (m, 1H), 1.73 (d, J=12.3 Hz, 5H), 1.46 (s, 9H) ppm.

### Steps 4 and 5: Preparation of (2R)-N-(4-chloro-3-((1-(2-(4-(4-(2-(2,6-dioxopiperidin-3-yl)-1,3-dioxoisoindolin-5-yl)piperazin-1-yl)piperidin-1-yl)acetyl)piperidin-4-yl)oxy)phenyl)-2-((1s,4S)-4-(6-fluoroquinolin-4-yl)cyclohexyl)propanamide, NU227168

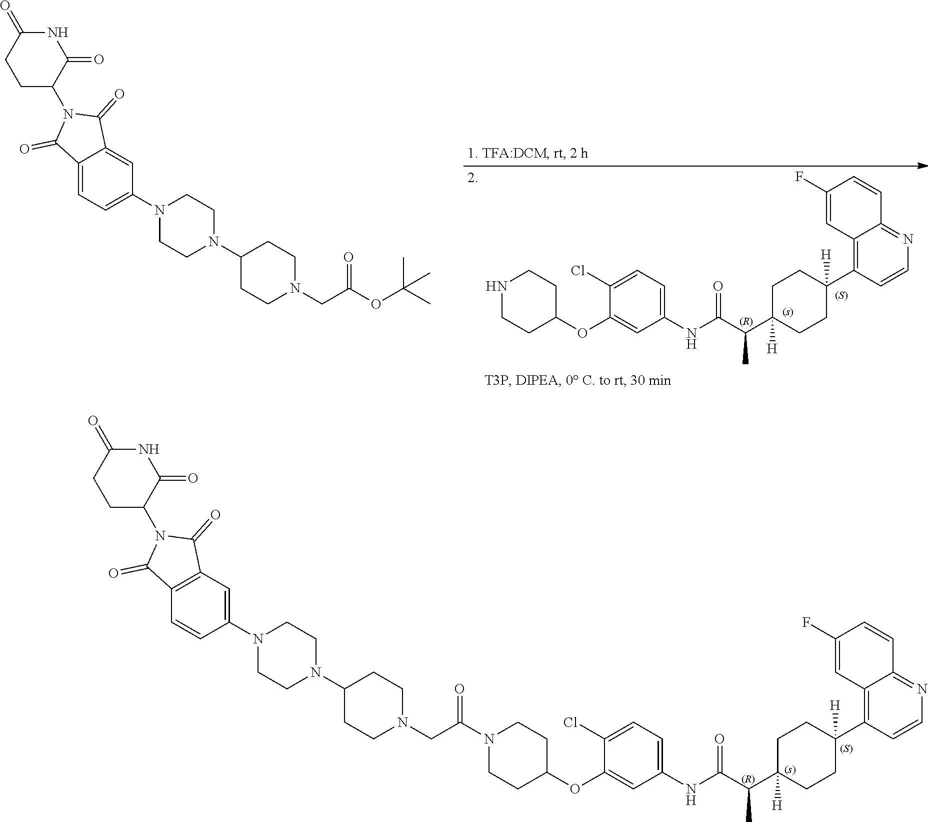

To tert-butyl 2-(4-(4-(2-(2,6-dioxopiperidin-3-yl)-1,3-dioxoisoindolin-5-yl)piperazin-1-yl)piperidin-1-yl)acetate (100 mg, 1.0 Eq, 185 μmol) was added DCM:TFA (1:1, 2.0 ml). The resulting mixture was stirred at rt for 2 h. The reaction mixture was concentrated to dryness under nitrogen atmosphere and the crude residue was triturated with Et₂O (2×5 mL) to yield pure 2-(4-(4-(2-(2,6-dioxopiperidin-3-yl)-1,3-dioxoisoindolin-5-yl)piperazin-1-yl)piperidin-1-yl)acetic acid, Trifluoroacetic acid (100 mg, 167 μmol, 90.3%) as a yellow solid. LCMS (m z): 485.1 [M+2]⁺. The product was taken forward without further purification. To a solution of the intermediate acid (150 mg, 1.0 Eq, 251 μmol) in DMF (2.5 mL) was added DIPEA (162 mg, 219 μL, 5.0 Eq, 1.26 mmol) and (R)-N-(4-chloro-3-(piperidin-4-yloxy)phenyl)-2-((1s,4S)-4-(6-fluoroquinolin-4-yl)cyclohexyl)propanamide, HCl (137 mg, 1.0 Eq, 251 μmol). The reaction mixture was cooled to 0° C. and 2,4,6-tripropyl-1,3,5,2,4,6-trioxatriphosphinane 2,4,6-trioxide (480 mg, 449 μL, 50% Wt in EtOAc, 3.0 Eq, 754 μmol) was added slowly. The reaction mixture was allowed to warm to rt and stirred for 30 min. The reaction was quenched with cold DI water (2 mL), extracted with EtOAc (3×5 mL), and dried via filtration through a phase separator. The filtrated was concentrated under reduced pressure and immediately purified with reverse phase preparative HPLC eluting from 10 to 90% ACN in water (0.1% TFA). The desired fractions were lyophilized (3d) to provide pure (2R)-N-(4-chloro-3-((1-(2-(4-(4-(2-(2,6-dioxopiperidin-3-yl)-1,3-dioxoisoindolin-5-yl)piperazin-1-yl)piperidin-1-yl)acetyl)piperidin-4-yl)oxy)phenyl)-2-((1s,4S)-4-(6-fluoroquinolin-4-yl)cyclohexyl)propanamide, 2Trifluoracetate (150 mg, 125 μmol, 49.6%) as a yellow solid.

### Synthesis of (2R)-2-((1s,4S)-4-(6-fluoroquinolin-4-yl)cyclohexyl)-N-(3-((1-(2-(4-(4-(2-(1-methyl-2,6-dioxopiperidin-3-yl)-1,3-dioxoisoindolin-5-yl)piperazin-1-yl)piperidin-1-yl)acetyl)piperidin-4-yl)oxy)phenyl)propanamide, 2Trifluoracetate (NU227428)

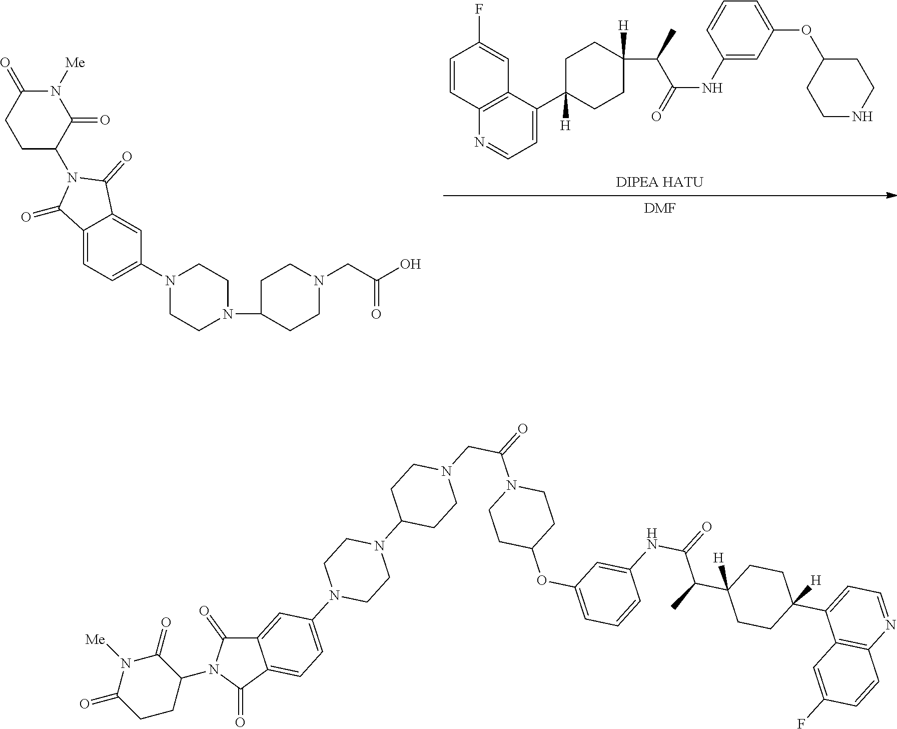

To a solution of 2-(4-(4-(2-(1-methyl-2,6-dioxopiperidin-3-yl)-1,3-dioxoisoindolin-5-yl)piperazin-1-yl)piperidin-1-yl)acetic acid, Trifluoracetate (20 mg, 1 Eq, 33 mol) and DIPEA (13 mg, 17 μL, 3 Eq, 98 mol) in DMF (2 mL) was added HATU (10 mg, 0.8 Eq, 26 μmol) at 0° C. The resulting mixture was allowed to stir at rt for 30 min. To the resulting mixture was added (R)-2-((1s,4S)-4-(6-fluoroquinolin-4-yl)cyclohexyl)-N-(3-(piperidin-4-yloxy)phenyl)propanamide, HCl (12 mg, 0.7 Eq, 23 μmol) at 0° C. The reaction mixture was allowed to stir at rt for 15 min at which time the reaction mixture was quenched with DI water (0.5 mL) and concentrated under nitrogen atmosphere. The crude residue was taken up in a 1:1 mixture of acetonitrile:water and prepared for purification. The reaction mixture was purified using RP HPLC eluting with 10 to 90% ACN in water (0.1% TFA) long column. The fractions with desired product were concentrated using lypholyzer (2 d) to provide pure (2R)-2-((1s,4S)-4-(6-fluoroquinolin-4-yl)cyclohexyl)-N-(3-((1-(2-(4-(4-(2-(1-methyl-2,6-dioxopiperidin-3-yl)-1,3-dioxoisoindolin-5-yl)piperazin-1-yl)piperidin-1-yl)acetyl)piperidin-4-yl)oxy)phenyl)propanamide, 2Trifluoracetate (15 mg, 13 mol, 39%) as a yellow solid.

### Synthesis of (2R)-N-(3-((1-(3-(4-(2-(2,6-dioxopiperidin-3-yl)-1,3-dioxoisoindolin-5-yl)piperazin-1-yl)propanoyl)piperidin-4-yl)oxy)phenyl)-2-((1s,4S)-4-(6-fluoroquinolin-4-yl)cyclohexyl)propanamide, Trifluoracetate (NU227427)

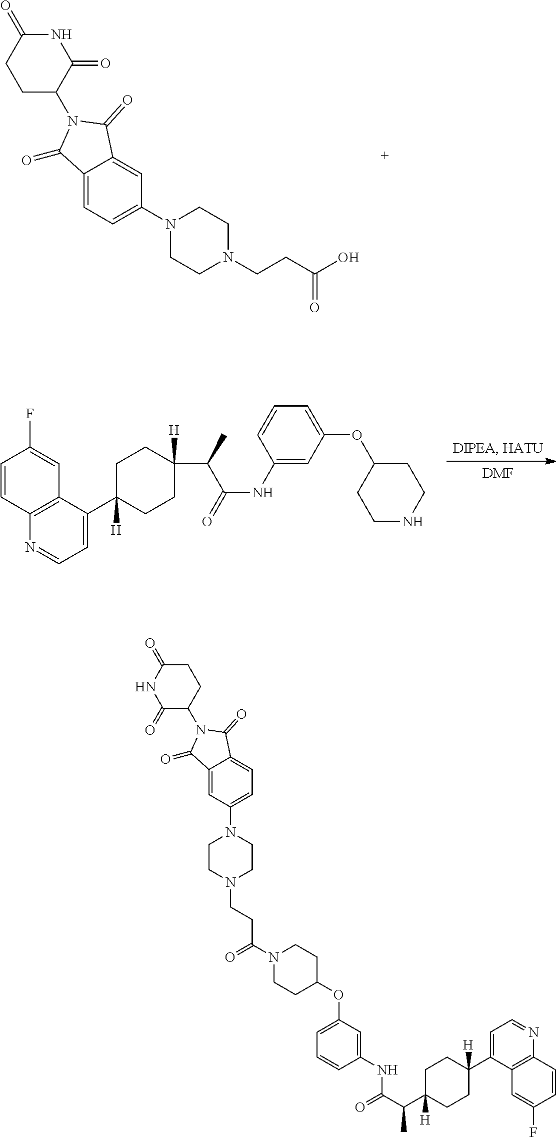

To a solution of 3-(4-(2-(2,6-dioxopiperidin-3-yl)-1,3-dioxoisoindolin-5-yl)piperazin-1-yl)propanoic acid in DMF (1 mL) was added DIPEA (19 mg, 25 μL, 3 Eq, 0.14 mmol) and 2-(3H-[1,2,3]triazolo[4,5-b]pyridin-3-yl)-1,1,3,3-tetramethylisouronium hexafluorophosphate(V) (15 mg, 0.8 Eq, 39 mol) at 0° C. The resulting reaction mixture was allowed to stir at rt for 15 min at which time (R)-2-((1s,4S)-4-(6-fluoroquinolin-4-yl)cyclohexyl)-N-(3-(piperidin-4-yloxy)phenyl)propanamide, HCl (20 mg, 0.8 Eq, 39 mol) was added at 0° C. The resulting mixture was stirred at rt for 30 min. The mixture was quenched with DI water (1 mL) and concentrated under nitrogen atmosphere. The crude residue was taken up in a 1:1 mixture of acetonitrile:water and prepared for purification. The reaction mixture was purified using RP HPLC eluting with 10 to 90% ACN in water (0.1% TFA) long column. The fractions with desired product were concentrated using lypholyzer (2 d) to provide pure (2R)-N-(3-((1-(3-(4-(2-(2,6-dioxopiperidin-3-yl)-1,3-dioxoisoindolin-5-yl)piperazin-1-yl)propanoyl)piperidin-4-yl)oxy)phenyl)-2-((1s,4S)-4-(6-fluoroquinolin-4-yl)cyclohexyl)propanamide, Trifluoracetate (10 mg, 10 mol, 21%)as a yellow solid.

### Synthesis of (2R)-N-(3-((1-(2-(4-(2-(2,6-dioxopiperidin-3-yl)-1,3-dioxoisoindolin-5-yl)piperazin-1-yl)acetyl)piperidin-4-yl)oxy)phenyl)-2-((1s,4S)-4-(6-fluoroquinolin-4-yl)cyclohexyl)propanamide, Trifluoracetate (NU227426)

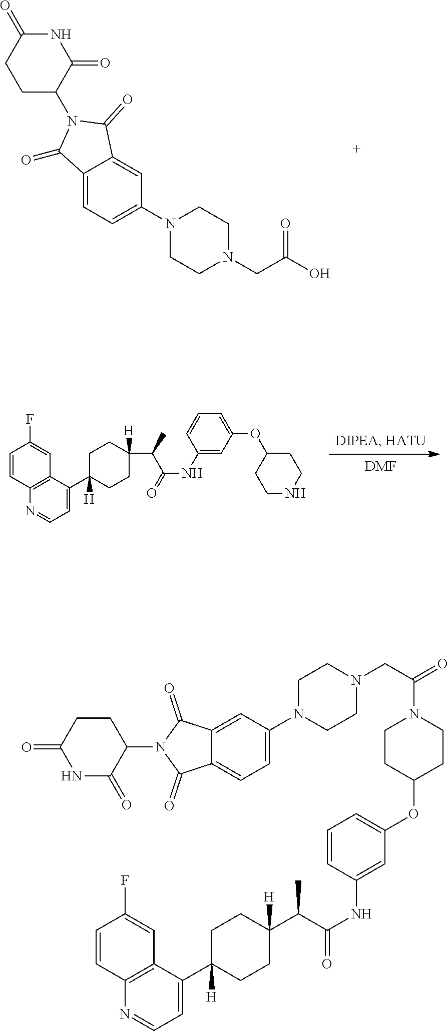

To a solution of 2-(4-(2-(2,6-dioxopiperidin-3-yl)-1,3-dioxoisoindolin-5-yl)piperazin-1-yl)acetic acid, Trifluoracetate (20 mg, 1 Eq, 39 mol) in DMF (1 mL) was added DIPEA (15 mg, L, 3 Eq, 0.12 mmol) and HATU (18 mg, 1.2 Eq, 47 mol) at 0° C. The resulting mixture was stirred at rt for 15 min at which time (R)-2-((1s,4S)-4-(6-fluoroquinolin-4-yl)cyclohexyl)-N-(3-(piperidin-4-yloxy)phenyl)propanamide, HCl (16 mg, 0.8 Eq, 31 mol) was added and the resulting mixture was stirred at rt. After 30 min the reaction was complete and the reaction mixture was quenched with DI water (1 mL) and concentrated under nitrogen atmosphere. The crude residue was taken up in a 1:1 mixture of acetonitrile:water and prepared for purification. The reaction mixture was purified using RP HPLC eluting with 10 to 90% ACN in water (0.1% TFA) long column. The fractions with desired product were concentrated using lypholyzer (2 d) to provide pure (2R)-N-(3-((1-(2-(4-(2-(2,6-dioxopiperidin-3-yl)-1,3-dioxoisoindolin-5-yl)piperazin-1-yl)acetyl)piperidin-4-yl)oxy)phenyl)-2-((1s,4S)-4-(6-fluoroquinolin-4-yl)cyclohexyl)propanamide, Trifluoracetate (10 mg, 10 μmol, 26%)as a yellow solid.

### Synthesis of (2R)-N-(1-(3-(2-(2-((2-(2,6-dioxopiperidin-3-yl)-1,3-dioxoisoindolin-4-yl)amino)ethoxy)ethoxy)propanoyl)piperidin-4-yl)-2-((1s,4S)-4-(6-fluoroquinolin-4-yl)cyclohexyl)propanamide, Trifluoracetate (NU227425)

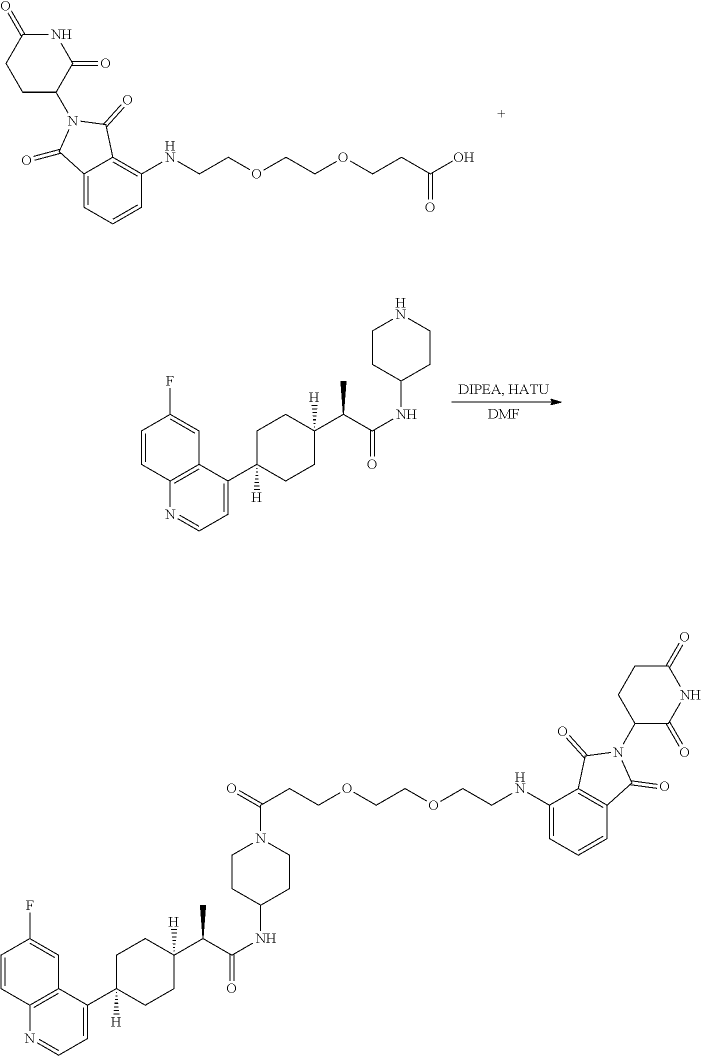

To a solution of 3-(2-(2-((2-(2,6-dioxopiperidin-3-yl)-1,3-dioxoisoindolin-4-yl)amino)ethoxy)ethoxy)propanoic acid, Trifluoracetate (30 mg, 1 Eq, 55 μmol) in DMF (1 mL) was added DTPEA (21 mg, 29 μL, 3 Eq, 0.16 mmol) and HATU (25 mg, 1.2 Eq, 66 μmol) at 0° C. The resulting mixture was stirred for 15 min at rt which time (R)-2-((1s,4S)-4-(6-fluoroquinolin-10 4-yl)cyclohexyl)-N-(piperidin-4-yl)propanamide, HCl (23 mg, 1 Eq, 55 μmol) was added. The resulting mixture was allowed to stir at rt for 1 h at which time the reaction mixture was quenched with DI water (0.5 mL). The mixture was immediately filtered for purification with RP HTPLC eluting with 10 to 900% ACN in water (0.1 00 TFA) with long column. The desired fractions were concentrated with lyophylyzer (3d) to provide (2R)-N-(1-(3-(2-(2-((2-(2,6-dioxopiperidin-3-yl)-15 1,3-dioxoisoindolin-4-yl)amino)ethoxy)ethoxy)propanoyl)piperidin-4-yl)-2-((1s,4S)-4-(6-fluoroquinolin-4-yl)cyclohexyl)propanamide, Trifluoracetate as a yellow solid.

### Synthesis of (2R)-N-(1-(2-(4-((4-(2-(2,6-dioxopiperidin-3-yl)-1,3-dioxoisoindolin-5-yl)piperazin-1-yl)methyl)piperidin-1-yl)acetyl)piperidin-4-yl)-2-((1s,4S)-4-(6-fluoroquinolin-4-yl)cyclohexyl)propanamide, 2Trifluoroacetic acid (NU227424)

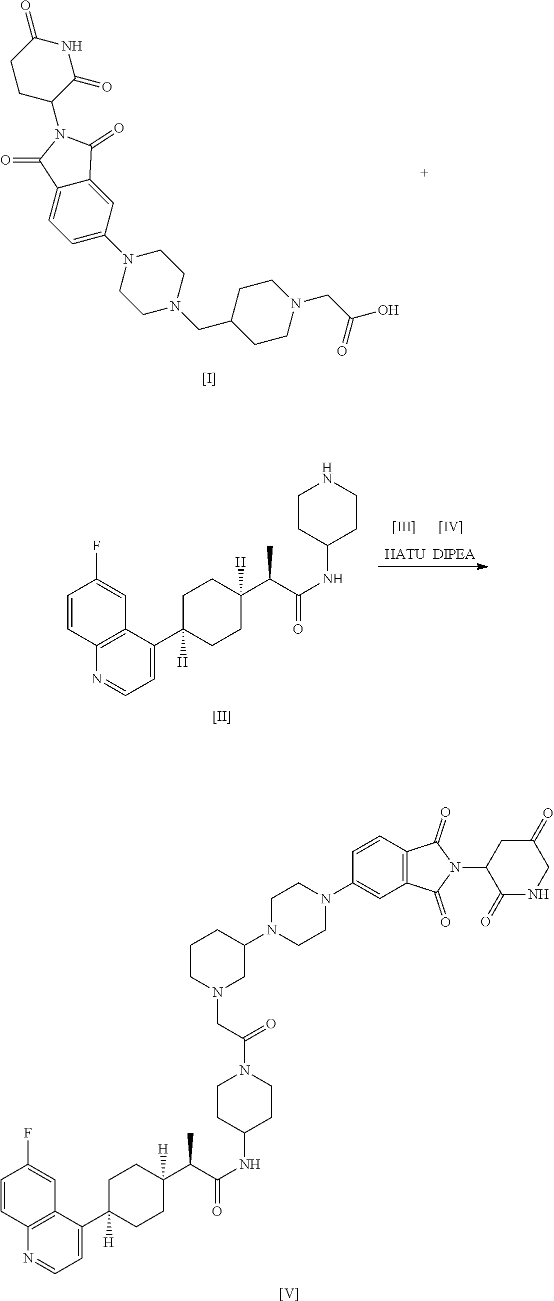

To a solution of 2-(4-((4-(2-(2,6-dioxopiperidin-3-yl)-1,3-dioxoisoindolin-5-yl)piperazin-1-yl)methyl)piperidin-1-yl)acetic acid (20 mg, 1 Eq, 40 μmol) in DMF (1 mL) was added HATU (18 mg, 1.2 Eq, 48 mol) and DIPEA (16 mg, 21 μL, 3 Eq, 0.12 mmol). The reaction mixture was stirred for 30 min at 0° C. and then (R)-2-((1s,4S)-4-(6-fluoroquinolin-4-yl)cyclohexyl)-N-(piperidin-4-yl)propanamide (15 mg, 1 Eq, 40 μmol) was added. The reaction mixture was allowed to stir at rt for 1 h. The reaction mixture was quenched with DI water (1 mL) and immediately filtered for PREP purification. The crude product was purified with RP HPLC eluting with 10 to 90% ACN in water (0.1% TFA) with long column to provide (2R)-N-(1-(2-(4-((4-(2-(2,6-dioxopiperidin-3-yl)-1,3-dioxoisoindolin-5-yl)piperazin-1-yl)methyl)piperidin-1-yl)acetyl)piperidin-4-yl)-2-((1 s,4S)-4-(6-fluoroquinolin-4-yl)cyclohexyl)propanamide, 2Trifluoroacetic acid (15 mg, 14 mol, 34%) as a yellow solid.

### PROTAC-Induced IDO1 Degradation in U87 Cells Using Western Blot Analysis:

### Western Blotting General Procedures:

Protein samples were prepared by lysing the cells in RIPA buffer (Sigma-Aldrich, Cat #R0278) supplemented with protease and phosphatase inhibitors on ice for 30 min. Cell lysates were clarified by centrifugation at 16,000×g for 15 min at 4° C. Equal amounts of proteins as quantified by bicinchoninic acid assay (Pierce) were separated on an SDS-polyacrylamide gel electrophoresis and proteins were electrophoretically transferred onto a polyvinylidene fluoride (PVDF) membrane using a Trans-Blot Turbo Transfer System (Bio-Rad, Hercules, CA, USA). The blotted membranes were blocked for 1 h at room temperature in blocking buffer containing 5% (w/v) nonfat dry milk in Tris-buffered saline and 0.1% Tween 20 (TBS-T) followed by incubating the membranes overnight at 4° C. with primary antibodies against a target protein diluted at a standardized concentration in blocking buffer. The blots were washed three times with TBS-T and incubated for 1 h at room temperature with horseradish peroxidase-conjugated secondary antibody generated against the host antigen in which the primary antibody was generated. The protein bands were detected using an enhanced chemiluminescence reagent (SuperSignal West Femto Maximum Sensitivity substrate) and blots were visualized with Bio-Rad ImageLab software on a Bio-Rad ChemIDO1c MP imaging system. All blots were stripped and re-probed with glyceraldehyde phosphate dehydrogenase (GAPDH) to ensure the proteins were loaded equally across all the samples in a particular blot. Western blotting analysis for proteins of interest used antibodies at optimized concentrations.

### Kynurenine Assay General Procedures:

Kynurenine levels in cell culture supernatants were measured using Erhlich's reagent. Briefly, cell culture supernatants were incubated with a 10% final concentration of trichloroacetic acid in Eppendorf tubes for 20 min at 60° C. to release kynurenine from cells and to precipitate proteins. After 20 minutes, samples were centrifuged for 20 min at 2500×g and supernatants were mixed with 20 mg/mL 4-dimethylamino benzaldehyde in acetic acid (Sigma Aldrich) at a 1:1 ratio and absorbance was measured at 480 nm using a plate reader.

### Extended Dose Range of Selected PROTACs:

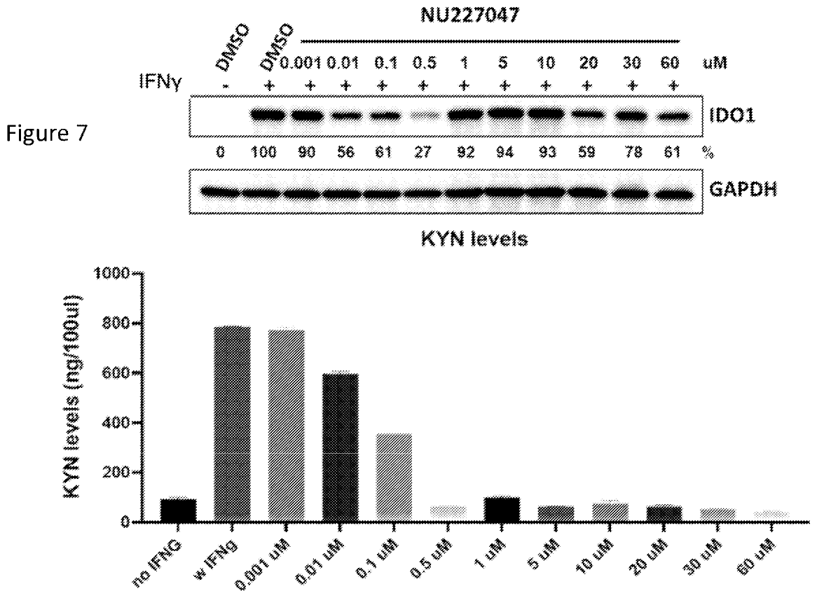

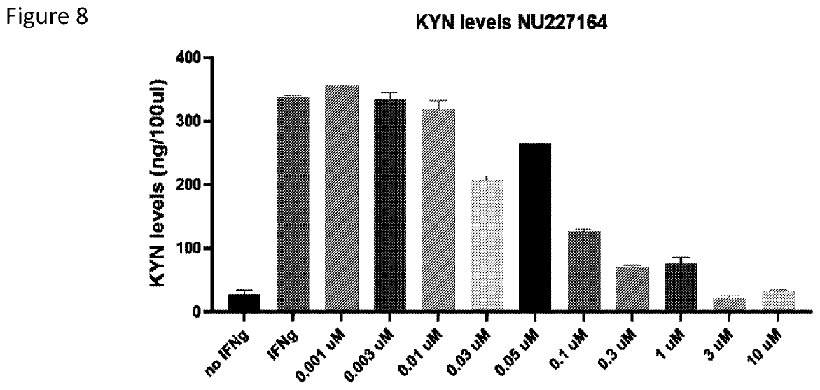

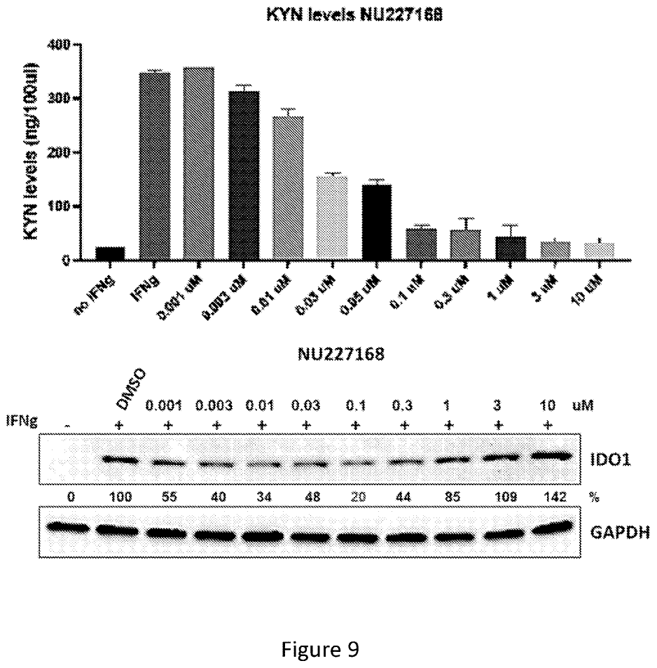

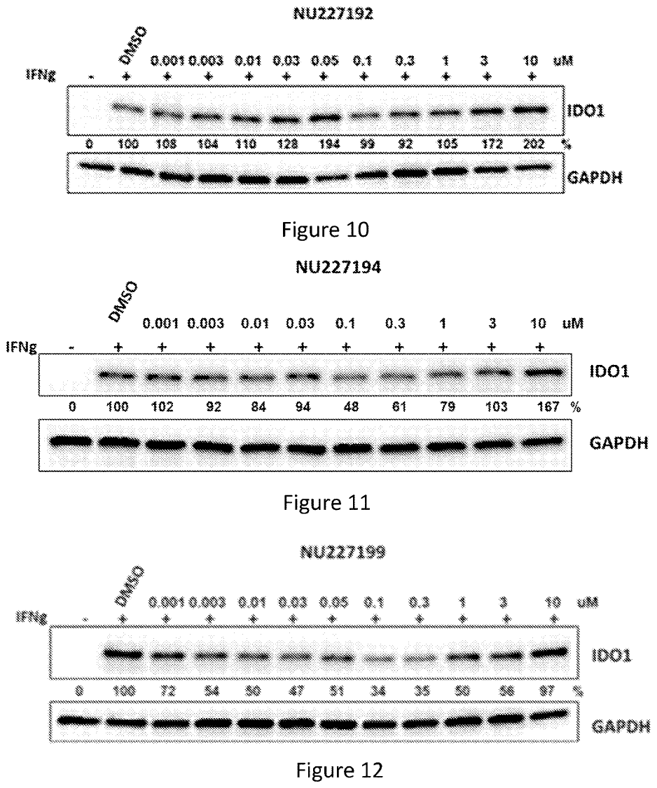

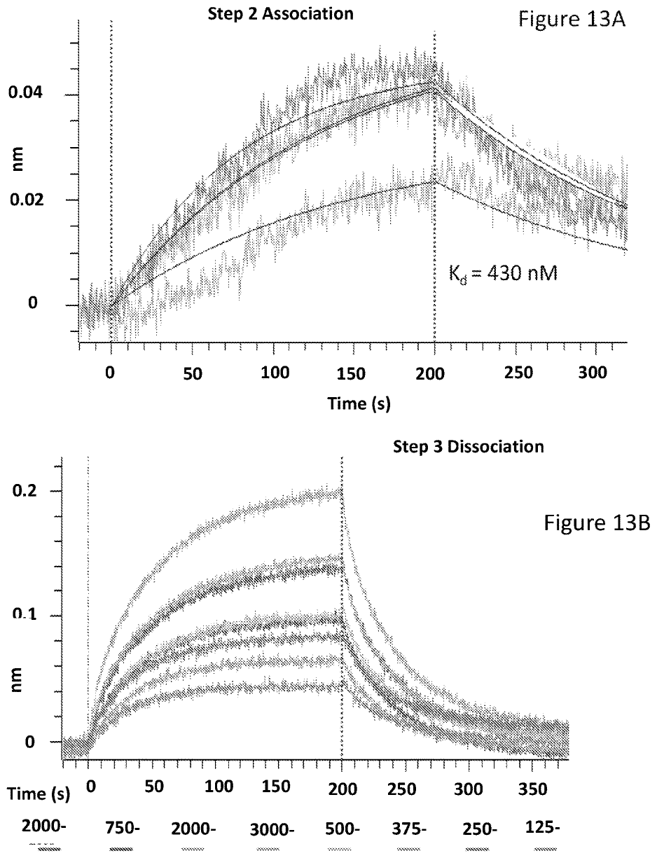

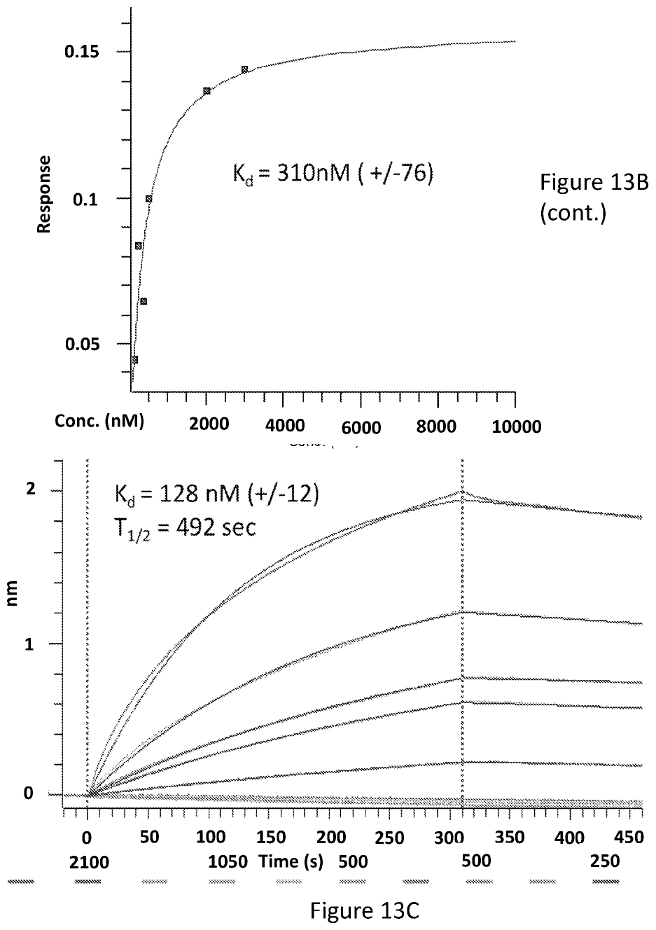

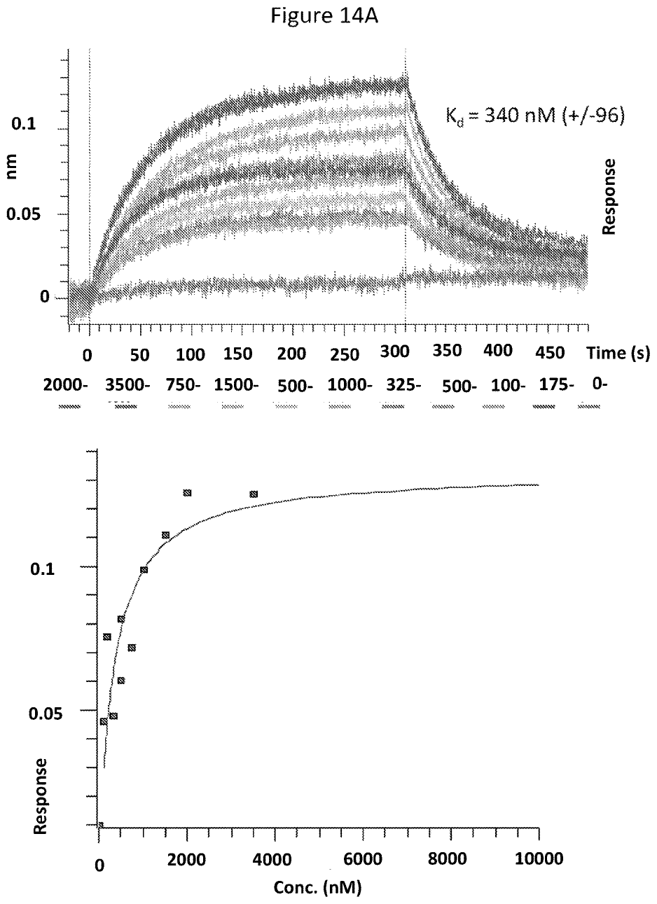

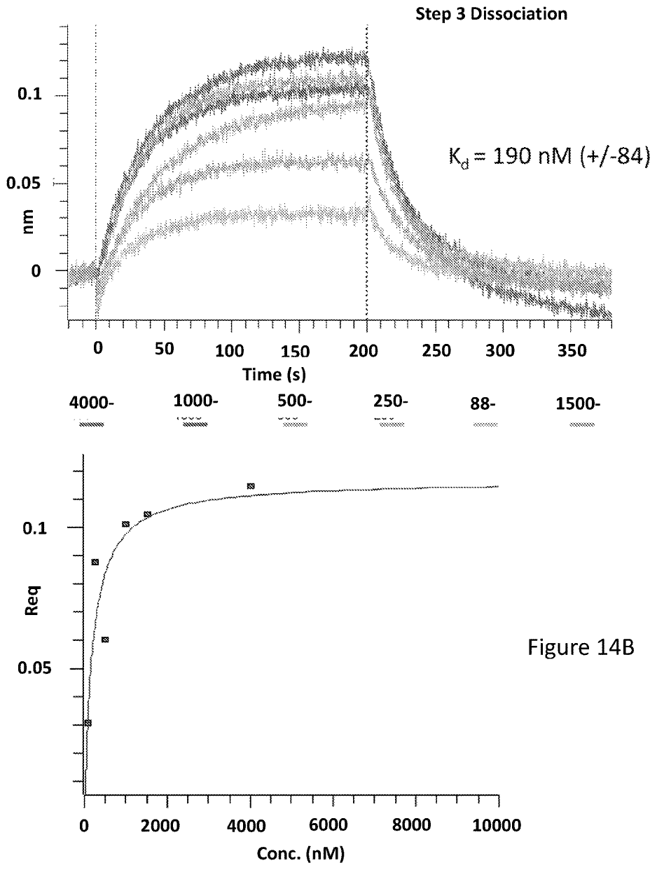

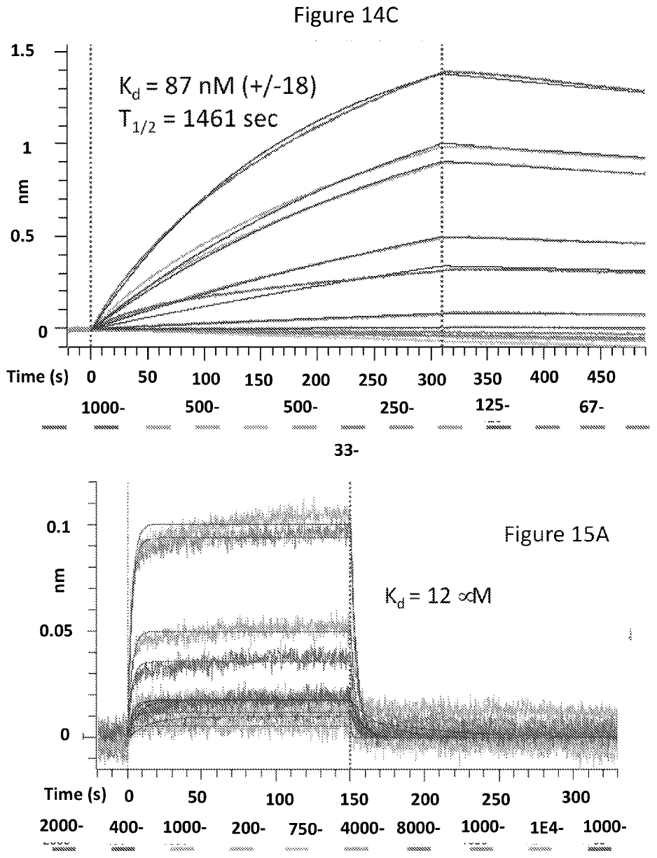

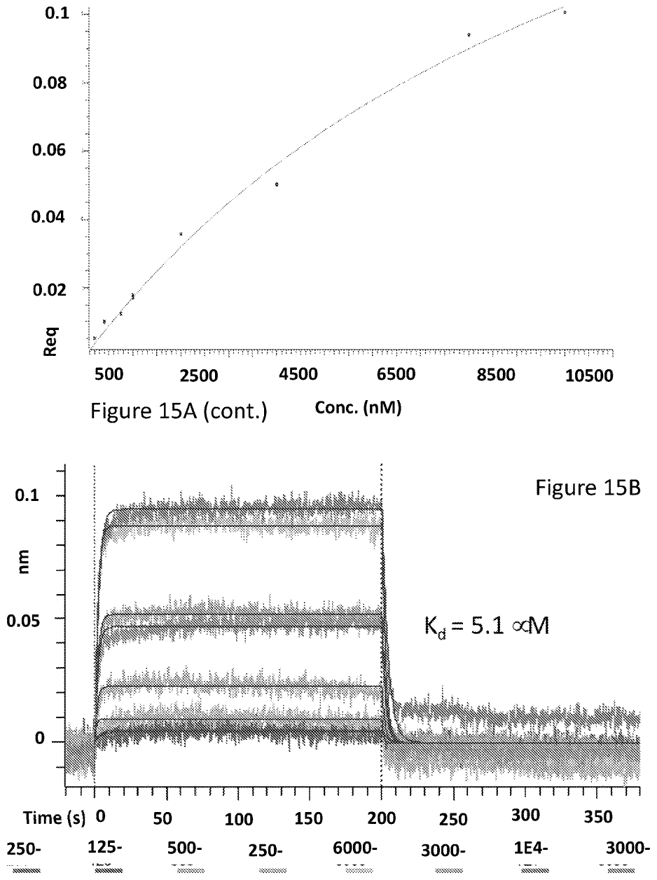

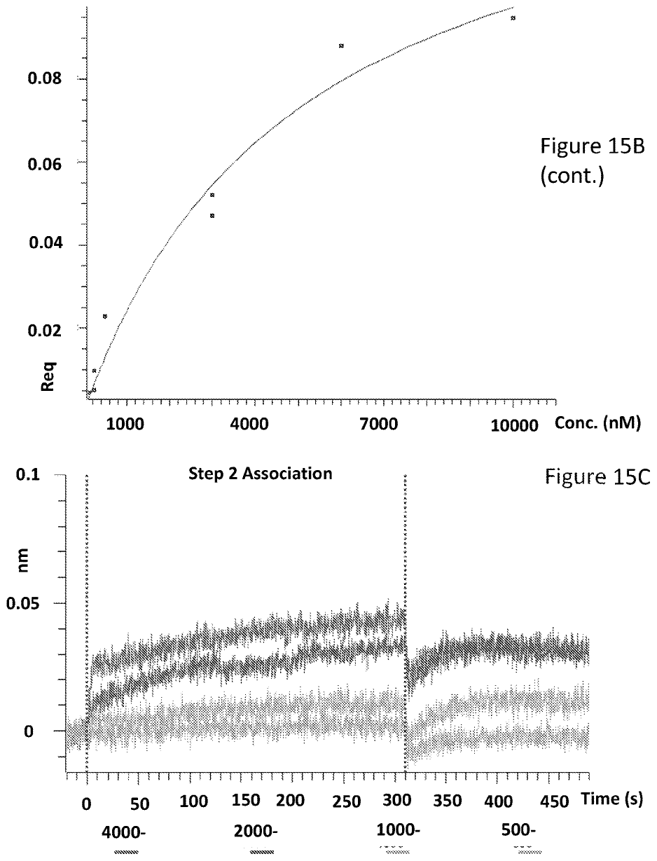

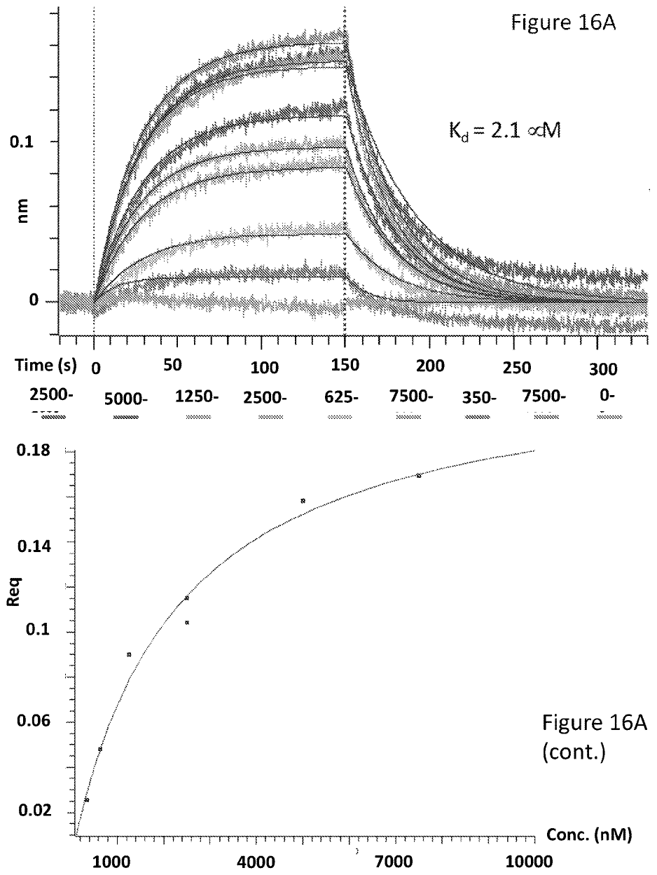

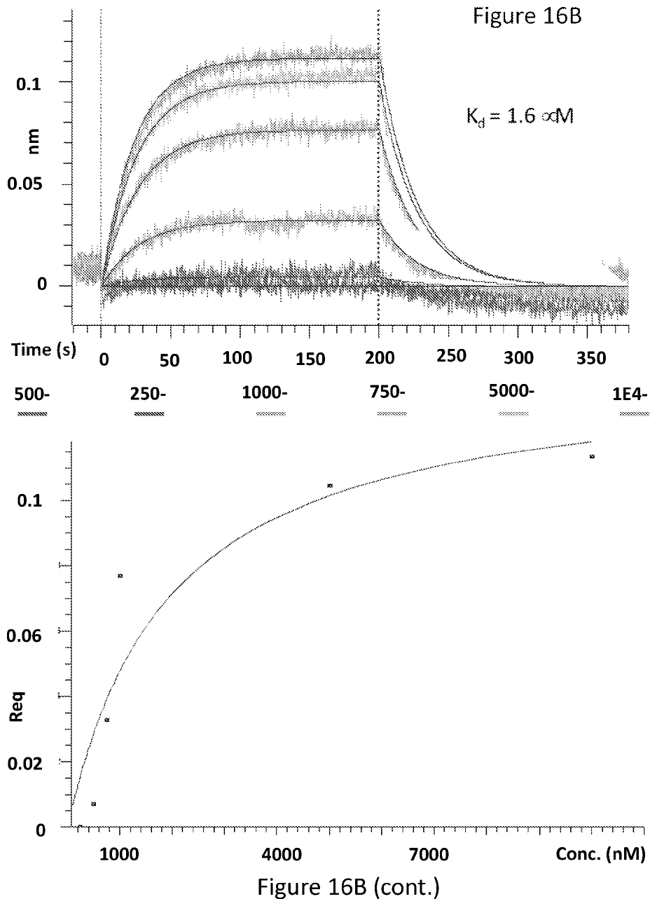

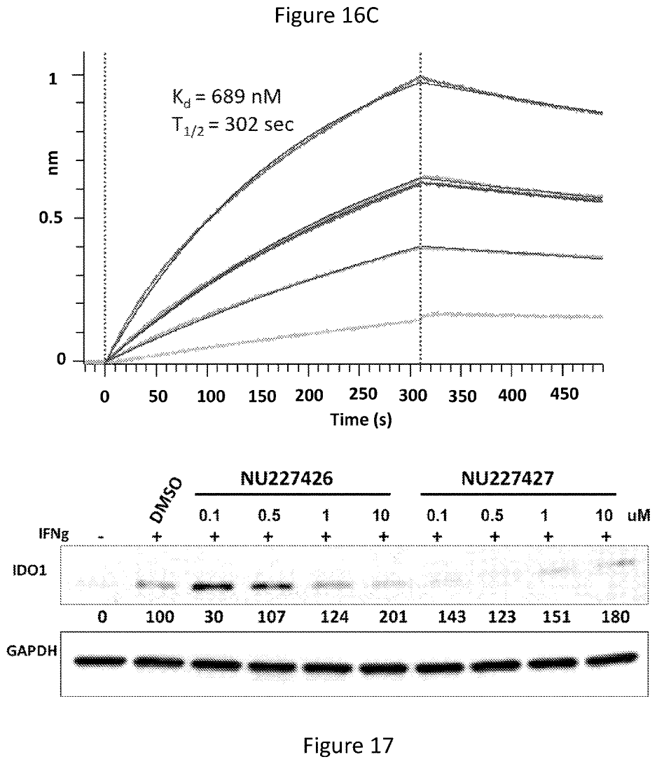

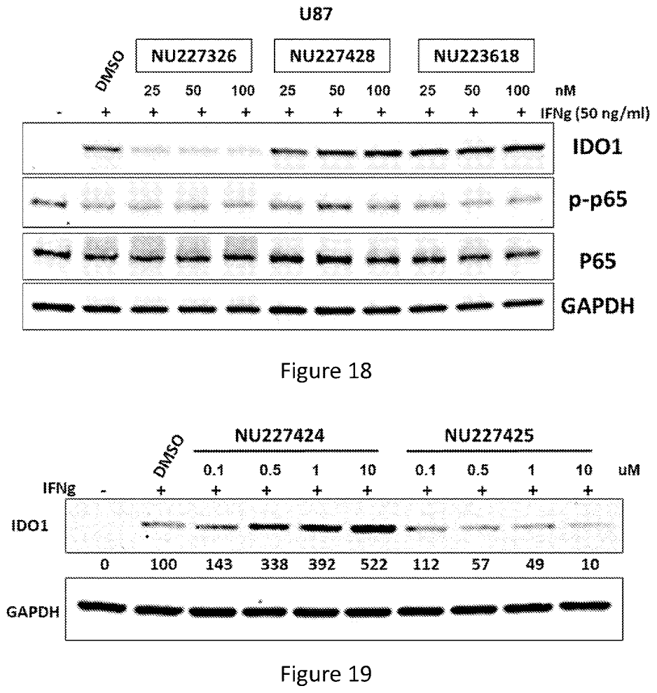

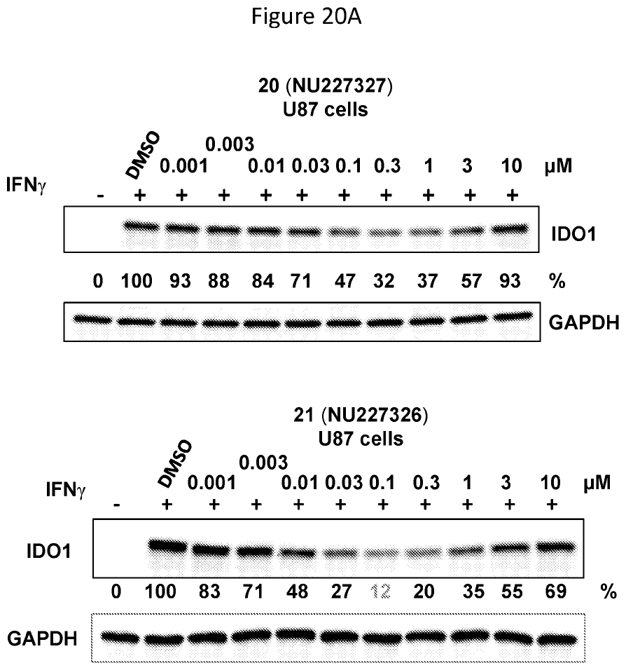

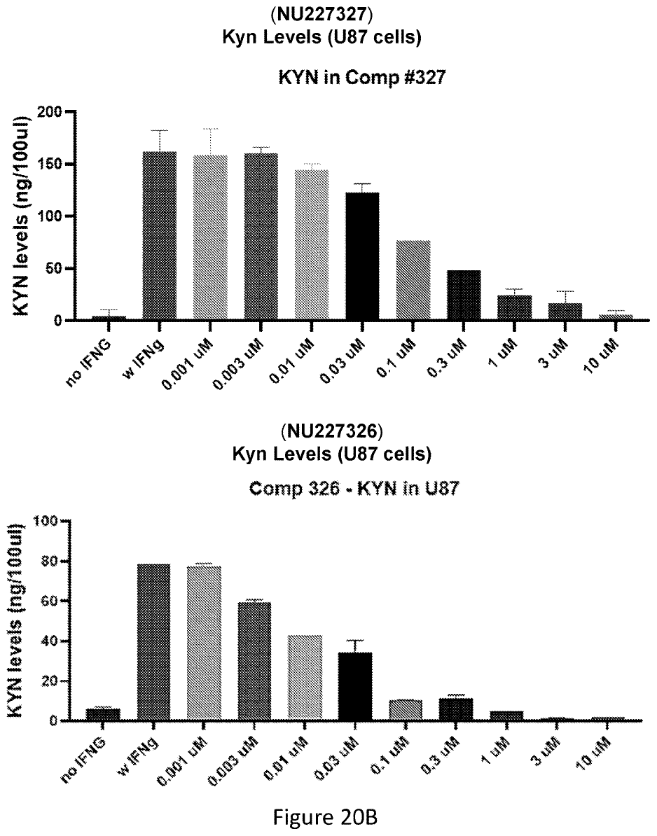

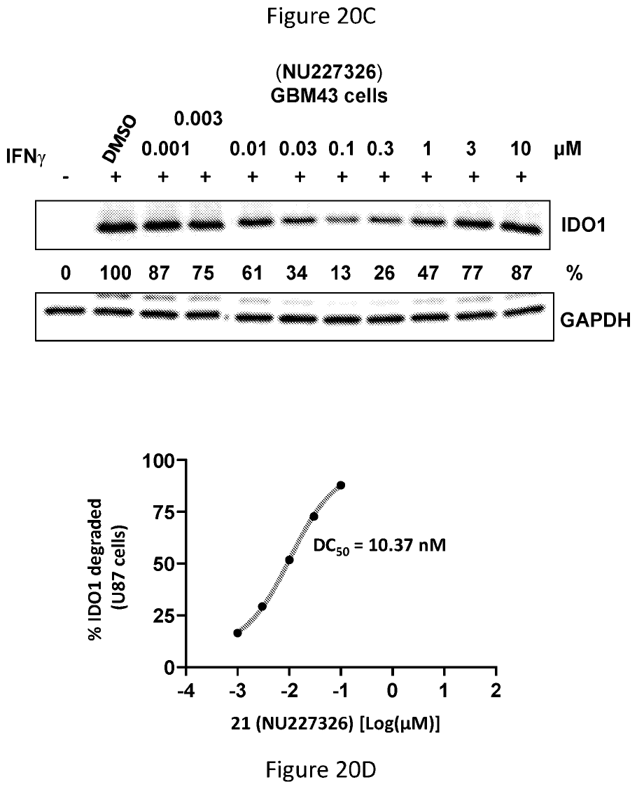

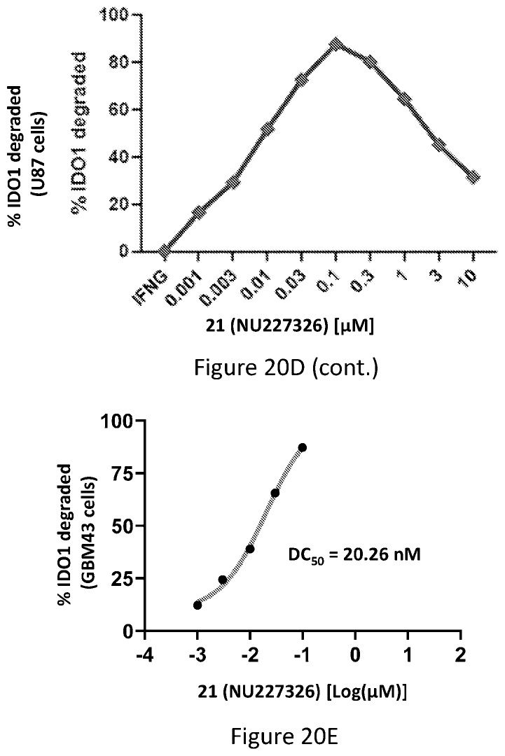

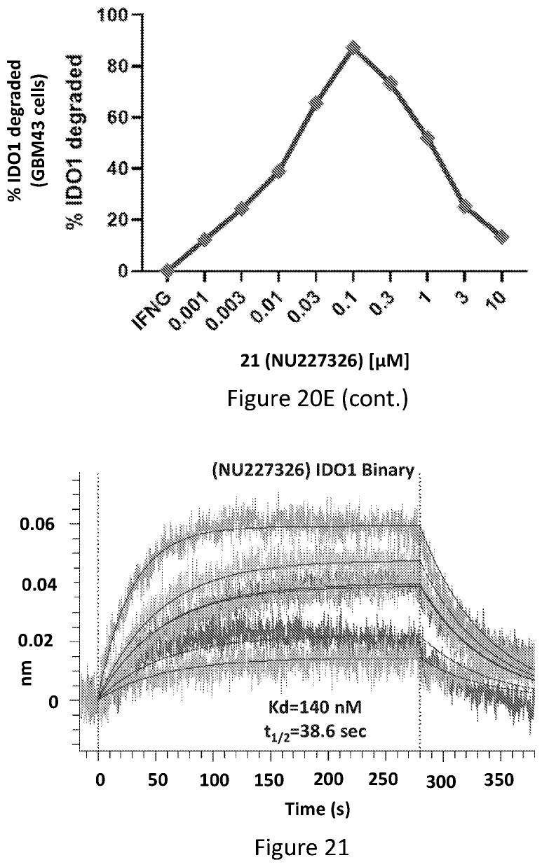

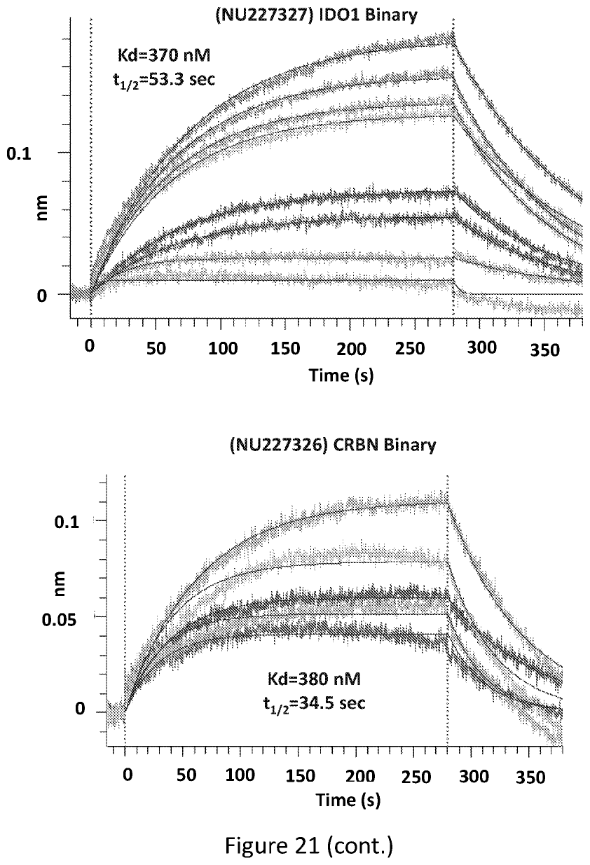

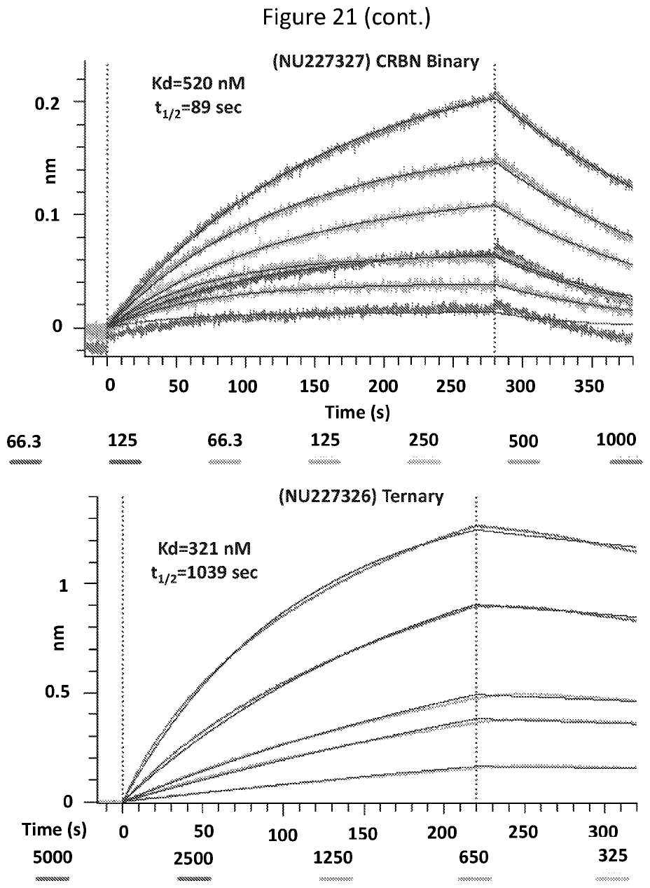

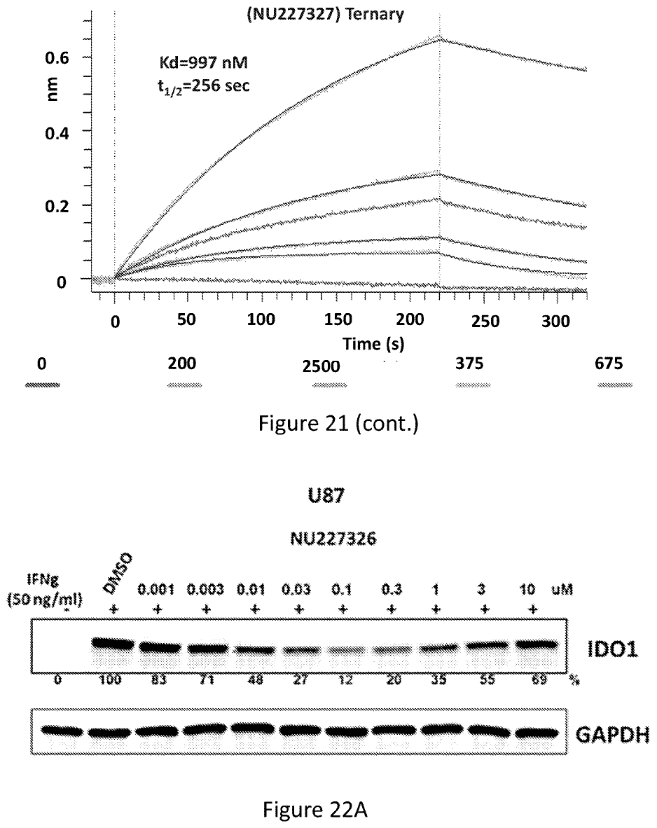

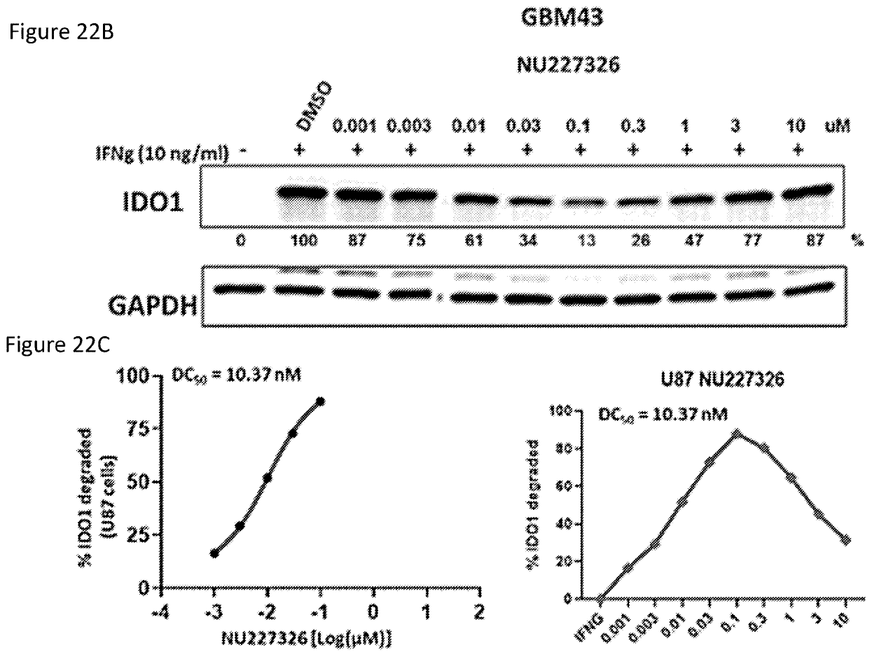

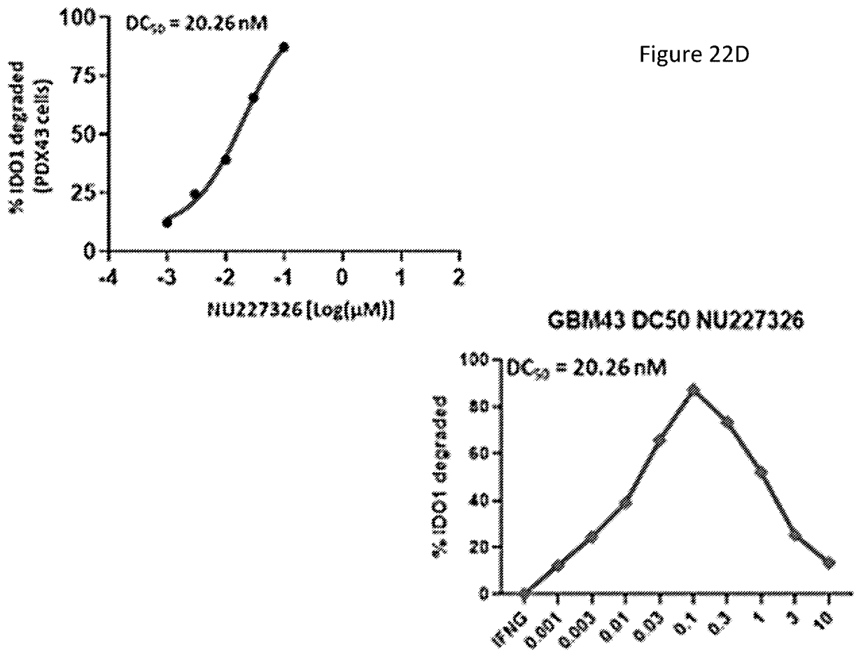

See FIGS. 7-11 for extended dosage range of selected PROTACs.

### BLI Data:

### Bio-Layer Interferometry (BLI) Assays

A ForteBio® Octet K2 BLI instrument (Sartorius) was used for studying the interactions of IDO1 PROTACs with the IDO1 and cereblon (CRBN) proteins. 6×His tagged recombinant IDO1 protein (purchased from Active Motif, Carlsbad, CA, Cat #81031) was reconstituted at 80 μg/mL and treated with human apo-myoglobin (Prospec Cat #PRO-374) in order to remove residual heme groups. This procedure consisted of incubating IDO1 with 5-fold excess apo-myoglobin for 1 hr at 37° C. The IDO1 protein was then loaded on pre-hydrated Ni-NTA biosensors (Sartorius, ForteBio®, Cat #18-5103). The concentration of IDO1 in the loading step was 55ug/ml, and the duration of the loading step was 360 seconds. The BLI signal stabilized at a value of 6 nm after approximately 220 seconds. No dissociation of the IDO1 protein was observed for 30 minutes when the sensors were moved into buffer (PBS+0.5% DMSO at pH 7.4). at 30° C. For studies of the binary complexes between IDO1-PROTAC and IDO1, compounds were diluted in reaction buffer to obtain a stock concentration of 80 μM. After baseline equilibration in reaction buffer, the kinetics of association were monitored by moving individual sensors into wells containing 200 μL of analyte solutions for each complex. After the association step, the sensors were placed in reaction buffer to monitor dissociation. During the entirety of the kinetic assay, the 96-well sample plate was kept at 30° C. and was shaken at 1,000 RPM. Biosensors without ligand were titrated with analyte and used as a parallel reference control. Ligand-loaded biosensors without analyte were used as baseline. Double-referenced data were fitted globally with a steady state 1:1 model using the Data Analysis HT 11.0.0.50 (FortdBio) software suite. For examining the interaction of CRBN with IDO1-PROTAC, BLI experiments and data analysis was performed as described above, except sensors with immobilized CRBN were used instead. To prepare the sensors, CRBN protein (Sino Biological, Wayne, PA) was reconstituted in Acetate Buffer at pH 6 and amine coupled to AR2G sensors (Sartorius, ForteBio®, Cat #18-5092) following the manufacturer recommendations. For experiments examining the formation of a ternary complex, IDO1 was immobilized on NiNTA sensors as described above from 30 μg/mL solutions. To form the PROTAC-CRBN complex, PROTAC at 1.4 μM was pre-incubated with 25 molar excess of CRBN and allowed to equilibrate for 15 minutes at 30° C. A series of 2-fold dilutions of this stock solution were made; these dilutions were allowed to equilibrate for an additional 15 minutes at 30° C. before binding reactions between IDO and the PROTAC-CRBN complex were monitored. Separately, experiments with identical sequences and preparation were performed using PROTAC without CRBN, and with CRBN without PROTAC. For ternary and binary complexes, each dataset was fitted globally with a 1:1 kinetic model using the Data Analysis HT 11.0.0.50 (FortéBio) software.

TABLE 3
Summary of binding data from BLI experiments.

| | | | IDO1-PROTAC- |
|---|---|---|---|
| ID | IDO1 Binary | CRBN Binary | CRBN Ternary |
| NU227164 | K_{d} = 430 nM | K_{d} = 310 nM | K_{d} = 128 nM (+/−12) |
| | | (+/−76) | T_{1/2} = 492 sec |
| NU227168 | K_{d} = 340 nM | K_{d} = 190 nM | K_{d} = 87 nM (+/−18) |
| | (+/−96) | (+/−84) | T_{1/2} = 1461 sec |
| NU227199 | K_{d} =2.1 μM | K_{d} = 1.6 μM | K_{d} = 689 nM |
| | | | T_{1/2} = 302 sec |
| NU227191 | K_{d} = 12 μM | K_{d} = 5.1 μM | could not obtain |
| NU227326 | K_{d} = 140 nM | K_{d} = 380 nM | K_{d} = 321 nM |
| | | | t_{1/2} = 1039 sec |
| NU227327 | K_{d} = 370 nM | K_{d} = 520 nM | K_{d} = 997 nM |
| | | | t_{1/2} = 256 sec |

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A molecule having a formula: M_{1DO1}-L-M_{E3}, or a pharmaceutically acceptable salt thereof, wherein M_{1DO1} is a moiety that binds to indoleamine 2,3-dioxygenase 1 (IDO1), L is a linker covalently attaching M_{1DO1} and M_{E3}, and M_{E3} is a moiety that binds to an E3 ubiquitin ligase;
wherein
L comprises a piperidinylene moiety;
with the proviso that M_{E3} does not comprise 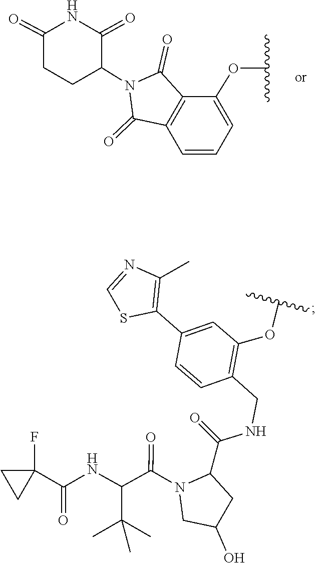 [SMILES: *Oc1cccc2c1C(=O)N(C1CCC(=O)NC1=O)C2=O ; *Oc1cc(-c2scnc2C)ccc1CNC(=O)C1CC(O)CN1C(=O)C(NC(=O)C1(F)CC1)C(C)(C)C]
with the proviso that the molecule is not (2R)-N-(4-((1-(3-(2-(2-((2-(2,6-dioxopiperidin-3-yl)-1,3-dioxoisoindolin-4-yl)amino)ethoxy)ethoxy)propanoyl)piperidin-4-yl)oxy)phenyl)-2-((1s,4S)-4-(6-fluoroquinolin-4-yl)cyclohexyl)propanamide or (2R)-N-(4-((1-(3-(2-((2-(2,6-dioxopiperidin-3-yl)-1,3-dioxoisoindolin-4-yl)amino)ethoxy)propanoyl)piperidin-4-yl)oxy)phenyl)-2-((1s,4S)-4-(6-fluoroquinolin-4-yl)cyclohexyl)propanamide.

2. The molecule of claim 1, wherein M_{1DO1} has a formula I: 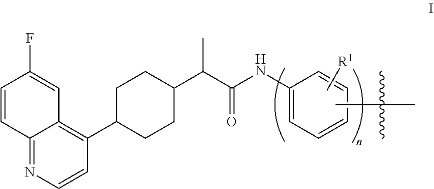
wherein
R¹ is hydrogen or halo; and
n is 0 or 1.

3. The molecule of claim 1 or 2, wherein M_{1DO1} has a formula I(a):

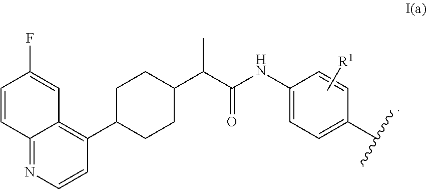

4. The molecule of claim 3, wherein R¹ is hydrogen.

5. The molecule of claim 3, wherein R¹ is chloro.

6. The molecule of claim 1 or 2, wherein M_{1DO1} has a formula I(b):

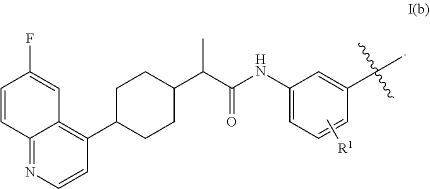

7. The molecule of claim 6, wherein R¹ is hydrogen.

8. The molecule of claim 6, wherein R¹ is chloro.

9. The molecule of claim 1 or 2, wherein M_{1DO1} has a formula I(c):

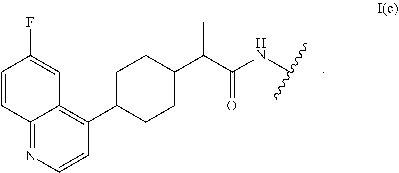 [SMILES: *NC(=O)C(C)C1CCC(c2ccnc3ccc(F)cc23)CC1 ; "."]

10. The molecule of any one of claims 1-9, wherein L is selected from the group consisting of 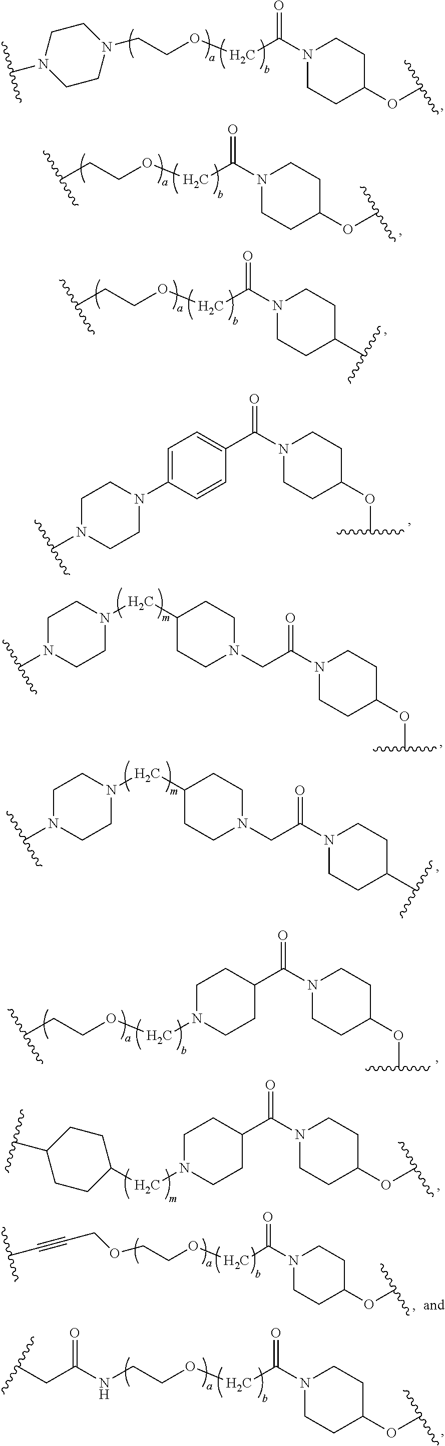
wherein each of a, b, and m is independently an integer selected from 0 to 20.

11. The molecule of any one of claims 1-10, wherein L is selected from the group consisting of

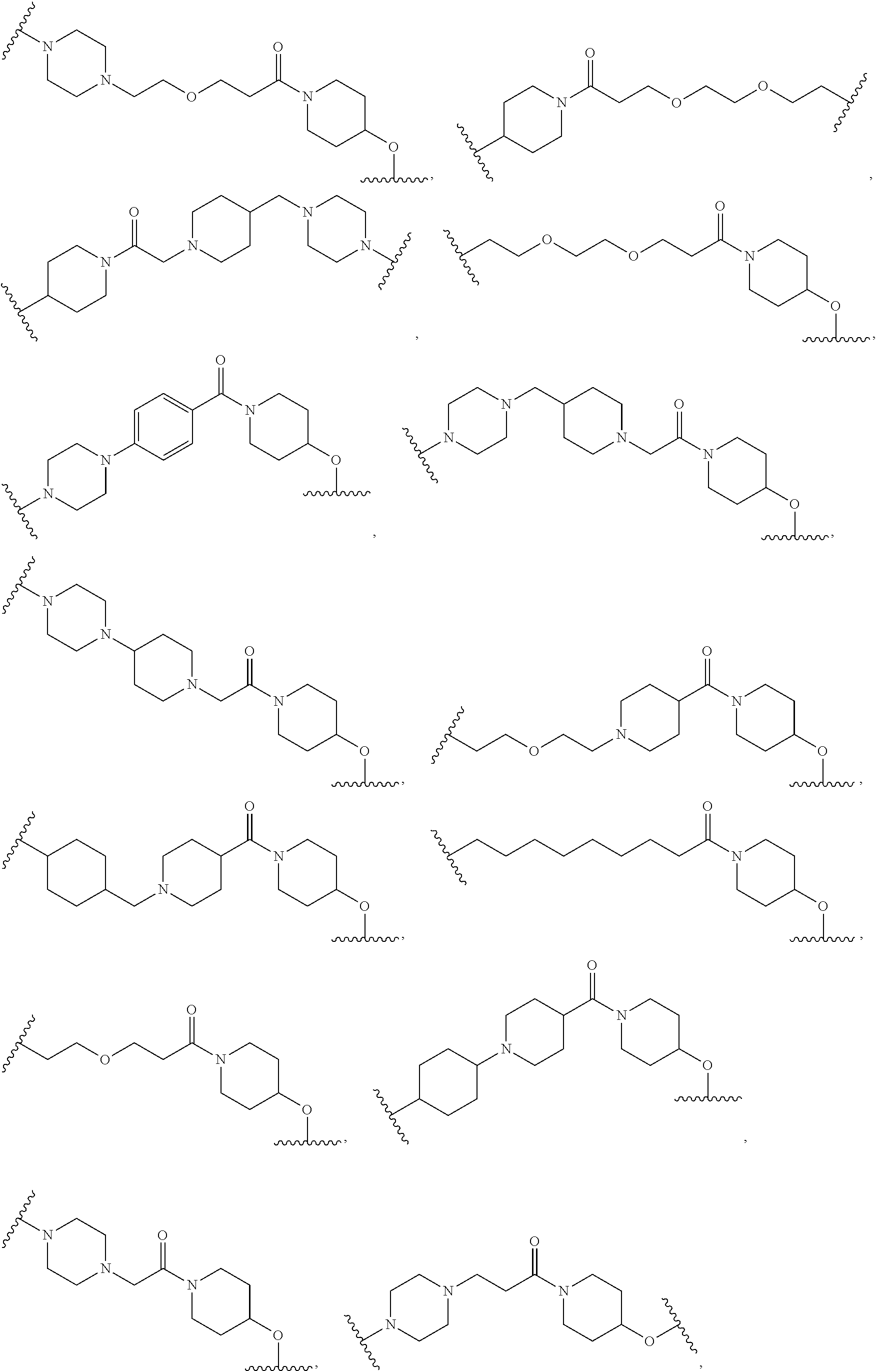

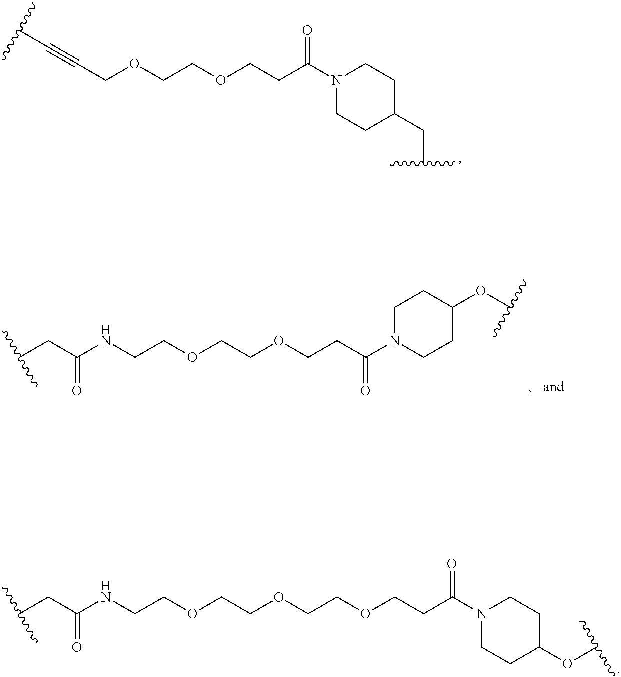 [SMILES: *C#CCOCCOCCC(=O)N1CCC(C*)CC1 ; *CC(=O)NCCOCCOCCC(=O)N1CCC(O*)CC1 ; *CC(=O)NCCOCCOCCOCCC(=O)N1CCC(O*)CC1]

12. The molecule of any one of claims 1-11, wherein M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

13. The molecule of any one of claims 1-12, wherein M_{E3} is a moiety derived from thalidomide, pomalidomide, lenalidomide, iberdomide, (S,R,S)-AHPC-Me hydrochloride, (S,R,S)-AHPC-Me dihydrochloride, cereblon modulator 1, thalidomide-propargyl, (S,R,S)-AHPC-propargyl, (S,R,S)-AHPC hydrochloride, CC-885, thalidomide-O—COOH, lenalidomide hemihydrate, thalidomide fluoride, thalidomide-OH, lenalidomide-Br, thalidomide D4, lenalidomide hydrochloride, (S,R,S)-AHPC-Me, c1AP1 ligand 1, TD-106, E3 ligase Ligand 8, E3 ligase Ligand 9, E3 ligase Ligand 10, E3 ligase Ligand 13, E3 ligase Ligand 14, E3 ligase Ligand 18, BC-1215, VHL ligand 1 (VHL-1), VHL ligand 2 (VHL-2), VHL Ligand 8 (VHL-8), VH032, VH032-cyclopropane-F, VH032 thiol, VH-298, VL-269, VL-285, LCL161, hydroxyproline-based ligands, HIF-1α-derived (R)-hydroxyproline, Nutlin carboxylic acid, (4R,5S)-Nutlin carboxylic acid, (S,R,S)-AHPC-Boc, AR antagonist 1, NV03, (S,R,S)-AHPC TFA, (S,R,S)-AHPC, β-Naphthoflavone-CH2—Br, β-Naphthoflavone-CH2—OH, Bestatin-amido-Me, MV-1-NH-Me, (S,S,S)-AHPC hydrochloride, and clAP1 ligand 2.

14. The molecule of any one of claims 1-13, wherein M_{E3} has a formula selected from the group consisting of: 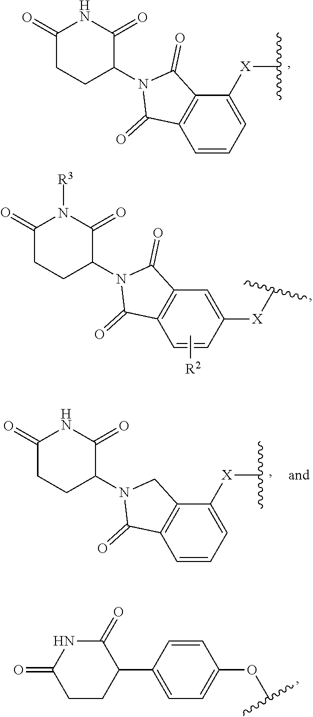
wherein
X is a bond, NH, or NMe;
R² is hydrogen or halo; and
R³ is hydrogen or methyl.

15. The molecule of any one of claims 1-14, wherein M_{E3} has a formula selected from the group consisting of

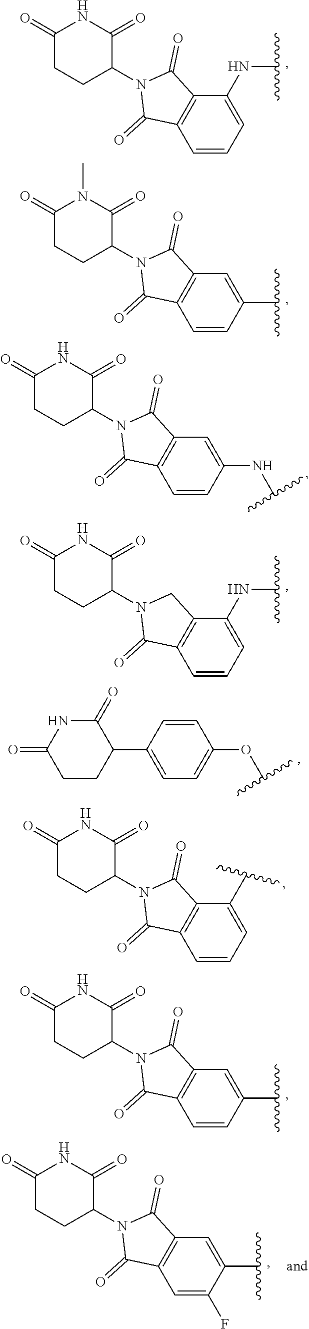

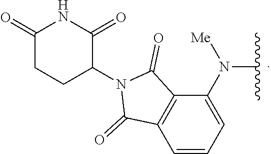 [SMILES: *N(C)c1cccc2c1C(=O)N(C1CCC(=O)NC1=O)C2=O]

16. The molecule of any one of claims 1-15, wherein the molecule has a formula selected from the group consisting of

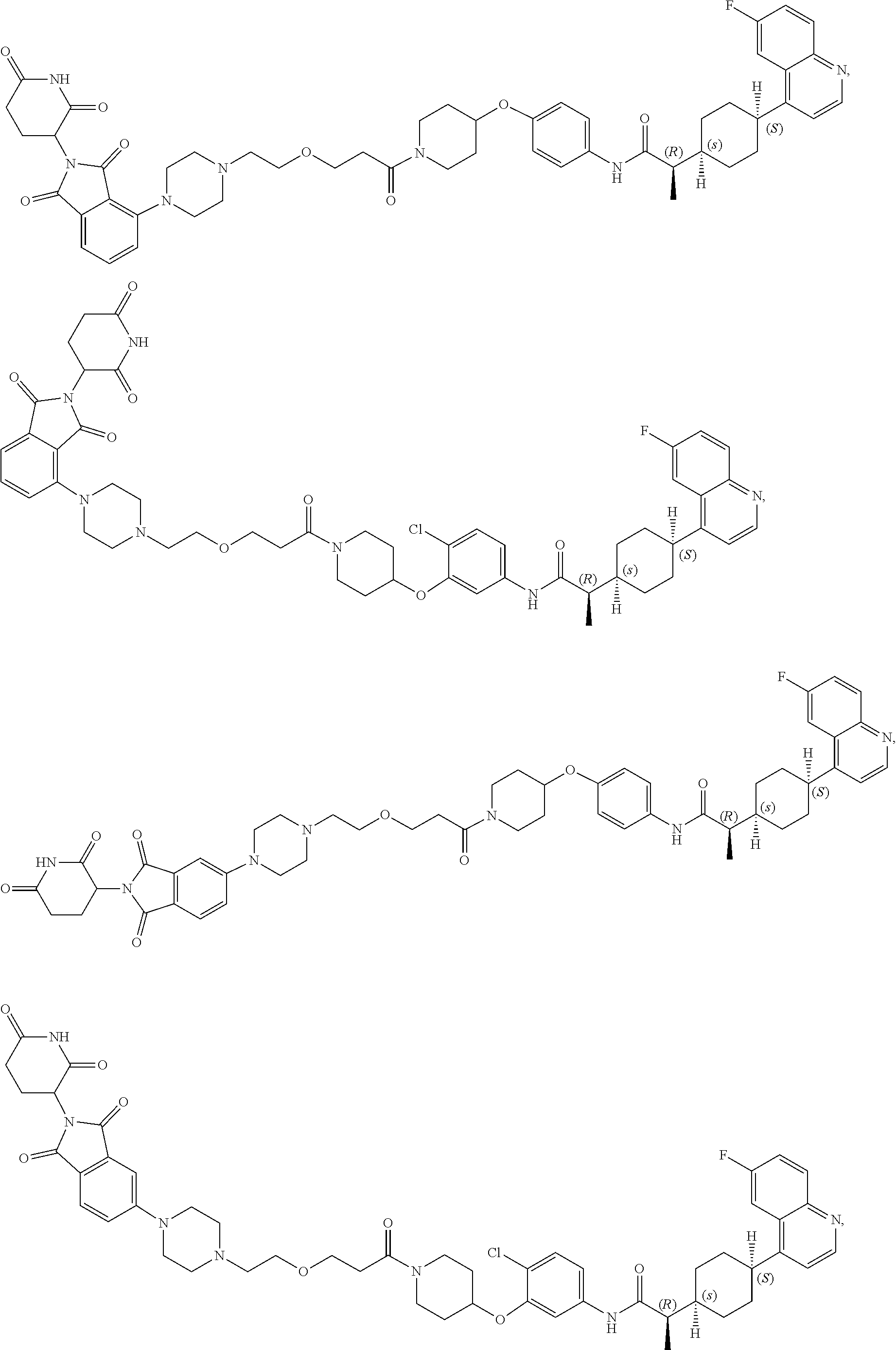

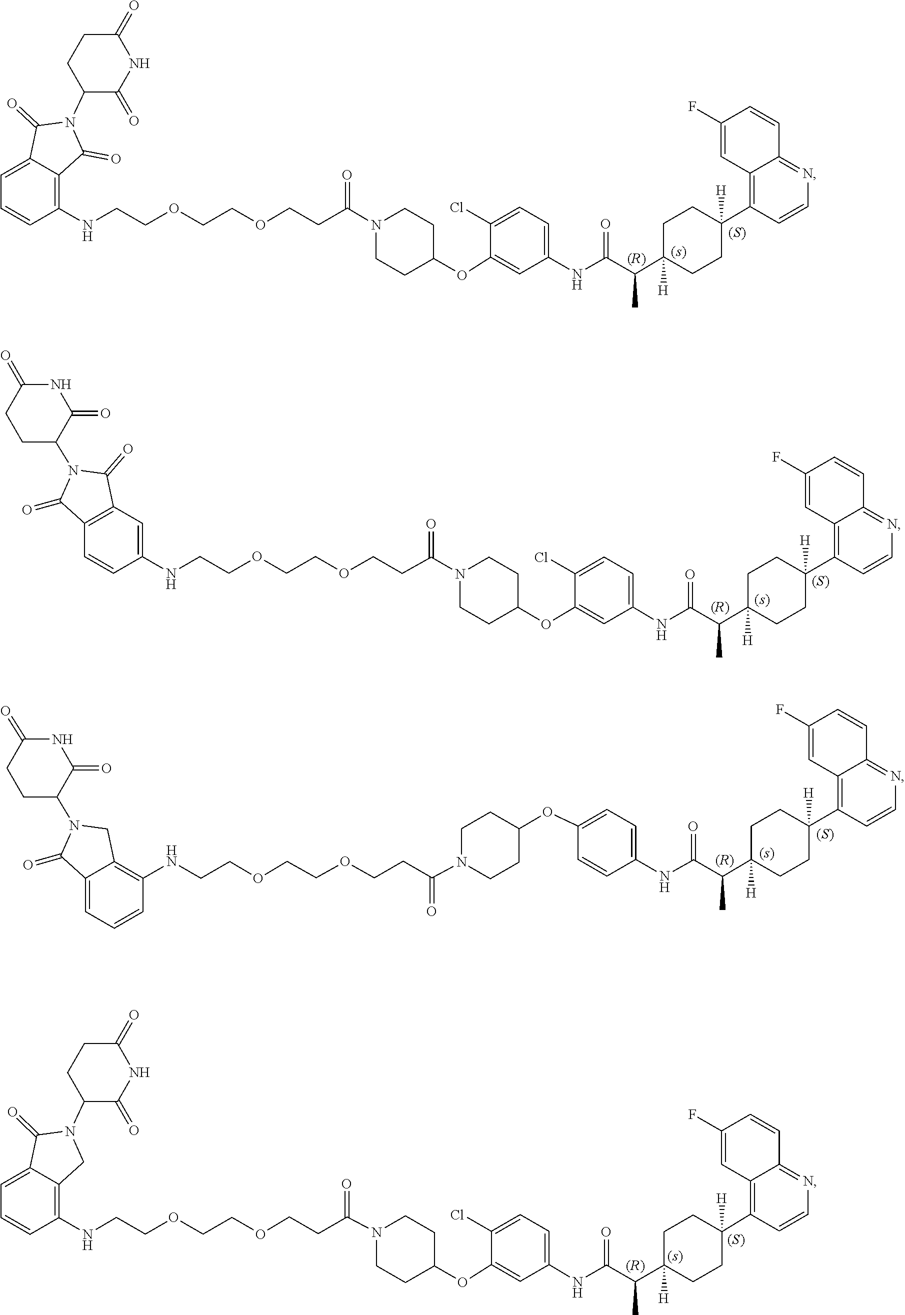

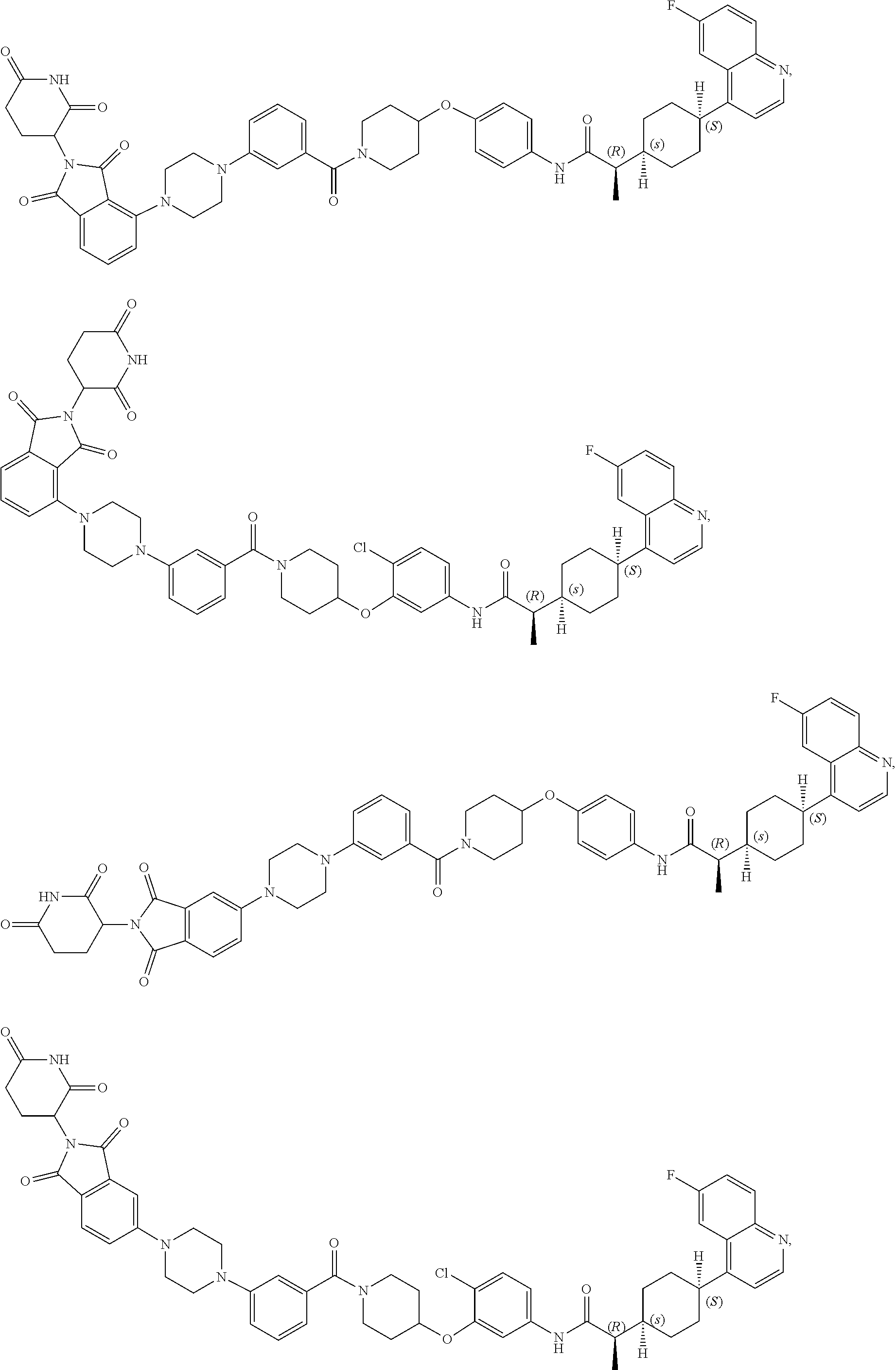

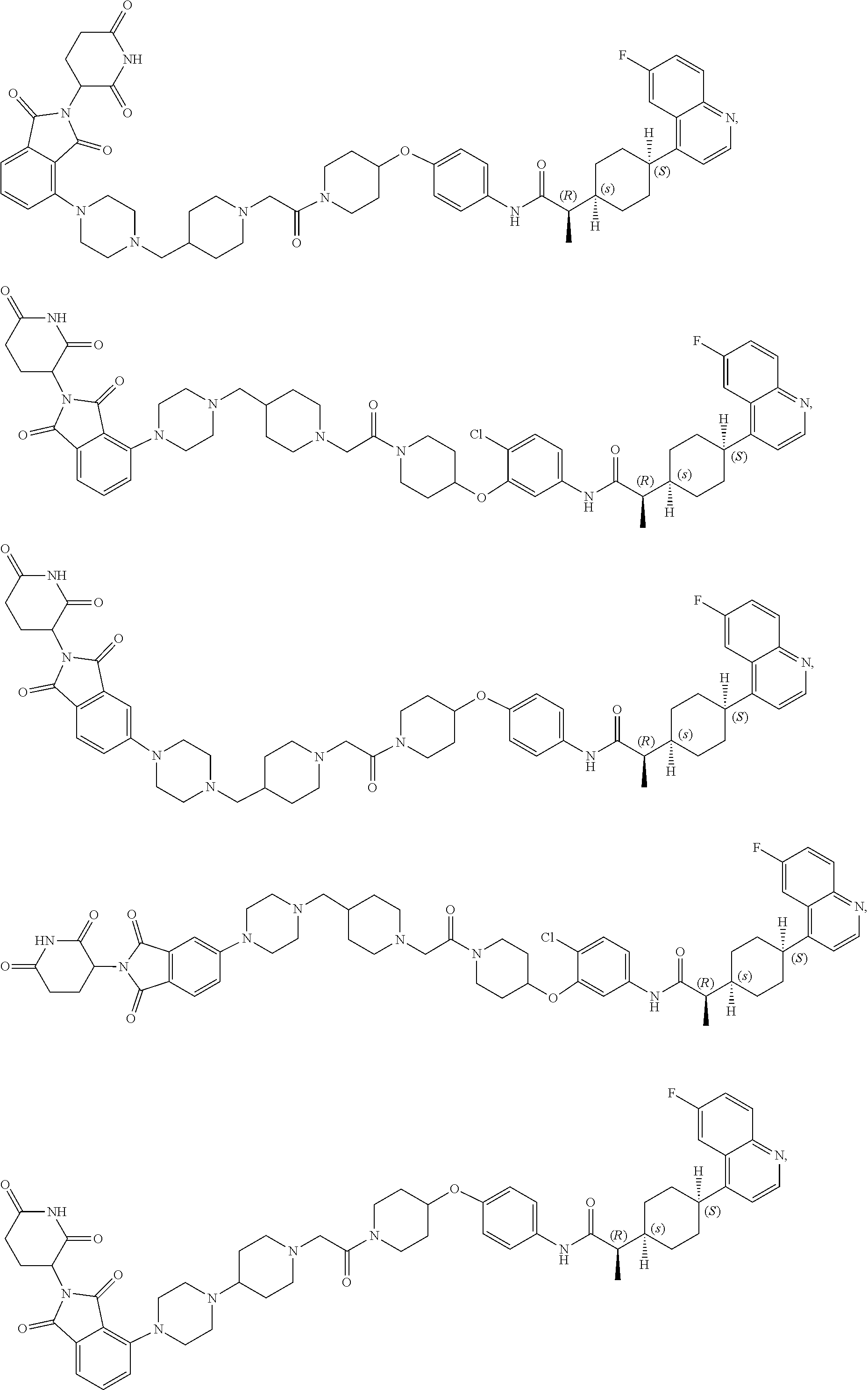

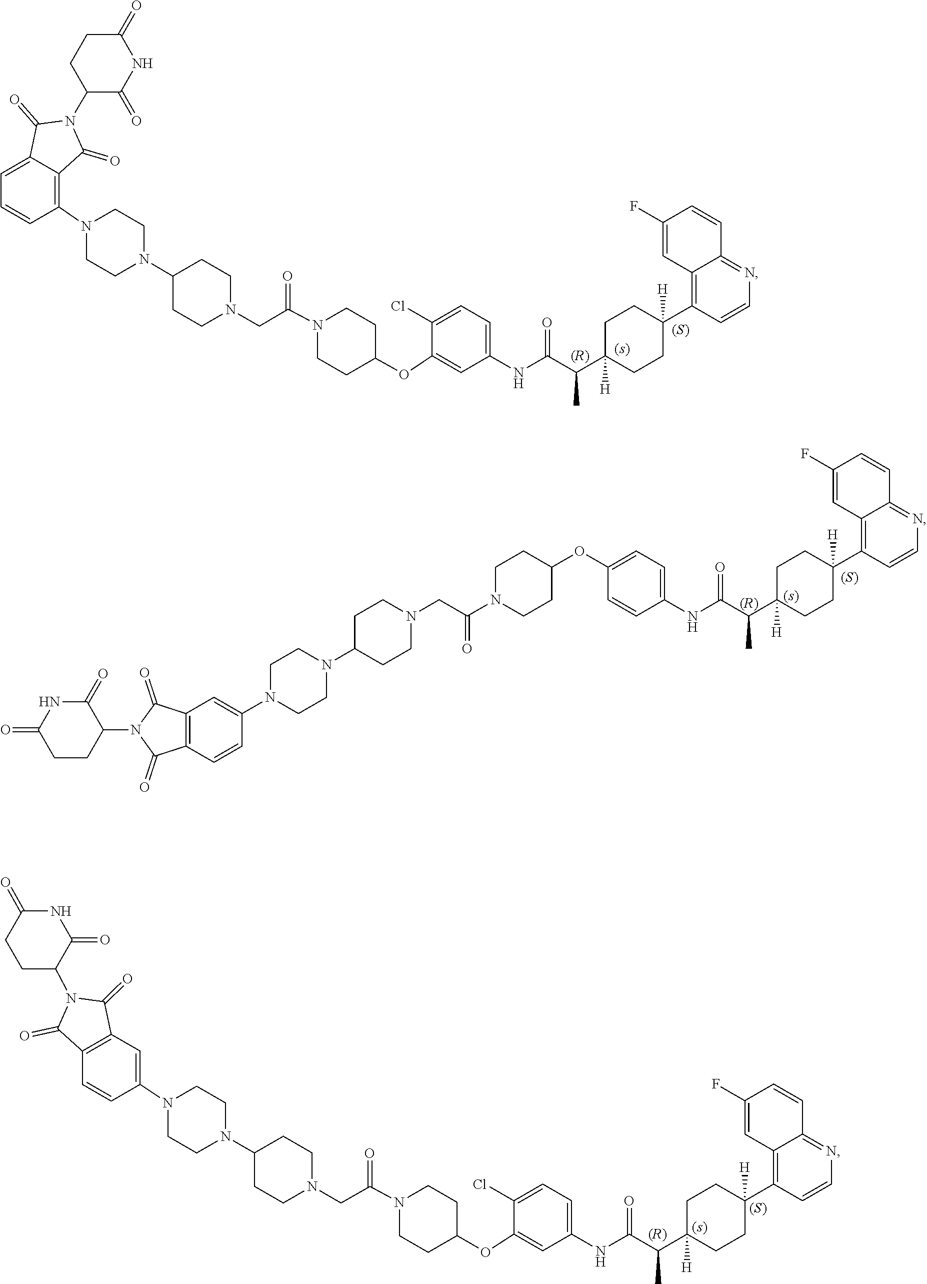

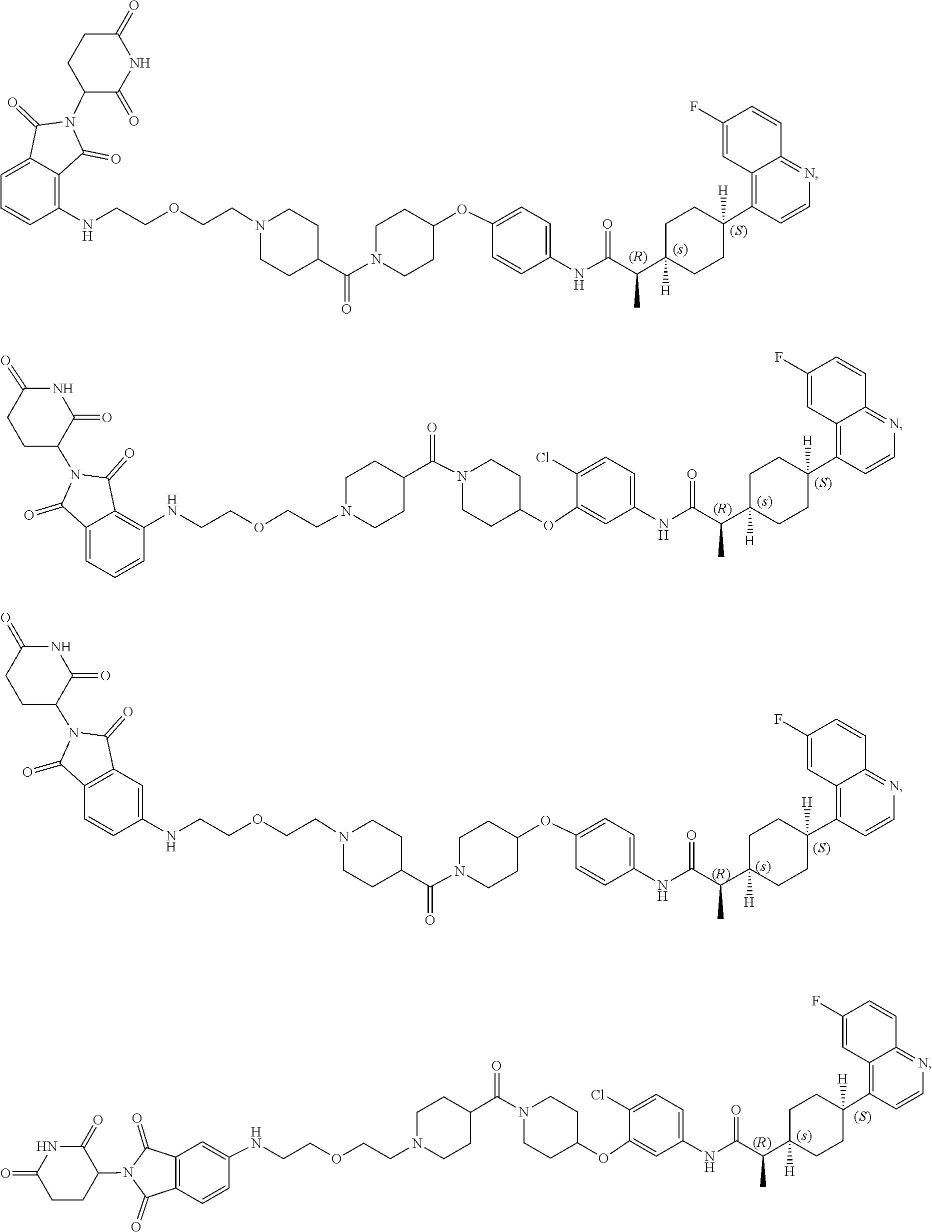

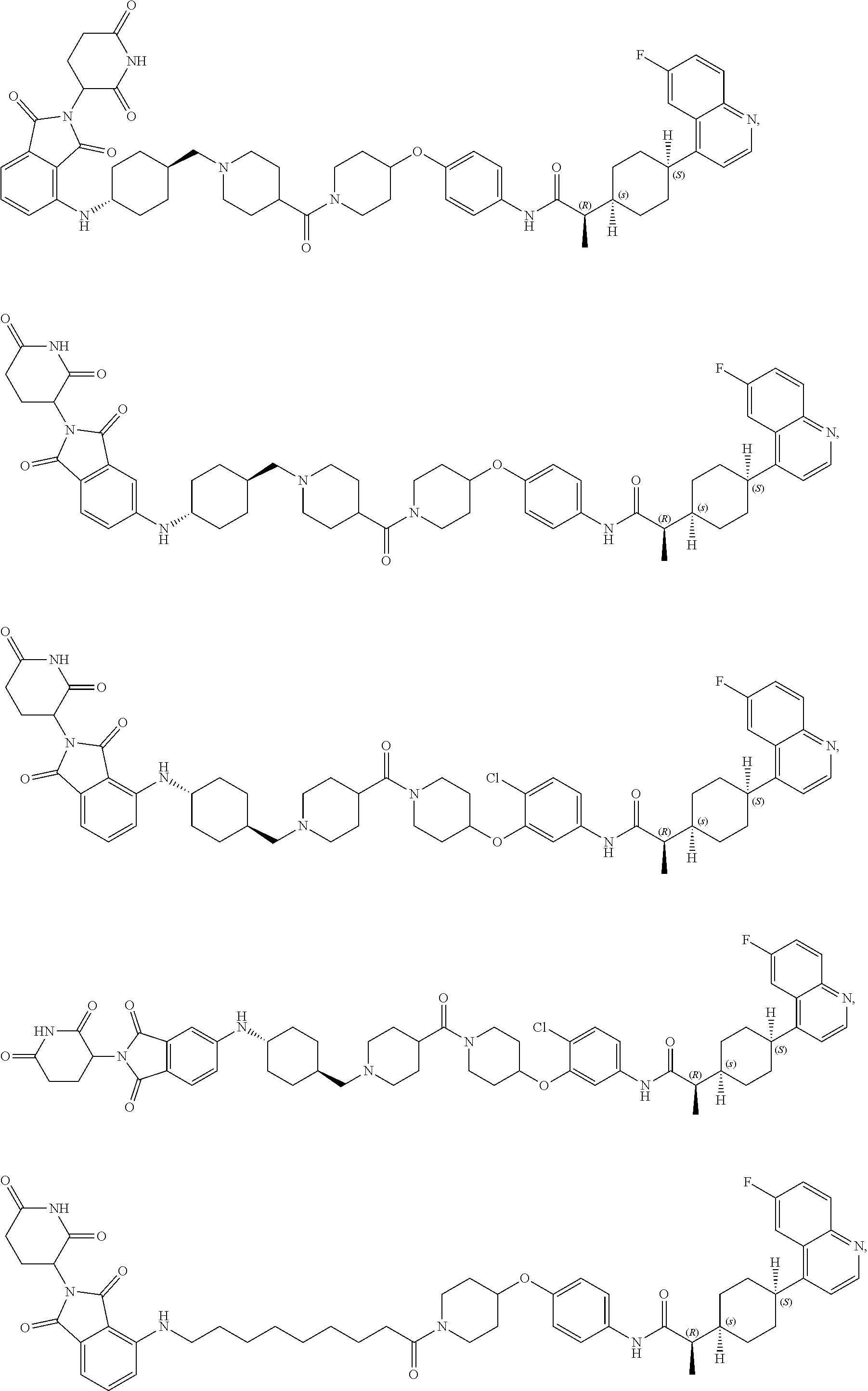

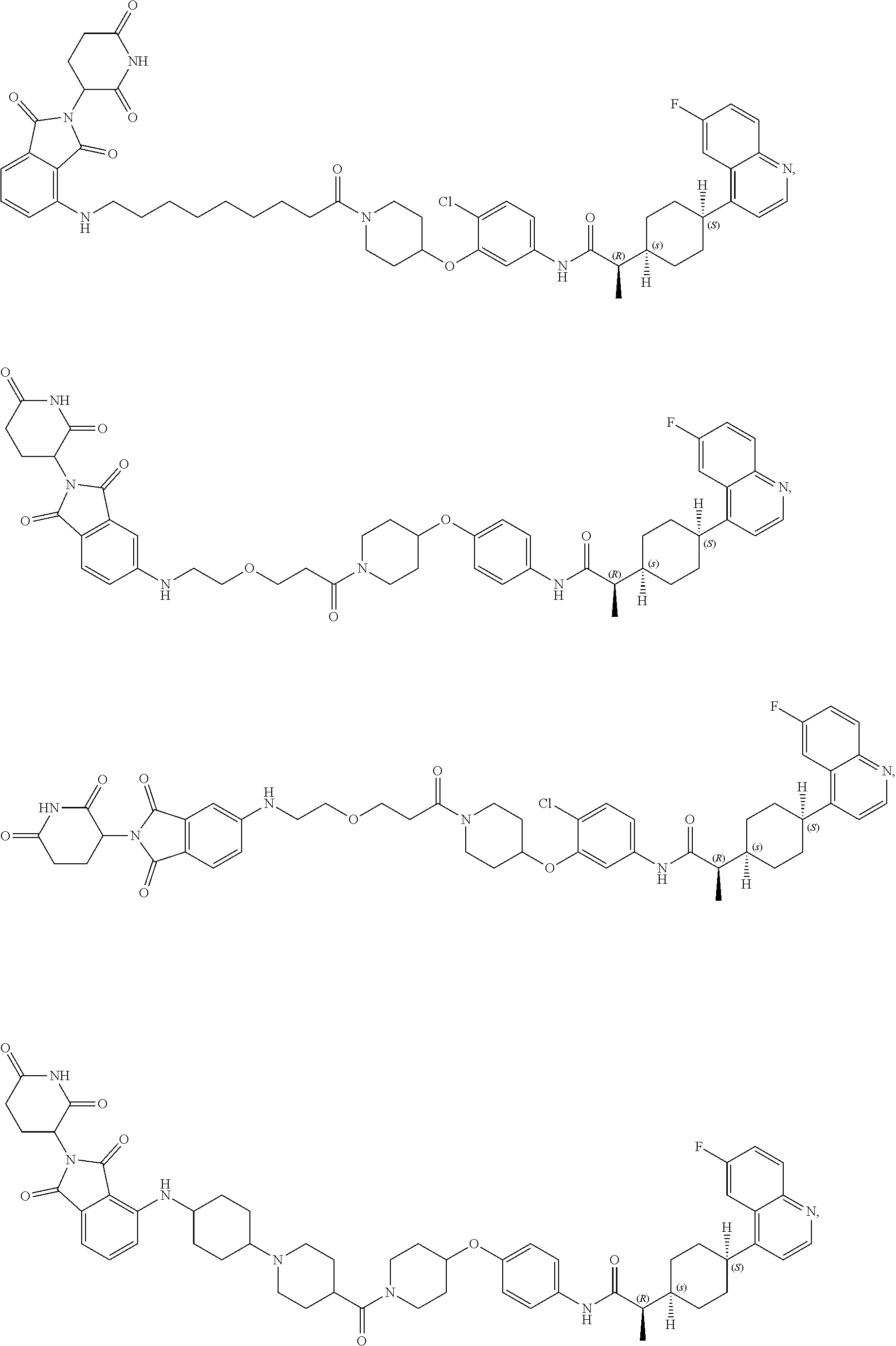

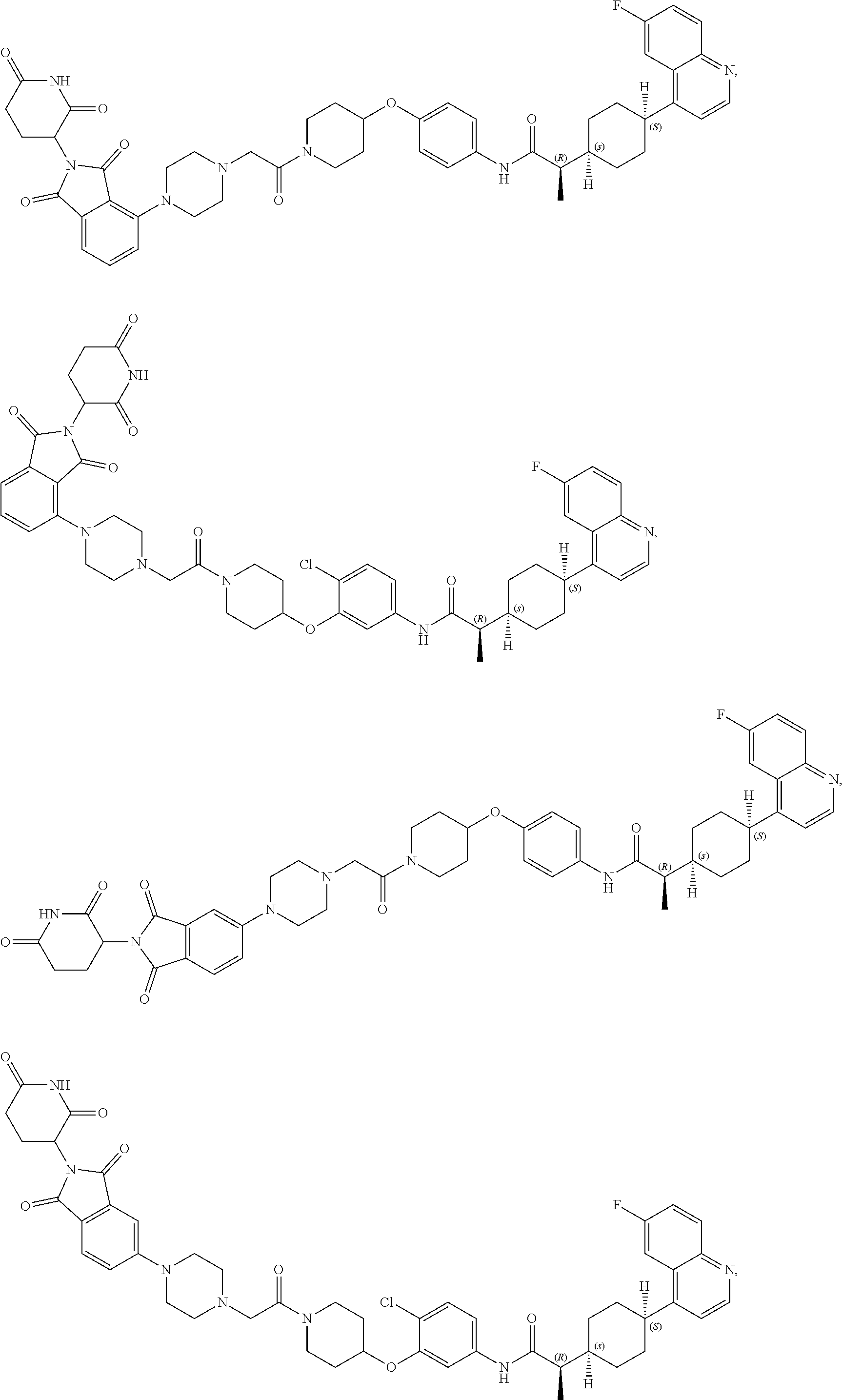

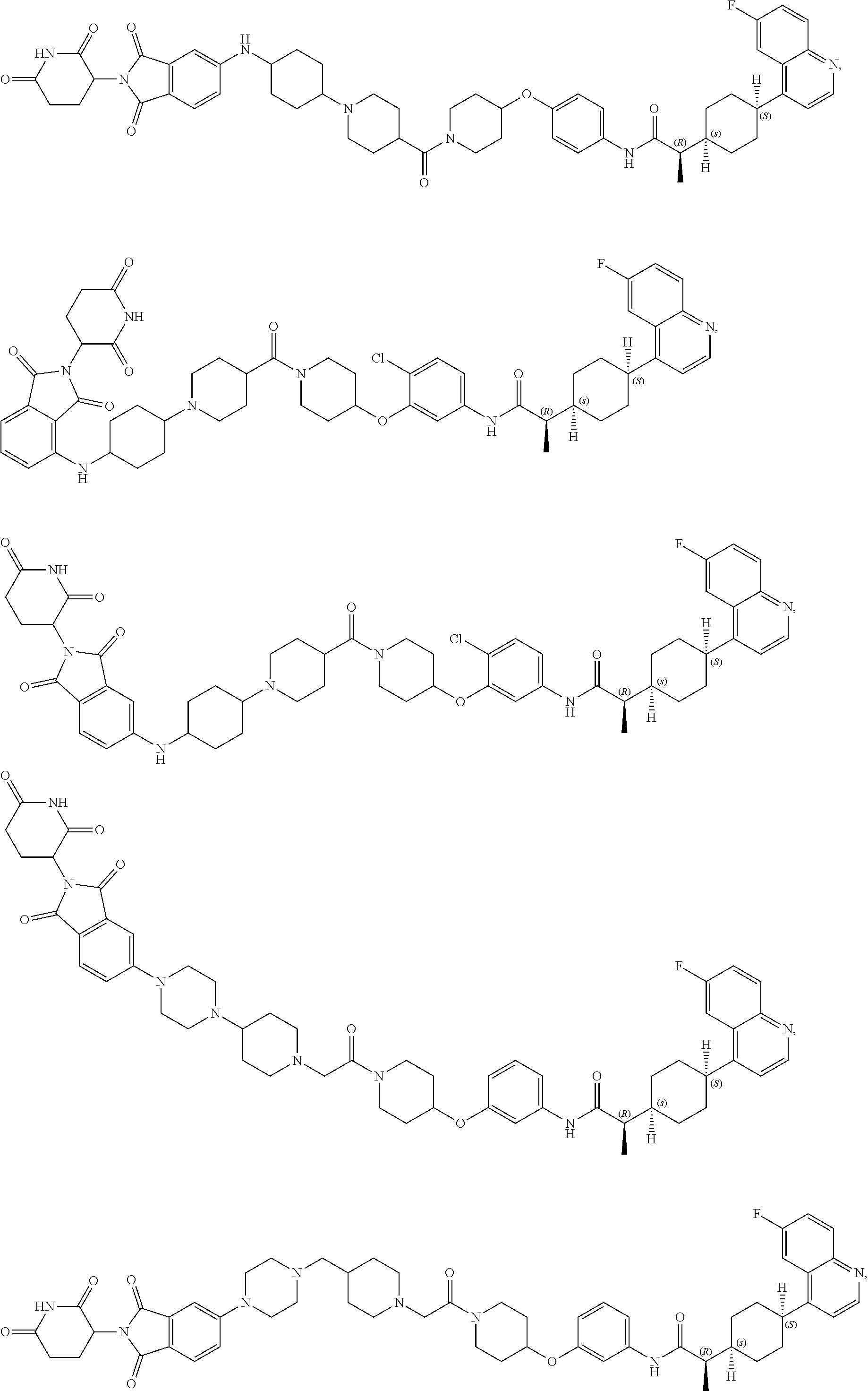

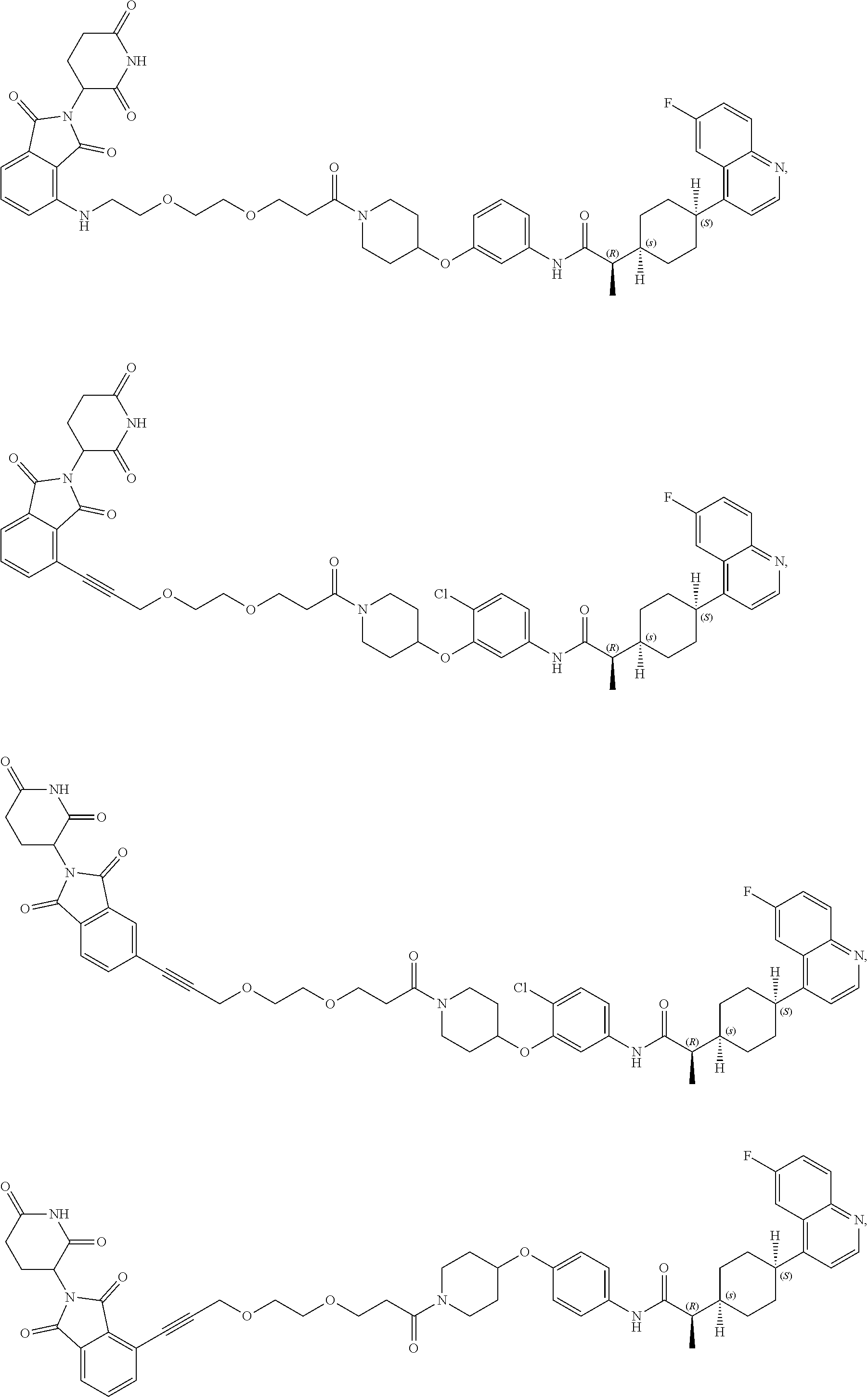

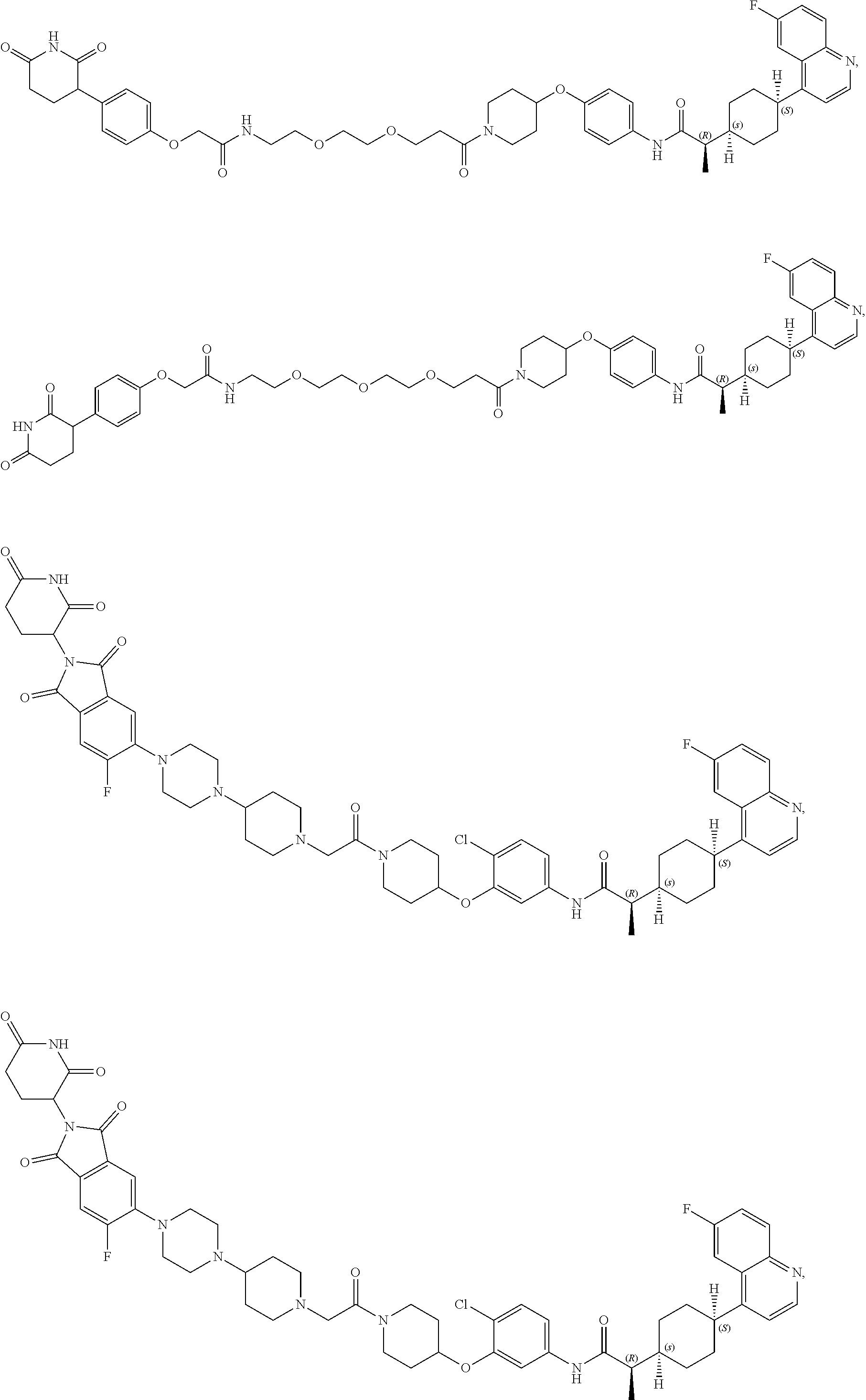

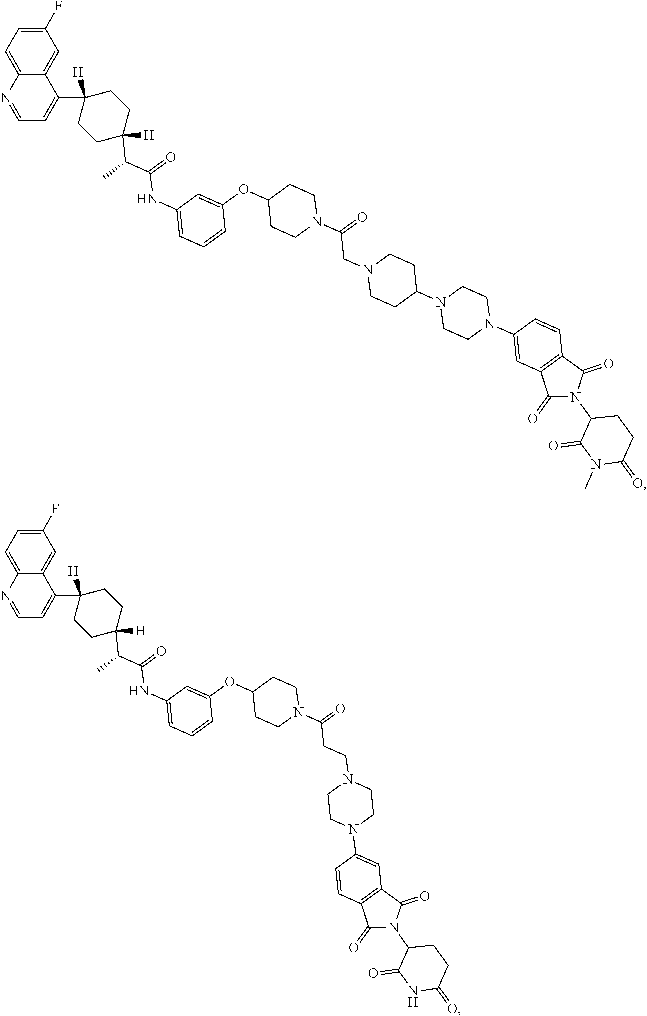 [SMILES: C[C@@H](C(=O)Nc1cccc(OC2CCN(C(=O)CN3CCC(N4CCN(c5ccc6c(c5)C(=O)N(C5CCC(=O)N(C)C5=O)C6=O)CC4)CC3)CC2)c1)[C@H]1CC[C@@H](c2ccnc3ccc(F)cc32)CC1 ; C[C@@H](C(=O)Nc1cccc(OC2CCN(C(=O)CCN3CCN(c4ccc5c(c4)C(=O)N(C4CCC(=O)NC4=O)C5=O)CC3)CC2)c1)[C@H]1CC[C@@H](c2ccnc3ccc(F)cc32)CC1]

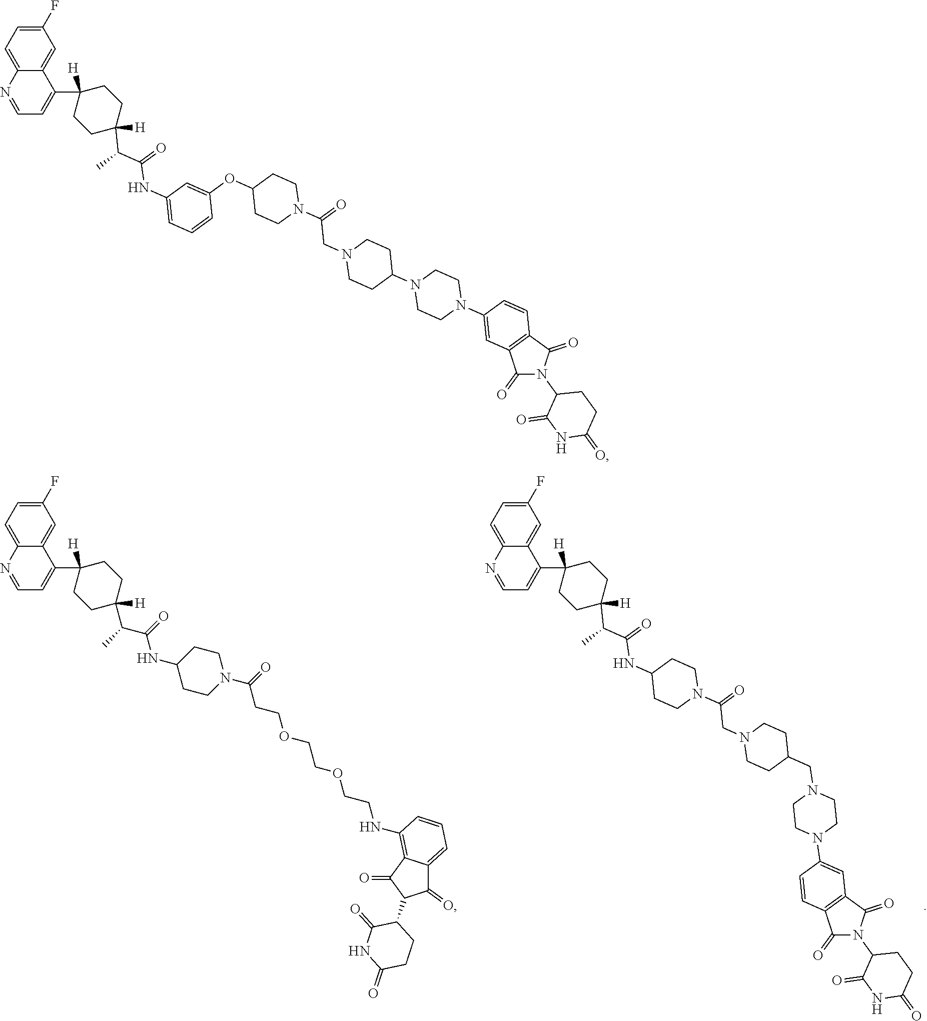 [SMILES: C[C@@H](C(=O)Nc1cccc(OC2CCN(C(=O)CN3CCC(N4CCN(c5ccc6c(c5)C(=O)N(C5CCC(=O)NC5=O)C6=O)CC4)CC3)CC2)c1)[C@H]1CC[C@@H](c2ccnc3ccc(F)cc32)CC1 ; C[C@@H](C(=O)NC1CCN(C(=O)CCOCCOCCNc2cccc3c2C(=O)C([C@@H]2CCC(=O)NC2=O)C3=O)CC1)[C@H]1CC[C@@H](c2ccnc3ccc(F)cc32)CC1 ; C[C@@H](C(=O)NC1CCN(C(=O)CN2CCC(CN3CCN(c4ccc5c(c4)C(=O)N(C4CCC(=O)NC4=O)C5=O)CC3)CC2)CC1)[C@H]1CC[C@@H](c2ccnc3ccc(F)cc32)CC1]

17. The molecule of any one of claims 1-16 wherein the molecule has a formula selected from the group consisting of

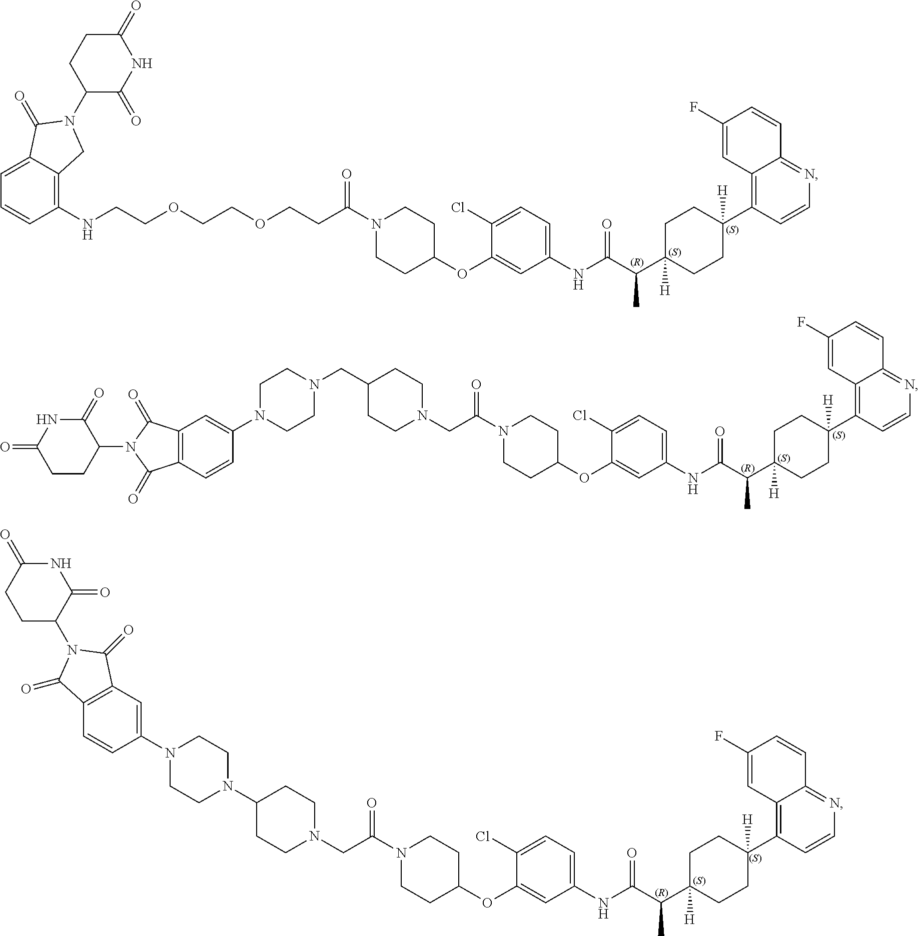

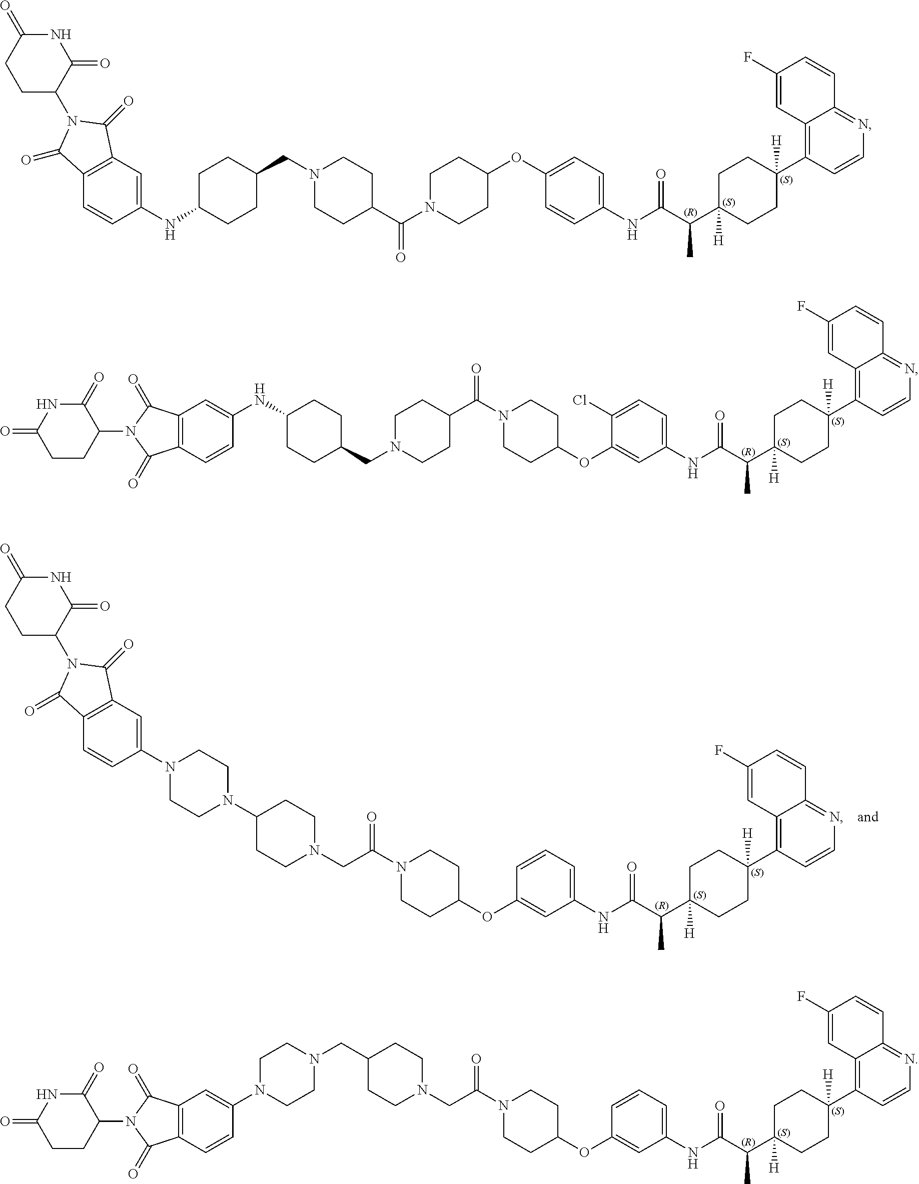

18. The molecule of any one of claims 1-17, wherein the molecule has a formula

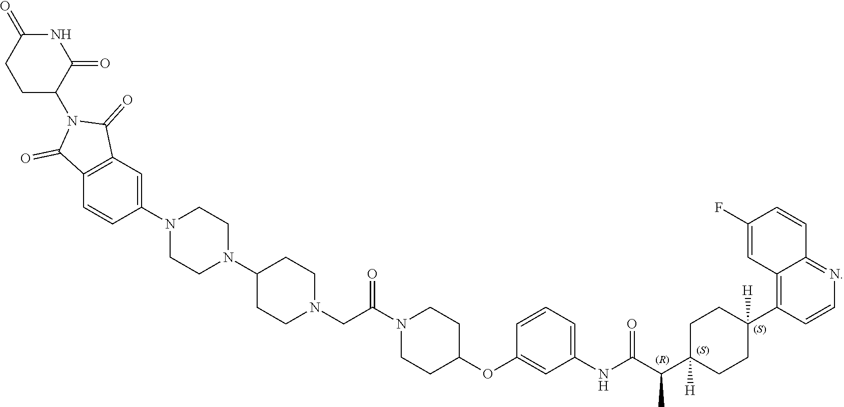

19. The molecule of any one of claims 1-18, wherein, under the same conditions, the molecule is more potent than the compound having a formula

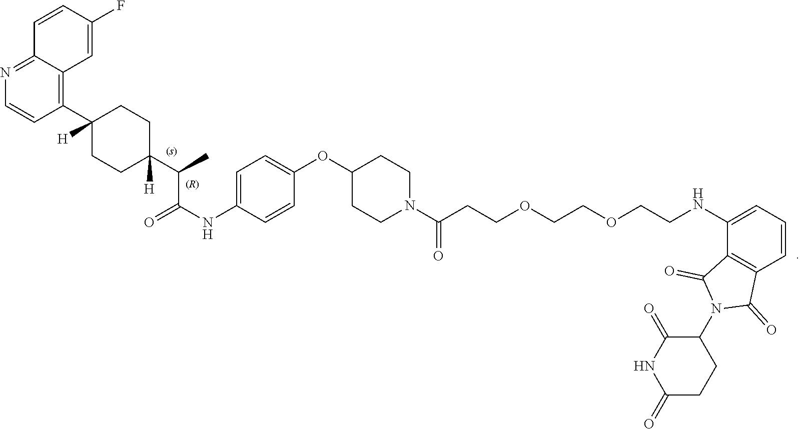

20. A pharmaceutical composition comprising a therapeutically effective amount of the molecule of any one of claims 1-19, or a pharmaceutically acceptable salt thereof, and a pharmaceutically acceptable carrier, excipient, or diluent.

21. A method of treating cancer in a subject in need thereof, the method comprising administering the molecule of any one of claims 1-19, or a pharmaceutically acceptable salt thereof, or the pharmaceutical composition of claim 20 to the subject having the cancer.

22. The method of claim 21, wherein the cancer is selected from multiple glioblastoma, colorectal cancer, myeloma, leukemia, non-small cell lung cancer, colon cancer, cancer of the central nervous system, melanoma, ovarian cancer, renal cancer, prostate cancer, uterine cancer, pancreatic cancer, and breast cancer.

23. The method of claim 21 or 22, wherein the cancer is glioblastoma.

24. The method of any one of claims 21-23, wherein the method further comprises administering a therapy comprising chemotherapy, radiation, checkpoint inhibitors, or immunotherapy to the subject, wherein the therapy is administered before, concurrently with, or after administering the pharmaceutical composition of claim 20.

25. The method of any one of claims 21-24, wherein the method further comprises administering an anti-PD1 agent to the subject, before, concurrently with, or after administering the pharmaceutical composition of claim 20.

26. The method of any one of claims 21-25, wherein the method further comprises administering an anti-PD-L1 agent to the subject, before, concurrently with, or after administering the pharmaceutical composition of claim 20.
